(19) United States
(12) Patent Application Publication
(10) Pub. No.: US 2014/0363454 A1
(43) Pub. Date: Dec. 11, 2014

(54) Antibody-Drug Conjugates, Compositions and Methods of Use

(71) Applicant: Igenica Biotherapeutics, Inc., Burlingame, CA (US)

(72) Inventors: David Y. Jackson, Belmont, CA (US); Edward Ha, San Francisco, CA (US); Gary D. Probst, San Francisco, CA (US)

(73) Assignee: Igenica Biotherapeutics, Inc., Burlingame, CA (US)

(21) Appl. No.: 14/298,798

(22) Filed: Jun. 6, 2014

Related U.S. Application Data
(60) Provisional application No. 61/832,068, filed on Jun. 6, 2013.

(51) Int. Cl. A61K 47/48; A61K 31/454

(52) U.S. Cl. CPC A61K 47/48384; A61K 31/454
USPC 424/181.1; 530/391.9; 546/256; 546/209; 548/547

(57) ABSTRACT

Antibody-cytotoxin antibody-drug conjugates and related compounds, such as linker-cytotoxin conjugates and the linkers used to make them, tubulysin analogs, and intermediates in their synthesis; compositions; and methods, including methods of treating cancers.

### RELATED APPLICATION

This application claims the benefit of U.S. Provisional Application No. 61/832,068, filed Jun. 6, 2013, which is incorporated herein by reference.

### BACKGROUND OF THE INVENTION

1. Field of the Invention

This invention relates to antibody-drug conjugates (ADCs) and related compounds, such as linkers used to make them and intermediates in their synthesis; compositions; and methods, including methods of treating cancers.

2. Description of the Related Art

Cancer is the second most prevalent cause of death in the U.S., yet there are few effective treatment options beyond surgical resection. Of the medical treatments for cancers, the use of monoclonal antibodies targeting antigens present on the cancer cells has become common. Anticancer antibodies approved for therapeutic use in the USA include alemtuzumab (CAMPATH®), a humanized anti-CD52 antibody used in the treatment of chronic lymphocytic leukemia; bevacizumab (AVASTIN®), a humanized anti-VEGF antibody used in colorectal cancer; cetuximab (ERBITUX®), a chimeric anti-epidermal growth factor antibody used in colorectal cancer, head and neck cancer, and squamous cell carcinoma; ipilimumab (YERVOY®), a human anti-CTLA-4 antibody used in melanoma; ofatumumab (ARZERRA®), a human anti-CD20 antibody used in chronic lymphocytic leukemia; panitumumab (VECTIBIX®), a human anti-epidermal growth factor receptor antibody used in colorectal cancer; rituximab (RITUXAN®), a chimeric anti-CD20 antibody used in non-Hodgkin lymphoma; tositumomab (BEXXAR®), a murine anti-CD20 antibody used in non-Hodgkin lymphoma; and trastuzumab (HERCEPTIN®), a humanized anti-HER2 antibody used in breast cancer. While these antibodies have proven useful in the treatments of the cancers for which they are indicated, they are rarely curative as single agents, and are generally used in combination with standard chemotherapy for the cancer.

As an example, trastuzumab is a recombinant DNA-derived humanized monoclonal antibody that selectively binds with high affinity to the extracellular domain of the human epidermal growth factor receptor2 protein, HER2 (ErbB2) (Coussens et al., Science 1985, 230, 1132-9; Salmon et al., Science 1989, 244, 707-12), thereby inhibiting the growth of HER2-positive cancerous cells. Although HERCEPTIN is useful in treating patients with HER2-overexpressing breast cancers that have received extensive prior anti-cancer therapy, some patients in this population fail to respond or respond only poorly to HERCEPTIN treatment. Therefore, there is a significant clinical need for developing further HER2-directed cancer therapies for those patients with HER2-overexpressing tumors or other diseases associated with HER2 expression that do not respond, or respond poorly to HERCEPTIN treatment.

Antibody drug conjugates (ADCs), a rapidly growing class of targeted therapeutics, represent a promising new approach toward improving both the selectivity and the cytotoxic activity of cancer drugs. See, for example, Trail et al., “Monoclonal antibody drug immunoconjugates for targeted treatment of cancer”, Cancer Immunol. Immunother. 2003, 52, 328-337; and Chari, “Targeted Cancer Therapy Conferring Specificity to Cytotoxic Drugs”, Acc. Chem. Res., 2008, 41(1), 98-107. These ADCs have three components: (1) a monoclonal antibody conjugated through a (2) linker to a (3) cytotoxin. The cytotoxins are attached to either lysine or cysteine sidechains on the antibody through linkers that react selectively with primary amines on lysine or with sulfhydryl groups on cysteine. The maximum number of linkers/drugs that can be conjugated depends on the number of reactive amino or sulfhydryl groups that are present on the antibody. A typical antibody contains up to 90 lysines as potential conjugation sites; however, the optimal number of cytotoxins per antibody for most ADCs is typically between 2 and 4 due to aggregation of ADCs with higher numbers of cytotoxins. As a result, conventional lysine linked ADCs currently in clinical development are heterogeneous mixtures that contain from 0 to 10 cytotoxins per antibody conjugated to different amino groups on the antibody. Key factors in the success of an ADC include that the monoclonal antibody is cancer antigen specific, non-immunogenic, low toxicity, and internalized by cancer cells; the cytotoxin is highly potent and is suitable for linker attachment; while the linker may be specific for cysteine (S) or lysine (N) binding, is stable in circulation, may be protease cleavable and/or pH sensitive, and is suitable for attachment to the cytotoxin.

Anticancer ADCs approved for therapeutic use in the USA include brentuximab vedotin (ADCETRIS®), a chimeric anti-CD30 antibody conjugated to monomethylauristatin E used in anaplastic large cell lymphoma and Hodgkin lymphoma; and gemtuzumab ozogamicin (MYLOTARG®), a humanized anti-CD33 antibody conjugated to calicheamicin γ used in acute myelogeneous leukemia—though this was withdrawn in 2010 for lack of efficacy.

Although several ADCs have demonstrated recent clinical success, the utility of most ADCs currently in development may be limited by cumbersome synthetic processes resulting in high cost of goods, insufficient anti-tumor activity associated with limited potency of the cytotoxic drug, and questionable safety due to linker instability and ADC heterogeneity. See, for example, Ducry et al., “Antibody-Drug Conjugates: Linking Cytotoxic Payloads to Monoclonal Antibodies”, Bioconjugate Chem. 2010, 21, 5-13; Chari, “Targeted Cancer Therapy: Conferring Specificity to Cytotoxic Drugs”, Acc. Chem. Res. 2008, 41, 98-107; and Senter, “Recent advancements in the use of antibody drug conjugates for cancer therapy”, Biotechnol.: Pharma. Aspects, 2010, 11, 309-322.

As an example, trastuzumab has been conjugated to the maytansinoid drug mertansine to form the ADC trastuzumab emtansine, also called trastuzumab-DM1 or trastuzumab-MC-DM1, abbreviated T-DM1 (LoRusso et al., “Trastuzumab Emtansine: A Unique Antibody-Drug Conjugate in Development for Human Epidermal Growth Factor Receptor 2-Positive Cancer”, Clin. Cancer Res. 2011, 17, 6437-6447; Burris et al., “Trastuzumab emtansine: a novel antibody-drug conjugate for HER2-positive breast cancer”, Expert Opin. Biol. Ther. 2011, 11, 807-819). It is now in Phase III studies in the US for that indication. The mertansine is conjugated to the trastuzumab through a maleimidocaproyl (MC) linker which bonds at the maleimide to the 4-thiovaleric acid terminus of the mertansine side chain and forms an amide bond between the carboxyl group of the linker and a lysine basic amine of the trastuzumab. Trastuzumab has 88 lysines (and 32 cysteines). As a result, trastuzumab emtansine is highly heterogeneous, containing dozens of different molecules containing from 0 to 8 mertansine units per trastuzumab, with an average mertansine/trastuzumab ratio of 3.4.

Antibody cysteines can also be used for conjugation to cytotoxins through linkers that contain maleimides or other thiol specific functional groups. A typical antibody contains 4, or sometimes 5, interchain disulfide bonds (2 between the heavy chains and 2 between heavy and light chains) that covalently bond the heavy and light chains together and contribute to the stability of the antibodies in vivo. These interchain disulfides can be selectively reduced with dithiothreitol, tris(2-carboxyethyl)phosphine, or other mild reducing agents to afford 8 reactive sulfhydryl groups for conjugation. Cysteine linked ADCs are less heterogeneous than lysine linked ADCs because there are fewer potential conjugation sites; however, they also tend to be less stable due to partial loss of the interchain disulfide bonds during conjugation, since current cysteine linkers bond to only one sulfur atom. The optimal number of cytotoxins per antibody for cysteine linked ADCs is also 2 to 4. For example, ADCETRIS is a heterogeneous mixture that contains 0 to 8 monomethylauristatin E residues per antibody conjugated through cysteines.

The tubulysins, first isolated by the Hofle/Reichenbach group from myxobacterial cultures (Sasse et al., J. Antibiot. 2000, 53, 879-885), are exceptionally potent cell-growth inhibitors that act by inhibiting tubulin polymerization and thereby induce apoptosis. (Khalil et al., Chem. Biochem. 2006, 7, 678-683; and Kaur et al., Biochem. J. 2006, 396, 235-242). The tubulysins, of which tubulysin D is the most potent, have activity that exceeds most other tubulin modifiers including, the epothilones, vinblastine, and paclitaxel (TAXOL®), by 10- to 1000-fold. (Steinmetz et al., Angew. Chem. 2004, 116, 4996-5000; Steinmetz et al., Angew. Chem. Int. Ed. 2004, 43, 4888-4892; and Hale et al., Pure App. Chem. 2003, 75, 167-178). Paclitaxel and vinblastine are current treatments for a variety of cancers, and epothilone derivatives are under active evaluation in clinical trials. Synthetic derivatives of tubulysin D would provide essential information about the mechanism of inhibition and key binding interactions, and could have superior properties as anticancer agents either as isolated entities or as chemical warheads on targeted antibodies or ligands.

Tubulysin D is a complex pseudo-tetrapeptide that can be divided into four regions, Mep (D-N-methylpipecolinic acid), Ile (isoleucine), Tuv (tubuvaline), and Tup (tubuphenylalanine), as shown in the formula:

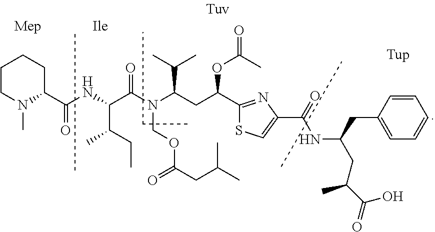 [SMILES: CC[C@H](C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccccc2)C[C@H](C)C(=O)O)cs1)C(C)C]

Most of the more potent derivatives of tubulysin, including tubulysin D, also incorporate the interesting O-acyl N,O-acetal functionality, which has rarely been observed in natural products. This reactive functionality is labile in both acidic and basic reaction conditions, and therefore may play a key role in the function of the tubulysins. (Iley et al., Pharm. Res. 1997, 14, 1634-1639). Recently, the total synthesis of tubulysin D was reported, which represents the first synthesis of any member of the tubulysin family that incorporates the O-acyl N,O-acetal functionality. (Peltier et al., J. Am. Chem. Soc. 2006, 128, 16018-16019). Other tubulysins, including tubulysins U and V, have been synthesized by Donating et al., “Total Synthesis of Tubulysins U and V”, Angew. Chem. Int. Ed. 2006, 45, 7235-7239; including the synthesis of tubulysins via multi-component reactions; i.e. using the Ugi or Passerinni methods.

US Patent Application Publication No. US2011/0021568 A1 (Ellman et al.) discloses the synthesis and activities of a number of tubulysin analogs, including compounds (40) and (10), referred to here as T1 and T2, respectively:

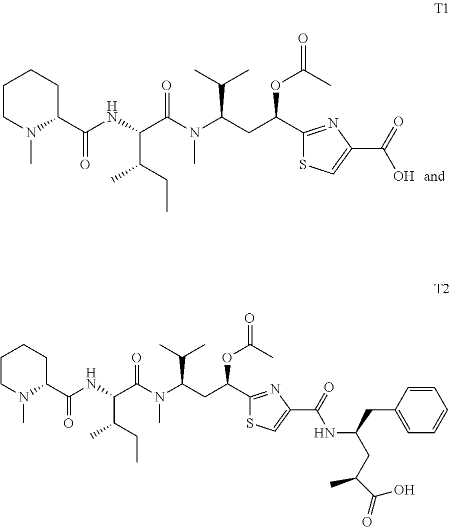 [SMILES: CC[C@H](C)[C@H](NC(=O)[C@H]1CCCCN1C)C(=O)N(C)[C@H](C[C@@H](OC(C)=O)c1nc(C(=O)O)cs1)C(C)C ; "and" ; CC[C@H](C)[C@H](NC(=O)[C@H]1CCCCN1C)C(=O)N(C)[C@H](C[C@@H](OC(C)=O)c1nc(C(=O)N[C@@H](Cc2ccccc2)C[C@H](C)C(=O)O)cs1)C(C)C]

Schumacher et al., “In Situ Maleimide Bridging of Disulfides and a New Approach to Protein PEGylation”, Bioconjugate Chem. 2011, 22, 132-136, disclose the synthesis of 3,4-disubstituted maleimides such as 3,4-bis(2-hydroxyethylsulfanyl)pyrrole-2,5-dione [referred to by Schumacher et al. as “dimercaptoethanolmaleimide”] and 3,4-bis(phenylsulfanyl)pyrrole-2,5-dione [“dithiophenolmaleimide”], and their N-PEGylated derivatives as PEGylating agents for somatostatin, where the substituted maleimide bonds to the two sulfur atoms of an opened cysteine-cysteine disulfide bond.

It would be desirable to develop potent, homogeneous ADCs, compositions containing them and methods for their use in treating cancers, and methods and intermediates in their preparation.

The disclosures of the documents referred to in this application are incorporated into this application by reference.

### SUMMARY OF THE INVENTION

In one embodiment, the present application discloses antibody-cytotoxin antibody-drug conjugates (ADCs) of the formula:

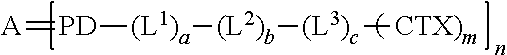

wherein:

A is an antibody;

PD is a pyrrole-2,5-dione or derivative thereof, a pyrrolidine-2,5-dione or derivative thereof;

CTX is a cytotoxin;

each L¹, L² and L³ is independently a linker selected from the group consisting of —O—, —C(O)—, —S—, —S(O)—, —S(O)₂—, —NH—, —NCH₃—, —(CH₂)_{q}—, —NH(CH₂)₂NH—, —OC(O)—, —CO₂—, —NHCH₂CH₂C(O)—, —C(O)NHCH₂CH₂NH—, —NHCH₂C(O)—, —NHC(O)—, —C(O)NH—, —NCH₃C(O)—, —C(O)NCH₃—, —(CH₂CH₂O)ₚ—, —(CH₂CH₂O)ₚCH₂CH₂—, —CH₂CH₂—(CH₂CH₂O)ₚ—, —OCH(CH₂O—)₂—, cyclopentanyl, cyclohexanyl, unsubstituted phenylenyl, phenylenyl substituted by 1 or 2 substituents selected from the group consisting of halo, CF₃—, CF₃O—, CH₃O—, —C(O)OH, —C(O)OC₁₋₃alkyl, —C(O)CH₃, —CN, —NH₂, —OH, —NHCH₃, —N(CH₃)₂, C₁₋₃alkyl and -(AA)ᵣ-;

a, b and c are each independently 0, 1, 2 or 3, provided that at least one of a, b or c is 1;

each p is independently an integer of 1 to 14;

each q is independently an integer from 1 to 12;

each AA is independently an amino acid;

each r is 1 to 12; and

m is an integer of 1 to 4; and n is an integer of 1 to 4;

with the proviso that when -(L¹)ₐ-(L²)_{b}-(L³)_{c}- together is —(CH₂)₁₋₁₂— or —(CH₂CH₂O)₁₋₁₂CH₂CH₂— then L¹, L² and L³ are not bonded to CTX by an amide bond.

In one aspect of the linkers of the present application, the cyclopentanyl, cyclohexanyl, and phenylenyl may be divalent linkers or trivalent linkers that may be attached to one, two or more CTX residues. In another aspect of the ADC of the present application, the linker is attached to the CTX by a group selected from the group consisting of NHC(O)—, —NHC(O)O—, —N(C₁₋₃alkyl)C(O)O—, —NH—, —N(C₁₋₃alkyl)-, —N(C₁₋₃alkyl)C(O)NH— and —N(C₁₋₃alkyl)C(O)N(C₁₋₃alkyl)-.

Because of the bidentate binding of the PD to the two sulfur atoms of an opened cysteine-cysteine disulfide bond in the antibodies, these ADCs are homogeneous and have enhanced stability over ADCs with monodentate linkers. They will therefore have increased half-lives in vivo, reducing the amount of cytotoxin released systemically, and be safer than ADCs with monodentate linkers linking one antibody amino acid to one linkage point which may attach one or more drug entities.

In another embodiment, there is provided pharmaceutical compositions containing ADCs as disclosed herein, and methods of treatment of cancers targeted by the relevant antibodies by administering ADCs of the present application or pharmaceutical compositions thereof.

In another embodiment, there is provided a linker-cytotoxin conjugate of formula A, B or C:

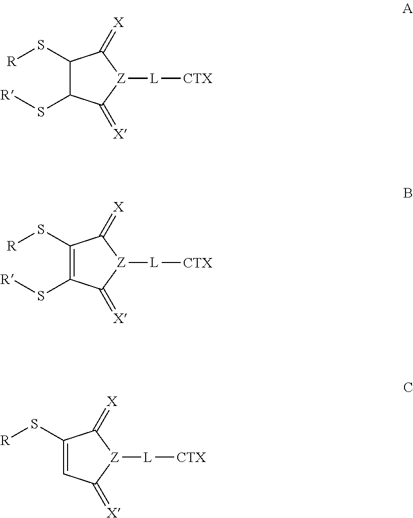

where each R and R′ is independently selected from the group consisting of C₁₋₆alkyl optionally substituted with halo or hydroxyl; phenyl optionally substituted with halo, hydroxyl, carboxyl, C₁₋₃alkoxycarbonyl, or C₁₋₃alkyl; naphthyl optionally substituted with halo, hydroxyl, carboxyl, C₁₋₃alkoxycarbonyl, or C₁₋₃alkyl; 2-pyridyl optionally substituted with halo, hydroxyl, carboxyl, C₁₋₃alkoxycarbonyl or C₁₋₃alkyl; C₁₋₆alkylsulfonyloxy, C₂₋₄₀cycloalkylsulfonyloxy, C₆₋₁₀arylsulfonyloxy; C₁₋₆alkyl-S—, C₆₋₁₀aryl-S— and C₆₋₁₀heteroaryl-S—;

X is O, S or NR¹ where R¹ is H or C₁₋₃alkyl;

X′ is O, S or NR² where R² is H or C₁₋₃alkyl;

Z is selected from the group consisting of N—, CH—, CR³— and CR³—CR⁴R⁵— where R³, R⁴ and R⁵ are each independently H or C₁₋₃alkyl.

L is a linker defined by L¹-L²-L³, wherein each L¹, L² and L³ is independently a linker selected from the group consisting of —O—, —C(O)—, —S—, —S(O)—, —S(O)₂—, —NH—, —NCH₃—, —(CH₂)_{q}—, —NH(CH₂)₂NH—, —OC(O)—, —CO₂—, —NHCH₂CH₂C(O)—, —C(O)NHCH₂CH₂NH—, —NHCH₂C(O)—, —NHC(O)—, —C(O)NH—, —NCH₃C(O)—, —C(O)NCH₃—, —(CH₂CH₂O)ₚ—, —(CH₂CH₂O)ₚCH₂CH₂—, —CH₂CH₂—(CH₂CH₂O)ₚ—, —OCH(CH₂O—)₂—, cyclopentanyl, cyclohexanyl, unsubstituted phenylenyl, phenylenyl substituted by 1 or 2 substituents selected from the group consisting of halo, CF₃—, CF₃O—, CH₃O—, —C(O)OH, —C(O)OC₁₋₃alkyl, —C(O)CH₃, —CN, —NH₂, —OH, —NHCH₃, —N(CH₃)₂, —C₁₋₃alkyl and -(AA)ᵣ-;

a, b and c are each independently 0, 1, 2 or 3, provided that at least one of a, b or c is 1;

each p is independently an integer of 1 to 14;

each q is independently an integer from 1 to 12;

each AA is independently an amino acid; each r is 1 to 12; and

CTX is a cytotoxin bonded to L by an amide bond; with the proviso that when L or -(L¹)ₐ-(L²)₆-(L³)_{c}- together is (CH₂)₁₋₁₂— or —(CH₂CH₂O)₁₋₁₂CH₂CH₂— then L¹, L² and L³ are not bonded to CTX by an amide bond. In one aspect of the above, L is (CH₂)ₘ or (CH₂CH₂O)ₘCH₂CH₂.

In another aspect, the C₁₋₆alkyl-S—, C₆₋₁₀aryl-S— and C₆₋₁₀heteroaryl-S— is selected from the group consisting of:

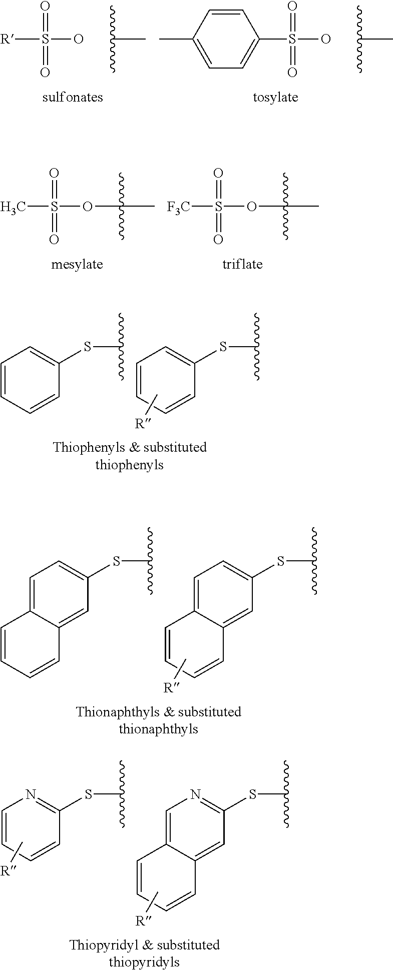

wherein R′ is C₁₋₆alkyl, C₆₋₁₀aryl, C₆₋₁₀heteroaryl, each of which is optionally substituted by R″ that is selected from the group consisting of halo, CF₃—, CF₃O—, CH₃O—, —C(O)OH, —C(O)OC₁₋₃alkyl, —C(O)CH₃, —CN, —NH₂, —OH, —NHCH₃, —N(CH₃)₂ and C₁₋₃alkyl.

These bidentate linkers are also useful in preparing the linker-cytotoxin conjugates of the present application, and are useful in preparing the linkers as disclosed herein.

In another embodiment, there is provided novel auristatins, derivatives of the auristatins, tubulysin and derivatives of the tubulysins, wherein the auristatins, tubulysins and their derivatives represented as their respective residues are selected from the group consisting of CTX-I, CTX-II, CTX-III, CTX-IV, CTX-V, CTX-VI, CTX-VII and CTX-VIII, wherein the squiggly line (˜) on the bond of the residue is attached to a hydrogen.

In another embodiment, there is provided a linker of formula AA, BB or CC:

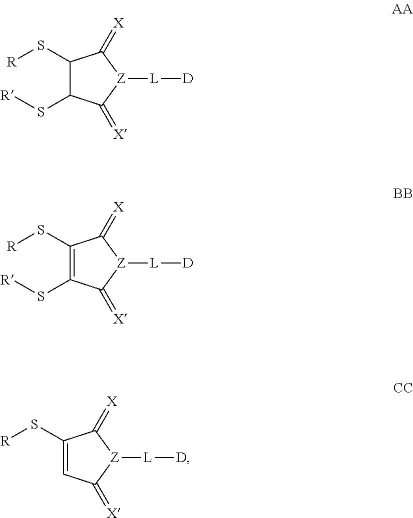

where each R and R′ is independently selected from the group consisting of C₁₋₆alkyl optionally substituted with halo or hydroxyl; phenyl optionally substituted with halo, hydroxyl, carboxyl, C₁₋₃alkoxycarbonyl or C₁₋₃alkyl; naphthyl optionally substituted with halo, hydroxyl, carboxyl, C₁₋₃alkoxycarbonyl or C₁₋₃alkyl; or 2-pyridyl optionally substituted with halo, hydroxyl, carboxyl, C₁₋₃alkoxycarbonyl or C₁₋₃alkyl; C₁₋₆alkylsulfonyloxy, C₂₋₁₀cycloalkylsulfonyloxy and C₆₋₁₀arylsulfonyloxy;

L is a linker defined by -(L¹)ₐ-(L²)_{b}-(L³)_{c}-, wherein each L¹, L² and L³ is independently a linker selected from the group consisting of —O—, —C(O)—, —S—, —S(O)—, —S(O)₂—, —NH—, —NCH₃—, —(CH₂)_{q}—, —NH(CH₂)₂NH—, —OC(O)—, —CO₂—, —NHCH₂CH₂C(O)—, —C(O)NHCH₂CH₂NH—, —NHCH₂C(O)—, —NHC(O)—, —C(O)NH—, —NCH₃C(O)—, —C(O)NCH₃—, —(CH₂CH₂O)ₚ—, —(CH₂CH₂O)ₚCH₂CH₂—, —CH₂CH₂—(CH₂CH₂O)ₚ—, —OCH(CH₂O—)₂—, cyclopentanyl, cyclohexanyl, unsubstituted phenylenyl, phenylenyl substituted by 1 or 2 substituents selected from the group consisting of halo, CF₃—, CF₃O—, CH₃O—, —C(O)OH, —C(O)OC₁₋₃alkyl, —C(O)CH₃, —CN, —NH₂, —OH, —NHCH₃, —N(CH₃)₂, —C₁₋₃alkyl and -(AA)ᵣ-;

a, b and c are each independently 0, 1, 2 or 3, provided that at least one of a, b or c is 1;

each p is independently an integer of 1 to 14;

each q is independently an integer from 1 to 12;

each AA is independently an amino acid; each r is 1 to 12;

D is carboxyl, C₁₋₆alkoxycarbonyl or amino, and m is an integer of 1 to 12. In one aspect of the above, L is —(CH₂)ₘ— or —(CH₂CH₂O)ₘCH₂CH₂—.

In one embodiment, there is provided a linker of formula AAA, BBB, CCC or DDD:

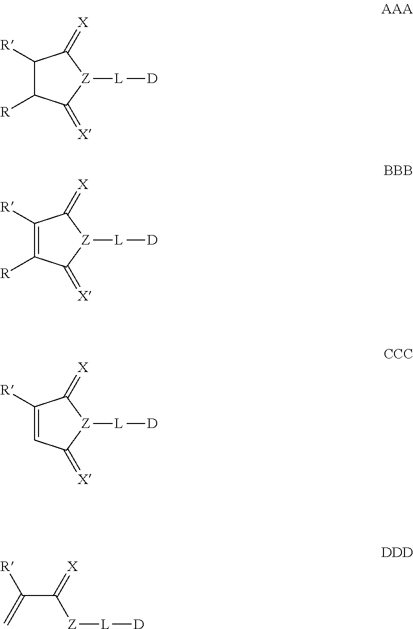

where each Rand R′ is independently selected from the group consisting of chloro, bromo, iodo, C₁₋₆alkylsulfonyloxy, C₂₋₁₀cycloalkylsulfonyloxy, C₆₋₁₀arylsulfonyloxy;

L is a linker defined by -(L¹)ₐ-(L²)_{b}-(L³)_{c}-, wherein each L¹, L² and L³ is independently a linker selected from the group consisting of —O—, —C(O)—, —S—, —S(O)—, —S(O)₂—, —NH—, —NCH₃—, —(CH₂)_{q}—, —NH(CH₂)₂NH—, —OC(O)—, —CO₂—, —NHCH₂CH₂C(O)—, —C(O)NHCH₂CH₂NH—, —NHCH₂C(O)—, —NHC(O)—, —C(O)NH—, —NCH₃C(O)—, —C(O)NCH₃—, —(CH₂CH₂O)ₚ—, —(CH₂CH₂O)ₚCH₂CH₂—, —CH₂CH₂—(CH₂CH₂O)ₚ—, —OCH(CH₂O—)₂—, cyclopentanyl, cyclohexanyl, unsubstituted phenylenyl, phenylenyl substituted by 1 or 2 substituents selected from the group consisting of halo, CF₃—, CF₃O—, CH₃O—, —C(O)OH, —C(O)OC₁₋₃alkyl, —C(O)CH₃, —CN, —NH₂, —OH, —NHCH₃, —N(CH₃)₂, C₁₋₃alkyl and -(AA)ᵣ-; a, b and c are each independently 0, 1, 2 or 3, provided that at least one of a, b or c is 1; each p is independently an integer of 1 to 14; each q is independently an integer from 1 to 12; each AA is independently an amino acid; each r is 1 to 12; and D is carboxyl, C₁₋₆alkoxycarbonyl or amino.

In one aspect of the above, each R and R′ is independently selected from the group consisting of H, Cl, Br and I and iodo; and L is selected from the group consisting of —(CH₂)₁₋₅C(O)-Val-Ala-NH-(p-C₆H₄)—CH₂OC(O)-(p-C₆H₄)—NO₂, —(CH₂CH₂O)₁₋₁₂—(CH₂CH₂)C(O)-Val-Ala-NH-(p-C₆H₄)—CH₂OC(O)-(p-C₆H₄)—NO₂, —(CH₂)₁₋₅C(O)-Val-Cit-NH-(p-C₆H₄)—CH₂OC(O)-(p-C₆H₄)—NO₂, —(CH₂CH₂O)₁₋₁₂—CH₂CH₂)C(O)-Val-Cit-NH-(p-C₆H₄)—CH₂OC(O)-(p-C₆H₄)—NO₂.

In one aspect of the above, R and R′ is selected from the group consisting of trifluoromethanesulfonyloxy, benzenesulfonyloxy and 4-toluenesulfonyloxy. In another aspect of the above, L is (CH₂), or (CH₂CH₂O)ₘCH₂CH₂ and m is an integer of 1 to 12.

Preferred embodiments of this invention are characterized by the specification and by the features of the claims of this application as filed, and of corresponding pharmaceutical compositions, methods and uses of these compounds.

### DETAILED DESCRIPTION OF THE INVENTION

### BRIEF DESCRIPTION OF THE DRAWINGS

FIG. 1: Antibody Only; RT=7.12

FIG. 2: Antibody+dibromosuccinimide−RT=7.24

FIG. 3: Antibody+dibromo-N-benzyl succinimide−RT=7.58

FIG. 4: Conventional mc-MMAF ADC

FIG. 5: “Stapled” or “Snapped” dts-ADC

FIG. 6: 18-2A Antibody only

FIG. 7: 18-2A-mc-MMAF (conventional ADC)

FIG. 8: 18-2A-dts-MMAF (“stapled” or “snapped” ADC)

FIG. 9: Potency of T2 and T4 ADCs in Tubulin Polymerization Assay.

FIG. 10: Potency of T2 ADCs in Tubulin Polymerization Assay.

FIG. 11: T2 and T4 Tubulin Polymerization Assays.

FIG. 12: T2 and T4 Assays.

FIG. 13: T2 ADCs Inhibit microtubule formation in vitro and are more potent to T4 ADCs.

FIG. 14: T2 ADC Tubulin Assay.

FIG. 15: ADC Conjugation Protocol for “Stapled” or “Snapped” Linkers.

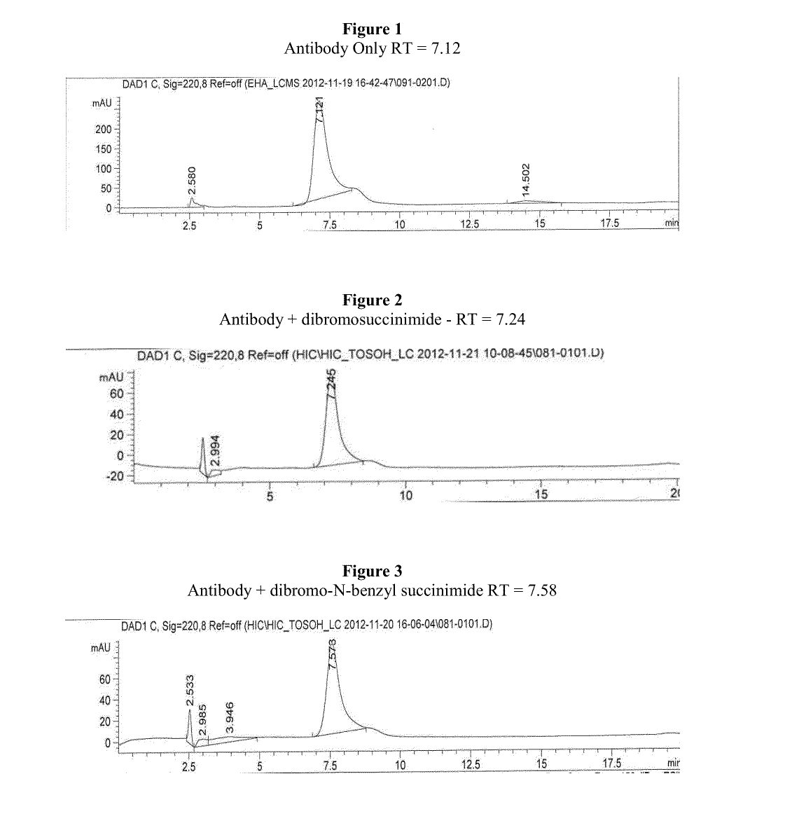

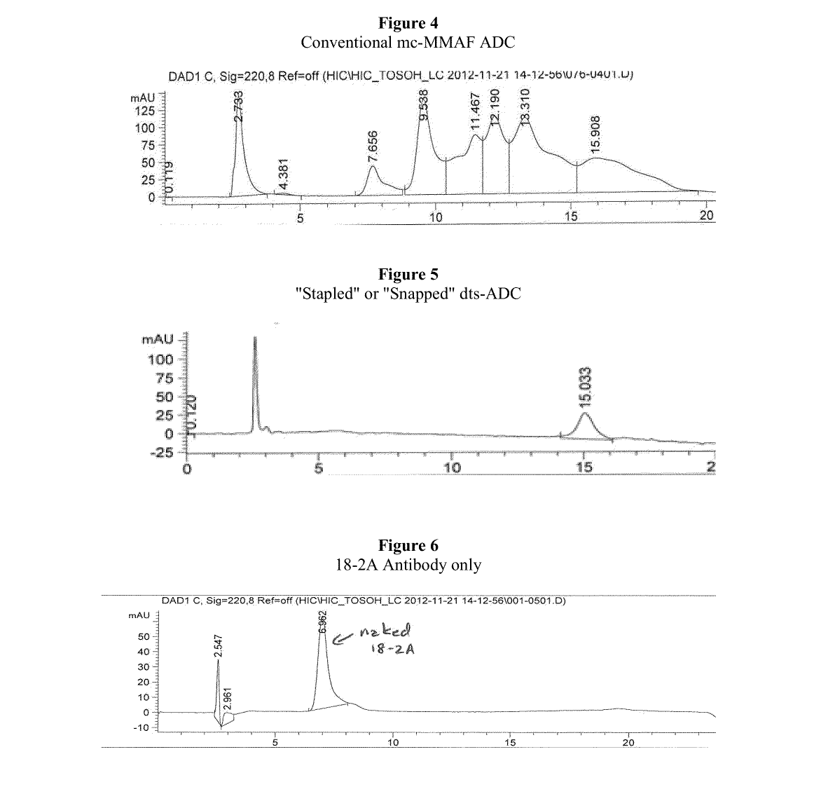

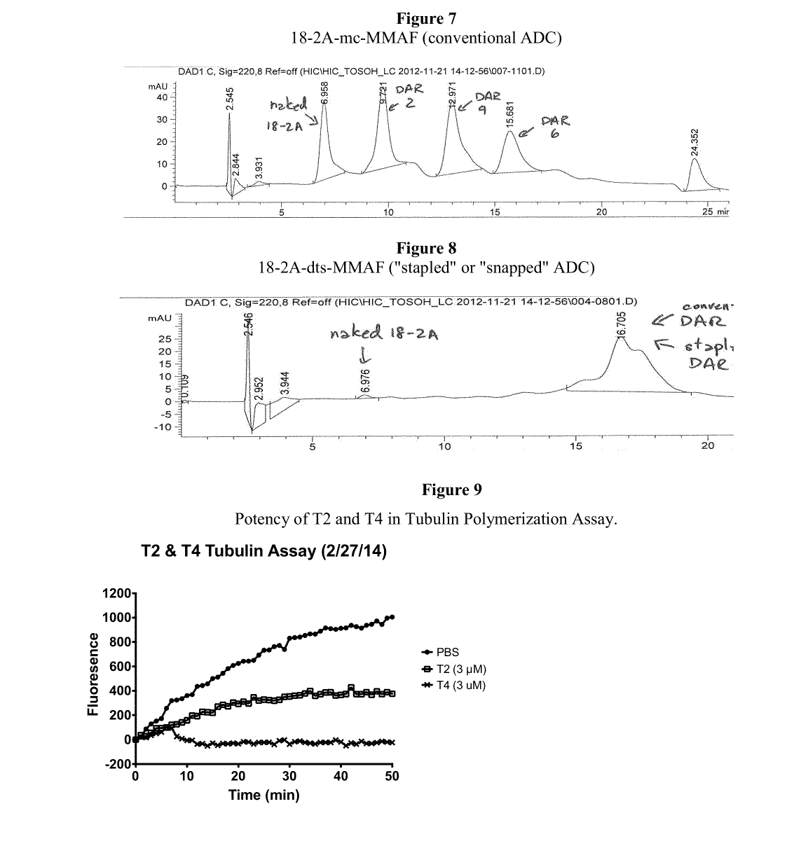

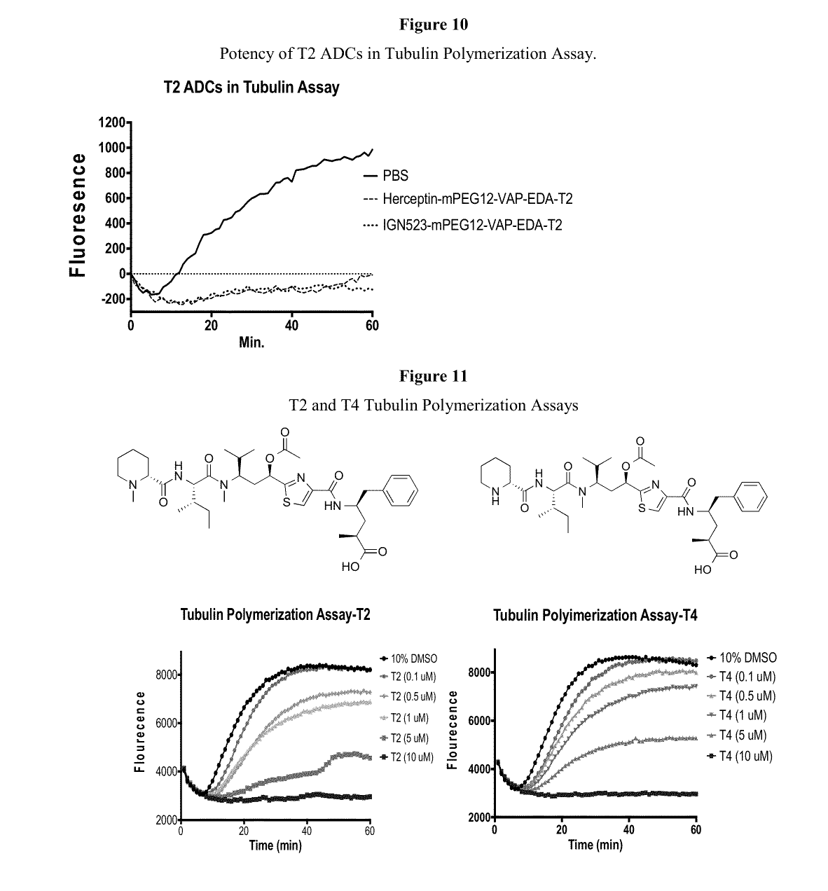

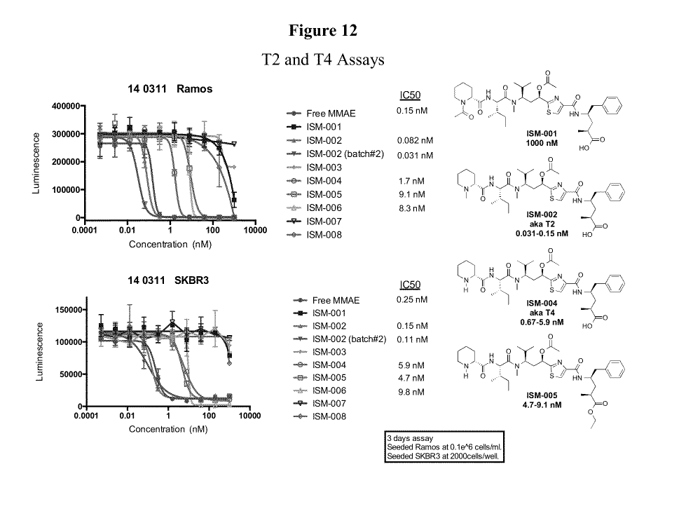

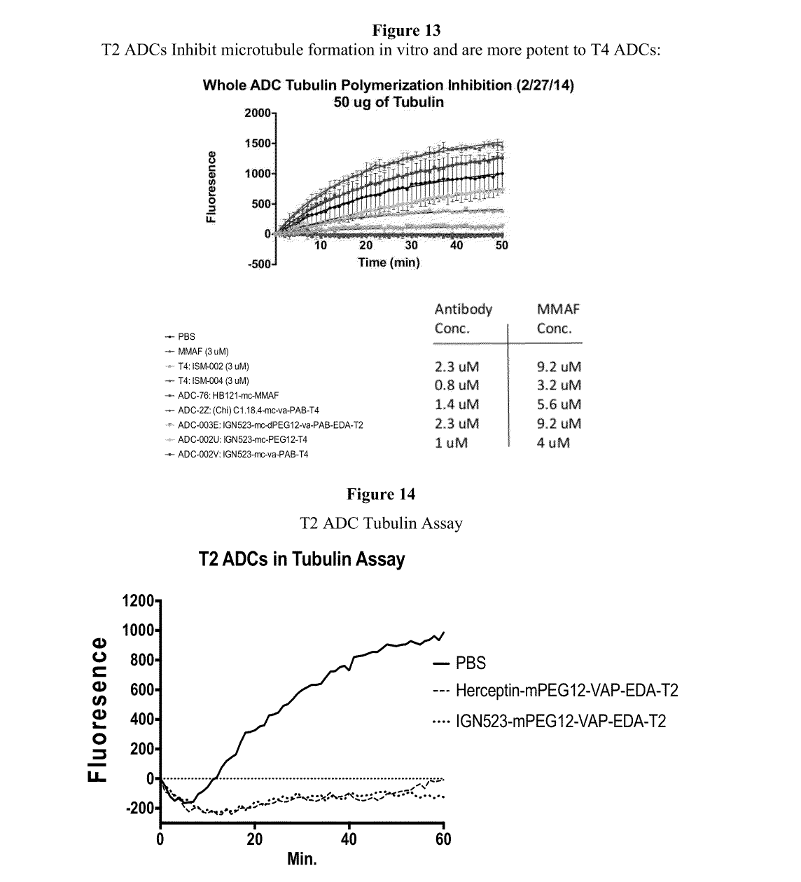

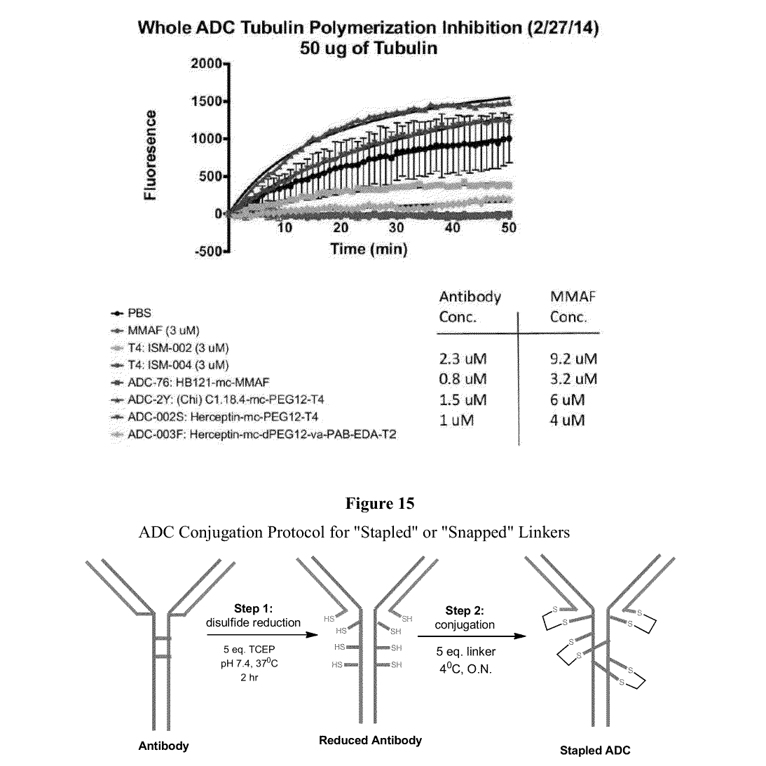

### DEFINITIONS

An “antibody”, also known as an immunoglobulin, is a large Y-shaped protein used by the immune system to identify and neutralize foreign objects such as bacteria and viruses. The antibody recognizes a unique part of the foreign target, called an antigen, because each tip of the “Y” of the antibody contains a site that is specific to a site on an antigen, allowing these two structures to bind with precision. An antibody consists of four polypeptide chains, two identical heavy chains and two identical light chains connected by cysteine disulfide bonds. A “monoclonal antibody” is a monospecific antibody where all the antibody molecules are identical because they are made by identical immune cells that are all clones of a unique parent cell. Initially, monoclonal antibodies are typically prepared by fusing myeloma cells with the spleen cells from a mouse (or B-cells from a rabbit) that has been immunized with the desired antigen, then purifying the resulting hybridomas by such techniques as affinity purification. Recombinant monoclonal antibodies are prepared in viruses or yeast cells rather than in mice, through technologies referred to as repertoire cloning or phage display/yeast display, the cloning of immunoglobulin gene segments to create libraries of antibodies with slightly different amino acid sequences from which antibodies with desired specificities may be obtained. The resulting antibodies may be prepared on a large scale by fermentation. “Chimeric” or “humanized” antibodies are antibodies containing a combination of the original (usually mouse) and human DNA sequences used in the recombinant process, such as those in which mouse DNA encoding the binding portion of a monoclonal antibody is merged with human antibody-producing DNA to yield a partially-mouse, partially-human monoclonal antibody. Full-humanized antibodies are produced using transgenic mice (engineered to produce human antibodies) or phage display libraries. Antibodies (Abs) and “immunoglobulins” (Igs) are glycoproteins having similar structural characteristics. While antibodies exhibit binding specificity to a specific antigen, immunoglobulins include both antibodies and other antibody-like molecules which generally lack antigen specificity. Polypeptides of antibody-like molecules are produced at low levels by the lymph system and at increased levels by myelomas. The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity). These antibodies may also include certain antibody fragments. An antibody can be chimeric, human, hunanized and/or affinity matured. Antibodies of particular interest in this invention are those that are specific to cancer antigens, are non-immunogenic, have low toxicity, and are readily internalized by cancer cells; and suitable antibodies include alemtuzumab, bevacizumab, brentuximab, cetuximab, gemtuzumab, ipilimumab, ofatumumab, panitumumab, rituximab, tositumomab, inotuzumab, glembatumumab, lovortuzumab and trastuzumab. Antibodies also include adecatumumab, afutuzumab, bavituximab, belimumab, bivatuzumab, cantuzumab, citatuzumab, cixutumumab, conatumumab, dacetuzumab, elotuzumab, etaracizumab, farletuzumab, figitumumab, iratumumab, labetuzumab, lexatumumab, lintuzumab, lucatumumab, mapatumumab, matuzumab, milatuzumab, necitumumab, nimotuzumab, olaratumab, oportuzumab, pertuzumab, pritumumab, ranibizumab, robatumumab, sibrotuzumab, siltuximab, tacatuzumab, tigatuzumab, tucotuzumab, veltuzumab, votumumab and zalutumumab.

The terms “full length antibody,” “intact antibody” and “whole antibody” are used herein interchangeably to refer to an antibody in its substantially intact form, and are not antibody fragments as defined below. The terms particularly refer to an antibody with heavy chains that contain the Fc region.

“Antibody fragments” comprise only a portion of an intact antibody, wherein the portion retains at least one, two, three and as many as most or all of the functions normally associated with that portion when present in an intact antibody. In one aspect, an antibody fragment comprises an antigen binding site of the intact antibody and thus retains the ability to bind antigen. In another aspect, an antibody fragment, such as an antibody fragment that comprises the Fc region, retains at least one of the biological functions normally associated with the Fc region when present in an intact antibody. Such functions may include FcRn binding, antibody half life modulation, ADCC function and complement binding. In another aspect, an antibody fragment is a monovalent antibody that has an in vivo half life substantially similar to an intact antibody. For example, such an antibody fragment may comprise on antigen binding arm linked to an Fc sequence capable of conferring in vivo stability to the fragment.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. The modifier term “monoclonal” indicates the character of the antibody as not being a mixture of discrete antibodies. In certain aspects, such a monoclonal antibody may include an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. In addition to their specificity, monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. (See Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), and technologies for producing human or human-like antibodies in animals that have parts or all of the human immunoglobulin loci or genes encoding human immunoglobulin sequences (see, WO98/24893; WO96/34096; WO96/33735 and WO91/10741). The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567). “Humanized” forms of non-human (e.g., murine) antibodies are chimeric antibodies that contain minimal sequence derived from non-human immunoglobulin. In one aspect, a humanized antibody is a human immunoglobulin (recipient antibody) in which residues from a hypervariable region of the recipient are replaced by residues from a hypervariable region of a non-human species (donor antibody) such as mouse, rat, rabbit, or nonhuman primate having the desired specificity, affinity, and/or capacity. In another aspect, framework region (FR) residues of the human immunoglobulin are replaced by corresponding non-human residues. In general, a humanized antibody will comprise substantially all of at least one, and typically two, variable domains, in which all or substantially all of the hypervariable loops correspond to those of a non-human immunoglobulin, and all or substantially all the FRs are those of a human immunoglobulin sequence. The humanized antibody may comprise at least a portion of an immunoglobulin constant region (Fc), typically that of a human immunoglobulin. See Vaswani and Hamilton, Ann Allergy, Asthma & Immunol. 1:105-115 (1998); Harris, Biochem. Soc. Transactions 23:1035-1038 (1995); Hurle and Gross, Curr. Op. Biotech. 5:428-433 (1994).

“Framework” or “FR” residues are those variable domain residues other than the hypervariable region residues. “Fc receptor” or “FcR” is a receptor that binds to the Fc region of an antibody. In some embodiments, an FcR is a native human FcR. In one aspect, an FcR is one which binds an IgG antibody (a gamma receptor) and includes receptors of the FcγRI, FcγRII and FcγRIII subclasses. (See Daeron, Annu. Rev. Immunol. 15:203-234 (1997)).

An “amino acid” (or AA) or amino acid residue include but are not limited to the 20 naturally occurring amino acids acids commonly designated by three letter symbols and also includes citrulline (Cit), 4-hydroxyproline, hydroxylysine, demosine, isodemosine, 3-methylhistidine, norvalin, beta-alanine, gamma-aminobutyric acid, homocysteine, homoserine, ornithine and methionine sulfone. The amino acid residue of the present application also include the corresponding N-methyl amino acids, such as —N(CH₃)CH₂C(O)O—, —NHC(O)CH₂CH₂CH(NHCH₃)C(O)O— etc. . . . The amino acids, dipeptides, tripeptides, oligomers and polypeptides designated as -(AA)ᵣ- of the present application may include the corresponding non-N-alkylated amino acids and peptides (such as non-N-methylated amino acids in the peptides), as well as a mixture of the non-N-alkylated amino acids and the N-alkylated amino acids of the peptides.

A “cytotoxin” (CTX) is a molecule that has a cytotoxic effect on cells (e.g., when released within a cancer cell, is toxic to that cell). Cytotoxins of particular interest in this invention are the tubulysins (such as the tubulysins of the formulae T3 and T4, and CTX-I′, CTX-II′, CTX-III′, CTX-IV′, CTX-V′, CTX-VI′, CTX-VII′ and CTX-VIII′ disclosed herein), the auristatins (such as monomethylauristatin E and monomethylauristatin F), the maytansinoids (such as mertansine), the calicheamicins (such as calicheamicin γ); those cytotoxins that, like the tubulysins of the formulae T3 and T4, and those disclosed herein are capable of coordination through an amide bond to a linker, such as by possessing a basic amine or a carboxyl group.

A “linker” (noted as L or L¹, L² and L³) is a molecule with two reactive termini, one for conjugation to an antibody or to another linker and the other for conjugation to a cytotoxin. The antibody conjugation reactive terminus of the linker is typically a site that is capable of conjugation to the antibody through a cysteine thiol or lysine amine group on the antibody, and so is typically a thiol-reactive group such as a double bond (as in maleimide) or a leaving group such as a chloro, bromo or iodo or an R-sulfanyl group or sulfonyl group, or an amine-reactive group such as a carboxyl group or as defined herein; while the antibody conjugation reactive terminus of the linker is typically a site that is capable of conjugation to the cytotoxin through formation of an amide bond with a basic amine or carboxyl group on the cytotoxin, and so is typically a carboxyl or basic amine group. In one embodiment, when the term “linker” is used in describing the linker in conjugated form, one or both of the reactive termini will be absent (such as the leaving group of the thiol-reactive group) or incomplete (such as the being only the carbonyl of the carboxylic acid) because of the formation of the bonds between the linker and/or the cytotoxin.

The term “leaving group,” or “LG”, as used herein, refers to any group that leaves in the course of a chemical reaction involving the group as described herein and includes but is not limited to halogen, sulfonates (brosylate, mesylate, tosylate, triflate etc. . . . ), p-nitrobenzoate and phosphonate groups, for example.

An “antibody-drug conjugate” (ADC) is an antibody that is conjugated to one or more cytotoxins, through one or more linkers. The antibody is typically a monoclonal antibody specific to a therapeutic target such as a cancer antigen.

“Phenyl” means a C₆H₅ group as known in the art. “Phenylene” means a divalent phenyl group, wherein the phenyl group is substituted at two positions on the phenyl ring that may be ortho (o-C₆H₄) or para (p-C₆H₄).

“Tubulysin” includes both the natural products described as tubulysins, such as by Sasse et al. and other authors mentioned in the Description of the related art, and also the tubulysin analogs described in US Patent Application Publication No. US 2011/0021568 A1. Tubulysins disclosed in the present application are noted herein and may include the tubulysins of the formulae T3 and T4, and CTX-I′, CTX-II′, CTX-III′, CTX-IV′, CTX-V′, CTX-VI′, CTX-VII′ and CTX-VIII′ and other tubulysins where the terminal N-methylpiperidine has been replaced by an unsubstituted piperidine (the des-methyl analogs), allowing amide bond formation with a linker.

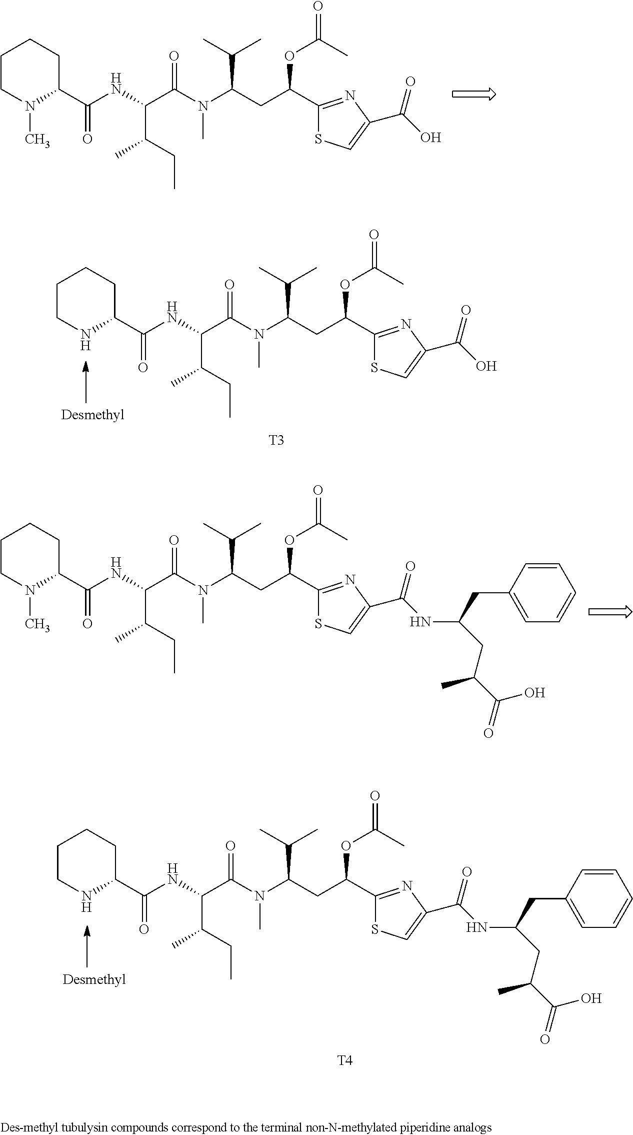

The terms “cell proliferative disorder” and “proliferative disorder” refer to disorders that are associated with some degree of abnormal cell proliferation. In one aspect, the cell-proliferative disorder is cancer.

“Tumor,” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive. The terms “cancer” and “cancerous” refer to the physiological condition in mammals that is typically characterized by unregulated cell growth. Examples of cancer include, but are not limited to, carcinoma, lymphoma, blastoma, sarcoma and leukemia or lymphoid malignancies.

A “basic amine”, such as the amine forming a part of the terminal piperidine group of the tubulysins, such as that of the formulae T3 and T4, CTX-I′, CTX-II′, CTX-III′, CTX-IV′, CTX-V′, CTX-VI′, CTX-VII′ and CTX-VIII′, is a primary or secondary amine that is not part of an amide

A “therapeutically effective amount” means that amount of an ADC of the first aspect of this invention or composition of the second aspect of this invention which, when administered to a human suffering from a cancer, is sufficient to effect treatment for the cancer. “Treating” or “treatment” of the cancer includes one or more of:

(1) limiting/inhibiting growth of the cancer, i.e. limiting its development;
(2) reducing/preventing spread of the cancer, i.e. reducing/preventing metastases;
(3) relieving the cancer, i.e. causing regression of the cancer,
(4) reducing/preventing recurrence of the cancer; and
(5) palliating symptoms of the cancer.

As used herein, the term “pharmaceutically acceptable salt” refers to those salts of the ADCs formed by the process of the present application which are suitable for use in contact with the tissues of humans and lower animals without undue toxicity, irritation, allergic response and the like. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the ADC compounds, or separately by reacting the free base function or group of a compound with a suitable organic acid. Examples of pharmaceutically acceptable salts include, but are not limited to, nontoxic acid addition salts, or salts of an amino group formed with inorganic acids such as hydrochloric acid, hydrobromic acid, phosphoric acid etc. . . . or with organic acids such as acetic acid, maleic acid, tartaric acid, citric acid, succinic acid or malonic acid. Other pharmaceutically acceptable salts include, but are not limited to, adipate, alginate, ascorbate, benzenesulfonate, benzoate, bisulfate, citrate, digluconate, dodecylsulfate, ethanesulfonate, formate, fumarate, gluconate, 2-hydroxy-ethanesulfonate, lactate, laurate, malate, maleate, malonate, methanesulfonate, oleate, oxalate, palmitate, phosphate, propionate, stearate, succinate, sulfate, tartrate, p-toluenesulfonate, valerate salts, and the like. Representative alkali or alkaline earth metal salts include sodium, lithium, potassium, calcium, or magnesium salts, and the like. Further pharmaceutically acceptable salts include, nontoxic ammonium, quaternary ammonium, and amine cations formed using counterions such as halide, hydroxide, carboxylate, sulfate, phosphate, nitrate, alkyl groups having from 1 to 6 carbon atoms (i.e., C₁₋₆alkyl), sulfonate and aryl sulfonate.

Cancers of interest for treatment include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, oral cancer, liver cancer, bladder cancer, cancer of the urinary tract, hepatoma, breast cancer including, for example, HER2-positive breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, acute myeloid leukemia (AML), chronic lymphocytic leukemia (CML), multiple myeloma and B-cell lymphoma, brain cancer, head and neck cancers and associated metastases.

### Abbreviations/Acronyms

ADC: antibody-drug conjugate; DEA: diethylamine; DCC: 1,3-dicyclohexylcarbodiimide; DIAD: diisopropyl azodicarboxylate; DIPC: 1,3-diisopropylcarbodiimide; DIPEA: diisopropylethylamine; DMF: N,N-dimethylformamide; DPBS: Dulbecco's phosphate-buffered saline; DTPA: diethylenetriaminepentaacetic acid; DTT: dithiothreitol; EDC: ethyl 3-(3-dimethylaminopropyl)carbodiimide; HATU: O-(7-azabenzotriazol-1-yl)-N,N,N′,N′-tetramethyluronium hexafluorophosphate; HOBT: N-hydroxybenzotriazole; NHS: N-hydroxysuccinimide; NMM: N-methylmorpholine; MMAE: monomethylauristatin E; MMAF: monomethylauristatin F, monomethylauristatin phenylalanine; MC: maleimidocaproyl, 6-(2,5-dioxopyrrolyl)hexanoyl; PBS: phosphate-buffered saline; PEG: poly(ethyleneglycol); TBTU: 2-(1H-bentotriatol-1-yl)-1,1,3,3-tetrasethyluronium tetrafluoroborate TCEP: tris(2-carboxyethyl)phosphine; TGI: tumor growth inhibition.

### The ADCs of the Invention

As mentioned in the Description of the related art, ADCs of the prior art that coordinate to cysteine thiols of the antibody have employed monofunctional linkers, of which the MC linker is an example. Reduction and opening of the cysteine-cysteine disulfide bonds to give free thiols for conjugation decreases the stability of the antibody, and the formation of the ADC by reaction of the reduced thiols does not re-form a bond, as illustrated in the general scheme below:

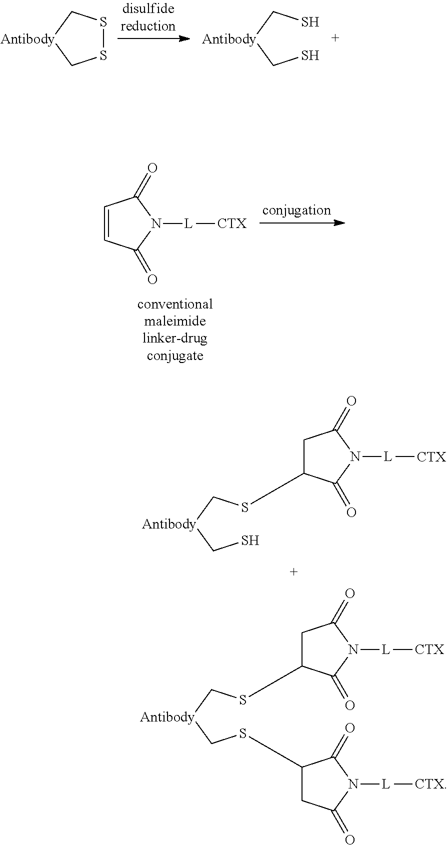

However, the bifunctional pyrrole-2,5-dione- and pyrrolidine-2,5-dione-based linkers of this invention contain two reactive functional groups (X in the scheme below) that react with the two sulfur atoms of an opened cysteine-cysteine disulfide bond. Reaction of the bifunctional linker with the two cysteines gives a “stapled” or “snapped” dithiosuccinimide or dithiomaleimide antibody conjugate with one linker per disulfide connected through two thioether bonds, as shown in the scheme below (double bond absent from the ring: succinimide linkers of formulae AA and AAA; double bond present in the ring: maleimide linkers of formulae BB and BBB).

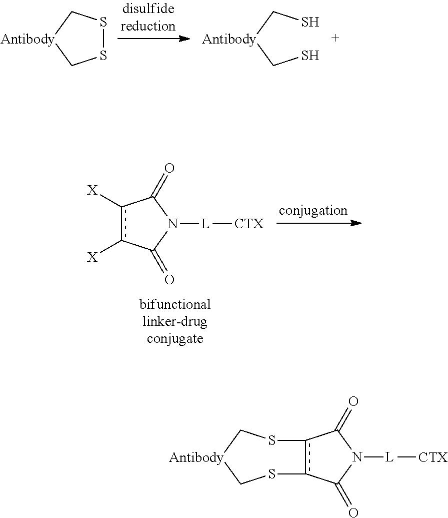

Unlike conventional methods for cysteine conjugation, the reaction re-forms a covalently bonded structure between the 2 cysteine sulfur atoms and therefore does not compromise the overall stability of the antibody. The method also enables conjugation of an optimal 4 drugs per antibody to afford a homogeneous ADC since the reactive cysteines are used. The overall result is replacement of a relatively labile disulfide with a stable “staple” or “snapp” between the cysteines. The monosubstituted maleimide linkers (formulae CC and CCC) are also effectively bifunctional in conjugation with the antibody because the double bond of the maleimide is capable of conjugation to one of the cysteine sulfur atoms and the X group with the other.

### Preparation of the Compounds of the Invention

The compounds of the invention, such as ADCs, linker-cytotoxin conjugates, linkers, and tubulysins, are prepared by conventional methods of organic and bio-organic chemistry. See, for example, Larock, “Comprehensive Organic Transformations”, Wiley-VCH, New York, N.Y., U.S.A. Suitable protective groups and their methods of addition and removal, where appropriate, are described in Greene et al., “Protective Groups in Organic Synthesis”, 2^{nd} ed., 1991, John Wiley and Sons, New York, N.Y., US. Reference may also be made to the documents referred to elsewhere in the application, such as to the Schumacher et al. article referred to earlier for the synthesis of linkers, US Patent Application Publication No. US 2011/0021568 A1 for the preparation of tubulysins, etc.

### Preparation of the Tubulysins

Tubulysins T3 and T4, CTX-I′, CTX-II′, CTX-III′, CTX-IV′, CTX-V′, CTX-VI′, CTX-VII′ and CTX-VIII′, are prepared by methods analogous to those of Peltier et al. and US Patent Application Publication No. US 2011/0021568 A1, by substituting D-pipecolinic acid for the D-N-methylpipecolinic acid, protecting and deprotecting if appropriate. Tubulysin analogues may be prepared using conventional synthetic procedures known in the art, such as those described by Larock, above.

### Preparation of the Linkers

The comparator MC linker is prepared by methods known to the art for its preparation.

Linkers of this invention are prepared by methods analogous to those of Schumacher et al., as follows (in this reaction scheme, R, L and Z have the meanings given them in the discussion of the fifth and sixth aspects of the invention above):

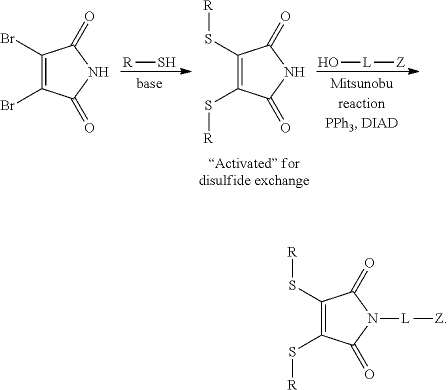

2,3-Dibromomaleimide, 1 equivalent, and a base such as sodium bicarbonate, about 5 equivalents, are dissolved in methanol, and a solution of 2-pyridinethiol, slightly more than 1 equivalent, in methanol, is added. The reaction is stirred for 15 min at ambient temperature. The solvent is removed under vacuum and the residue is purified, such as by flash chromatography on silica gel (petroleum ether:ethyl acetate, gradient elution from 9:1 to 7:3, to give 3,4-bis(2-pyridylsulfanyl)pyrrole-2,5-dione.

The coupling of the 3,4-bis(2-pyridylsulfanyl)pyrrole-2,5-dione with the sidechain is performed under strictly dry conditions. To the 3,4-bis(2-pyridylsulfanyl)pyrrole-2,5-dione, 1 equivalent, and triphenylphosphine, 1 equivalent, in a mixture of tetrahydrofuran and dichloromethane, is added dropwise DIAD, 1 equivalent, at −78° C. The reaction is stirred for 5 min and the sidechain, 0.5 equivalent, in dichloromethane is added dropwise. After stirring for 5 min, neopentyl alcohol, 1 equivalent, in tetrahydrofuran and dichloromethane is added, and stirred for a further 5 min, then the 3,4-bis(2-pyridylsulfanyl)pyrrole-2,5-dione, 1 equivalent, is added and stirred for another 5 min. The reaction is allowed to warm to ambient temperature with stirring for 20 hr, then the solvents are removed under vacuum. The residue is purified, such as by flash chromatography on silica gel (methanol: dichloromethane, gradient elution from 0-10% methanol), to give the linker. The sidechain may be used in protected form, and deprotected following the Mitsunobu reaction, if appropriate.

Alternatively, the sidechain, optionally protected if appropriate, may be coupled to a 3,4-dibromomaleimide by Mitsunobu coupling; and the resulting compound activated for disulfide exchange by reaction with an R-thiol in the presence of base; in the reverse of the synthesis described in the two previous paragraphs.

A similar method may be used for linkers containing the pyrrolidine-2,5-dione moiety rather than the pyrrole-2,5-dione moiety shown above, by starting with 2,3-dibromosuccinimide; but more usually these linkers are prepared by preparing the linker with an unsubstituted maleimide and brominating the linker to give the dibromosuccinimide moiety after coupling with the sidechain, and then “activating” the linker with the R-thiol as a last step.

Mono-substituted maleimide linkers are conveniently prepared by dehydrobromination of the dibromosuccinimide linkers under basic conditions, and related methods.

### Preparation of the Linker-Cytotoxin Conjugates

Linker-cytotoxin conjugates may be prepared by methods analogous to those of Doronina et al., Bioconjugate Chem. 2006, 17, 114-124, and similar documents. The linker, 1 equivalent, and HATU, 1 equivalent, are dissolved in anhydrous DMF, followed by the addition of DIPEA, 2 equivalents. The resulting solution is added to the cytotoxin, 0.5 equivalents, dissolved in DMF, and the reaction stirred at ambient temperature for 3 hr. The linker-cytotoxin conjugate is purified by reverse phase HPLC on a C-18 column

### Preparation of ADCs

Antibodies, typically monoclonal antibodies are raised against a specific cancer target (antigen), and purified and characterized. Therapeutic ADCs containing that antibody are prepared by standard methods for cysteine conjugation, such as by methods analogous to those of Hamblett et al., “Effects of Drug Loading on the Antitumor Activity of a Monoclonal Antibody Drug Conjugate”, Clin. Cancer Res. 2004, 10, 7063-7070; Doronina et al., “Development of potent and highly efficacious monoclonal antibody auristatin conjugates for cancer therapy”, Nat. Biotechnol., 2003, 21(7), 778-784; and Francisco et al., “cAC10-vcMMAE, an anti-CD30-monomethylauristatin E conjugate with potent and selective antitumor activity”, Blood, 2003, 102, 1458-1465. Antibody-drug conjugates with four drugs per antibody are prepared by partial reduction of the antibody with an excess of a reducing reagent such as DTT or TCEP at 37° C. for 30 min, then the buffer exchanged by elution through SEPHADEX® G-25 resin with 1 mM DTPA in DPBS. The eluent is diluted with further DPBS, and the thiol concentration of the antibody may be measured using 5,5′-dithiobis(2-nitrobenzoic acid) [Ellman's reagent]. An excess, for example 5-fold, of the linker-cytotoxin conjugate is added at 4° C. for 1 hr, and the conjugation reaction may be quenched by addition of a substantial excess, for example 20-fold, of cysteine. The resulting ADC mixture may be purified on SEPHADEX G-25 equilibrated in PBS to remove unreacted linker-cytotoxin conjugate, desalted if desired, and purified by size-exclusion chromatography. The resulting ADC may then be then sterile filtered, for example, through a 0.2 μM filter, and lyophilized if desired for storage.

The formation of an ADC of this invention is illustrated by the reaction scheme below, where the “Y”-shaped structure denotes the antibody, only one disulfide bond is shown, and details of the linker-cytotoxin conjugate are omitted for simplicity in showing the concept of the ADC.

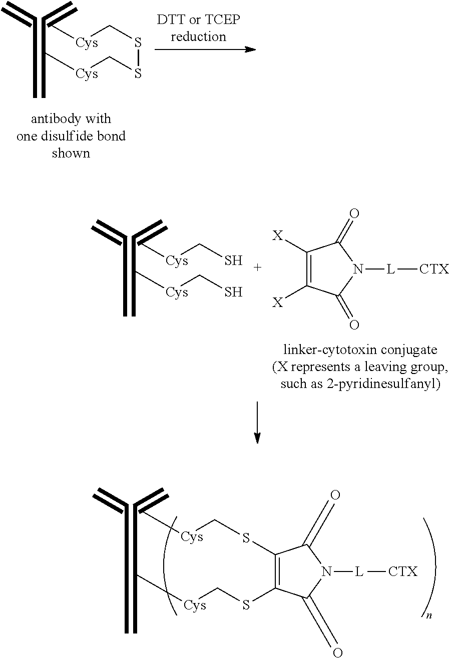

Typically, n will be 4, where all of the reactive cysteine disulfide bonds are replaced by linker-drug conjugates.

### The Antibody-Drug Conjugates (ADC) of the Present Application:

In one embodiment, there is provided an ADC of the formula:

A [symbol] PD-LCTX)ₘ]ₙ,

wherein A is an antibody, the double bond (═) represents bonds from the 3- and 4-positions of the PD wherein PD is a pyrrole-2,5-dione or derivative thereof, a pyrrolidine-2,5-dione or derivative thereof; L is a linker as defined herein, and CTX is a cytotoxin bonded to L.

### The Antibody (A):

In one embodiment, the antibody (A) is a monoclonal antibody or a humanized antibody. In another embodiment, the antibody is specific to a cancer antigen. In another embodiment, the antibody employed in the ADC of the present application is selected from the group consisting of alemtuzumab, bevacizumab, cetuximab, ipilimumab, ofatumumab, anitumumab, rituximab, tositumomab, inotuzumab, glembatumumab, lovortuzumab, milatuzumab and trastuzumab.

### The PD Group:

In one embodiment, PD is a pyrrole-2,5-dione or derivative thereof, a pyrrolidine-2,5-dione or derivative thereof. In another embodiment, the PD group is selected from the group consisting of:

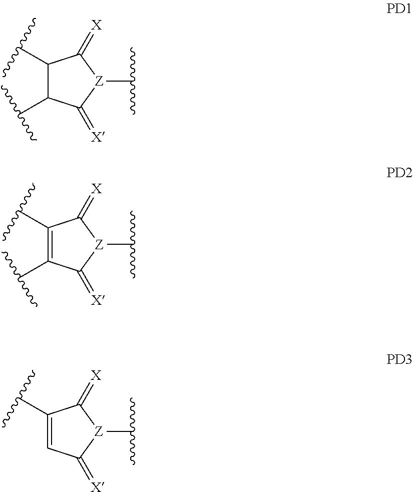

where:

X is O, S or NR¹ where R¹ is H or C₁₋₃alkyl;

X′ is O, S or NR² where R² is H or C₁₋₃alkyl; and

Z is selected from the group consisting of N—, CH—, CR³— and CR³—CR⁴R⁵— where R³, R⁴ and

R⁵ are each independently H or C₁₋₃alkyl.

In another embodiment of the PD group PD1, PD2 or PD3, X and X′ are 0, and Z is N. In another embodiment of the PD group, X and X′ are S, and Z is N. In another embodiment of the PD group, X and X′ are NCH₃, and Z is N. In another embodiment of the PD group, X and X′ are 0, and Z is CH—. In another embodiment of the PD group, X and X′ are S, and Z is CH—. In another embodiment of the PD group, X and X′ are NCH₃, and Z is CH—.

In one aspect of the above ADC, when PD is a pyrrole-2,5-dione or a pyrrolidine-2,5-dione, L is —(CH₂)ₚ— or —(CH₂CH₂O)ₚCH₂CH₂— and then L is not attached to CTX by an amide bond.

### The Linker L:

In one embodiment, there is provided an antibody-drug conjugate (ADC) of the formula:

A [symbol] PD-(L¹)ₐ-(L²)_{b}-(L³)_{c}CTX)ₘ]ₙ

wherein A is an antibody, PD is a pyrrole-2,5-dione or derivative thereof, a pyrrolidine-2,5-dione or derivative thereof; CTX is a cytotoxin;

each L¹, L² and L³ is independently a linker selected from the group consisting of —O—, —C(O)—, —S—, —S(O)—, —S(O)₂—, —NH—, —NCH₃—, —(CH₂)_{q}—, —NH(CH₂)₂NH—, —OC(O)—, —CO₂—, —NHCH₂CH₂C(O)—, —C(O)NHCH₂CH₂NH—, —NHCH₂C(O)—, —NHC(O)—, —C(O)NH—, —NCH₃C(O)—, —C(O)NCH₃—, —(CH₂CH₂O)ₚ—, —(CH₂CH₂O)ₚCH₂CH₂—, —CH₂CH₂—(CH₂CH₂O)ₚ—, —OCH(CH₂O—)₂—, cyclopentanyl, cyclohexanyl, unsubstituted phenylenyl, phenylenyl substituted by 1 or 2 substituents selected from the group consisting of halo, CF₃—, CF₃O—, CH₃O—, —C(O)OH, —C(O)OC₁₋₃alkyl, —C(O)CH₃, —CN, —NH—, —NH₂, —O—, —OH, —NHCH₃, —N(CH₃)₂, —C₁₋₃alkyl and -(AA)ᵣ-;

a, b and c are each independently 0, 1, 2 or 3, provided that at least one of a, b or c is 1;

each p is independently an integer of 1 to 14;

each q is independently an integer from 1 to 12;

each AA is independently an amino acid;

each r is 1 to 12; and

m is an integer of 1 to 4; and n is an integer of 1 to 4; with the proviso that when -(L¹)ₐ-(L²)_{b}-(L³)_{c}- together is —(CH₂)₁₋₁₂— or —(CH₂CH₂O)₁₋₁₂CH₂CH₂— then L¹, L² and L³ are not bonded to CTX by an amide bond.

In another embodiment of the above ADC, each L¹, L² and L³ is independently selected from the group consisting of —(CH₂)_{q}—, —NH(CH₂)₂NH—, —OC(O)—, —CO₂—, NHCH₂CH₂C(O)—, —C(O)NHCH₂CH₂NH—, —C(O)NHCH₂CH₂—, —NHCH₂C(O)—, —NHC(O)—, —C(O)NH—, —NCH₃C(O)—, —C(O)NCH₃—, —C(O)CH₂CH₂—, —(CH₂CH₂O)ₚ—, —(OCH₂CH₂)ₚ—, —(CH₂CH₂O)ₚCH₂CH₂—, —CH₂CH₂—(CH₂CH₂O)ₚ—, —OCH₂(p-C₆H₄)—NH—, —OCH₂(o-C₆H₄)—NH—, —NH-(p-C₆H₄)—CH₂O—, —NH-(o-C₆H₄)—CH₂O—, —OCH(CH₂O—)₂— and -(AA)ᵣ-; a, b and c are each independently 0, 1 or 2; each p, q and r is independently 1, 2, 3 or 4; m is 1; and n is an integer of 1 to 4. In another embodiment of the ADC of the present application, the linker is attached to the CTX by a group selected from the group consisting of NHC(O)—, —NHC(O)O—, —N(C₁₋₃alkyl)C(O)O—, —NH—, —N(C₁₋₃alkyl)-, —N(C₁₋₃alkyl)C(O)NH— and —N(C₁₋₃alkyl)C(O)N(C₁₋₃alkyl)-.

In another embodiment of the above ADC, each L¹, L² and L³ is independently selected from the group consisting of (CH₂)_{q}—, —NH(CH₂)₂NH—, —OC(O)—, —OC₂—, —NHCH₂CH₂C(O)—, —C(O)NHCH₂CH₂NH—, —NHCH₂C(O)—, —NHC(O)—, —C(O)NH—, —NCH₃C(O)—, —OCH(CH₂O—)₂— and —C(O)NCH₃—; a, b and c are each independently 0, 1 or 2; each p and q is independently 1 or 2; m is 1; and n is an integer of 1 to 4.

In another embodiment of the above ADC, each L¹, L² and L³ is independently selected from the group consisting of —NH(CH₂)₂NH—, —NHCH₂CH₂C(O)—, —C(O)NHCH₂CH₂NH—, —NHCH₂C(O)—, —NHC(O)—, —C(O)NH—, —NCH₃C(O)—, —OCH(CH₂O—)₂— and —C(O)NCH₃—; a, b and c are each independently 0 or 1; m is 1; and n is an integer of 1 to 4.

In another embodiment of the above ADC, each L¹, L² and L³ is independently selected from the group consisting of —NHC(O)—, —C(O)NH—, —(CH₂CH₂O)ₚ—, —(CH₂CH₂O)ₚCH₂CH₂—, —CH₂CH₂—(CH₂CH₂O)ₚ—, —OCH(CH₂O—)₂— and -(AA)ᵣ-; a, b and c are each independently 0 or 1; each p and r is independently 1, 2 or 3; m is 1; and n is an integer of 1 to 4.

In another embodiment of the above ADC, each AA is an amino acid selected from the group consisting of Ala, Arg, Asn, Asp, Cit, Cys, Glu, Gln, Gly, His, Ile, Lys, Met, Phe, Pro, Ser, Thr, Trp, Tyr and Val. In one variation of the above, (AA)ᵣ is a single amino acid selected from the group consisting of Cit, Gly, Arg, Val, Ala, Cys, Gln, Leu, Ile, Lys and Ser or their N-methylated analogues. In another variation of the above, (AA)ᵣ is selected from the group consisting of Ala-Val, Val-Ala, Gly-Gly, Gly-Arg, Gly-Val, Gly-Ala, Gly-Cys, Gly-Gln, Gly-Ile, Lys-Leu, Gly-Lys, Val-Arg, Ala-Cit, Val-Cit and Gly-Ser or their N-methylated analogues.

In another variation of the above, (AA)ᵣ is selected from the group consisting of Gly-Gly-Gly, Gly-Arg-Gly, Gly-Val-Gly, Gly-Ala-Gly, Gly-Cys-Gly, Gly-Gln-Gly, Gly-Ile-Gly, Lys-Leu-Gly, Gly-Lys-Gly and Gly-Ser-Gly or their N-methylated analogues. In another variation of the above, (AA)ᵣ is selected from the group consisting of Ala-Ala, Ala-Gly, Ala-Arg, Ala-Val, Ala-Ala, Ala-Cys, Ala-Gln, Ala-Ile, Ala-Leu, Ala-Lys, Ala-Cit and Ala-Ser or their N-methylated analogues. In another variation of the above, (AA), is selected from the group consisting of Ala-Ala-Ala, Ala-Gly-ALa, Ala-Arg-Ala, Ala-Val-Ala, Ala-Ala-Ala, Ala-Cys-Ala, Ala-Gln-Ala, Ala-Ile-Ala, Ala-Leu-Ala, Ala-Lys-Ala and Ala-Ser-Ala or their N-methylated analogues.

### The CTX Residue:

In one embodiment, the CTX residue is a tubulysin residue of the formula T3 or T4:

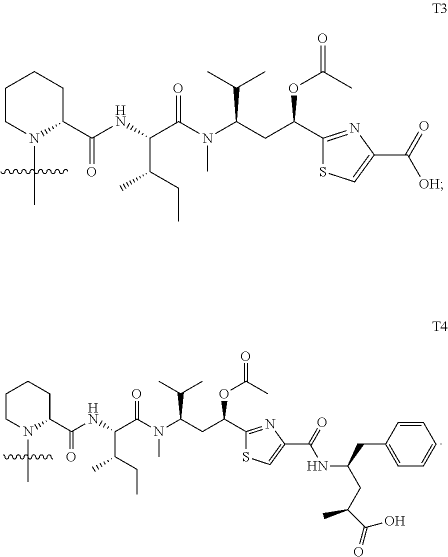 [SMILES: *N1CCCC[C@@H]1C(=O)N[C@H](C(=O)N(C)[C@H](C[C@@H](OC(C)=O)c1nc(C(=O)O)cs1)C(C)C)[C@@H](C)CC ; *N1CCCC[C@@H]1C(=O)N[C@H](C(=O)N(C)[C@H](C[C@@H](OC(C)=O)c1nc(C(=O)N[C@@H](Cc2ccccc2)C[C@H](C)C(=O)O)cs1)C(C)C)[C@@H](C)CC]

In one embodiment, the CTX residue comprises the formula:

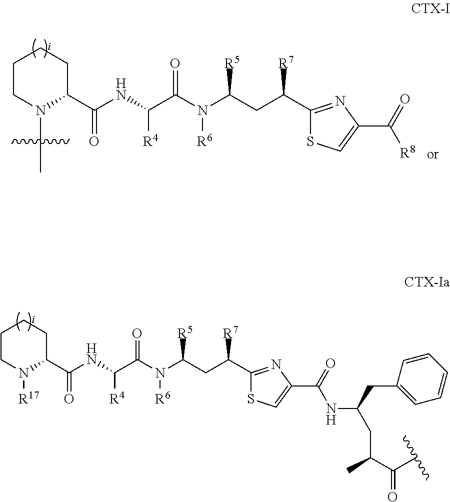

wherein:

i is 0 or 1;

R⁴ is a C₁₋₆alkyl; R⁵ is a C₁₋₆alkyl; R⁶ is C₁₋₆alkyl;

R⁷ is selected from the group consisting of C₁₋₆alkyl, —OC₁₋₆alkyl, —OC(O)C₁₋₆alkyl, OC(O)NHC₁₋₆alkyl and —OC(O)NHC₆₋₁₀aryl; and

R⁸ is selected from the group consisting of OH, —OC₁₋₆alkyl, —CO₂C₁₋₆alkyl, —CO₂C₆₋₁₀aryl, —CH(C₁₋₆alkyl)CO₂R^{c}, —CH(C₆₋₁₀aryl)CO₂R^{c}, —NH—CH(C₅H₆)₂, —NHC₁₋₆alkyl, —NH(CH₂)₃—CO₂R^{c}, —NH(CH₂CH₂)₂C₆₋₁₀aryl, —NHCH(CH₂C₆₋₁₀aryl)CH₂CH(CH₃)CO₂R^{c} and —NHCH(CH₂CO₂R^{c})CH₂-p-C₆H₄—NHC₁₋₆alkyl; where each R^{c} is independently H or C₁₋₆alkyl; and R¹⁷ is selected from the group consisting of H, —CH₃ and —C(O)CH₃.

In one embodiment, the CTX residue comprises the formula:

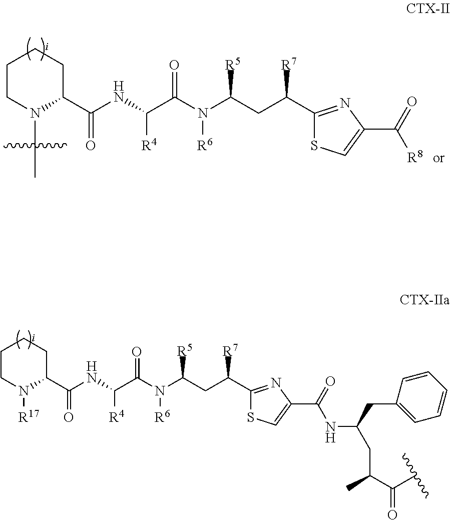

wherein:

i is 0 or 1;

R⁴ is a C₁₋₆alkyl; R⁵ is a C₁₋₆alkyl;

R⁶ is selected from the group consisting of C₁₋₆alkyl, C₆₋₁₀aryl;

R⁷ is selected from the group consisting of C₁₋₆alkyl, —OC₁₋₆alkyl, —NHC(O)C₁₋₆alkyl, —OC(O)C₁₋₆alkyl, —OC(O)C₆₋₁₀aryl, —OC(O)NHC₁₋₆alkyl and —C(O)NHC₆₋₁₀aryl; and

R⁸ is selected from the group consisting of OH, —OC₁₋₆alkyl, —CO₂C₁₋₆alkyl, —CO₂C₆₋₁₀aryl, —CH(C₁₋₆alkyl)CO₂R^{c}, —CH(C₆₋₁₀aryl)CO₂R^{c}, —NH—CH(C₅H₆)₂, —NHC₁₋₆alkyl, —NH(CH₂)₃—CO₂R^{c}, —NH(CH₂CH₂)₂C₆₋₁₀aryl, —NHCH(CH₂C₆₋₁₀aryl)CH₂CH(CH₃)CO₂R^{c}, —NHCH(CO₂R^{c})CH₂-p-C₆H₄—NH₂, —NHCH(CO₂R^{c})CH₂-p-C₆H₄—NHC₁₋₆alkyl and —NHCH(CH₂CO₂R^{c})CH₂-p-C₆H₄—NHC₁₋₆alkyl;

where each R^{c} is independently selected from the group consisting of H, C₁₋₆alkyl and C₆₋₁₀aryl, and R¹⁷ is selected from the group consisting of H, —CH₃ and —C(O)CH₃.

In one embodiment, the CTX residue comprises the formula:

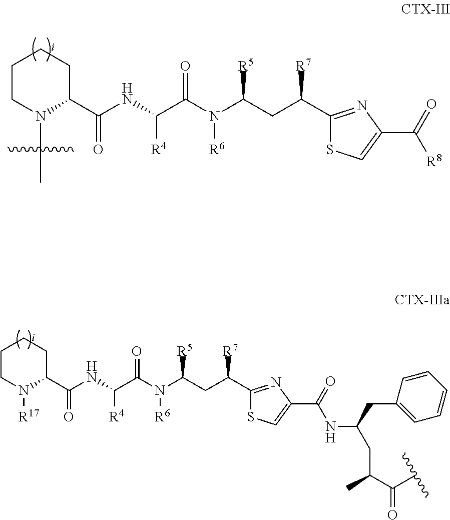

wherein: i is 0 or 1; R⁴ is a C₁₋₆alkyl or C₆₋₁₀aryl;

R⁵ is a C₁₋₆alkyl or C₆₋₁₀aryl;

R⁶ is selected from the group consisting of C₁₋₆alkyl-Y, —C₆₋₁₀aryl-Y, —CH₂OCOC₁₋₆alkyl-Y, —C₆₋₁₂aryl-Y, —CH₂CO₂C₁₋₆alkyl-Y, —CH₂CONHC₁₋₆alkyl-Y, —CO₂C₁₋₆alkyl-Y, —CH(—CO₂H)(C₁₋₆alkyl)-Y, —CH(—CO₂C₁₋₃alkyl)(C₁₋₆alkyl)-Y and —CH(C₁₋₆alkyl)CO₂C₁₋₆alkyl-Y, wherein Y is H or is selected from the group consisting of —NH₂, —OH, —SH and —COOH wherein, with the exception where Y is H, Y is optionally attached to the linker L¹, L² and/or L³;

R⁷ is selected from the group consisting of C₁₋₆alkyl, —OC₁₋₆alkyl, —NHC(O)C₁₋₆alkyl, —OC(O)C₁₋₆alkyl, —OC(O)C₆₋₁₀aryl, —OC(O)NHC₁₋₆alkyl and —OC(O)NHC₆₋₁₀aryl; or R⁷ is a bond to the linker L¹, L² and/or L³; and

R⁸ is selected from the group consisting of —OH, —OC₁₋₆alkyl, —CO₂C₁₋₆alkyl, —CO₂C₆₋₁₀aryl, —CH(C₁₋₆alkyl)CO₂R^{c}, —CH(C₆₋₁₀aryl)CO₂R^{c}, —NH—CH(C₅H₆)₂, —NHC₁₋₆alkyl, —NH(CH₂)₃—CO₂R^{c}, —NH(CH₂CH₂)₂C₆₋₁₀aryl, —NHCH(CH₂C₆₋₁₀aryl)CH₂CH(CH₃)CO₂R^{c}, —NHCH(CH₂CH(CH₃)COOR^{c})CH₂-p-C₆H₄—NHC(O)CH(NHC(O)(CH₂)₅NHR^{c})(CH₂)₄NHRc, —NHCH(CO₂R^{c})CH₂-p-C₆H₄—NH₂, —NHCH(CO₂R^{c})CH₂-p-C₆H₄—NHC₁₋₆alkyl, —NHCH(CH₂CO₂R^{c})CH₂-p-C₆H₄—NHC₁₋₆alkyl, —NHCH(CH₂CH₂CO₂R^{c})CH₂-p-C₆H₄—NHC₁₋₆alkyl and —NHCH(CH₂CH(CH₃)CO₂R^{c})CH₂-p-C₆H₄—NHC₁₋₆alkyl; where each R^{c} is independently selected from the group consisting of H, C₁₋₆alkyl and C₆₋₁₀aryl; and R¹⁷ is selected from the group consisting of H, —CH₃ and —C(O)CH₃. In one embodiment, as provided herein, where R⁷ is a bond to the linker L (or -(L¹)ₐ-(L²)₆-(L³)_{c}-), then the CTX is bonded to the linker from both at the squiggly line (˜) and at the bond that is R⁷; or the CTX is bonded to the linker only from the bond that is R⁷ and not on the squiggly line bond at the amine nitrogen of the CTX and the squiggly line is bonded to hydrogen.

In one aspect, there is provided the CTX residue of the formula CTX-III or CTX-IIIa:

where i is 1; R⁴ is a C₁₋₆alkyl; R⁵ is a C₁₋₃alkyl;

R⁶ is selected from the group consisting of C₁₋₃alkyl, —CH₂OCOC₁₋₃alkyl, —CH₂CO₂C₁₋₃alkyl, —CH₂CONHC₁₋₃alkyl, —CH(C₁₋₃alkyl)CO₂H and —CH(C₁₋₃alkyl)CO₂C₁₋₃alkyl;

R⁷ is selected from the group consisting of —OC₁₋₃alkyl, —NHC(O)C₁₋₃alkyl, —OC(O)C₁₋₃alkyl, —OC(O)-phenyl, —OC(O)NHC₁₋₆alkyl and —OC(O)NHC₆₋₁₀aryl; and

for CTX-III, R⁸ is selected from the group consisting of —NH(CH₂CH₂)₂-phenyl, —NHCH(CH₂-phenyl)CH₂CH(CH₃)CO₂R^{c}, —NHCH(CO₂R^{c})CH₂-p-C₆H₄—NHC₁₋₃alkyl, —NHCH(CH₂CO₂R^{c})CH₂-p-C₆H₄—NHC₁₋₃alkyl, —NHCH(CH₂CH₂CO₂R^{c})CH₂-p-C₆H₄—NHC₁₋₃alkyl and —NHCH(CH₂CH(CH₃)CO₂R^{c})CH₂-p-C₆H₄—NHC₁₋₃alkyl; and wherein R^{c} is H or C₁₋₃alkyl.

In one aspect, there is provided the CTX residue of the formula CTX-III or CTX-IIIa:

where i is 1; R⁴ is a C₁₋₆alkyl; R⁵ is a C₁₋₃alkyl;

R⁶ is selected from the group consisting of C₁₋₃alkyl, —CH₂CO₂C₁₋₃alkyl and CH(C₁₋₃alkyl)CO₂C₁₋₃alkyl;

R⁷ is selected from the group consisting of —OC₁₋₃alkyl, —NHC(O)C₁₋₃alkyl, —OC(O)C₁₋₃alkyl, —OC(O)-phenyl, —OC(O)NHC₁₋₆alkyl and —OC(O)NHC₆₋₁₀aryl; and

for CTX-III, R⁸ is selected from the group consisting of —NH(CH₂CH₂)₂-phenyl, —NHCH(CO₂R^{c})CH₂-p-C₆H₄—NHC₁₋₃alkyl and —NHCH(CH₂CH₂CO₂R^{c})CH₂-p-C₆H₄—NHC₁₋₃alkyl; and wherein R^{c} is H or C₁₋₃alkyl.

In another embodiment, the CTX residue comprises the formula:

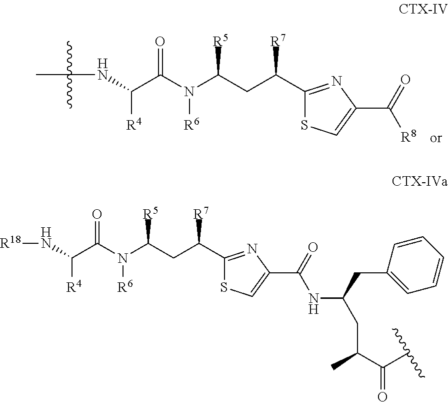

where:

R⁴ is a C₁₋₆alkyl or C₆₋₁₀aryl; R⁵ is a C₁₋₆alkyl or C₆₋₁₀aryl;

R⁶ is selected from the group consisting of C₁₋₆alkyl, C₆₋₁₀aryl, —CH₂OCOC₁₋₆alkyl, —CH₂CO₂C₁₋₆alkyl, —CH₂CONHC₁₋₆alkyl, —CO₂C₁₋₆alkyl, CH(C₁₋₆alkyl)CO₂H and CH(C₁₋₆alkyl)CO₂C₁₋₆alkyl;

R⁷ is selected from the group consisting of halo, C₁₋₆alkyl, —OC₁₋₆alkyl, —NHC(O)C₁₋₆alkyl, —OC(O)C₁₋₆alkyl, —OC(O)C₆₋₁₀aryl, —OC(O)NHC₁₋₆alkyl and —OC(O)NHC₆₋₁₀aryl; or R⁷ is a bond to the linker L¹, L² and/or L³; and

R⁸ is selected from the group consisting of OH, —OC₁₋₆alkyl, —CH(C₁₋₆alkyl)CO₂R^{c}, —CH(C₆₋₁₀aryl)CO₂R^{c}, —NH—CH(C₅H₆)₂, —NHC₁₋₆alkyl, —NH(CH₂)₃—CO₂R^{c}, —NH(CH₂CH₂)₂C₆₋₁₀aryl, —NHCH(CH₂C₆₋₁₀aryl)CH₂CH(CH₃)CO₂R^{c}, —NHCH(CH₂CH(CH₃)CO₂R^{c})CH₂-p-C₆H₄—NHC(O)CH(NHC(O)(CH₂)₅NHR^{c})(CH₂)₄NHR^{c}, —NHCH(CO₂R^{c})CH₂-p-C₆H₄, —NHCH(CO₂R^{c})CH₂-p-C₆H₄—NH₂, —NHCH(CH₂CO₂R^{c})CH₂-phenyl, —NHCH(CH₂CO₂R^{c})CH₂-p-C₆H₄—NH₂, —NHCH(CH₂CH₂CO₂R^{c})CH₂-phenyl, —NHCH(CH₂CH₂CO₂R^{c})CH₂-p-C₆H₄—NH₂, —NHCH(CH₂CH(CH₃)CO₂R^{c})CH₂-phenyl, —NHCH(CH₂CH(CH₃)CO₂R^{c})CH₂-p-C₆H₄—NH₂, —NHCH(CO₂R^{c})CH₂-p-C₆H₄—NH₂, —NHCH(CO₂R^{c})CH₂-p-C₆H₄—NHC₁₋₆alkyl, —NHCH(CH₂CO₂R^{c})CH₂-p-C₆H₄—NHC₁₋₆alkyl, —NHCH(CH₂CH₂CO₂R^{c})CH₂-p-C₆H₄—NHC₁₋₆alkyl and —NHCH(CH₂CH(CH₃)CO₂R^{c})CH₂-p-C₆H₄—NHC₁₋₆alkyl; wherein each R^{c} is independently selected from the group consisting of H, C₁₋₆alkyl and C₆₋₁₀aryl; and R¹⁸ is selected from the group consisting of H, —CH₃ and —C(O)CH₃.

In one aspect, there is provided the CTX residue of the formula CTX-IV or CTX-IVa:

wherein: R⁴ is a C₁₋₆alkyl; R⁵ is a C₁₋₆alkyl;

R⁶ is selected from the group consisting of C₁₋₃alkyl, —CH₂OCOC₁₋₃alkyl, —CH₂CO₂C₁₋₃alkyl, —CH₂CONHC₁₋₃alkyl and CH(C₁₋₆ alkyl)CO₂C₁₋₃alkyl;

R⁷ is selected from the group consisting of C₁₋₆alkyl, —OC₁₋₆alkyl, —NHC(O)C₁₋₃alkyl, —OC(O)C₁₋₃alkyl, —OC(O)phenyl, —OC(O)NHC₁₋₆alkyl and —OC(O)NHC₆₋₁₀aryl; and

R⁸ is selected from the group consisting of —NH—CH(C₅H₆)₂, —NHC₁₋₆alkyl, —NH(CH₂)₃—CO₂R^{c}, —NH(CH₂CH₂)₂-phenyl, —NHCH(CH₂-phenyl)CH₂CH(CH₃)CO₂R^{c}, —NHCH(CO₂R^{c})CH₂-phenyl, —NHCH(CH₂CO₂R^{c})CH₂-phenyl and —NHCH(CO₂R^{c})CH₂-p-C₆H₄—NHC₁₋₃alkyl; wherein each R^{c} is independently selected from the group consisting of H and C₁₋₃alkyl.

In another aspect, there is provided the CTX residue of the formula CTX-IV or CTX-IVa:

wherein: R⁴ is a C₁₋₆alkyl; R⁵ is a C₁₋₆alkyl; R⁶ is C₁₋₃alkyl;

R⁷ is selected from the group consisting of C₁₋₆alkyl, —OC₁₋₆alkyl and —OC(O)C₁₋₃alkyl; and

R⁸ is selected from the group consisting of —NH—CH(C₅H₆)₂, —NH(CH₂CH₂)₂-phenyl, —NHCH(CO₂R^{c})CH₂-phenyl and —NHCH(CO₂R^{c})CH₂-p-C₆H₄—NHC₁₋₃alkyl; wherein each R^{c} is independently selected from the group consisting of H and C₁₋₃alkyl.

In another embodiment, the CTX residue comprises the structure:

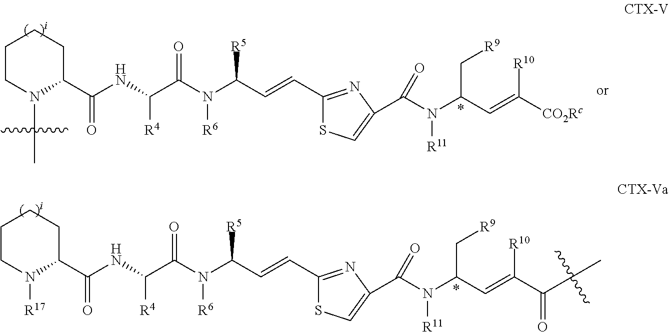

wherein:

R⁴ is a C₁₋₆alkyl or C₆₋₁₀aryl; R⁵ is a C₁₋₆alkyl or C₆₋₁₀aryl;

R⁶ is H or is selected from the group consisting of C₁₋₆alkyl, C₆₋₁₀aryl, —CH₂OCOC₁₋₆alkyl, —CH₂CO₂C₁₋₆alkyl, —CH₂CONHC₁₋₆alkyl, —CO₂C₁₋₆alkyl, —CH(C₁₋₆alkyl)CO₂H and —CH(C₁₋₆alkyl)CO₂C₁₋₆alkyl;

R⁹ is selected from the group consisting C₁₋₆alkyl, -phenyl, 1-naphthyl and 2-napthyl, wherein each -phenyl, 1-naphthyl and 2-naphthyl group is unsubstituted or substituted by 1 or 2 substituents selected from the group consisting of halo, cyano, nitro, CF₃—, CF₃O—, CH₃O—, —C(O)CH₃, —NH₂, —OH, —SH, —NHCH₃, —N(CH₃)₂, —SMe and C₁₋₃alkyl; and

R¹⁰ is selected from the group consisting of C₁₋₃alkyl, C₂₋₆alkenyl, —O—C₁₋₃alkyl and —OC₆₋₁₀aryl; R¹¹ is H or C₁₋₃alkyl;

wherein R^{c} is selected from the group consisting of H, C₁₋₆alkyl and C₆₋₁₀aryl; and

wherein * designates an R chiral center, an S chiral center or a mixture of R and S isomers.

In one aspect, there is provided the CTX residue of the formula CTX-V or CTX-Va wherein: R⁴ is a C₁₋₃alkyl; R⁵ is a C₁₋₃alkyl;

R⁶ is selected from the group consisting of C₁₋₃alkyl, —CH₂OCOC₁₋₃alkyl, —CH₂CO₂C₁₋₃alkyl, —CO₂C₁₋₃alkyl and CH(C₁₋₃alkyl)CO₂C₁₋₃alkyl;

R⁹ is selected from the group consisting C₁₋₆alkyl, -phenyl, 1-naphthyl and 2-napthyl, wherein each -phenyl, 1-naphthyl and 2-naphthyl is unsubstituted or substituted by 1 or 2 substituents selected from the group consisting of CF₃—, CH₃O—, —C(O)CH₃, —NHCH₃, —N(CH₃)₂ and C₁₋₃alkyl;

R¹⁰ is selected from the group consisting of C₁₋₃alkyl, C₂₋₆alkenyl, —O—C₁₋₃alkyl and —O-phenyl; and R¹⁷ is selected from the group consisting of H, —CH₃ and —C(O)CH₃.

In another aspect, there is provided the CTX residue of the formula CTX-V or CTX-Va:

wherein: R⁴ is a C₁₋₃alkyl; R⁵ is a C₁₋₃alkyl; R⁶ is C₁₋₃alkyl;

R⁹ is selected from the group consisting C₁₋₆alkyl, -phenyl, 1-naphthyl and 2-napthyl; and

R¹⁰ is selected from the group consisting of C₁₋₃alkyl and C₂₋₆alkenyl.

In another embodiment, the CTX residue comprises the formula:

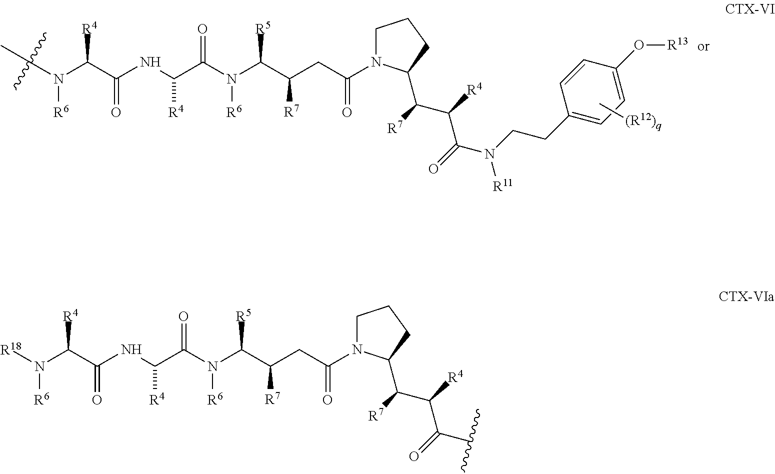

wherein:

each R⁴ is independently a C₁₋₆alkyl or C₆₋₁₀aryl;

R⁵ is a C₁₋₆alkyl or C₆₋₁₀aryl;

each R⁶ is independently selected from the group consisting of H, C₁₋₆alkyl, C₆₋₁₀aryl, —CH₂OCOC₁₋₆alkyl, —CH₂CO₂C₁₋₆alkyl, —CH₂CONHC₁₋₆alkyl, —CO₂C₁₋₆alkyl, —CH(C₁₋₆alkyl)CO₂H and —CH(C₁₋₆alkyl)CO₂C₁₋₆alkyl;

each R⁷ is independently selected from the group consisting of —CN, —OC₁₋₆alkyl, C₁₋₆alkyl, —NHC(O)C₁₋₆alkyl, —OC(O)C₁₋₆alkyl, —OC(O)C₆₋₁₀aryl, —OC(O)NHC₁₋₆alkyl and —OC(O)NHC₆₋₁₀aryl;

R¹¹ is H or C₁₋₃alkyl;

each R¹² is independently selected from the group consisting of halo, cyano, nitro, CF₃—, CF₃O—, CH₃O—, —CO₂H, —NH₂, —OH, —SH, —NHCH₃, —N(CH₃)₂, —SMe, C₁₋₃alkyl and C₆₋₁₀aryl;

R¹³ is H or is selected from the group consisting of C₁₋₃alkyl, —CF₃, —C₁₋₃alkyl-phenyl and C₆₋₁₀aryl;

R¹⁸ is selected from the group consisting of H, —CH₃ and —C(O)CH₃; and q is 0, 1 or 2.

In one aspect, there is provided the CTX residue of the formula CTX-VI or CTX-VIa: wherein: each R⁴ is independently a C₁₋₃alkyl; R⁵ is a C₁₋₃alkyl;

each R⁶ is independently selected from the group consisting of H, C₁₋₆alkyl, —CH₂OCOC₁₋₆ alkyl, —CH₂CO₂C₁₋₃alkyl, CH(C₁₋₃alkyl)CO₂H and CH(C₁₋₃ alkyl)CO₂C₁₋₃alkyl;

each R⁷ is independently selected from the group consisting of —OC₁₋₃alkyl, C₁₋₃alkyl, —NHC(O)C₁₋₃alkyl, —OC(O)C₁₋₃alkyl and —OC(O)C₆₋₁₀aryl; R″ is H or C₁₋₃alkyl;

each R¹² is independently selected from the group consisting of halo, CF₃—, CF₃O—, CH₃O—, —NHCH₃, —N(CH₃)₂, and C₁₋₃alkyl;

R¹³ is H or is selected from the group consisting of C₁₋₃alkyl, —CF₃, —C₁₋₃alkyl-phenyl.

In another aspect, there is provided the CTX residue of the formula CTX-VI or CTX-VIa wherein: each R⁴ is independently a C₁₋₃alkyl; R⁵ is a C₁₋₃alkyl;

each R⁶ is independently H or C₁₋₆alkyl;

each R⁷ is independently selected from the group consisting of —OC₁₋₃alkyl, —OC(O)C₁₋₃alkyl, —OC(O)C₆₋₁₀aryl, —OC(O)NHC₁₋₆alkyl and —OC(O)NHC₆₋₁₀aryl; R¹¹ is H or C₁₋₃alkyl;

each R¹² is independently selected from the group consisting of CF₃O—, CH₃O— and C₁₋₃alkyl; and R¹³ is H or is selected from the group consisting of C₁₋₃alkyl, —CF₃, —C₁₋₃alkyl-phenyl.

In another embodiment, the CTX residue comprises the structure of the formula:

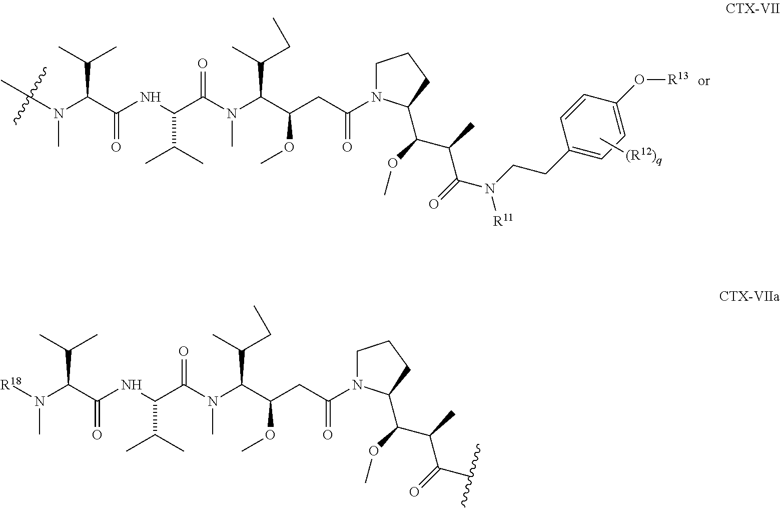

wherein:

R¹¹ is H or C₁₋₃alkyl;

each R¹² is independently selected from the group consisting of halo, cyano, nitro, CF₃—, CF₃O—, CH₃O—, —CO₂H, —NH₂, —OH, —SH, —NHCH₃, —N(CH₃)₂, —SMe, C₁₋₃alkyl and C₆₋₁₀aryl;

R¹³ is H or is selected from the group consisting of C₁₋₃alkyl, —CF₃, —C₁₋₂alkyl-phenyl and C₆₋₁₀aryl; and q is 0, 1 or 2.

In one aspect, there is provided the CTX residue of the formula CTX-VII: wherein: R¹¹ is H; R¹² is selected from the group consisting of CF₃—, CF₃O—, CH₃O—, —CO₂H, —NHCH₃, —N(CH₃)₂, —C₁₋₃alkyl and phenyl;

R¹³ is H or is selected from the group consisting of C₁₋₃alkyl, —C₁₋₂alkyl-phenyl and phenyl; R¹⁸ is selected from the group consisting of H, —CH₃ and —C(O)CH₃; and q is 1. In one aspect, there is provided the CTX residue of the formula CTX-VII: wherein: R¹¹ is H and R¹³ is H, C₁₋₃alkyl or —C₁₋₂alkyl-phenyl; and q is 0. In another embodiment, the CTX residue comprises the formula:

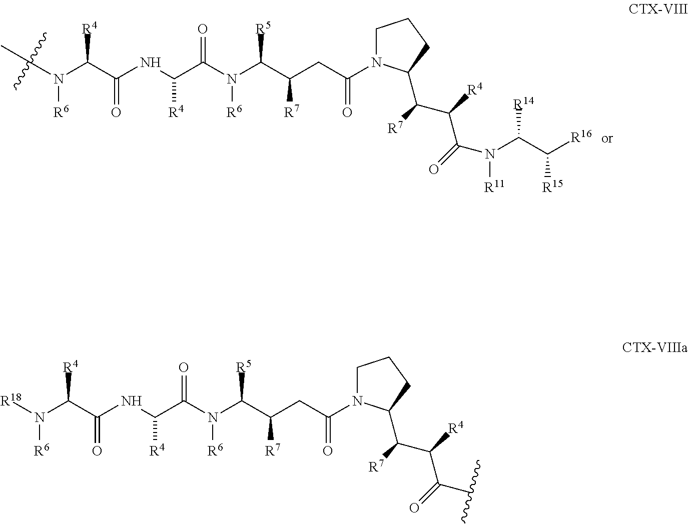

wherein:

each R⁴ is independently a C₁₋₆alkyl or C₆₋₁₀aryl; R⁵ is a C₁₋₆alkyl or C₆₋₁₀aryl;

each R⁶ is independently selected from the group consisting of H, C₁₋₆alkyl, C₆₋₁₀aryl, —CH₂OCOC₁₋₆alkyl, —CH₂CO₂C₁₋₆alkyl, —CH₂CONHC₁₋₆alkyl, —CO₂C₁₋₆alkyl, CH(C₁₋₆alkyl)CO₂H and CH(C₁₋₆alkyl)CO₂C₁₋₆alkyl;

each R⁷ is independently selected from the group consisting of —CN, —OC₁₋₆alkyl, C₁₋₆alkyl, —NHC(O)C₁₋₆alkyl, —OC(O)C₁₋₆alkyl, —OC(O)C₆₋₁₀aryl, —OC(O)NHC₁₋₆alkyl and —OC(O)NHC₆₋₁₀aryl;

R¹¹ is H or C₁₋₃alkyl; R¹⁴ is selected from the group consisting of C₁₋₃alkyl and C₆₋₁₀aryl;

R¹⁵ is H or is selected from the group consisting of —OH, NH₂, —NHCH₃, C₁₋₃alkyl, —OC₁₋₃alkyl and —OC₆₋₁₀aryl; R¹⁶ is selected from the group consisting of C₁₋₆alkyl, C₆₋₁₀aryl and heteroaryl; and R¹⁸ is selected from the group consisting of H, —CH₃ and —C(O)CH₃.

In one aspect, there is provided the CTX residue of the formula CTX-VIII or CTX-VIIIa, wherein: each R⁴ is independently a C₁₋₃alkyl; R⁵ is a C₁₋₃alkyl;

each R⁶ is independently selected from the group consisting of H, C₁₋₆alkyl, —CH₂OCOC₁₋₆ alkyl, —CH₂CO₂C₁₋₃ alkyl, CH(C₁₋₃alkyl)CO₂H and CH(C₁₋₃ alkyl)CO₂C₁₋₃ alkyl;

each R⁷ is independently selected from the group consisting of —OC₁₋₃alkyl, C₁₋₃alkyl, —NHC(O)C₁₋₃alkyl, —OC(O)C₁₋₃alkyl, —OC(O)C₆₋₁₀aryl, —OC(O)NHC₁₋₆alkyl and —OC(O)NHC₆₋₁₀aryl;

R¹¹ is H or C₁₋₃alkyl; R¹⁴ is C₁₋₃alkyl; R¹⁵ is H or is selected from the group consisting of OH, NH₂, —NHCH₃ and —OC₁₋₃alkyl; and R¹⁶ is C₆₋₁₀aryl.

In another aspect, there is provided the CTX residue of the formula CTX-VIII or CTX-VIIIa wherein: each R⁴ is independently a C₁₋₃alkyl; R⁵ is a C₁₋₃alkyl;

each R⁶ is independently H or C₁₋₆alkyl;

each R⁷ is independently selected from the group consisting of —OC₁₋₃alkyl, —OC(O)C₁₋₃alkyl, —OC(O)C₆₋₁₀aryl, —OC(O)NHC₁₋₆alkyl and —OC(O)NHC₆₋₁₀aryl;

R¹¹ is H or C₁₋₃alkyl; R¹⁴ is C₁₋₃alkyl; R¹⁵ is selected from the group consisting of —OH, NH₂ and —NHCH₃; and R¹⁶ is C₆₋₁₀aryl.

In one embodiment, there is provided the non-conjugated cytotoxins CTX-I′, CTX-II′, CTX-III′, CTX-IV′, CTX-V′, CTX-VI′, CTX-VII′ and CTX-VIII′ of the formulae:

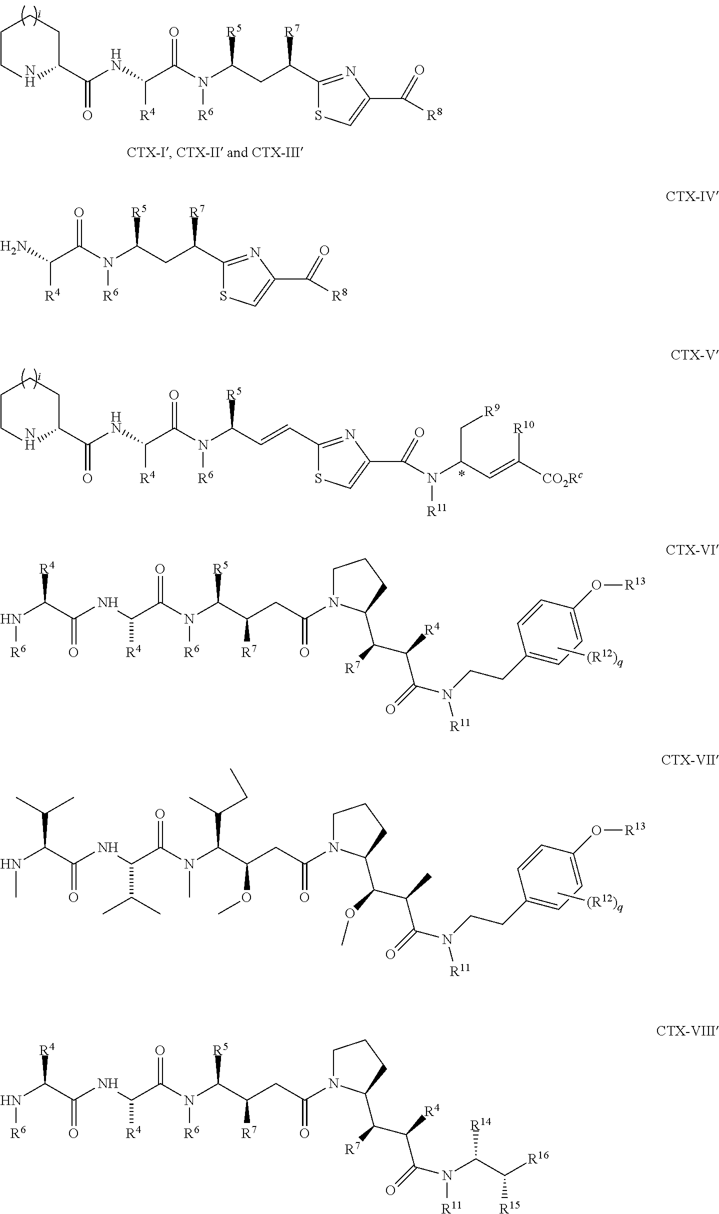

wherein the variables i, q, R⁴, R⁵, R⁶, R⁷, R⁸, R⁹, R¹⁰, R¹¹, R¹², R¹³, R¹⁴, R¹⁵ and R¹⁶ are as defined herein in the corresponding cytotoxin conjugated residues CTX-I, CTX-II, CTX-III, CTX-IV, CTX-V, CTX-VI, CTX-VII and CTX-VIII, respectively. In one embodiment of the above non-conjugated cytotoxins, the cytotoxin is not T3 or T4.

In one aspect of the above variables R¹ to R¹³, any designated aryl group, such as a C₆₋₁₀aryl, may be a phenyl group, a 1-naphthyl or 2-naphthyl group, and the aryl group is unsubstituted or substituted with 1 or 2 substituents selected from the group consisting of halo, cyano, nitro, CF₃—CF₃O—, CH₃O—, —CO₂H, —C(O)CH₃, —NH₂, —OH, —SH, —NHCH₃, —N(CH₃)₂, —SCH₃ and —C₁₋₃alkyl.

In one embodiment of the above ADC, A is selected from the group consisting of alemtuzumab, bevacizumab, cetuximab, ipilimumab, ofatumumab, anitumumab, rituximab, tositumomab, milatuzumab and trastuzumab;

PD is a pyrrole-2,5-dione, a pyrrolidine-2,5-dione;

each L¹, L² and L³ is independently selected from the group consisting of —NHC(O)—, —C(O)NH—, —(CH₂CH₂O)ₚ—, —(CH₂CH₂O)ₚCH₂CH₂—, —CH₂CH₂—(CH₂CH₂O)ₚ— and -(AA)ᵣ- where the AA is selected from the group consisting of Gly, Arg, Val, Ala, Cys, Gln, Leu, Ile, Lys and Ser or their N-methylated analogues; a, b and c are each independently 0 or 1; each p and r is independently 1 or 2; m is 1; and n is 1, 2, 3 or 4; and CTX is a tubulysin residue or derivative thereof, or an auristatin residue or a derivative thereof; with the proviso that when -(L¹)ₐ-(L²)_{b}-(L³)_{c}- together is (CH₂)₁₋₁₂— or —(CH₂CH₂O)₁₋₁₂CH₂CH₂— then L¹, L² and L³ are not bonded to CTX by an amide bond.

In one embodiment of the above ADC, A is selected from the group consisting of alemtuzumab, bevacizumab, cetuximab, ipilimumab, ofatumumab, anitumumab, rituximab, tositumomab, inotuzumab, glembatumumab, lovortuzumab and trastuzumab;

PD is a pyrrole-2,5-dione, a pyrrolidine-2,5-dione;

each L¹, L² and L³ is independently a linker selected from the group consisting of (CH₂)_{q}—, —NH(CH₂)₂NH—, —NH(CH₂CH₂)C(O)—, —C(O)NH(CH₂CH₂)NH—, —NHCH₂C(O)—, —NHC(O)—, —C(O)NH—, —NCH₃C(O)—, —C(O)NCH₃—, cyclopentanyl, cyclohexanyl, unsubstituted phenylenyl, phenylenyl substituted by 1 or 2 substituents selected from the group consisting of halo, CH₃O—, —C(O)OC₁₋₃alkyl, —C(O)CH₃, —NHCH₃, —N(CH₃)₂, —C₁₋₃alkyl; and -(AA)ᵣ-; where the AA is selected from the group consisting of Gly, Arg, Val, Ala, Cys, Gln, Leu, Ile, Lys and Ser or their N-methylated analogues; a, b and c are each independently 0 or 1;

each p and r is independently 1 or 2; m is 1; and n is 1, 2, 3 or 4; and CTX is a tubulysin residue or derivative thereof, or an auristatin residue or a derivative thereof. In one aspect, when -(L¹)ₐ-(L²)_{b}-(L³)_{c}- together is —(CH₂)₁₋₁₂— or —(CH₂CH₂O)₁₋₁₂CH₂CH₂— then L¹, L² and L³ are not bonded to CTX by an amide bond.

In one embodiment of the above ADC, A is selected from the group consisting of alemtuzumab, bevacizumab, cetuximab, ipilimumab, ofatumumab, anitumumab, rituximab, tositumomab, inotuzumab, glembatumumab, lovortuzumab, milatuzumab and trastuzumab;

PD is a pyrrole-2,5-dione, a pyrrolidine-2,5-dione;

each L¹, L² and L³ is independently selected from the group consisting of —NHC(O)—, —C(O)NH—, —(CH₂CH₂O)ₚ—, —(CH₂CH₂O)ₚCH₂CH₂—, —CH₂CH₂—(CH₂CH₂O)ₚ— and -(AA)ᵣ- where the AA is selected from the group consisting of Gly, Arg, Val, Ala, Cys, Gln, Leu, Ile, Lys and Ser or their N-methylated analogues; a, b and c are each independently 0 or 1; each p and r is independently 1 or 2; m is 1; and n is 1, 2, 3 or 4; and

CTX is a tubulysin residue selected from the compound of the formulae CTX-III, CTX-IIIa, CTX-IV, CTX-IVa, CTX-V, CTX-Va, CTX-VI, CTX-VIa, CTX-VII, CTX-VIIa, CTX-VIII and CTX-VIIIa; with the proviso that when -(L¹)ₐ-(L²)_{b}-(L³)_{c}- together is —(CH₂)₁₋₁₂— or —(CH₂CH₂O)₁₋₁₂CH₂CH₂— then L¹, L² and L³ are not bonded to CTX by an amide bond.

In one embodiment of the above ADC, A is selected from the group consisting of alemtuzumab, bevacizumab, cetuximab, ipilimumab, ofatumumab, anitumumab, rituximab, tositumomab, inotuzumab, glembatumumab, lovortuzumab, milatuzumab and trastuzumab;

PD is a pyrrole-2,5-dione, a pyrrolidine-2,5-dione;

each L¹, L² and L³ is independently selected from the group consisting of —NHC(O)—, —C(O)NH—, —(CH₂CH₂O)ₚ, —(CH₂CH₂O)ₚCH₂CH₂— and —CH₂CH₂—(CH₂CH₂O)ₚ—;

a, b and c are each 1; each p and r is independently 1 or 2; m is 1;

n is 1, 2 or 3; and CTX is a tubulysin residue selected from the compound of the formulae CTX-III, CTX-IIIa, CTX-IV, CTX-IVa, CTX-V, CTX-Va, CTX-VI, CTX-VIa, CTX-VII, CTX-VIIa, CTX-VIII and CTX-VIIIa.

TABLE 1
Antibody-Drug Conjugates

| En- | | | | | | i | R⁴ | R⁵ | R⁶ | R⁷ | R⁸ |
|---|---|---|---|---|---|---|---|---|---|---|---|
| try | A^{a} | PD | L¹ | L² | L³ | CTX—II | | | | | |
| 1 | TTZ | 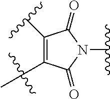 [SMILES: *C1=C(*)C(=O)N(*)C1=O] | — | —(CH₂)₂— | —OC(O)— | 1 | 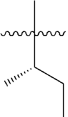 [SMILES: *[C@@H](C)CC] | —CH (CH₃)₂ | —CH₃ | —OC(O)CH₃ | —OCH₃ |
| 2 | TTZ | 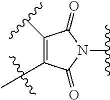 [SMILES: *C1=C(*)C(=O)N(*)C1=O] | — | — (CH₂CH₂O)₁₂ — | —CH₂CH₂C(O)— | 1 | 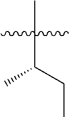 [SMILES: *[C@@H](C)CC] | —CH (CH₃)₂ | —CH₃ | —OC(O)CH₃ | —OCH₃ |
| 3 | TTZ | 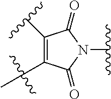 [SMILES: *C1=C(*)C(=O)N(*)C1=O] | — | — (CH₂CH₂O)₆— | —CH₂CH₂C(O)— | 1 | 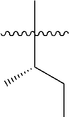 [SMILES: *[C@@H](C)CC] | —CH (CH₃)₂ | —CH₃ | —OC(O)CH₃ | —OCH₃ |
| 4 | TTZ | 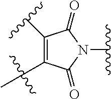 [SMILES: *C1=C(*)C(=O)N(*)C1=O] | —(CH₂)₂CO— | —NHCH₂CH₂— | —OC(O)— | 1 | 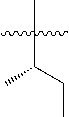 [SMILES: *[C@@H](C)CC] | —CH (CH₃)₂ | —CH₃ | —OC(O)CH₃ | —OCH₃ |
| 5 | TTZ | 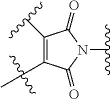 [SMILES: *C1=C(*)C(=O)N(*)C1=O] | —(CH₂)₄CO— | —NHCH₂CH₂— | —OC(O)— | 1 | 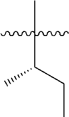 [SMILES: *[C@@H](C)CC] | —CH (CH₃)₂ | —CH₃ | —OC(O)CH₃ | —OCH₃ |
| 6 | TTZ | 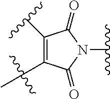 [SMILES: *C1=C(*)C(=O)N(*)C1=O] | —(CH₂)₅CO— | —NHCH₂CH₂— | —OC(O)— | 1 | 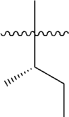 [SMILES: *[C@@H](C)CC] | —CH (CH₃)₂ | —CH₃ | —OC(O)CH₃ | —OCH₃ |
| 7 | TTZ | 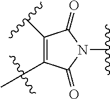 [SMILES: *C1=C(*)C(=O)N(*)C1=O] | — | —(CH₂)₂— | —NHC(O)— | 1 | 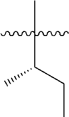 [SMILES: *[C@@H](C)CC] | —CH (CH₃)₂ | —CH₃ | —OC(O)CH₃ | —OCH₃ |
| 8 | TTZ | 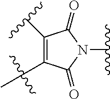 [SMILES: *C1=C(*)C(=O)N(*)C1=O] | — | — (CH₂CH₂O)₁₂ — | —NHC(O)— | 1 | 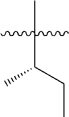 [SMILES: *[C@@H](C)CC] | —CH (CH₃)₂ | —CH₃ | —OC(O)CH₃ | —OCH₃ |
| 9 | TTZ | 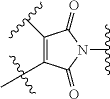 [SMILES: *C1=C(*)C(=O)N(*)C1=O] | — | — (CH₂CH₂O)₆— | —NHC(O)— | 1 | 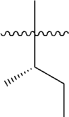 [SMILES: *[C@@H](C)CC] | —CH (CH₃)₂ | —CH₃ | —OC(O)CH₃ | —OCH₃ |
| 10 | TTZ | 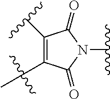 [SMILES: *C1=C(*)C(=O)N(*)C1=O] | —(CH₂)₂CO— | —NHCH₂CH₂— | —NHC(O)— | 1 | 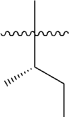 [SMILES: *[C@@H](C)CC] | —CH (CH₃)₂ | —CH₃ | —OC(O)CH₃ | —OCH₃ |
| 11 | TTZ | 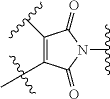 [SMILES: *C1=C(*)C(=O)N(*)C1=O] | —(CH₂)₄CO— | —NHCH₂CH₂— | —NHC(O)— | 1 | 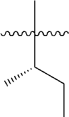 [SMILES: *[C@@H](C)CC] | —CH (CH₃)₂ | —CH₃ | —OC(O)CH₃ | —OCH₃ |
| 12 | TTZ | 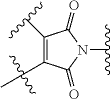 [SMILES: *C1=C(*)C(=O)N(*)C1=O] | —(CH₂)₅CO— | —NHCH₂CH₂— | —NHC(O)— | 1 | 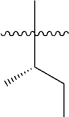 [SMILES: *[C@@H](C)CC] | —CH (CH₃)₂ | —CH₃ | —OC(O)CH₃ | —OCH₃ |
| 13 | TTZ | 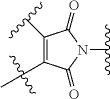 [SMILES: *C1=C(*)C(=O)N(*)C1=O] | — | —(CH₂)₂— | —NHCH3C(O)— | 1 | 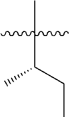 [SMILES: *[C@@H](C)CC] | —CH (CH₃)₂ | —CH₃ | —OC(O)CH₃ | —OCH₃ |
| 14 | TTZ | 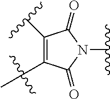 [SMILES: *C1=C(*)C(=O)N(*)C1=O] | — | — (CH₂CH₂O)₁₂ — | —NCH₃C(O)— | 1 | 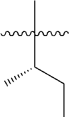 [SMILES: *[C@@H](C)CC] | —CH (CH₃)₂ | —CH₃ | —OC(O)CH₃ | —OCH₃ |
| 15 | TTZ | 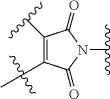 [SMILES: *C1=C(*)C(=O)N(*)C1=O] | — | — (CH₂CH₂O)₆— | —NCH₃C(O)— | 1 | 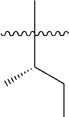 [SMILES: *[C@@H](C)CC] | —CH (CH₃)₂ | —CH₃ | —OC(O)CH₃ | —OCH₃ |
| 16 | TTZ | 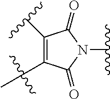 [SMILES: *C1=C(*)C(=O)N(*)C1=O] | —(CH₂)₂CO— | —NHCH₂CH₂— | —NCH₃C(O)— | 1 | 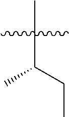 [SMILES: *[C@@H](C)CC] | —CH (CH₃)₂ | —CH₃ | —OC(O)CH₃ | —OCH₃ |
| 17 | TTZ | 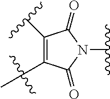 [SMILES: *C1=C(*)C(=O)N(*)C1=O] | —(CH₂)₄CO— | —NHCH₂CH₂— | —NCH₃C(O)— | 1 | 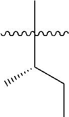 [SMILES: *[C@@H](C)CC] | —CH (CH₃)₂ | —CH₃ | —OC(O)CH₃ | —OCH₃ |
| 18 | TTZ | 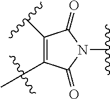 [SMILES: *C1=C(*)C(=O)N(*)C1=O] | —(CH₂)₅CO— | —NHCH₂CH₂— | —NCH₃C(O)— | 1 | 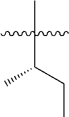 [SMILES: *[C@@H](C)CC] | —CH (CH₃)₂ | —CH₃ | —OC(O)CH₃ | —OCH₃ |
| 19 | TTZ | 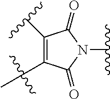 [SMILES: *C1=C(*)C(=O)N(*)C1=O] | — | —(CH₂)₂— | —OC(O)— | 1 | 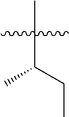 [SMILES: *[C@@H](C)CC] | —CH (CH₃)₂ | —CH₃ | —OC(O)CH₃ | —NH (CH₂)₂- Phenyl |
| 20 | TTZ | 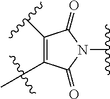 [SMILES: *C1=C(*)C(=O)N(*)C1=O] | — | — (CH₂CH₂O)₁₂ — | —CH₂CH₂C(O)— | 1 | 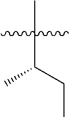 [SMILES: *[C@@H](C)CC] | —CH (CH₃)₂ | —CH₃ | —OC(O)CH₃ | —NH (CH₂)₂- Phenyl |
| 21 | TTZ | 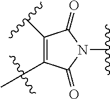 [SMILES: *C1=C(*)C(=O)N(*)C1=O] | — | — (CH₂CH₂O)₆— | —CH₂CH₂C(O)— | 1 | 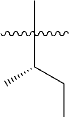 [SMILES: *[C@@H](C)CC] | —CH (CH₃)₂ | —CH₃ | —OC(O)CH₃ | —NH (CH₂)₂- Phenyl |
| 22 | TTZ | 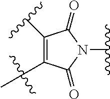 [SMILES: *C1=C(*)C(=O)N(*)C1=O] | —(CH₂)₂CO— | —NHCH₂CH₂— | —OC(O)— | 1 | 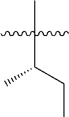 [SMILES: *[C@@H](C)CC] | —CH (CH₃)₂ | —CH₃ | —OC(O)CH₃ | —NH (CH₂)₂- Phenyl |
| 23 | TTZ | 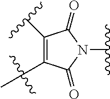 [SMILES: *C1=C(*)C(=O)N(*)C1=O] | —(CH₂)₄CO— | —NHCH₂CH₂— | —OC(O)— | 1 | 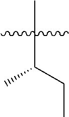 [SMILES: *[C@@H](C)CC] | —CH (CH₃)₂ | —CH₃ | —OC(O)CH₃ | —NH (CH₂)₂- Phenyl |
| 24 | TTZ | 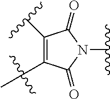 [SMILES: *C1=C(*)C(=O)N(*)C1=O] | —(CH₂)₅CO— | —NHCH₂CH₂— | —OC(O)— | 1 | 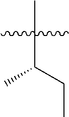 [SMILES: *[C@@H](C)CC] | —CH (CH₃)₂ | —CH₃ | —OC(O)CH₃ | —NH (CH₂)₂- Phenyl |
| 25 | TTZ | 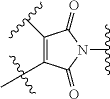 [SMILES: *C1=C(*)C(=O)N(*)C1=O] | — | —(CH₂)₂— | —NHC(O)— | 1 | 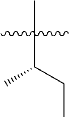 [SMILES: *[C@@H](C)CC] | —CH (CH₃)₂ | —CH₃ | —OC(O)CH₃ | —NH (CH₂)₂- Phenyl |
| 26 | TTZ | 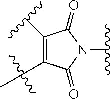 [SMILES: *C1=C(*)C(=O)N(*)C1=O] | — | — (CH₂CH₂O)₁₂ — | —NHC(O)— | 1 | 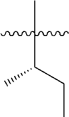 [SMILES: *[C@@H](C)CC] | —CH (CH₃)₂ | —CH₃ | —OC(O)CH₃ | —NH (CH₂)₂- Phenyl |
| 27 | TTZ | 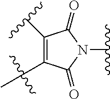 [SMILES: *C1=C(*)C(=O)N(*)C1=O] | — | — (CH₂CH₂O)₆— | —NHC(O)— | 1 | 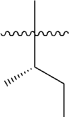 [SMILES: *[C@@H](C)CC] | —CH (CH₃)₂ | —CH₃ | —OC(O)CH₃ | —NH (CH₂)₂- Phenyl |
| 28 | TTZ | 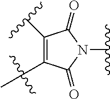 [SMILES: *C1=C(*)C(=O)N(*)C1=O] | —(CH₂)₂CO— | —NHCH₂CH₂— | —NHC(O)— | 1 | 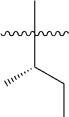 [SMILES: *[C@@H](C)CC] | —CH (CH₃)₂ | —CH₃ | —OC(O)CH₃ | —NH (CH₂)₂- Phenyl |
| 29 | TTZ | 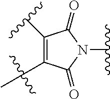 [SMILES: *C1=C(*)C(=O)N(*)C1=O] | —(CH₂)₄CO— | —NHCH₂CH₂— | —NHC(O)— | 1 | 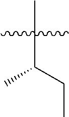 [SMILES: *[C@@H](C)CC] | —CH (CH₃)₂ | —CH₃ | —OC(O)CH₃ | —NH (CH₂)₂- Phenyl |
| 30 | TTZ | 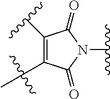 [SMILES: *C1=C(*)C(=O)N(*)C1=O] | —(CH₂)₅CO— | —NHCH₂CH₂— | —NHC(O)— | 1 | 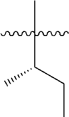 [SMILES: *[C@@H](C)CC] | —CH (CH₃)₂ | —CH₃ | —OC(O)CH₃ | —NH (CH₂)₂- Phenyl |
| 31 | TTZ | 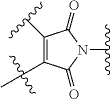 [SMILES: *C1=C(*)C(=O)N(*)C1=O] | — | —(CH₂)₂— | —NCH3C(O)— | 1 | 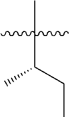 [SMILES: *[C@@H](C)CC] | —CH (CH₃)₂ | —CH₃ | —OC(O)CH₃ | —NH (CH₂)₂- Phenyl |
| 32 | TTZ | 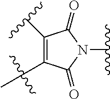 [SMILES: *C1=C(*)C(=O)N(*)C1=O] | — | — (CH₂CH₂O)₁₂ — | —NCH₃C(O)— | 1 | 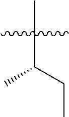 [SMILES: *[C@@H](C)CC] | —CH (CH₃)₂ | —CH₃ | —OC(O)CH₃ | —NH (CH₂)₂- Phenyl |
| 33 | TTZ | 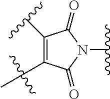 [SMILES: *C1=C(*)C(=O)N(*)C1=O] | — | — (CH₂CH₂O)₆— | —NCH₃C(O)— | 1 | 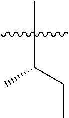 [SMILES: *[C@@H](C)CC] | —CH (CH₃)₂ | —CH₃ | —OC(O)CH₃ | —NH (CH₂)₂- Phenyl |
| 34 | TTZ | 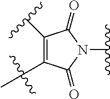 [SMILES: *C1=C(*)C(=O)N(*)C1=O] | —(CH₂)₂CO— | —NHCH₂CH₂— | —NCH₃C(O)— | 1 | 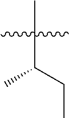 [SMILES: *[C@@H](C)CC] | —CH (CH₃)₂ | —CH₃ | —OC(O)CH₃ | —NH (CH₂)₂- Phenyl |
| 35 | TTZ | 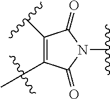 [SMILES: *C1=C(*)C(=O)N(*)C1=O] | —(CH₂)₄CO— | —NHCH₂CH₂— | —NCH₃C(O)— | 1 | 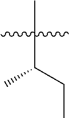 [SMILES: *[C@@H](C)CC] | —CH (CH₃)₂ | —CH₃ | —OC(O)CH₃ | —NH (CH₂)₂- Phenyl |
| 36 | TTZ | 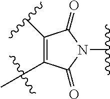 [SMILES: *C1=C(*)C(=O)N(*)C1=O] | —(CH₂)₅CO— | —NHCH₂CH₂— | —NCH₃C(O)— | 1 | 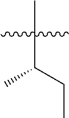 [SMILES: *[C@@H](C)CC] | —CH (CH₃)₂ | —CH₃ | —OC(O)CH₃ | —NH (CH₂)₂- Phenyl |
| 37 | TTZ | 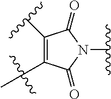 [SMILES: *C1=C(*)C(=O)N(*)C1=O] | — | —(CH₂)₂— | —OC(O)— | 1 | 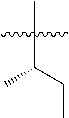 [SMILES: *[C@@H](C)CC] | —CH (CH₃)₂ | —CH₃ | —OC(O)CH₃ | —NH (CH₂)₂- p-MeO- phenyl |
| 38 | TTZ | 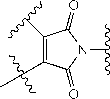 [SMILES: *C1=C(*)C(=O)N(*)C1=O] | — | — (CH₂CH₂O)₁₂ — | —CH₂CH₂C(O)— | 1 | 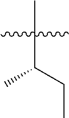 [SMILES: *[C@@H](C)CC] | —CH (CH₃)₂ | —CH₃ | —OC(O)CH₃ | —NH (CH₂)₂- p-MeO- phenyl |
| 39 | TTZ | 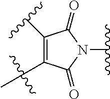 [SMILES: *C1=C(*)C(=O)N(*)C1=O] | — | — (CH₂CH₂O)₆— | —CH₂CH₂C(O)— | 1 | 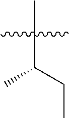 [SMILES: *[C@@H](C)CC] | —CH (CH₃)₂ | —CH₃ | —OC(O)CH₃ | —NH (CH₂)₂- p-MeO- phenyl |
| 40 | TTZ | 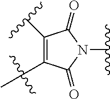 [SMILES: *C1=C(*)C(=O)N(*)C1=O] | —(CH₂)₂CO— | —NHCH₂CH₂— | —OC(O)— | 1 | 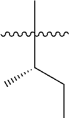 [SMILES: *[C@@H](C)CC] | —CH (CH₃)₂ | —CH₃ | —OC(O)CH₃ | —NH (CH₂)₂- p-MeO- phenyl |
| 41 | TTZ | 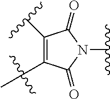 [SMILES: *C1=C(*)C(=O)N(*)C1=O] | —(CH₂)₄CO— | —NHCH₂CH₂— | —OC(O)— | 1 | 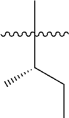 [SMILES: *[C@@H](C)CC] | —CH (CH₃)₂ | —CH₃ | —OC(O)CH₃ | —NH (CH₂)₂- p-MeO- phenyl |
| 42 | TTZ | 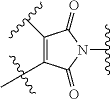 [SMILES: *C1=C(*)C(=O)N(*)C1=O] | —(CH₂)₅CO— | —NHCH₂CH₂— | —OC(O)— | 1 | 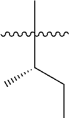 [SMILES: *[C@@H](C)CC] | —CH (CH₃)₂ | —CH₃ | —OC(O)CH₃ | —NH (CH₂)₂- p-MeO- phenyl |
| 43 | TTZ | 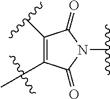 [SMILES: *C1=C(*)C(=O)N(*)C1=O] | — | —(CH₂)₂— | —NHC(O)— | 1 | 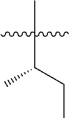 [SMILES: *[C@@H](C)CC] | —CH (CH₃)₂ | —CH₃ | —OC(O)CH₃ | —NH (CH₂)₂- p-MeO- phenyl |
| 44 | TTZ | 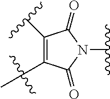 [SMILES: *C1=C(*)C(=O)N(*)C1=O] | — | — (CH₂CH₂O)₁₂ — | —NHC(O)— | 1 | 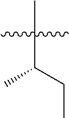 [SMILES: *[C@@H](C)CC] | —CH (CH₃)₂ | —CH₃ | —OC(O)CH₃ | —NH (CH₂)₂- p-MeO- phenyl |
| 45 | TTZ | 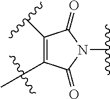 [SMILES: *C1=C(*)C(=O)N(*)C1=O] | — | — (CH₂CH₂O)₆— | —NHC(O)— | 1 | 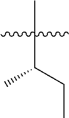 [SMILES: *[C@@H](C)CC] | —CH (CH₃)₂ | —CH₃ | —OC(O)CH₃ | —NH (CH₂)₂- p-MeO- phenyl |
| 46 | TTZ | 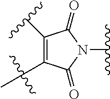 [SMILES: *C1=C(*)C(=O)N(*)C1=O] | —(CH₂)₂CO— | —NHCH₂CH₂— | —NHC(O)— | 1 | 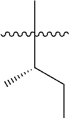 [SMILES: *[C@@H](C)CC] | —CH (CH₃)₂ | —CH₃ | —OC(O)CH₃ | —NH (CH₂)₂- p-MeO- phenyl |
| 47 | TTZ | 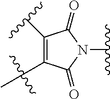 [SMILES: *C1=C(*)C(=O)N(*)C1=O] | —(CH₂)₄CO— | —NHCH₂CH₂— | —NHC(O)— | 1 | 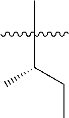 [SMILES: *[C@@H](C)CC] | —CH (CH₃)₂ | —CH₃ | —OC(O)CH₃ | —NH (CH₂)₂- p-MeO- phenyl |
| 48 | TTZ | 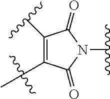 [SMILES: *C1=C(*)C(=O)N(*)C1=O] | —(CH₂)₅CO— | —NHCH₂CH₂— | —NHC(O)— | 1 | 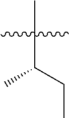 [SMILES: *[C@@H](C)CC] | —CH (CH₃)₂ | —CH₃ | —OC(O)CH₃ | —NH (CH₂)₂- p-MeO- phenyl |
| 49 | TTZ | 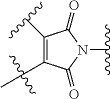 [SMILES: *C1=C(*)C(=O)N(*)C1=O] | — | —(CH₂)₂— | —NCH3C(O)— | 1 | 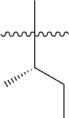 [SMILES: *[C@@H](C)CC] | —CH (CH₃)₂ | —CH₃ | —OC(O)CH₃ | —NH (CH₂)₂- p-MeO- phenyl |
| 50 | TTZ | 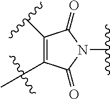 [SMILES: *C1=C(*)C(=O)N(*)C1=O] | — | — (CH₂CH₂O)₁₂ — | —NCH₃C(O)— | 1 | 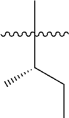 [SMILES: *[C@@H](C)CC] | —CH (CH₃)₂ | —CH₃ | —OC(O)CH₃ | —NH (CH₂)₂- p-MeO- phenyl |
| 51 | TTZ | 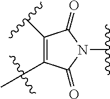 [SMILES: *C1=C(*)C(=O)N(*)C1=O] | — | — (CH₂CH₂O)₆— | —NCH₃C(O)— | 1 | 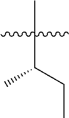 [SMILES: *[C@@H](C)CC] | —CH (CH₃)₂ | —CH₃ | —OC(O)CH₃ | —NH (CH₂)₂- p-MeO- phenyl |
| 52 | TTZ | 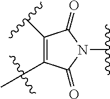 [SMILES: *C1=C(*)C(=O)N(*)C1=O] | —(CH₂)₂CO— | —NHCH₂CH₂— | —NCH₃C(O)— | 1 | 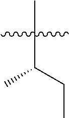 [SMILES: *[C@@H](C)CC] | —CH (CH₃)₂ | —CH₃ | —OC(O)CH₃ | —NH (CH₂)₂- p-MeO- phenyl |
| 53 | TTZ | 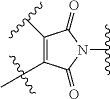 [SMILES: *C1=C(*)C(=O)N(*)C1=O] | —(CH₂)₄CO— | —NHCH₂CH₂— | —NCH₃C(O)— | 1 | 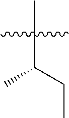 [SMILES: *[C@@H](C)CC] | —CH (CH₃)₂ | —CH₃ | —OC(O)CH₃ | —NH (CH₂)₂- p-MeO- phenyl |
| 54 | TTZ | 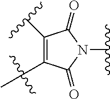 [SMILES: *C1=C(*)C(=O)N(*)C1=O] | —(CH₂)₅CO— | —NHCH₂CH₂— | —NCH₃C(O)— | 1 | 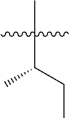 [SMILES: *[C@@H](C)CC] | —CH (CH₃)₂ | —CH₃ | —OC(O)CH₃ | —NH (CH₂)₂- p-MeO- phenyl |
| 55 | TTZ | 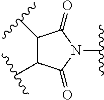 [SMILES: *C1C(=O)N(*)C(=O)C1*] | — | —(CH₂)₂— | —OC(O)— | 1 | 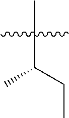 [SMILES: *[C@@H](C)CC] | —CH (CH₃)₂ | —CH₃ | —OC(O)CH₃ | —OCH₃ |
| 56 | TTZ | 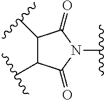 [SMILES: *C1C(=O)N(*)C(=O)C1*] | — | — (CH₂CH₂O)₁₂ — | —CH₂CH₂C(O)— | 1 | 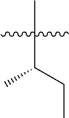 [SMILES: *[C@@H](C)CC] | —CH (CH₃)₂ | —CH₃ | —OC(O)CH₃ | —OCH₃ |
| 57 | TTZ | 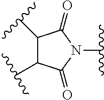 [SMILES: *C1C(=O)N(*)C(=O)C1*] | — | — (CH₂CH₂O)₆— | —CH₂CH₂C(O)— | 1 | 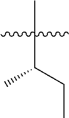 [SMILES: *[C@@H](C)CC] | —CH (CH₃)₂ | —CH₃ | —OC(O)CH₃ | —OCH₃ |
| 58 | TTZ | 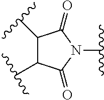 [SMILES: *C1C(=O)N(*)C(=O)C1*] | —(CH₂)₂CO— | —NHCH₂CH₂— | —OC(O)— | 1 | 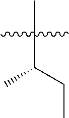 [SMILES: *[C@@H](C)CC] | —CH (CH₃)₂ | —CH₃ | —OC(O)CH₃ | —OCH₃ |
| 59 | TTZ | 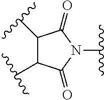 [SMILES: *C1C(=O)N(*)C(=O)C1*] | —(CH₂)₄CO— | —NHCH₂CH₂— | —OC(O)— | 1 | 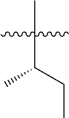 [SMILES: *[C@@H](C)CC] | —CH (CH₃)₂ | —CH₃ | —OC(O)CH₃ | —OCH₃ |
| 60 | TTZ | 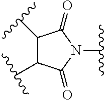 [SMILES: *C1C(=O)N(*)C(=O)C1*] | —(CH₂)₅CO— | —NHCH₂CH₂— | —OC(O)— | 1 | 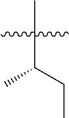 [SMILES: *[C@@H](C)CC] | —CH (CH₃)₂ | —CH₃ | —OC(O)CH₃ | —OCH₃ |
| 61 | TTZ | 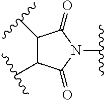 [SMILES: *C1C(=O)N(*)C(=O)C1*] | — | —(CH₂)₂— | —NHC(O)— | 1 | 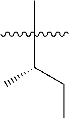 [SMILES: *[C@@H](C)CC] | —CH (CH₃)₂ | —CH₃ | —OC(O)CH₃ | —OCH₃ |
| 62 | TTZ | 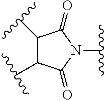 [SMILES: *C1C(=O)N(*)C(=O)C1*] | — | — (CH₂CH₂O)₁₂ — | —NHC(O)— | 1 | 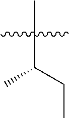 [SMILES: *[C@@H](C)CC] | —CH (CH₃)₂ | —CH₃ | —OC(O)CH₃ | —OCH₃ |
| 63 | TTZ | 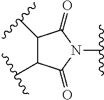 [SMILES: *C1C(=O)N(*)C(=O)C1*] | — | — (CH₂CH₂O)₆— | —NHC(O)— | 1 | 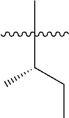 [SMILES: *[C@@H](C)CC] | —CH (CH₃)₂ | —CH₃ | —OC(O)CH₃ | —OCH₃ |
| 64 | TTZ | 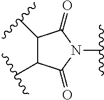 [SMILES: *C1C(=O)N(*)C(=O)C1*] | —(CH₂)₂CO— | —NHCH₂CH₂— | —NHC(O)— | 1 | 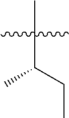 [SMILES: *[C@@H](C)CC] | —CH (CH₃)₂ | —CH₃ | —OC(O)CH₃ | —OCH₃ |
| 65 | TTZ | 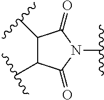 [SMILES: *C1C(=O)N(*)C(=O)C1*] | —(CH₂)₄CO— | —NHCH₂CH₂— | —NHC(O)— | 1 | 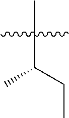 [SMILES: *[C@@H](C)CC] | —CH (CH₃)₂ | —CH₃ | —OC(O)CH₃ | —OCH₃ |
| 66 | TTZ | 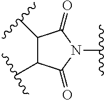 [SMILES: *C1C(=O)N(*)C(=O)C1*] | —(CH₂)₅CO— | —NHCH₂CH₂— | —NHC(O)— | 1 | 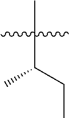 [SMILES: *[C@@H](C)CC] | —CH (CH₃)₂ | —CH₃ | —OC(O)CH₃ | —OCH₃ |
| 67 | TTZ | 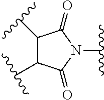 [SMILES: *C1C(=O)N(*)C(=O)C1*] | — | —(CH₂)₂— | —NCH3C(O)— | 1 | 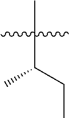 [SMILES: *[C@@H](C)CC] | —CH (CH₃)₂ | —CH₃ | —OC(O)CH₃ | —OCH₃ |
| 68 | TTZ | 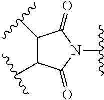 [SMILES: *C1C(=O)N(*)C(=O)C1*] | — | — (CH₂CH₂O)₁₂ — | —NCH₃C(O)— | 1 | 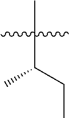 [SMILES: *[C@@H](C)CC] | —CH (CH₃)₂ | —CH₃ | —OC(O)CH₃ | —OCH₃ |
| 69 | TTZ | 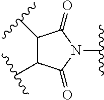 [SMILES: *C1C(=O)N(*)C(=O)C1*] | — | — (CH₂CH₂O)₆— | —NCH₃C(O)— | 1 | 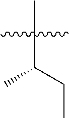 [SMILES: *[C@@H](C)CC] | —CH (CH₃)₂ | —CH₃ | —OC(O)CH₃ | —OCH₃ |
| 70 | TTZ | 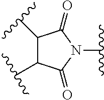 [SMILES: *C1C(=O)N(*)C(=O)C1*] | —(CH₂)₂CO— | —NHCH₂CH₂— | —NCH₃C(O)— | 1 | 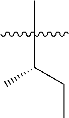 [SMILES: *[C@@H](C)CC] | —CH (CH₃)₂ | —CH₃ | —OC(O)CH₃ | —OCH₃ |
| 71 | TTZ | 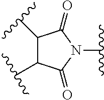 [SMILES: *C1C(=O)N(*)C(=O)C1*] | —(CH₂)₄CO— | —NHCH₂CH₂— | —NCH₃C(O)— | 1 | 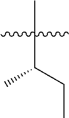 [SMILES: *[C@@H](C)CC] | —CH (CH₃)₂ | —CH₃ | —OC(O)CH₃ | —OCH₃ |
| 72 | TTZ | 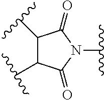 [SMILES: *C1C(=O)N(*)C(=O)C1*] | —(CH₂)₅CO— | —NHCH₂CH₂— | —NCH₃C(O)— | 1 | 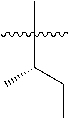 [SMILES: *[C@@H](C)CC] | —CH (CH₃)₂ | —CH₃ | —OC(O)CH₃ | —OCH₃ |
| 73 | TTZ | 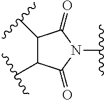 [SMILES: *C1C(=O)N(*)C(=O)C1*] | — | —(CH₂)₂— | —OC(O)— | 1 | 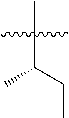 [SMILES: *[C@@H](C)CC] | —CH (CH₃)₂ | —CH₃ | —OC(O)CH₃ | —NH (CH₂)₂- Phenyl |
| 74 | TTZ | 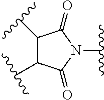 [SMILES: *C1C(=O)N(*)C(=O)C1*] | — | — (CH₂CH₂O)₁₂ — | —CH₂CH₂C(O)— | 1 | 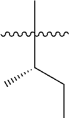 [SMILES: *[C@@H](C)CC] | —CH (CH₃)₂ | —CH₃ | —OC(O)CH₃ | —NH (CH₂)₂- Phenyl |
| 75 | TTZ | 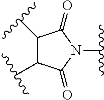 [SMILES: *C1C(=O)N(*)C(=O)C1*] | — | — (CH₂CH₂O)₆— | —CH₂CH₂C(O)— | 1 | 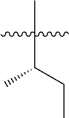 [SMILES: *[C@@H](C)CC] | —CH (CH₃)₂ | —CH₃ | —OC(O)CH₃ | —NH (CH₂)₂- Phenyl |
| 76 | TTZ | 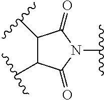 [SMILES: *C1C(=O)N(*)C(=O)C1*] | —(CH₂)₂CO— | —NHCH₂CH₂— | —OC(O)— | 1 | 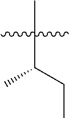 [SMILES: *[C@@H](C)CC] | —CH (CH₃)₂ | —CH₃ | —OC(O)CH₃ | —NH (CH₂)₂- Phenyl |
| 77 | TTZ | 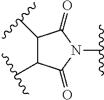 [SMILES: *C1C(=O)N(*)C(=O)C1*] | —(CH₂)₄CO— | —NHCH₂CH₂— | —OC(O)— | 1 | 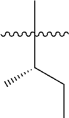 [SMILES: *[C@@H](C)CC] | —CH (CH₃)₂ | —CH₃ | —OC(O)CH₃ | —NH (CH₂)₂- Phenyl |
| 78 | TTZ | 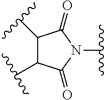 [SMILES: *C1C(=O)N(*)C(=O)C1*] | —(CH₂)₅CO— | —NHCH₂CH₂— | —OC(O)— | 1 | 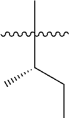 [SMILES: *[C@@H](C)CC] | —CH (CH₃)₂ | —CH₃ | —OC(O)CH₃ | —NH (CH₂)₂- Phenyl |
| 79 | TTZ | 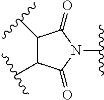 [SMILES: *C1C(=O)N(*)C(=O)C1*] | — | —(CH₂)₂— | —NHC(O)— | 1 | 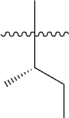 [SMILES: *[C@@H](C)CC] | —CH (CH₃)₂ | —CH₃ | —OC(O)CH₃ | —NH (CH₂)₂- Phenyl |
| 80 | TTZ | 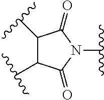 [SMILES: *C1C(=O)N(*)C(=O)C1*] | — | — (CH₂CH₂O)₁₂ — | —NHC(O)— | 1 | 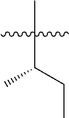 [SMILES: *[C@@H](C)CC] | —CH (CH₃)₂ | —CH₃ | —OC(O)CH₃ | —NH (CH₂)₂- Phenyl |
| 81 | TTZ | 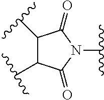 [SMILES: *C1C(=O)N(*)C(=O)C1*] | — | — (CH₂CH₂O)₆— | —NHC(O)— | 1 | 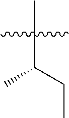 [SMILES: *[C@@H](C)CC] | —CH (CH₃)₂ | —CH₃ | —OC(O)CH₃ | —NH (CH₂)₂- Phenyl |
| 82 | TTZ | 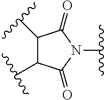 [SMILES: *C1C(=O)N(*)C(=O)C1*] | —(CH₂)₂CO— | —NHCH₂CH₂— | —NHC(O)— | 1 | 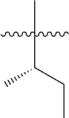 [SMILES: *[C@@H](C)CC] | —CH (CH₃)₂ | —CH₃ | —OC(O)CH₃ | —NH (CH₂)₂- Phenyl |
| 83 | TTZ | 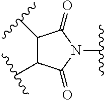 [SMILES: *C1C(=O)N(*)C(=O)C1*] | —(CH₂)₄CO— | —NHCH₂CH₂— | —NHC(O)— | 1 | 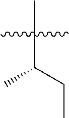 [SMILES: *[C@@H](C)CC] | —CH (CH₃)₂ | —CH₃ | —OC(O)CH₃ | —NH (CH₂)₂- Phenyl |
| 84 | TTZ | 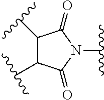 [SMILES: *C1C(=O)N(*)C(=O)C1*] | —(CH₂)₅CO— | —NHCH₂CH₂— | —NHC(O)— | 1 | 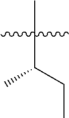 [SMILES: *[C@@H](C)CC] | —CH (CH₃)₂ | —CH₃ | —OC(O)CH₃ | —NH (CH₂)₂- Phenyl |
| 85 | TTZ | 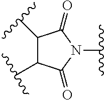 [SMILES: *C1C(=O)N(*)C(=O)C1*] | — | —(CH₂)₂— | —NCH3C(O)— | 1 | 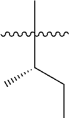 [SMILES: *[C@@H](C)CC] | —CH (CH₃)₂ | —CH₃ | —OC(O)CH₃ | —NH (CH₂)₂- Phenyl |
| 86 | TTZ | 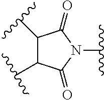 [SMILES: *C1C(=O)N(*)C(=O)C1*] | — | — (CH₂CH₂O)₁₂ — | —NCH₃C(O)— | 1 | 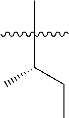 [SMILES: *[C@@H](C)CC] | —CH (CH₃)₂ | —CH₃ | —OC(O)CH₃ | —NH (CH₂)₂- Phenyl |
| 87 | TTZ | 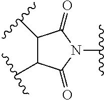 [SMILES: *C1C(=O)N(*)C(=O)C1*] | — | — (CH₂CH₂O)₆— | —NCH₃C(O)— | 1 | 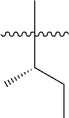 [SMILES: *[C@@H](C)CC] | —CH (CH₃)₂ | —CH₃ | —OC(O)CH₃ | —NH (CH₂)₂- Phenyl |
| 88 | TTZ | 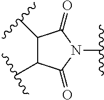 [SMILES: *C1C(=O)N(*)C(=O)C1*] | —(CH₂)₂CO— | —NHCH₂CH₂— | —NCH₃C(O)— | 1 | 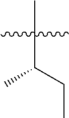 [SMILES: *[C@@H](C)CC] | —CH (CH₃)₂ | —CH₃ | —OC(O)CH₃ | —NH (CH₂)₂- Phenyl |
| 89 | TTZ | 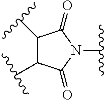 [SMILES: *C1C(=O)N(*)C(=O)C1*] | —(CH₂)₄CO— | —NHCH₂CH₂— | —NCH₃C(O)— | 1 | 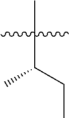 [SMILES: *[C@@H](C)CC] | —CH (CH₃)₂ | —CH₃ | —OC(O)CH₃ | —NH (CH₂)₂- Phenyl |
| 90 | TTZ | 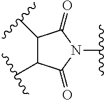 [SMILES: *C1C(=O)N(*)C(=O)C1*] | —(CH₂)₅CO— | —NHCH₂CH₂— | —NCH₃C(O)— | 1 | 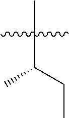 [SMILES: *[C@@H](C)CC] | —CH (CH₃)₂ | —CH₃ | —OC(O)CH₃ | —NH (CH₂)₂- Phenyl |
| 91 | TTZ | 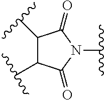 [SMILES: *C1C(=O)N(*)C(=O)C1*] | — | —(CH₂)₂— | —OC(O)— | 1 | 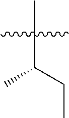 [SMILES: *[C@@H](C)CC] | —CH (CH₃)₂ | —CH₃ | —OC(O)CH₃ | —NH (CH₂)₂- p-MeO- phenyl |
| 92 | TTZ | 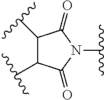 [SMILES: *C1C(=O)N(*)C(=O)C1*] | — | — (CH₂CH₂O)₁₂ — | —CH₂CH₂C(O)— | 1 | 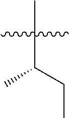 [SMILES: *[C@@H](C)CC] | —CH (CH₃)₂ | —CH₃ | —OC(O)CH₃ | —NH (CH₂)₂- p-MeO- phenyl |
| 93 | TTZ | 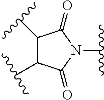 [SMILES: *C1C(=O)N(*)C(=O)C1*] | — | — (CH₂CH₂O)₆— | —CH₂CH₂C(O)— | 1 | 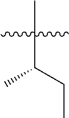 [SMILES: *[C@@H](C)CC] | —CH (CH₃)₂ | —CH₃ | —OC(O)CH₃ | —NH (CH₂)₂- p-MeO- phenyl |
| 94 | TTZ | 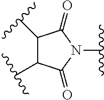 [SMILES: *C1C(=O)N(*)C(=O)C1*] | —(CH₂)₂CO— | —NHCH₂CH₂— | —OC(O)— | 1 | 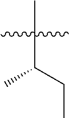 [SMILES: *[C@@H](C)CC] | —CH (CH₃)₂ | —CH₃ | —OC(O)CH₃ | —NH (CH₂)₂- p-MeO- phenyl |
| 95 | TTZ | 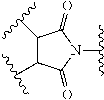 [SMILES: *C1C(=O)N(*)C(=O)C1*] | —(CH₂)₄CO— | —NHCH₂CH₂— | —OC(O)— | 1 | 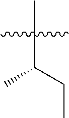 [SMILES: *[C@@H](C)CC] | —CH (CH₃)₂ | —CH₃ | —OC(O)CH₃ | —NH (CH₂)₂- p-MeO- phenyl |
| 96 | TTZ | 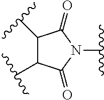 [SMILES: *C1C(=O)N(*)C(=O)C1*] | —(CH₂)₅CO— | —NHCH₂CH₂— | —OC(O)— | 1 | 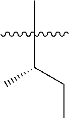 [SMILES: *[C@@H](C)CC] | —CH (CH₃)₂ | —CH₃ | —OC(O)CH₃ | —NH (CH₂)₂- p-MeO- phenyl |
| 97 | TTZ | 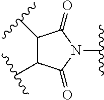 [SMILES: *C1C(=O)N(*)C(=O)C1*] | — | —(CH₂)₂— | —NHC(O)— | 1 | 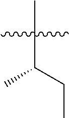 [SMILES: *[C@@H](C)CC] | —CH (CH₃)₂ | —CH₃ | —OC(O)CH₃ | —NH (CH₂)₂- p-MeO- phenyl |
| 98 | TTZ | 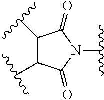 [SMILES: *C1C(=O)N(*)C(=O)C1*] | — | — (CH₂CH₂O)₁₂ — | —NHC(O)— | 1 | 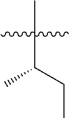 [SMILES: *[C@@H](C)CC] | —CH (CH₃)₂ | —CH₃ | —OC(O)CH₃ | —NH (CH₂)₂- p-MeO- phenyl |
| 99 | TTZ | 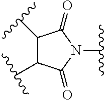 [SMILES: *C1C(=O)N(*)C(=O)C1*] | — | — (CH₂CH₂O)₆— | —NHC(O)— | 1 | 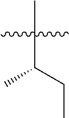 [SMILES: *[C@@H](C)CC] | —CH (CH₃)₂ | —CH₃ | —OC(O)CH₃ | —NH (CH₂)₂- p-MeO- phenyl |
| 100 | TTZ | 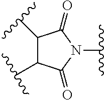 [SMILES: *C1C(=O)N(*)C(=O)C1*] | —(CH₂)₂CO— | —NHCH₂CH₂— | —NHC(O)— | 1 | 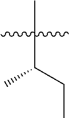 [SMILES: *[C@@H](C)CC] | —CH (CH₃)₂ | —CH₃ | —OC(O)CH₃ | —NH (CH₂)₂- p-MeO- phenyl |
| 101 | TTZ | 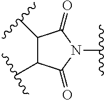 [SMILES: *C1C(=O)N(*)C(=O)C1*] | —(CH₂)₄CO— | —NHCH₂CH₂— | —NHC(O)— | 1 | 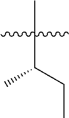 [SMILES: *[C@@H](C)CC] | —CH (CH₃)₂ | —CH₃ | —OC(O)CH₃ | —NH (CH₂)₂- p-MeO- phenyl |
| 102 | TTZ | 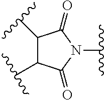 [SMILES: *C1C(=O)N(*)C(=O)C1*] | —(CH₂)₅CO— | —NHCH₂CH₂— | —NHC(O)— | 1 | 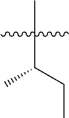 [SMILES: *[C@@H](C)CC] | —CH (CH₃)₂ | —CH₃ | —OC(O)CH₃ | —NH (CH₂)₂- p-MeO- phenyl |
| 103 | TTZ | 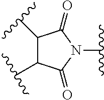 [SMILES: *C1C(=O)N(*)C(=O)C1*] | — | —(CH₂)₂— | —NCH3C(O)— | 1 | 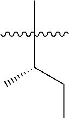 [SMILES: *[C@@H](C)CC] | —CH (CH₃)₂ | —CH₃ | —OC(O)CH₃ | —NH (CH₂)₂- p-MeO- phenyl |
| 104 | TTZ | 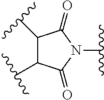 [SMILES: *C1C(=O)N(*)C(=O)C1*] | — | — (CH₂CH₂O)₁₂ — | —NCH₃C(O)— | 1 | 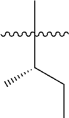 [SMILES: *[C@@H](C)CC] | —CH (CH₃)₂ | —CH₃ | —OC(O)CH₃ | —NH (CH₂)₂- p-MeO- phenyl |
| 105 | TTZ | 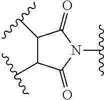 [SMILES: *C1C(=O)N(*)C(=O)C1*] | — | — (CH₂CH₂O)₆— | —NCH₃C(O)— | 1 | 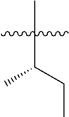 [SMILES: *[C@@H](C)CC] | —CH (CH₃)₂ | —CH₃ | —OC(O)CH₃ | —NH (CH₂)₂- p-MeO- phenyl |
| 106 | TTZ | 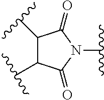 [SMILES: *C1C(=O)N(*)C(=O)C1*] | —(CH₂)₂CO— | —NHCH₂CH₂— | —NCH₃C(O)— | 1 | 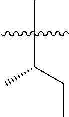 [SMILES: *[C@@H](C)CC] | —CH (CH₃)₂ | —CH₃ | —OC(O)CH₃ | —NH (CH₂)₂- p-MeO- phenyl |
| 107 | TTZ | 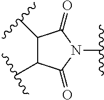 [SMILES: *C1C(=O)N(*)C(=O)C1*] | —(CH₂)₄CO— | —NHCH₂CH₂— | —NCH₃C(O)— | 1 | 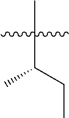 [SMILES: *[C@@H](C)CC] | —CH (CH₃)₂ | —CH₃ | —OC(O)CH₃ | —NH (CH₂)₂- p-MeO- phenyl |
| 108 | TTZ | 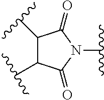 [SMILES: *C1C(=O)N(*)C(=O)C1*] | —(CH₂)₅CO— | —NHCH₂CH₂— | —NCH₃C(O)— | 1 | 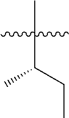 [SMILES: *[C@@H](C)CC] | —CH (CH₃)₂ | —CH₃ | —OC(O)CH₃ | —NH (CH₂)₂- p-MeO- phenyl |
| 109 | BTZ | 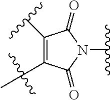 [SMILES: *C1=C(*)C(=O)N(*)C1=O] | — | —(CH₂)₂— | —OC(O)— | 1 | 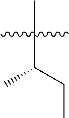 [SMILES: *[C@@H](C)CC] | —CH (CH₃)₂ | —CH₃ | —OC(O)CH₃ | —OCH₃ |
| 110 | BTZ | 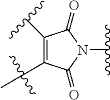 [SMILES: *C1=C(*)C(=O)N(*)C1=O] | — | — (CH₂CH₂O)₁₂ — | —CH₂CH₂C(O)— | 1 | 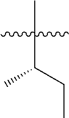 [SMILES: *[C@@H](C)CC] | —CH (CH₃)₂ | —CH₃ | —OC(O)CH₃ | —OCH₃ |
| 111 | BTZ | 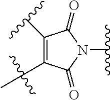 [SMILES: *C1=C(*)C(=O)N(*)C1=O] | — | — (CH₂CH₂O)₆— | —CH₂CH₂C(O)— | 1 | 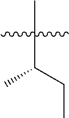 [SMILES: *[C@@H](C)CC] | —CH (CH₃)₂ | —CH₃ | —OC(O)CH₃ | —OCH₃ |
| 112 | BTZ | 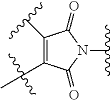 [SMILES: *C1=C(*)C(=O)N(*)C1=O] | —(CH₂)₂CO— | —NHCH₂CH₂— | —OC(O)— | 1 | 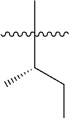 [SMILES: *[C@@H](C)CC] | —CH (CH₃)₂ | —CH₃ | —OC(O)CH₃ | —OCH₃ |
| 113 | BTZ | 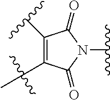 [SMILES: *C1=C(*)C(=O)N(*)C1=O] | —(CH₂)₄CO— | —NHCH₂CH₂— | —OC(O)— | 1 | 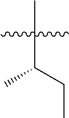 [SMILES: *[C@@H](C)CC] | —CH (CH₃)₂ | —CH₃ | —OC(O)CH₃ | —OCH₃ |
| 114 | BTZ | 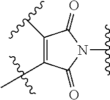 [SMILES: *C1=C(*)C(=O)N(*)C1=O] | —(CH₂)₅CO— | —NHCH₂CH₂— | —OC(O)— | 1 | 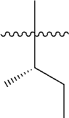 [SMILES: *[C@@H](C)CC] | —CH (CH₃)₂ | —CH₃ | —OC(O)CH₃ | —OCH₃ |
| 115 | BTZ | 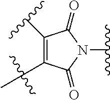 [SMILES: *C1=C(*)C(=O)N(*)C1=O] | — | —(CH₂)₂— | —NHC(O)— | 1 | 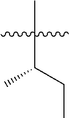 [SMILES: *[C@@H](C)CC] | —CH (CH₃)₂ | —CH₃ | —OC(O)CH₃ | —OCH₃ |
| 116 | BTZ | 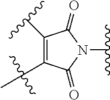 [SMILES: *C1=C(*)C(=O)N(*)C1=O] | — | — (CH₂CH₂O)₁₂ — | —NHC(O)— | 1 | 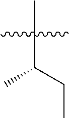 [SMILES: *[C@@H](C)CC] | —CH (CH₃)₂ | —CH₃ | —OC(O)CH₃ | —OCH₃ |
| 117 | BTZ | 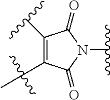 [SMILES: *C1=C(*)C(=O)N(*)C1=O] | — | — (CH₂CH₂O)₆— | —NHC(O)— | 1 | 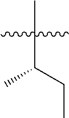 [SMILES: *[C@@H](C)CC] | —CH (CH₃)₂ | —CH₃ | —OC(O)CH₃ | —OCH₃ |
| 118 | BTZ | 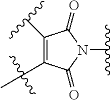 [SMILES: *C1=C(*)C(=O)N(*)C1=O] | —(CH₂)₂CO— | —NHCH₂CH₂— | —NHC(O)— | 1 | 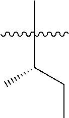 [SMILES: *[C@@H](C)CC] | —CH (CH₃)₂ | —CH₃ | —OC(O)CH₃ | —OCH₃ |
| 119 | BTZ | 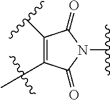 [SMILES: *C1=C(*)C(=O)N(*)C1=O] | —(CH₂)₄CO— | —NHCH₂CH₂— | —NHC(O)— | 1 | 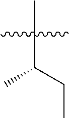 [SMILES: *[C@@H](C)CC] | —CH (CH₃)₂ | —CH₃ | —OC(O)CH₃ | —OCH₃ |
| 120 | BTZ | 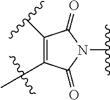 [SMILES: *C1=C(*)C(=O)N(*)C1=O] | —(CH₂)₅CO— | —NHCH₂CH₂— | —NHC(O)— | 1 | 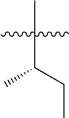 [SMILES: *[C@@H](C)CC] | —CH (CH₃)₂ | —CH₃ | —OC(O)CH₃ | —OCH₃ |
| 121 | BTZ | 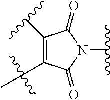 [SMILES: *C1=C(*)C(=O)N(*)C1=O] | — | —(CH₂)₂— | —NCH3C(O)— | 1 | 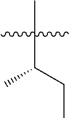 [SMILES: *[C@@H](C)CC] | —CH (CH₃)₂ | —CH₃ | —OC(O)CH₃ | —OCH₃ |
| 122 | BTZ | 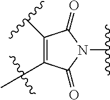 [SMILES: *C1=C(*)C(=O)N(*)C1=O] | — | — (CH₂CH₂O)₁₂ — | —NCH₃C(O)— | 1 | 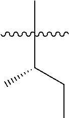 [SMILES: *[C@@H](C)CC] | —CH (CH₃)₂ | —CH₃ | —OC(O)CH₃ | —OCH₃ |
| 123 | BTZ | 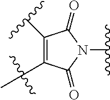 [SMILES: *C1=C(*)C(=O)N(*)C1=O] | — | — (CH₂CH₂O)₆— | —NCH₃C(O)— | 1 | 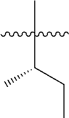 [SMILES: *[C@@H](C)CC] | —CH (CH₃)₂ | —CH₃ | —OC(O)CH₃ | —OCH₃ |
| 124 | BTZ | 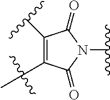 [SMILES: *C1=C(*)C(=O)N(*)C1=O] | —(CH₂)₂CO— | —NHCH₂CH₂— | —NCH₃C(O)— | 1 | 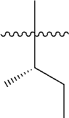 [SMILES: *[C@@H](C)CC] | —CH (CH₃)₂ | —CH₃ | —OC(O)CH₃ | —OCH₃ |
| 125 | BTZ | 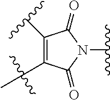 [SMILES: *C1=C(*)C(=O)N(*)C1=O] | —(CH₂)₄CO— | —NHCH₂CH₂— | —NCH₃C(O)— | 1 | 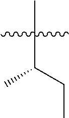 [SMILES: *[C@@H](C)CC] | —CH (CH₃)₂ | —CH₃ | —OC(O)CH₃ | —OCH₃ |
| 126 | BTZ | 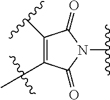 [SMILES: *C1=C(*)C(=O)N(*)C1=O] | —(CH₂)₅CO— | —NHCH₂CH₂— | —NCH₃C(O)— | 1 | 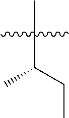 [SMILES: *[C@@H](C)CC] | —CH (CH₃)₂ | —CH₃ | —OC(O)CH₃ | —OCH₃ |
| 127 | BTZ | 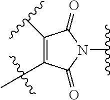 [SMILES: *C1=C(*)C(=O)N(*)C1=O] | — | —(CH₂)₂— | —OC(O)— | 1 | 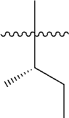 [SMILES: *[C@@H](C)CC] | —CH (CH₃)₂ | —CH₃ | —OC(O)CH₃ | —NH (CH₂)₂- Phenyl |
| 128 | BTZ | 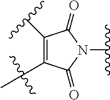 [SMILES: *C1=C(*)C(=O)N(*)C1=O] | — | — (CH₂CH₂O)₁₂ — | —CH₂CH₂C(O)— | 1 | 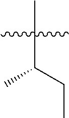 [SMILES: *[C@@H](C)CC] | —CH (CH₃)₂ | —CH₃ | —OC(O)CH₃ | —NH (CH₂)₂- Phenyl |
| 129 | BTZ | 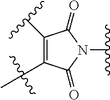 [SMILES: *C1=C(*)C(=O)N(*)C1=O] | — | — (CH₂CH₂O)₆— | —CH₂CH₂C(O)— | 1 | 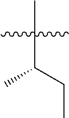 [SMILES: *[C@@H](C)CC] | —CH (CH₃)₂ | —CH₃ | —OC(O)CH₃ | —NH (CH₂)₂- Phenyl |
| 130 | BTZ | 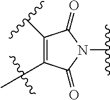 [SMILES: *C1=C(*)C(=O)N(*)C1=O] | —(CH₂)₂CO— | —NHCH₂CH₂— | —OC(O)— | 1 | 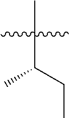 [SMILES: *[C@@H](C)CC] | —CH (CH₃)₂ | —CH₃ | —OC(O)CH₃ | —NH (CH₂)₂- Phenyl |
| 131 | BTZ | 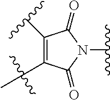 [SMILES: *C1=C(*)C(=O)N(*)C1=O] | —(CH₂)₄CO— | —NHCH₂CH₂— | —OC(O)— | 1 | 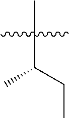 [SMILES: *[C@@H](C)CC] | —CH (CH₃)₂ | —CH₃ | —OC(O)CH₃ | —NH (CH₂)₂- Phenyl |
| 132 | BTZ | 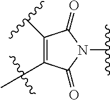 [SMILES: *C1=C(*)C(=O)N(*)C1=O] | —(CH₂)₅CO— | —NHCH₂CH₂— | —OC(O)— | 1 | 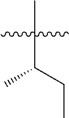 [SMILES: *[C@@H](C)CC] | —CH (CH₃)₂ | —CH₃ | —OC(O)CH₃ | —NH (CH₂)₂- Phenyl |
| 133 | BTZ | 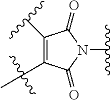 [SMILES: *C1=C(*)C(=O)N(*)C1=O] | — | —(CH₂)₂— | —NHC(O)— | 1 | 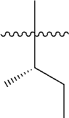 [SMILES: *[C@@H](C)CC] | —CH (CH₃)₂ | —CH₃ | —OC(O)CH₃ | —NH (CH₂)₂- Phenyl |
| 134 | BTZ | 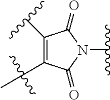 [SMILES: *C1=C(*)C(=O)N(*)C1=O] | — | — (CH₂CH₂O)₁₂ — | —NHC(O)— | 1 | 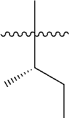 [SMILES: *[C@@H](C)CC] | —CH (CH₃)₂ | —CH₃ | —OC(O)CH₃ | —NH (CH₂)₂- Phenyl |
| 135 | BTZ | 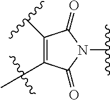 [SMILES: *C1=C(*)C(=O)N(*)C1=O] | — | — (CH₂CH₂O)₆— | —NHC(O)— | 1 | 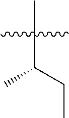 [SMILES: *[C@@H](C)CC] | —CH (CH₃)₂ | —CH₃ | —OC(O)CH₃ | —NH (CH₂)₂- Phenyl |
| 136 | BTZ | 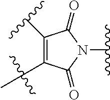 [SMILES: *C1=C(*)C(=O)N(*)C1=O] | —(CH₂)₂CO— | —NHCH₂CH₂— | —NHC(O)— | 1 | 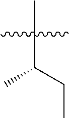 [SMILES: *[C@@H](C)CC] | —CH (CH₃)₂ | —CH₃ | —OC(O)CH₃ | —NH (CH₂)₂- Phenyl |
| 137 | BTZ | 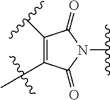 [SMILES: *C1=C(*)C(=O)N(*)C1=O] | —(CH₂)₄CO— | —NHCH₂CH₂— | —NHC(O)— | 1 | 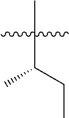 [SMILES: *[C@@H](C)CC] | —CH (CH₃)₂ | —CH₃ | —OC(O)CH₃ | —NH (CH₂)₂- Phenyl |
| 138 | BTZ | 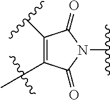 [SMILES: *C1=C(*)C(=O)N(*)C1=O] | —(CH₂)₅CO— | —NHCH₂CH₂— | —NHC(O)— | 1 | 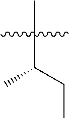 [SMILES: *[C@@H](C)CC] | —CH (CH₃)₂ | —CH₃ | —OC(O)CH₃ | —NH (CH₂)₂- Phenyl |
| 139 | BTZ | 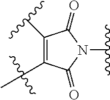 [SMILES: *C1=C(*)C(=O)N(*)C1=O] | — | —(CH₂)₂— | —NCH3C(O)— | 1 | 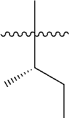 [SMILES: *[C@@H](C)CC] | —CH (CH₃)₂ | —CH₃ | —OC(O)CH₃ | —NH (CH₂)₂- Phenyl |
| 140 | BTZ | 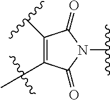 [SMILES: *C1=C(*)C(=O)N(*)C1=O] | — | — (CH₂CH₂O)₁₂ — | —NCH₃C(O)— | 1 | 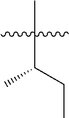 [SMILES: *[C@@H](C)CC] | —CH (CH₃)₂ | —CH₃ | —OC(O)CH₃ | —NH (CH₂)₂- Phenyl |
| 141 | BTZ | 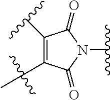 [SMILES: *C1=C(*)C(=O)N(*)C1=O] | — | — (CH₂CH₂O)₆— | —NCH₃C(O)— | 1 | 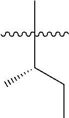 [SMILES: *[C@@H](C)CC] | —CH (CH₃)₂ | —CH₃ | —OC(O)CH₃ | —NH (CH₂)₂- Phenyl |
| 142 | BTZ | 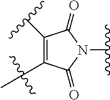 [SMILES: *C1=C(*)C(=O)N(*)C1=O] | —(CH₂)₂CO— | —NHCH₂CH₂— | —NCH₃C(O)— | 1 | 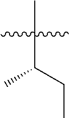 [SMILES: *[C@@H](C)CC] | —CH (CH₃)₂ | —CH₃ | —OC(O)CH₃ | —NH (CH₂)₂- Phenyl |
| 143 | BTZ | 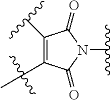 [SMILES: *C1=C(*)C(=O)N(*)C1=O] | —(CH₂)₄CO— | —NHCH₂CH₂— | —NCH₃C(O)— | 1 | 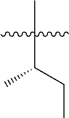 [SMILES: *[C@@H](C)CC] | —CH (CH₃)₂ | —CH₃ | —OC(O)CH₃ | —NH (CH₂)₂- Phenyl |
| 144 | BTZ | 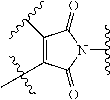 [SMILES: *C1=C(*)C(=O)N(*)C1=O] | —(CH₂)₅CO— | —NHCH₂CH₂— | —NCH₃C(O)— | 1 | 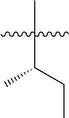 [SMILES: *[C@@H](C)CC] | —CH (CH₃)₂ | —CH₃ | —OC(O)CH₃ | —NH (CH₂)₂- Phenyl |
| 145 | BTZ | 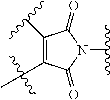 [SMILES: *C1=C(*)C(=O)N(*)C1=O] | — | —(CH₂)₂— | —OC(O)— | 1 | 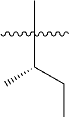 [SMILES: *[C@@H](C)CC] | —CH (CH₃)₂ | —CH₃ | —OC(O)CH₃ | —NH (CH₂)₂- p-MeO- phenyl |
| 146 | BTZ | 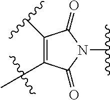 [SMILES: *C1=C(*)C(=O)N(*)C1=O] | — | — (CH₂CH₂O)₁₂ — | —CH₂CH₂C(O)— | 1 | 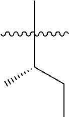 [SMILES: *[C@@H](C)CC] | —CH (CH₃)₂ | —CH₃ | —OC(O)CH₃ | —NH (CH₂)₂- p-MeO- phenyl |
| 147 | BTZ | 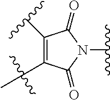 [SMILES: *C1=C(*)C(=O)N(*)C1=O] | — | — (CH₂CH₂O)₆— | —CH₂CH₂C(O)— | 1 | 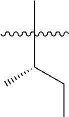 [SMILES: *[C@@H](C)CC] | —CH (CH₃)₂ | —CH₃ | —OC(O)CH₃ | —NH (CH₂)₂- p-MeO- phenyl |
| 148 | BTZ | 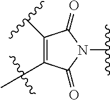 [SMILES: *C1=C(*)C(=O)N(*)C1=O] | —(CH₂)₂CO— | —NHCH₂CH₂— | —OC(O)— | 1 | 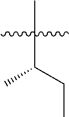 [SMILES: *[C@@H](C)CC] | —CH (CH₃)₂ | —CH₃ | —OC(O)CH₃ | —NH (CH₂)₂- p-MeO- phenyl |
| 149 | BTZ | 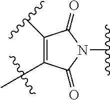 [SMILES: *C1=C(*)C(=O)N(*)C1=O] | —(CH₂)₄CO— | —NHCH₂CH₂— | —OC(O)— | 1 | 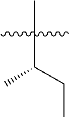 [SMILES: *[C@@H](C)CC] | —CH (CH₃)₂ | —CH₃ | —OC(O)CH₃ | —NH (CH₂)₂- p-MeO- phenyl |
| 150 | BTZ | 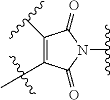 [SMILES: *C1=C(*)C(=O)N(*)C1=O] | —(CH₂)₅CO— | —NHCH₂CH₂— | —OC(O)— | 1 | 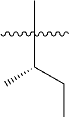 [SMILES: *[C@@H](C)CC] | —CH (CH₃)₂ | —CH₃ | —OC(O)CH₃ | —NH (CH₂)₂- p-MeO- phenyl |
| 151 | BTZ | 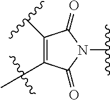 [SMILES: *C1=C(*)C(=O)N(*)C1=O] | — | —(CH₂)₂— | —NHC(O)— | 1 | 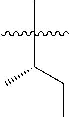 [SMILES: *[C@@H](C)CC] | —CH (CH₃)₂ | —CH₃ | —OC(O)CH₃ | —NH (CH₂)₂- p-MeO- phenyl |
| 152 | BTZ | 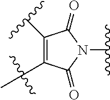 [SMILES: *C1=C(*)C(=O)N(*)C1=O] | — | — (CH₂CH₂O)₁₂ — | —NHC(O)— | 1 | 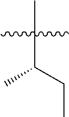 [SMILES: *[C@@H](C)CC] | —CH (CH₃)₂ | —CH₃ | —OC(O)CH₃ | —NH (CH₂)₂- p-MeO- phenyl |
| 153 | BTZ | 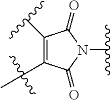 [SMILES: *C1=C(*)C(=O)N(*)C1=O] | — | — (CH₂CH₂O)₆— | —NHC(O)— | 1 | 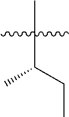 [SMILES: *[C@@H](C)CC] | —CH (CH₃)₂ | —CH₃ | —OC(O)CH₃ | —NH (CH₂)₂- p-MeO- phenyl |
| 154 | BTZ | 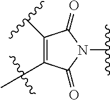 [SMILES: *C1=C(*)C(=O)N(*)C1=O] | —(CH₂)₂CO— | —NHCH₂CH₂— | —NHC(O)— | 1 | 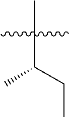 [SMILES: *[C@@H](C)CC] | —CH (CH₃)₂ | —CH₃ | —OC(O)CH₃ | —NH (CH₂)₂- p-MeO- phenyl |
| 155 | BTZ | 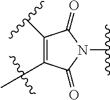 [SMILES: *C1=C(*)C(=O)N(*)C1=O] | —(CH₂)₄CO— | —NHCH₂CH₂— | —NHC(O)— | 1 | 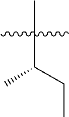 [SMILES: *[C@@H](C)CC] | —CH (CH₃)₂ | —CH₃ | —OC(O)CH₃ | —NH (CH₂)₂- p-MeO- phenyl |
| 156 | BTZ | 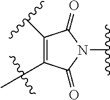 [SMILES: *C1=C(*)C(=O)N(*)C1=O] | —(CH₂)₅CO— | —NHCH₂CH₂— | —NHC(O)— | 1 | 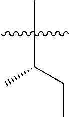 [SMILES: *[C@@H](C)CC] | —CH (CH₃)₂ | —CH₃ | —OC(O)CH₃ | —NH (CH₂)₂- p-MeO- phenyl |
| 157 | BTZ | 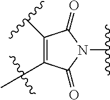 [SMILES: *C1=C(*)C(=O)N(*)C1=O] | — | —(CH₂)₂— | —NCH3C(O)— | 1 | 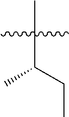 [SMILES: *[C@@H](C)CC] | —CH (CH₃)₂ | —CH₃ | —OC(O)CH₃ | —NH (CH₂)₂- p-MeO- phenyl |
| 158 | BTZ | 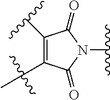 [SMILES: *C1=C(*)C(=O)N(*)C1=O] | — | — (CH₂CH₂O)₁₂ — | —NCH₃C(O)— | 1 | 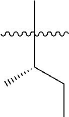 [SMILES: *[C@@H](C)CC] | —CH (CH₃)₂ | —CH₃ | —OC(O)CH₃ | —NH (CH₂)₂- p-MeO- phenyl |
| 159 | BTZ | 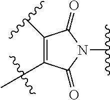 [SMILES: *C1=C(*)C(=O)N(*)C1=O] | — | — (CH₂CH₂O)₆— | —NCH₃C(O)— | 1 | 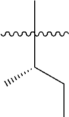 [SMILES: *[C@@H](C)CC] | —CH (CH₃)₂ | —CH₃ | —OC(O)CH₃ | —NH (CH₂)₂- p-MeO- phenyl |
| 160 | BTZ | 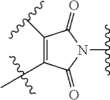 [SMILES: *C1=C(*)C(=O)N(*)C1=O] | —(CH₂)₂CO— | —NHCH₂CH₂— | —NCH₃C(O)— | 1 | 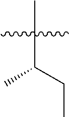 [SMILES: *[C@@H](C)CC] | —CH (CH₃)₂ | —CH₃ | —OC(O)CH₃ | —NH (CH₂)₂- p-MeO- phenyl |
| 161 | BTZ | 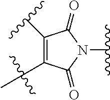 [SMILES: *C1=C(*)C(=O)N(*)C1=O] | —(CH₂)₄CO— | —NHCH₂CH₂— | —NCH₃C(O)— | 1 | 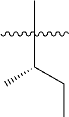 [SMILES: *[C@@H](C)CC] | —CH (CH₃)₂ | —CH₃ | —OC(O)CH₃ | —NH (CH₂)₂- p-MeO- phenyl |
| 162 | BTZ | 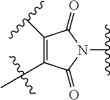 [SMILES: *C1=C(*)C(=O)N(*)C1=O] | —(CH₂)₅CO— | —NHCH₂CH₂— | —NCH₃C(O)— | 1 | 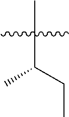 [SMILES: *[C@@H](C)CC] | —CH (CH₃)₂ | —CH₃ | —OC(O)CH₃ | —NH (CH₂)₂- p-MeO- phenyl |
| 163 | BTZ | 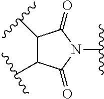 [SMILES: *C1C(=O)N(*)C(=O)C1*] | — | —(CH₂)₂— | —OC(O)— | 1 | 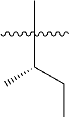 [SMILES: *[C@@H](C)CC] | —CH (CH₃)₂ | —CH₃ | —OC(O)CH₃ | —OCH₃ |
| 164 | BTZ | 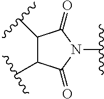 [SMILES: *C1C(=O)N(*)C(=O)C1*] | — | — (CH₂CH₂O)₁₂ — | —CH₂CH₂C(O)— | 1 | 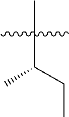 [SMILES: *[C@@H](C)CC] | —CH (CH₃)₂ | —CH₃ | —OC(O)CH₃ | —OCH₃ |
| 165 | BTZ | 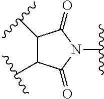 [SMILES: *C1C(=O)N(*)C(=O)C1*] | — | — (CH₂CH₂O)₆— | —CH₂CH₂C(O)— | 1 | 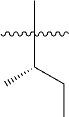 [SMILES: *[C@@H](C)CC] | —CH (CH₃)₂ | —CH₃ | —OC(O)CH₃ | —OCH₃ |
| 166 | BTZ | 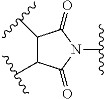 [SMILES: *C1C(=O)N(*)C(=O)C1*] | —(CH₂)₂CO— | —NHCH₂CH₂— | —OC(O)— | 1 | 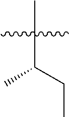 [SMILES: *[C@@H](C)CC] | —CH (CH₃)₂ | —CH₃ | —OC(O)CH₃ | —OCH₃ |
| 167 | BTZ | 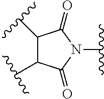 [SMILES: *C1C(=O)N(*)C(=O)C1*] | —(CH₂)₄CO— | —NHCH₂CH₂— | —OC(O)— | 1 | 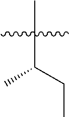 [SMILES: *[C@@H](C)CC] | —CH (CH₃)₂ | —CH₃ | —OC(O)CH₃ | —OCH₃ |
| 168 | BTZ | 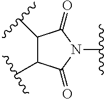 [SMILES: *C1C(=O)N(*)C(=O)C1*] | —(CH₂)₅CO— | —NHCH₂CH₂— | —OC(O)— | 1 | 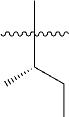 [SMILES: *[C@@H](C)CC] | —CH (CH₃)₂ | —CH₃ | —OC(O)CH₃ | —OCH₃ |
| 169 | BTZ | 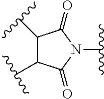 [SMILES: *C1C(=O)N(*)C(=O)C1*] | — | —(CH₂)₂— | —NHC(O)— | 1 | 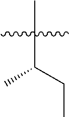 [SMILES: *[C@@H](C)CC] | —CH (CH₃)₂ | —CH₃ | —OC(O)CH₃ | —OCH₃ |
| 170 | BTZ | 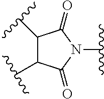 [SMILES: *C1C(=O)N(*)C(=O)C1*] | — | — (CH₂CH₂O)₁₂ — | —NHC(O)— | 1 | 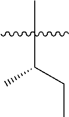 [SMILES: *[C@@H](C)CC] | —CH (CH₃)₂ | —CH₃ | —OC(O)CH₃ | —OCH₃ |
| 171 | BTZ | 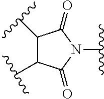 [SMILES: *C1C(=O)N(*)C(=O)C1*] | — | — (CH₂CH₂O)₆— | —NHC(O)— | 1 | 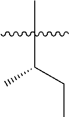 [SMILES: *[C@@H](C)CC] | —CH (CH₃)₂ | —CH₃ | —OC(O)CH₃ | —OCH₃ |
| 172 | BTZ | 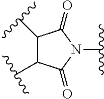 [SMILES: *C1C(=O)N(*)C(=O)C1*] | —(CH₂)₂CO— | —NHCH₂CH₂— | —NHC(O)— | 1 | 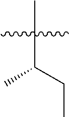 [SMILES: *[C@@H](C)CC] | —CH (CH₃)₂ | —CH₃ | —OC(O)CH₃ | —OCH₃ |
| 173 | BTZ | 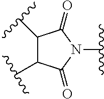 [SMILES: *C1C(=O)N(*)C(=O)C1*] | —(CH₂)₄CO— | —NHCH₂CH₂— | —NHC(O)— | 1 | 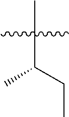 [SMILES: *[C@@H](C)CC] | —CH (CH₃)₂ | —CH₃ | —OC(O)CH₃ | —OCH₃ |
| 174 | BTZ | 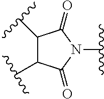 [SMILES: *C1C(=O)N(*)C(=O)C1*] | —(CH₂)₅CO— | —NHCH₂CH₂— | —NHC(O)— | 1 | 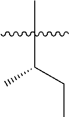 [SMILES: *[C@@H](C)CC] | —CH (CH₃)₂ | —CH₃ | —OC(O)CH₃ | —OCH₃ |
| 175 | BTZ | 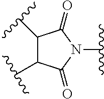 [SMILES: *C1C(=O)N(*)C(=O)C1*] | — | —(CH₂)₂— | —NCH3C(O)— | 1 | 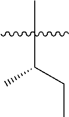 [SMILES: *[C@@H](C)CC] | —CH (CH₃)₂ | —CH₃ | —OC(O)CH₃ | —OCH₃ |
| 176 | BTZ | 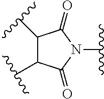 [SMILES: *C1C(=O)N(*)C(=O)C1*] | — | — (CH₂CH₂O)₁₂ — | —NCH₃C(O)— | 1 | 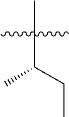 [SMILES: *[C@@H](C)CC] | —CH (CH₃)₂ | —CH₃ | —OC(O)CH₃ | —OCH₃ |
| 177 | BTZ | 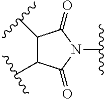 [SMILES: *C1C(=O)N(*)C(=O)C1*] | — | — (CH₂CH₂O)₆— | —NCH₃C(O)— | 1 | 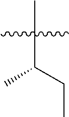 [SMILES: *[C@@H](C)CC] | —CH (CH₃)₂ | —CH₃ | —OC(O)CH₃ | —OCH₃ |
| 178 | BTZ | 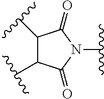 [SMILES: *C1C(=O)N(*)C(=O)C1*] | —(CH₂)₂CO— | —NHCH₂CH₂— | —NCH₃C(O)— | 1 | 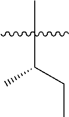 [SMILES: *[C@@H](C)CC] | —CH (CH₃)₂ | —CH₃ | —OC(O)CH₃ | —OCH₃ |
| 179 | BTZ | 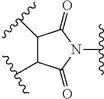 [SMILES: *C1C(=O)N(*)C(=O)C1*] | —(CH₂)₄CO— | —NHCH₂CH₂— | —NCH₃C(O)— | 1 | 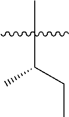 [SMILES: *[C@@H](C)CC] | —CH (CH₃)₂ | —CH₃ | —OC(O)CH₃ | —OCH₃ |
| 180 | BTZ | 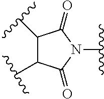 [SMILES: *C1C(=O)N(*)C(=O)C1*] | —(CH₂)₅CO— | —NHCH₂CH₂— | —NCH₃C(O)— | 1 | 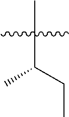 [SMILES: *[C@@H](C)CC] | —CH (CH₃)₂ | —CH₃ | —OC(O)CH₃ | —OCH₃ |
| 181 | BTZ | 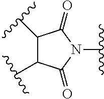 [SMILES: *C1C(=O)N(*)C(=O)C1*] | — | —(CH₂)₂— | —OC(O)— | 1 | 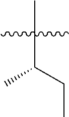 [SMILES: *[C@@H](C)CC] | —CH (CH₃)₂ | —CH₃ | —OC(O)CH₃ | —NH (CH₂)₂- Phenyl |
| 182 | BTZ | 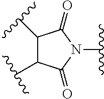 [SMILES: *C1C(=O)N(*)C(=O)C1*] | — | — (CH₂CH₂O)₁₂ — | —CH₂CH₂C(O)— | 1 | 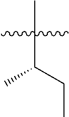 [SMILES: *[C@@H](C)CC] | —CH (CH₃)₂ | —CH₃ | —OC(O)CH₃ | —NH (CH₂)₂- Phenyl |
| 183 | BTZ | 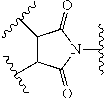 [SMILES: *C1C(=O)N(*)C(=O)C1*] | — | — (CH₂CH₂O)₆— | —CH₂CH₂C(O)— | 1 | 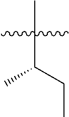 [SMILES: *[C@@H](C)CC] | —CH (CH₃)₂ | —CH₃ | —OC(O)CH₃ | —NH (CH₂)₂- Phenyl |
| 184 | BTZ | 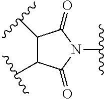 [SMILES: *C1C(=O)N(*)C(=O)C1*] | —(CH₂)₂CO— | —NHCH₂CH₂— | —OC(O)— | 1 | 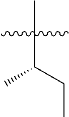 [SMILES: *[C@@H](C)CC] | —CH (CH₃)₂ | —CH₃ | —OC(O)CH₃ | —NH (CH₂)₂- Phenyl |
| 185 | BTZ | 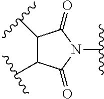 [SMILES: *C1C(=O)N(*)C(=O)C1*] | —(CH₂)₄CO— | —NHCH₂CH₂— | —OC(O)— | 1 | 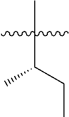 [SMILES: *[C@@H](C)CC] | —CH (CH₃)₂ | —CH₃ | —OC(O)CH₃ | —NH (CH₂)₂- Phenyl |
| 186 | BTZ | 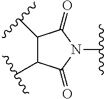 [SMILES: *C1C(=O)N(*)C(=O)C1*] | —(CH₂)₅CO— | —NHCH₂CH₂— | —OC(O)— | 1 | 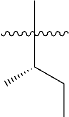 [SMILES: *[C@@H](C)CC] | —CH (CH₃)₂ | —CH₃ | —OC(O)CH₃ | —NH (CH₂)₂- Phenyl |
| 187 | BTZ | 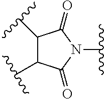 [SMILES: *C1C(=O)N(*)C(=O)C1*] | — | —(CH₂)₂— | —NHC(O)— | 1 | 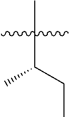 [SMILES: *[C@@H](C)CC] | —CH (CH₃)₂ | —CH₃ | —OC(O)CH₃ | —NH (CH₂)₂- Phenyl |
| 188 | BTZ | 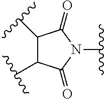 [SMILES: *C1C(=O)N(*)C(=O)C1*] | — | — (CH₂CH₂O)₁₂ — | —NHC(O)— | 1 | 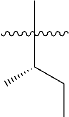 [SMILES: *[C@@H](C)CC] | —CH (CH₃)₂ | —CH₃ | —OC(O)CH₃ | —NH (CH₂)₂- Phenyl |
| 189 | BTZ | 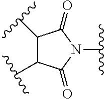 [SMILES: *C1C(=O)N(*)C(=O)C1*] | — | — (CH₂CH₂O)₆— | —NHC(O)— | 1 | 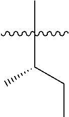 [SMILES: *[C@@H](C)CC] | —CH (CH₃)₂ | —CH₃ | —OC(O)CH₃ | —NH (CH₂)₂- Phenyl |
| 200 | BTZ | 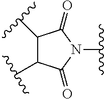 [SMILES: *C1C(=O)N(*)C(=O)C1*] | —(CH₂)₂CO— | —NHCH₂CH₂— | —NHC(O)— | 1 | 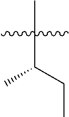 [SMILES: *[C@@H](C)CC] | —CH (CH₃)₂ | —CH₃ | —OC(O)CH₃ | —NH (CH₂)₂- Phenyl |
| 201 | BTZ | 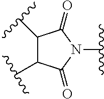 [SMILES: *C1C(=O)N(*)C(=O)C1*] | —(CH₂)₄CO— | —NHCH₂CH₂— | —NHC(O)— | 1 | 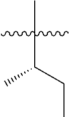 [SMILES: *[C@@H](C)CC] | —CH (CH₃)₂ | —CH₃ | —OC(O)CH₃ | —NH (CH₂)₂- Phenyl |
| 202 | BTZ | 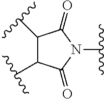 [SMILES: *C1C(=O)N(*)C(=O)C1*] | —(CH₂)₅CO— | —NHCH₂CH₂— | —NHC(O)— | 1 | 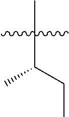 [SMILES: *[C@@H](C)CC] | —CH (CH₃)₂ | —CH₃ | —OC(O)CH₃ | —NH (CH₂)₂- Phenyl |
| 203 | BTZ | 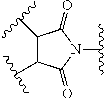 [SMILES: *C1C(=O)N(*)C(=O)C1*] | — | —(CH₂)₂— | —NCH3C(O)— | 1 | 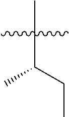 [SMILES: *[C@@H](C)CC] | —CH (CH₃)₂ | —CH₃ | —OC(O)CH₃ | —NH (CH₂)₂- Phenyl |
| 204 | BTZ | 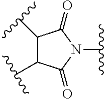 [SMILES: *C1C(=O)N(*)C(=O)C1*] | — | — (CH₂CH₂O)₁₂ — | —NCH₃C(O)— | 1 | 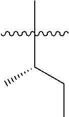 [SMILES: *[C@@H](C)CC] | —CH (CH₃)₂ | —CH₃ | —OC(O)CH₃ | —NH (CH₂)₂- Phenyl |
| 205 | BTZ | 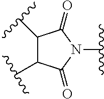 [SMILES: *C1C(=O)N(*)C(=O)C1*] | — | — (CH₂CH₂O)₆— | —NCH₃C(O)— | 1 | 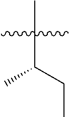 [SMILES: *[C@@H](C)CC] | —CH (CH₃)₂ | —CH₃ | —OC(O)CH₃ | —NH (CH₂)₂- Phenyl |
| 206 | BTZ | 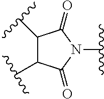 [SMILES: *C1C(=O)N(*)C(=O)C1*] | —(CH₂)₂CO— | —NHCH₂CH₂— | —NCH₃C(O)— | 1 | 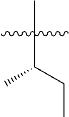 [SMILES: *[C@@H](C)CC] | —CH (CH₃)₂ | —CH₃ | —OC(O)CH₃ | —NH (CH₂)₂- Phenyl |
| 207 | BTZ | 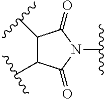 [SMILES: *C1C(=O)N(*)C(=O)C1*] | —(CH₂)₄CO— | —NHCH₂CH₂— | —NCH₃C(O)— | 1 | 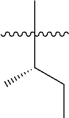 [SMILES: *[C@@H](C)CC] | —CH (CH₃)₂ | —CH₃ | —OC(O)CH₃ | —NH (CH₂)₂- Phenyl |
| 208 | BTZ | 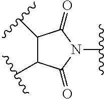 [SMILES: *C1C(=O)N(*)C(=O)C1*] | —(CH₂)₅CO— | —NHCH₂CH₂— | —NCH₃C(O)— | 1 | 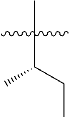 [SMILES: *[C@@H](C)CC] | —CH (CH₃)₂ | —CH₃ | —OC(O)CH₃ | —NH (CH₂)₂- Phenyl |
| 209 | BTZ | 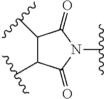 [SMILES: *C1C(=O)N(*)C(=O)C1*] | — | —(CH₂)₂— | —OC(O)— | 1 | 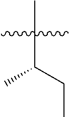 [SMILES: *[C@@H](C)CC] | —CH (CH₃)₂ | —CH₃ | —OC(O)CH₃ | —NH (CH₂)₂- p-MeO- phenyl |
| 210 | BTZ | 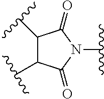 [SMILES: *C1C(=O)N(*)C(=O)C1*] | — | — (CH₂CH₂O)₁₂ — | —CH₂CH₂C(O)— | 1 | 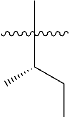 [SMILES: *[C@@H](C)CC] | —CH (CH₃)₂ | —CH₃ | —OC(O)CH₃ | —NH (CH₂)₂- p-MeO- phenyl |
| 211 | BTZ | 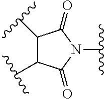 [SMILES: *C1C(=O)N(*)C(=O)C1*] | — | — (CH₂CH₂O)₆— | —CH₂CH₂C(O)— | 1 | 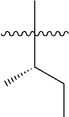 [SMILES: *[C@@H](C)CC] | —CH (CH₃)₂ | —CH₃ | —OC(O)CH₃ | —NH (CH₂)₂- p-MeO- phenyl |
| 212 | BTZ | 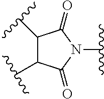 [SMILES: *C1C(=O)N(*)C(=O)C1*] | —(CH₂)₂CO— | —NHCH₂CH₂— | —OC(O)— | 1 | 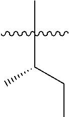 [SMILES: *[C@@H](C)CC] | —CH (CH₃)₂ | —CH₃ | —OC(O)CH₃ | —NH (CH₂)₂- p-MeO- phenyl |
| 213 | BTZ | 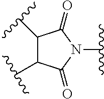 [SMILES: *C1C(=O)N(*)C(=O)C1*] | —(CH₂)₄CO— | —NHCH₂CH₂— | —OC(O)— | 1 | 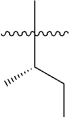 [SMILES: *[C@@H](C)CC] | —CH (CH₃)₂ | —CH₃ | —OC(O)CH₃ | —NH (CH₂)₂- p-MeO- phenyl |
| 214 | BTZ | 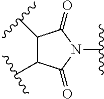 [SMILES: *C1C(=O)N(*)C(=O)C1*] | —(CH₂)₅CO— | —NHCH₂CH₂— | —OC(O)— | 1 | 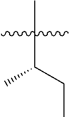 [SMILES: *[C@@H](C)CC] | —CH (CH₃)₂ | —CH₃ | —OC(O)CH₃ | —NH (CH₂)₂- p-MeO- phenyl |
| 215 | BTZ | 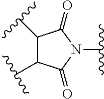 [SMILES: *C1C(=O)N(*)C(=O)C1*] | — | —(CH₂)₂— | —NHC(O)— | 1 | 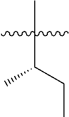 [SMILES: *[C@@H](C)CC] | —CH (CH₃)₂ | —CH₃ | —OC(O)CH₃ | —NH (CH₂)₂- p-MeO- phenyl |
| 216 | BTZ | 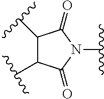 [SMILES: *C1C(=O)N(*)C(=O)C1*] | — | — (CH₂CH₂O)₁₂ — | —NHC(O)— | 1 | 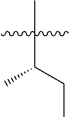 [SMILES: *[C@@H](C)CC] | —CH (CH₃)₂ | —CH₃ | —OC(O)CH₃ | —NH (CH₂)₂- p-MeO- phenyl |
| 217 | BTZ | 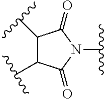 [SMILES: *C1C(=O)N(*)C(=O)C1*] | — | — (CH₂CH₂O)₆— | —NHC(O)— | 1 | 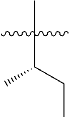 [SMILES: *[C@@H](C)CC] | —CH (CH₃)₂ | —CH₃ | —OC(O)CH₃ | —NH (CH₂)₂- p-MeO- phenyl |
| 218 | BTZ | 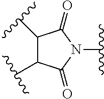 [SMILES: *C1C(=O)N(*)C(=O)C1*] | —(CH₂)₂CO— | —NHCH₂CH₂— | —NHC(O)— | 1 | 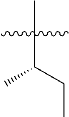 [SMILES: *[C@@H](C)CC] | —CH (CH₃)₂ | —CH₃ | —OC(O)CH₃ | —NH (CH₂)₂- p-MeO- phenyl |
| 219 | BTZ | 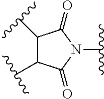 [SMILES: *C1C(=O)N(*)C(=O)C1*] | —(CH₂)₄CO— | —NHCH₂CH₂— | —NHC(O)— | 1 | 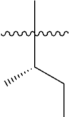 [SMILES: *[C@@H](C)CC] | —CH (CH₃)₂ | —CH₃ | —OC(O)CH₃ | —NH (CH₂)₂- p-MeO- phenyl |
| 220 | BTZ | 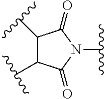 [SMILES: *C1C(=O)N(*)C(=O)C1*] | —(CH₂)₅CO— | —NHCH₂CH₂— | —NHC(O)— | 1 | 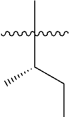 [SMILES: *[C@@H](C)CC] | —CH (CH₃)₂ | —CH₃ | —OC(O)CH₃ | —NH (CH₂)₂- p-MeO- phenyl |
| 221 | BTZ | 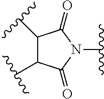 [SMILES: *C1C(=O)N(*)C(=O)C1*] | — | —(CH₂)₂— | —NCH3C(O)— | 1 | 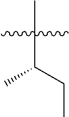 [SMILES: *[C@@H](C)CC] | —CH (CH₃)₂ | —CH₃ | —OC(O)CH₃ | —NH (CH₂)₂- p-MeO- phenyl |
| 222 | BTZ | 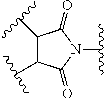 [SMILES: *C1C(=O)N(*)C(=O)C1*] | — | — (CH₂CH₂O)₁₂ — | —NCH₃C(O)— | 1 | 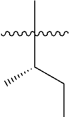 [SMILES: *[C@@H](C)CC] | —CH (CH₃)₂ | —CH₃ | —OC(O)CH₃ | —NH (CH₂)₂- p-MeO- phenyl |
| 223 | BTZ | 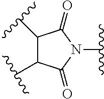 [SMILES: *C1C(=O)N(*)C(=O)C1*] | — | — (CH₂CH₂O)₆— | —NCH₃C(O)— | 1 | 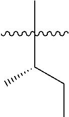 [SMILES: *[C@@H](C)CC] | —CH (CH₃)₂ | —CH₃ | —OC(O)CH₃ | —NH (CH₂)₂- p-MeO- phenyl |
| 224 | BTZ | 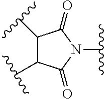 [SMILES: *C1C(=O)N(*)C(=O)C1*] | —(CH₂)₂CO— | —NHCH₂CH₂— | —NCH₃C(O)— | 1 | 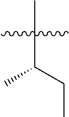 [SMILES: *[C@@H](C)CC] | —CH (CH₃)₂ | —CH₃ | —OC(O)CH₃ | —NH (CH₂)₂- p-MeO- phenyl |
| 225 | BTZ | 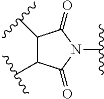 [SMILES: *C1C(=O)N(*)C(=O)C1*] | —(CH₂)₄CO— | —NHCH₂CH₂— | —NCH₃C(O)— | 1 | 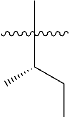 [SMILES: *[C@@H](C)CC] | —CH (CH₃)₂ | —CH₃ | —OC(O)CH₃ | —NH (CH₂)₂- p-MeO- phenyl |
| 226 | BTZ | 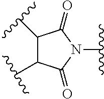 [SMILES: *C1C(=O)N(*)C(=O)C1*] | —(CH₂)₅CO— | —NHCH₂CH₂— | —NCH₃C(O)— | 1 | 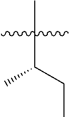 [SMILES: *[C@@H](C)CC] | —CH (CH₃)₂ | —CH₃ | —OC(O)CH₃ | —NH (CH₂)₂- p-MeO- phenyl |
| 228 | GTZ | 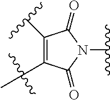 [SMILES: *C1=C(*)C(=O)N(*)C1=O] | — | —(CH₂)₂— | —OC(O)— | 1 | 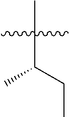 [SMILES: *[C@@H](C)CC] | —CH (CH₃)₂ | —CH₃ | —OC(O)CH₃ | —OCH₃ |
| 229 | TTZ | 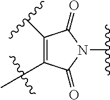 [SMILES: *C1=C(*)C(=O)N(*)C1=O] | — | —(CH₂)₂— | —OC(O)— | 1 | 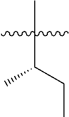 [SMILES: *[C@@H](C)CC] | —CH (CH₃)₂ | —CH₃ | —OC(O)CH₃ | —OCH₃ |
| 230 | GBZ | 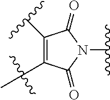 [SMILES: *C1=C(*)C(=O)N(*)C1=O] | — | —(CH₂)₂— | —OC(O)— | 1 | 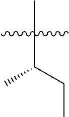 [SMILES: *[C@@H](C)CC] | —CH (CH₃)₂ | —CH₃ | —OC(O)CH₃ | —OCH₃ |
| 231 | LVT | 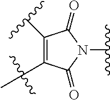 [SMILES: *C1=C(*)C(=O)N(*)C1=O] | — | —(CH₂)₂— | —OC(O)— | 1 | 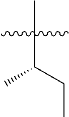 [SMILES: *[C@@H](C)CC] | —CH (CH₃)₂ | —CH₃ | —OC(O)CH₃ | —OCH₃ |

A^{a} (Antibodies):
TZ (trastuzumab),
BTX (brentuximab),
GTZ (gemtuzumab),
ITZ (inotuzumab),
GBT (glembatumumab) and
LVT (lovortuzumab).

### Assays

The ADCs of the present application may be assayed for binding affinity to and specificity for the desired antigen by any of the methods conventionally used for the assay of antibodies; and they may be assayed for efficacy as anticancer agents by any of the methods conventionally used for the assay of cytostatic/cytotoxic agents, such as assays for potency against cell cultures, xenograft assays, and the like. A person of ordinary skill in the art will have no difficulty, considering that skill and the literature available, in determining suitable assay techniques; from the results of those assays, in determining suitable doses to test in humans as anticancer agents, and, from the results of those tests, in determining suitable doses to use to treat cancers in humans.

### Formulation and Administration

The ADCs of the first aspect of this invention will typically be formulated as solutions for intravenous administration, or as lyophilized concentrates for reconstitution to prepare intravenous solutions (to be reconstituted, e.g., with normal saline, 5% dextrose, or similar isotonic solutions). They will typically be administered by intravenous injection or infusion. A person of ordinary skill in the art of pharmaceutical formulation, especially the formulation of anticancer antibodies, will have no difficulty, considering that skill and the literature available, in developing suitable formulations.

### EXAMPLES

### Synthesis of Linkers

The following procedures may be employed for the preparation of the compounds of the present invention, such as the compounds described in Table 1. The starting materials and reagents used in preparing these compounds are either available from commercial suppliers such as the Aldrich Chemical Company (Milwaukee, Wis.), Bachem (Torrance, Calif.), Sigma (St. Louis, Mo.), or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

In some cases, protective groups may be introduced and finally removed. Suitable protective groups for amino, hydroxy and carboxy groups are described in Greene et al., Protective Groups in Organic Synthesis, Second Edition, John Wiley and Sons, New York, 1991. Standard organic chemical reactions can be achieved by using a number of different reagents, for examples, as described in Larock: Comprehensive Organic Transformations, VCH Publishers, New York, 1989.

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope.

### Example 1

### Synthesis of 3,4-bis(2-pyridylsulfanyl)pyrrole-2,5-dione

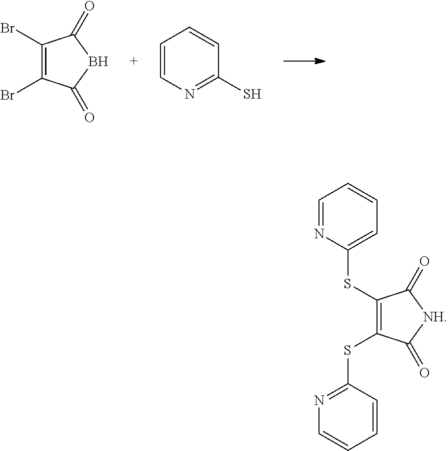

3,4-Dibromopyrrole-2,5-dione [2,3-dibromomaleimide], 1 g, was added to a clean 100 mL round bottom flask with a rubber stopper and bubbler, and dissolved in 50 mL HPLC grade methanol. 2-Pyridinethiol, 2 equivalents, was added to a 20 mL scintillation vial, and dissolved in 10 mL methanol. Under nitrogen and with stirring, the 2-pyridinethiol/methanol solution was added dropwise to the 3,4-dibromopyrrole-2,5-dione via a 20 mL syringe with a 16 gauge needle, and the reaction mixture was stirred for an additional 3-4 hours. The methanol was evaporated and the crude product was dissolved in ethyl acetate and loaded onto about 2 g silica gel. The silica gel-loaded crude product was eluted through a 12 g silica gel cartridge with a hexane:ethyl acetate gradient from 9:1 to 0:1 over 25 column volumes. The enriched fractions were identified, pooled and lyophilized to dryness. The final product was recrystallized from ethyl acetate and diethyl ether to provide yellow needle crystals which were collected by filtration.

Similar syntheses may be performed using the methods of Schumacher et al. for other 3,4-di(R-sulfanyl)pyrrole-2,5-diones (see the Supplementary Materials at pages S17-S18). Similar syntheses may also be performed starting with (3,4-dibromo-2,5-dioxopyrrolyl)-terminated linkers [i.e. compounds where a sidechain has already been added to the pyrrole nitrogen] to give the corresponding (2,5-dioxo-3,4-di(R-sulfanyl)pyrrolyl)-terminated linkers; and/or with other thiols (such as the benzenethiol and 2-hydroxyethanethiol of Schumacher et al.) to give the corresponding linkers; and/or with other pyrrolediones or pyrrolidinediones, such as 3,4-dichloropyrrole-2,5-dione or 3,4-dibromopyrrolidine-2,5-dione, or based on them, to give the corresponding 3,4-di(R-sulfanyl) pyrrole-2,5-diones or 3,4-di(R-sulfanyl)pyrrolidine-2,5-diones or linkers based on them.

General procedures for the synthesis of the linkers (L, L^{I}, L² and L³) may be performed using standard synthetic procedures as described in Larock, above, or Modern Synthetic Reactions, Second Edition, H. O. House, The Benjamin/Cummings Publishing Company, Menlo Park, Calif., 1972; the chemistry of amino acids and peptide synthesis described in The Chemistry of the Amino Acids, J. P. Greenstein, M. Winitz, Robert E. Krieger Publishing Company, Malabar, Fla., 1986, Volumes 1, 2 and 3.

### Example 2

### Synthesis of 39-(3,4-dibromo-2,5-dioxopyrrolyl)-3,6,9,12,15,18,21,24,27,30,33,36-dodecaoxanonatriacontanoic acid

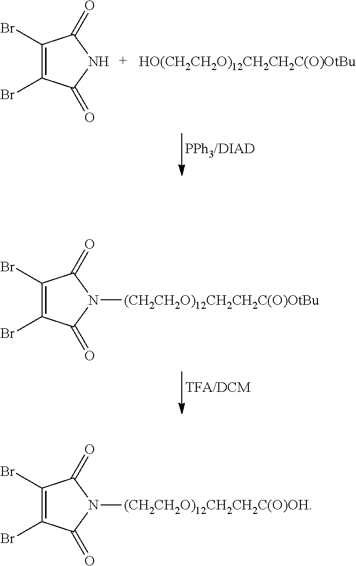

A 100 mL two-necked round bottom flask was flame dried and cooled under nitrogen. The cooled flask was charged with 200 mg (0.296 mmol) of tert-butyl 39-hydroxy-3,6,9,12,15,18,21,24,27,30,33,36-dodecaoxanonatriacontanoate. Triphenylphosphine, 106 mg, was dissolved in about 5 mL anhydrous tetrahydrofuran in a vial, and the solution was added to the 100 mL flask via cannula under nitrogen. The 100 mL flask was cooled in an ice-water bath for 15 minutes. To the cooled solution was added 55 mg (0.217 mmol) 3,4-dibromopyrrole-2,5-dione with stirring until a clear solution was observed. DIAD, 58.3 μL, was added to the cooled reaction mixture, which was stirred in the ice bath for an additional 10 minutes. The reaction mixture was stirred and allowed to reach room temperature over about 20 hours, then concentrated on a rotary evaporator until dry, giving a yellow viscous oil, which was absorbed onto about 1 g silica gel and dry-loaded onto a Reveleris normal phase chromatography unit. The oil was eluted over a 12 g silica gel cartridge with a methanol:dichloromethane gradient from 1:0 to 9:1 over 28 column volumes. The fractions containing the desired product were pooled and concentrated to dryness. The purified product was suspended in 50:50 acetonitrile:water and lyophilized overnight to provide a clear light yellow viscous oil. By LC-MS analysis, the tert-butyl-protected carboxylic acid product had been partially deprotected during the work-up. To fully deprotect the material to the free acid, the lyophilized material was treated with 5% trifluoroacetic acid in dichloromethane, concentrated to dryness and lyophilized in acetonitrile:water (50:50) overnight.

Similar syntheses may be performed starting with 3,4-bis(2-pyridylsulfanyl)pyrrole-2,5-dione to give 39-(2,5-dioxo-3,4-bis(2-pyridylsulfanyl)pyrrolyl)-3,6,9,12,15,18,21,24,27,30,33,36-dodecaoxanonatriacontanoic acid, or starting with other 3,4-di(R-sulfanyl)pyrrole-2,5-diones to give the corresponding linkers; and/or starting with other hydroxyl-terminated sidechains, e.g. using tert-butyl 6-hydroxyhexanoate to give 6-(3,4-dibromo-2,5-dioxopyrrolyl)hexanoic acid, etc. Similar syntheses starting with maleimide rather than 2,3-dibromomaleimide give comparator linkers of the prior art, such as 6-(2,5-dioxopyrrolyl)hexanoic acid, the MC linker.

### Example 3

### Synthesis of 39-(3,4-dibromo-2,5-dioxopyrrolidinyl)-3,6,9,12,15,18,21,24,27,30,33,36-dodecaoxanonatriacontanoic acid [the dBrPEG linker]

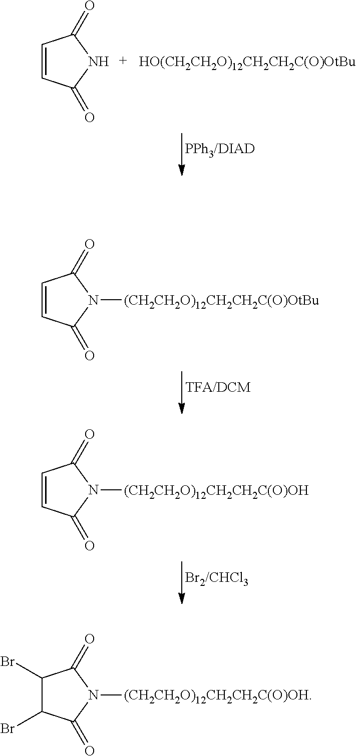

39-(2,5-dioxopyrrolyl)-3,6,9,12,15,18,21,24,27,30,33,36-dodecaoxanonatriacontanoic acid was prepared in the same manner as the 39-(3,4-dibromo-2,5-dioxopyrrolyl)-3,6,9,12,15,18,21,24,27,30,33,36-dodecaoxanonatriacontanoic acid of Example 2, but starting with maleimide rather than 2,3-dibromomaleimide. The acid was treated with 0.5 equivalents of bromine in chloroform followed by refluxing overnight to give 39-(3,4-dibromo-2,5-dioxopyrrolidinyl)-3,6,9,12,15,18,21,24,27,30,33,36-dodecaoxanonatriacontanoic acid after flash purification on silica gel.

Similar syntheses may be performed using other hydroxyl-terminated sidechains, e.g. using tert-butyl 6-hydroxyhexanoate to give 6-(3,4-dibromo-2,5-dioxopyrrolidinyl)hexanoic acid, etc. The dibrominated linkers that are products of this synthesis may be dehydrobrominated with base in an additional step to give (3-bromo-2,5-dioxopyrrolyl)-terminated linkers, such as 6-(3-bromo-2,5-dioxopyrrolyl)hexanoic acid.

### Synthesis of Linker-Cytotoxin Conjugates:

### Synthesis of T2

Different methods for the preparation of T2 are shown in the Schemes.

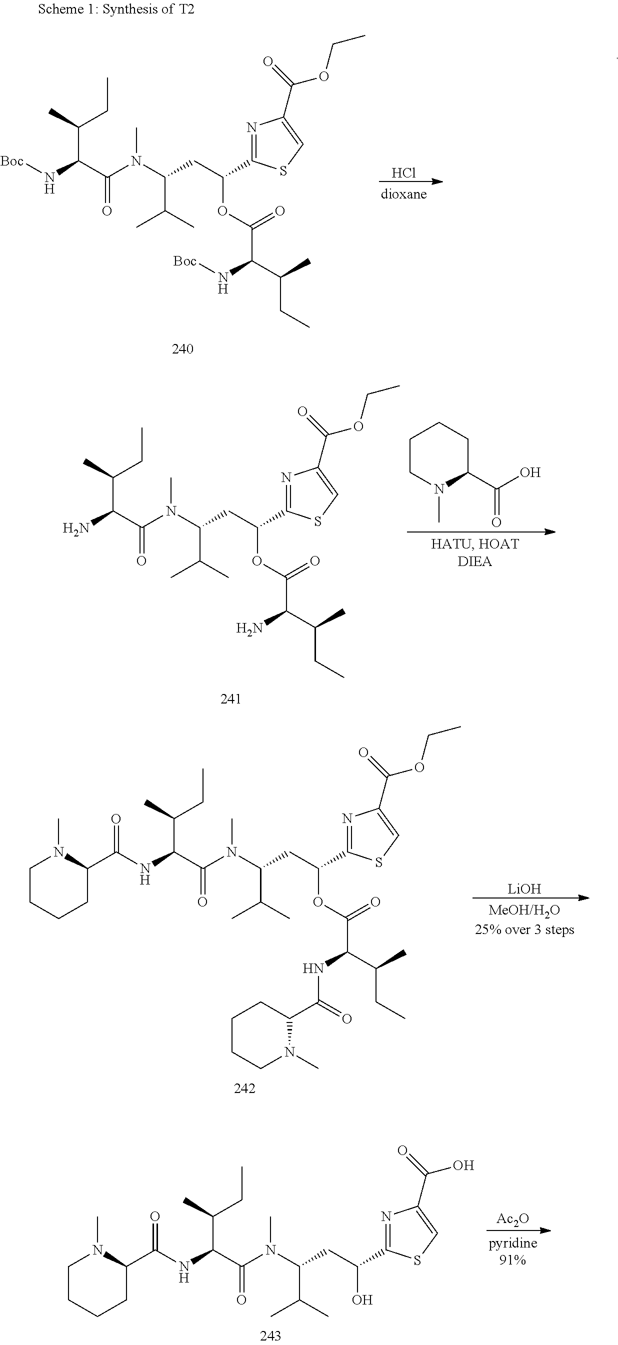

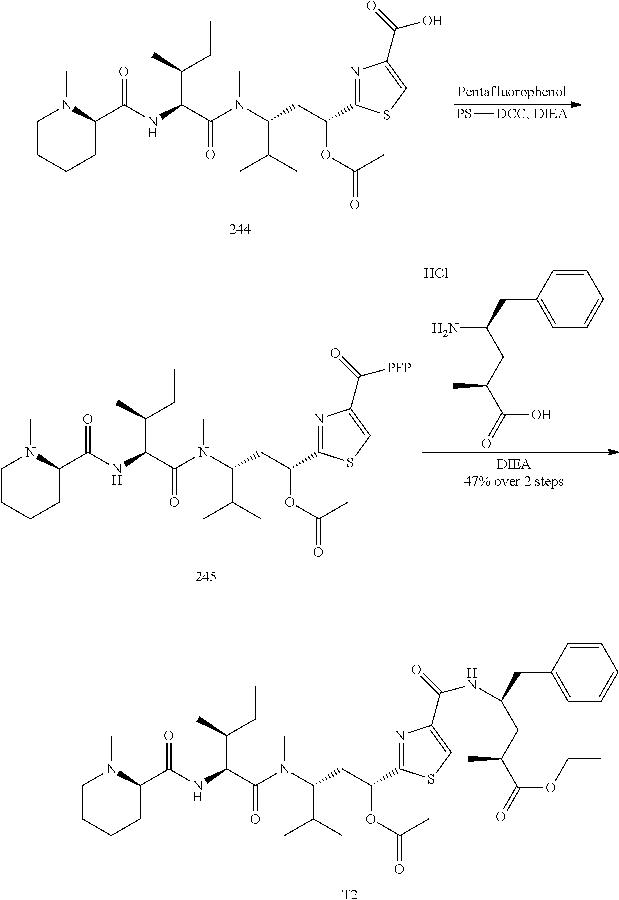

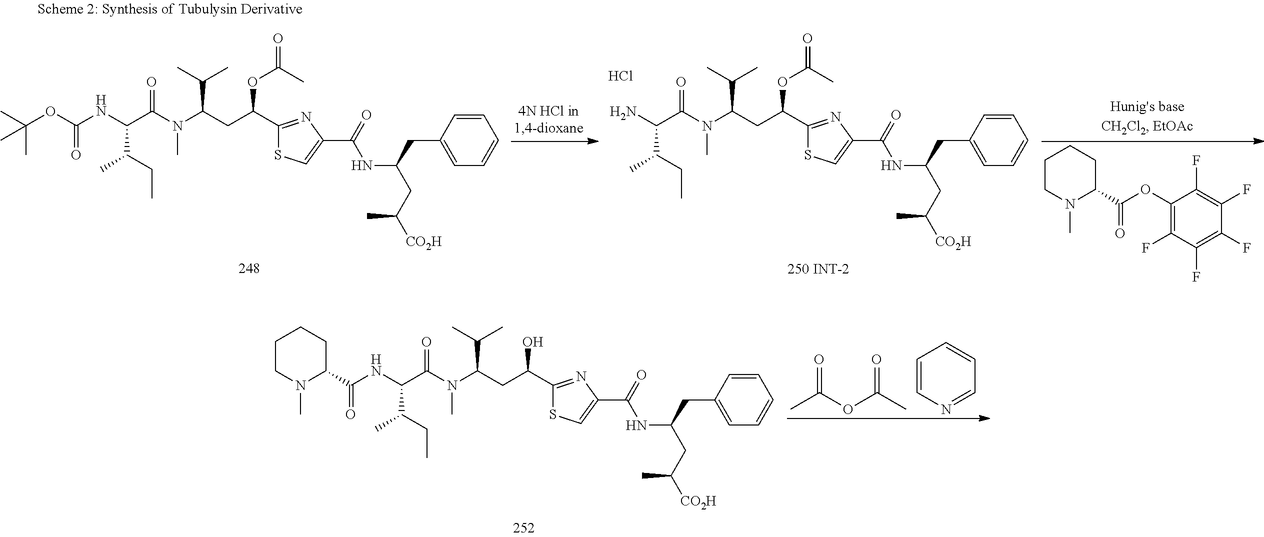

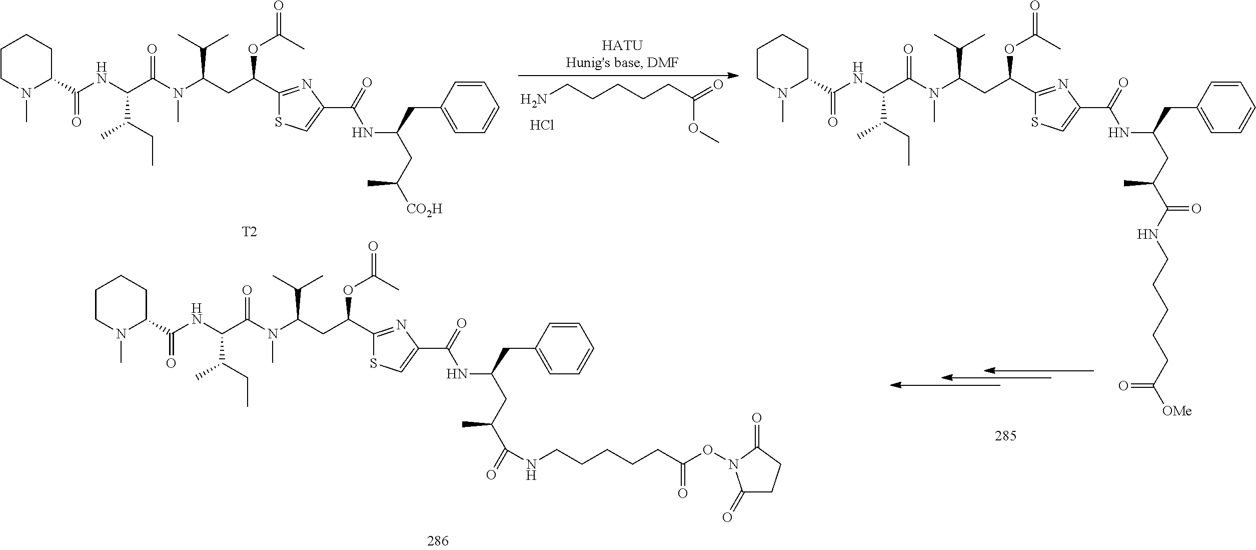

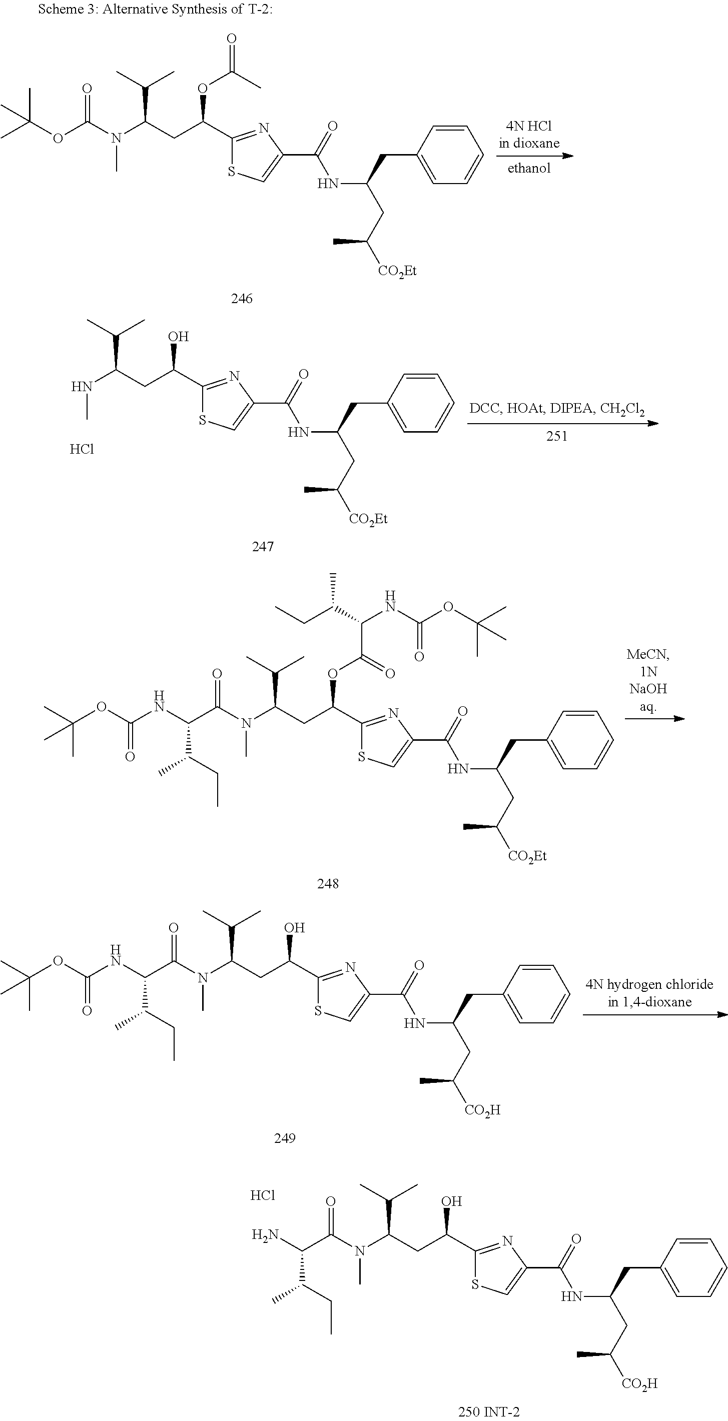

### Example 4

Ethyl (2S,4R)-4-(2-((1R,3R)-1-acetoxy-3-((tert-butoxycarbonyl)(methyl)amino)-4-methylpentyl)thiazole-4-carboxamido)-2-methyl-5-phenylpentanoate (246, 323 mg, 523 μmol) in 4 N HCl in 1,4-dioxane (6.0 ml) was stirred for 30 min Ethanol (1.0 ml) was added and stirring for was continued for an additional 24 h. The solution was blown dry with a stream of air then diluted with 1:1 acetonitrile:water, frozen and lyophilized to afford ethyl (2S,4R)-4-(2-((1R,3R)-1-hydroxy-4-methyl-3-(methylamino)pentyl)thiazole-4-carboxamido)-2-methyl-5-phenylpentanoate hydrochloride (247) as a light yellow solid.

Ethyl (2S,4R)-4-(2-((1R,3R)-1-hydroxy-4-methyl-3-(methylamino)pentyl)thiazole-4-carboxamido)-2-methyl-5-phenylpentanoate hydrochloride (247, material from GDP-131-66, ca. 523 μmol), dicyclohexylmethanediimine (2265 mg, 11.0 mmol), (tert-butoxycarbonyl)-L-isoleucine (251, 2654 mg, 11.5 mmol), 3H-[1,2,3]triazolo[4,5-b]pyridin-3-ol (44 mg, 323 μmol), and diisopropylethylamine (0.20 ml, 1.15 mmol) in methylene chloride (20 ml) was stirred for 18 h. The heterogeneous mixture was filtered and the filtrate was concentrated under reduced pressure. The residue was dissolved into methylene chloride and the solid was removed by filtration two additional times. The residue was flash chromatographed on silica (80 g) with methylene chloride:ethyl acetate 100:0 to 50:50 as the eluent over 10 min to afford 214 mg (45% yield over two steps) of ethyl (2S,4R)-4-(2-((6S,9R,11R,14S)-6,14-di((S)-sec-butyl)-9-isopropyl-2,2,8,18,18-pentamethyl-4,7,13,16-tetraoxo-3,12,17-trioxa-5,8,15-triazanonadecan-11-yl)thiazole-4-carboxamido)-2-methyl-5-phenylpentanoate (248) as a white solid.

Ethyl (2S,4R)-4-(2-((6S,9R,11R,14S)-6,14-di((S)-sec-butyl)-9-isopropyl-2,2,8,18,18-pentamethyl-4,7,13,16-tetraoxo-3,12,17-trioxa-5,8,15-triazanonadecan-11-yl)thiazole-4-carboxamido)-2-methyl-5-phenylpentanoate (248, 214 mg, 237 μmol) and 1 N aqueous sodium hydroxide (0.35 ml, 350 μmol) in 1:1 acetonitrile:water (2 ml) was stirred for 4 h. The solution was brought to a pH=2 with 1 N aqueous hydrogen chloride, then frozen and lyophilized. The residue was flash chromatographed on silica gel (12 g) with methylene chloride:ethyl acetate as the eluent 100:0 to 0:100 over 20 minutes to afford 30 mg (18% yield) ethyl (2S,4R)-4-(2-((1R,3R)-3-((2S,3S)-2-((tert-butoxycarbonyl)amino)-N,3-dimethylpentanamido)-1-hydroxy-4-methylpentyl)thiazole-4-carboxamido)-2-methyl-5-phenylpentanoate (251), 96 mg (61% yield) of (2S,4R)-4-(2-((1R,3R)-3-((2S,3S)-2-((tert-butoxycarbonyl)amino)-N,3-dimethylpentanamido)-1-hydroxy-4-methylpentyl)thiazole-4-carboxamido)-2-methyl-5-phenylpentanoic acid (249), and 45 mg (21% recovery) of ethyl (2S,4R)-4-(2-((6S,9R,11R,14S)-6,14-di((S)-sec-butyl)-9-isopropyl-2,2,8,18,18-pentamethyl-4,7,13,16-tetraoxo-3,12,17-trioxa-5,8,15-triazanonadecan-11-yl)thiazole-4-carboxamido)-2-methyl-5-phenylpentanoate (248) as white solids after lyophilization.

(R)-1-methylpiperidine-2-carboxylic acid (92 mg, 643 μmol), 2,3,4,5,6-pentafluorophenol (122 mg, 662 μmol), and dicyclohexylmethanediimine (198 mg, 960 μmol) in ethyl acetate (1.0 ml) was stirred for 24 h. The heterogeneous mixture was filtered and the solid was washed with ethyl acetate. The perfluorophenyl (R)-1-methylpiperidine-2-carboxylate contained in the filtrate was used crude in the subsequent reaction.

(2S,4R)-4-(2-((1R,3R)-3-((2S,3S)-2-((tert-butoxycarbonyl)amino)-N,3-dimethylpentanamido)-1-hydroxy-4-methylpentyl)thiazole-4-carboxamido)-2-methyl-5-phenylpentanoic acid (249, 96 mg, 145 μmol) in 4 N hydrogen chloride in 1,4-dioxane (2 ml) was stirring for 1 h. The solution was concentrated under a stream of air then diluted with 1:1 acetonitrile:water and lyophilized to yield 87 mg (100% yield) of (2S,4R)-4-(2-((1R,3R)-3-((2S,3S)-2-amino-N,3-dimethylpentanamido)-1-hydroxy-4-methylpentyl)thiazole-4-carboxamido)-2-methyl-5-phenylpentanoic acid hydrochloride (250, INT-2) as a white solid.

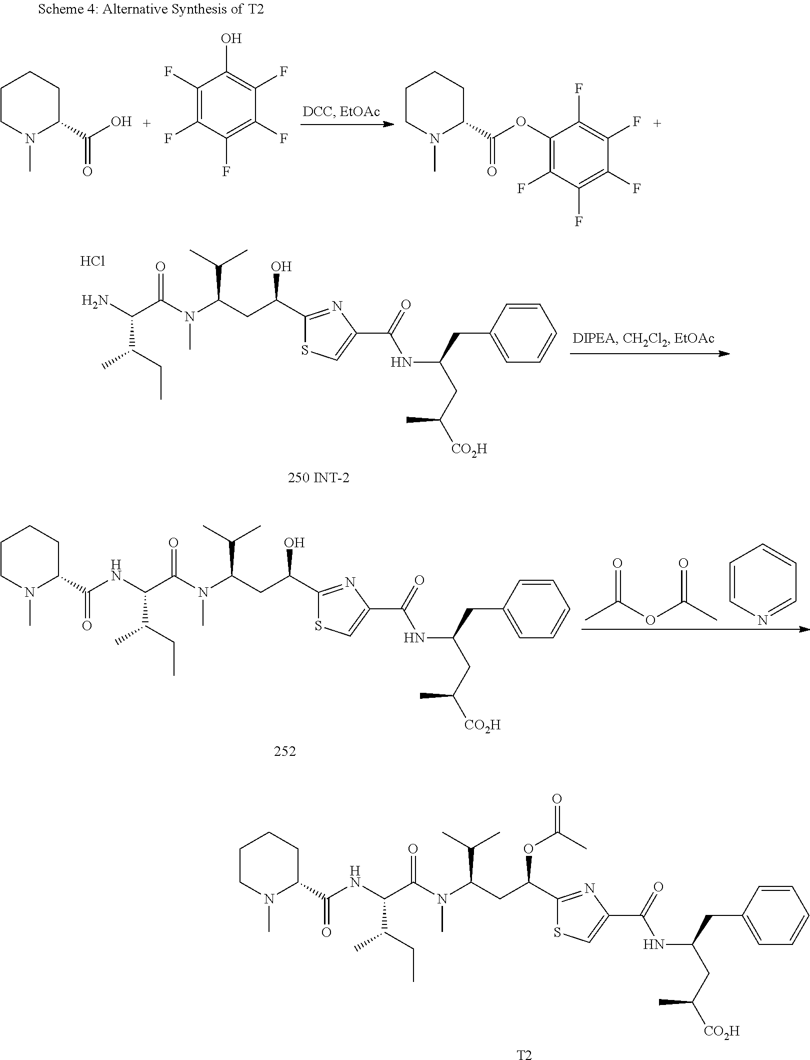

### Example 5

Perfluorophenyl (R)-1-methylpiperidine-2-carboxylate (crude material from GDP-131-071, ca. 643 μmol) in ethyl acetate (2.0 ml), (2S,4R)-4-(2-((1R,3R)-3-((2S,3S)-2-amino-N,3-dimethylpentanamido)-1-hydroxy-4-methylpentyl)thiazole-4-carboxamido)-2-methyl-5-phenylpentanoic acid hydrochloride (250, material from GDP-131-070, ca. 145 μmol), and diisopropylethylamine (0.05 ml, 287 μmol) in methylene chloride (2.0 ml) was stirred for 24 h. The solution was concentrated under a stream of air and the residue was flash chromatographed on silica (12 g) with methylene chloride:methanol as the eluent with a 100:0 to 80:20 gradient over 20 min to furnish 36 mg (36% yield over two steps) of (2S,4R)-4-(2-((1R,3R)-3-((2S,3S)—N,3-dimethyl-2-((R)-1-methylpiperidine-2-carboxamido)pentanamido)-1-hydroxy-4-methylpentyl)thiazole-4-carboxamido)-2-methyl-5-phenylpentanoic acid (252) as a white solid. 30 mg of impure product was also recovered.

Acetic anhydride (2.0 ml) was added to a solution of (2S,4R)-4-(2-((1R,3R)-3-((2S,3S)—N,3-dimethyl-2-((R)-1-methylpiperidine-2-carboxamido)pentanamido)-1-hydroxy-4-methylpentyl)thiazole-4-carboxamido)-2-methyl-5-phenylpentanoic acid (252, material from GDP-01-079) in pyridine (2.0 ml). After stirring for 16 h, the solution was concentrated under reduced pressure and the residue was purified by HPLC to yield 1.1 mg of (2S,4R)-4-(2-((1R,3R)-1-acetoxy-34(2S,3S)—N,3-dimethyl-2-((R)-1-methylpiperidine-2-carboxamido)pentanamido)-4-methylpentyl)thiazole-4-carboxamido)-2-methyl-5-phenylpentanoic acid (T2).

### Analogs of T2 Prepared:

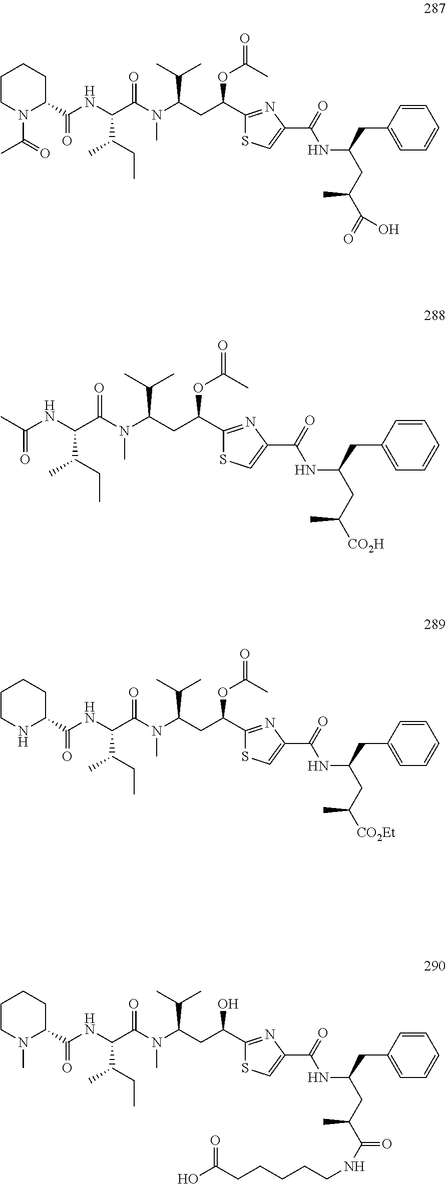 [SMILES: CC[C@H](C)[C@H](NC(=O)[C@H]1CCCCN1C(C)=O)C(=O)N(C)[C@H](C[C@@H](OC(C)=O)c1nc(C(=O)N[C@@H](Cc2ccccc2)C[C@H](C)C(=O)O)cs1)C(C)C ; CC[C@H](C)[C@H](NC(C)=O)C(=O)N(C)[C@H](C[C@@H](OC(C)=O)c1nc(C(=O)N[C@@H](Cc2ccccc2)C[C@H](C)C(=O)O)cs1)C(C)C ; CCOC(=O)[C@@H](C)C[C@H](Cc1ccccc1)NC(=O)c1csc([C@@H](C[C@H](C(C)C)N(C)C(=O)[C@@H](NC(=O)[C@H]2CCCCN2)[C@@H](C)CC)OC(C)=O)n1 ; CC[C@H](C)[C@H](NC(=O)[C@H]1CCCCN1C)C(=O)N(C)[C@H](C[C@@H](O)c1nc(C(=O)N[C@@H](Cc2ccccc2)C[C@H](C)C(=O)NCCCCCC(=O)O)cs1)C(C)C]

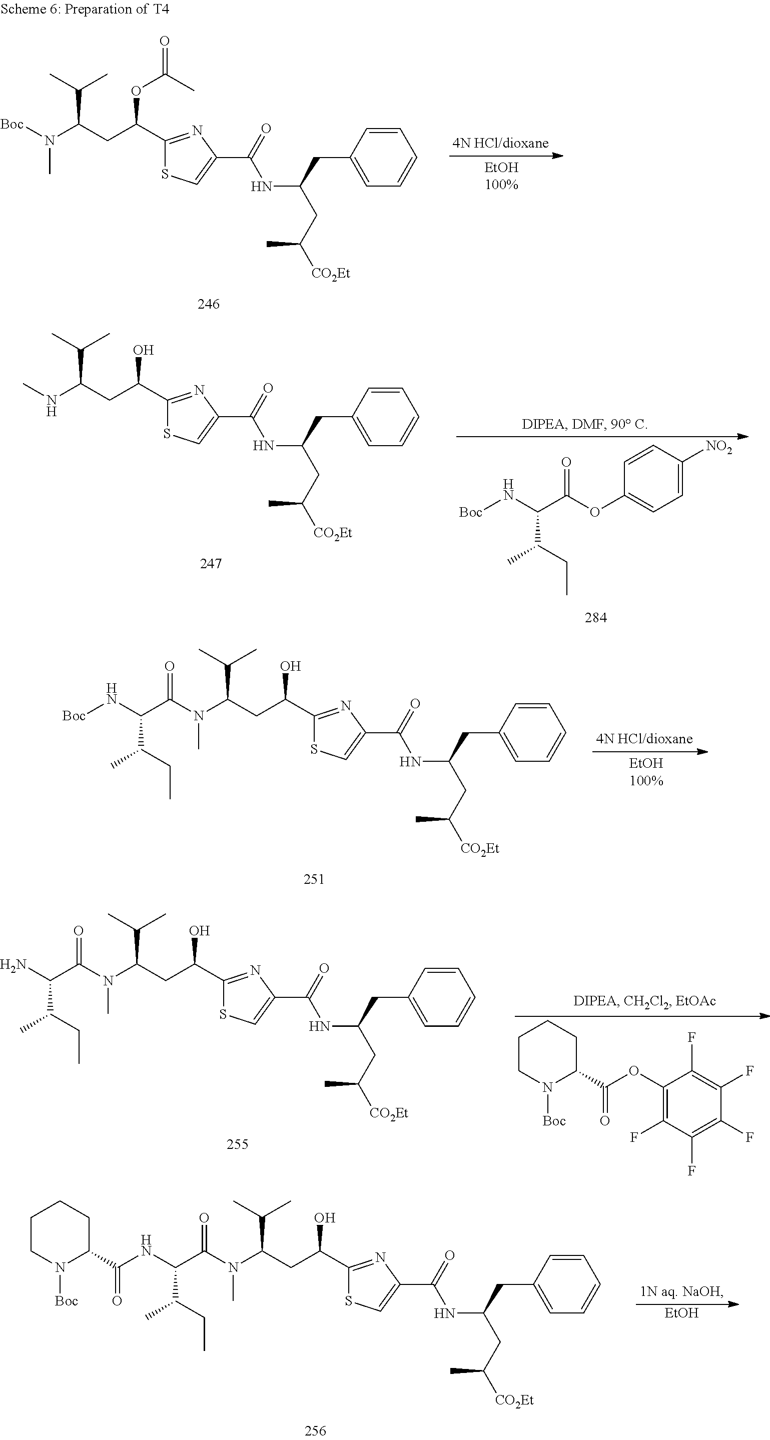

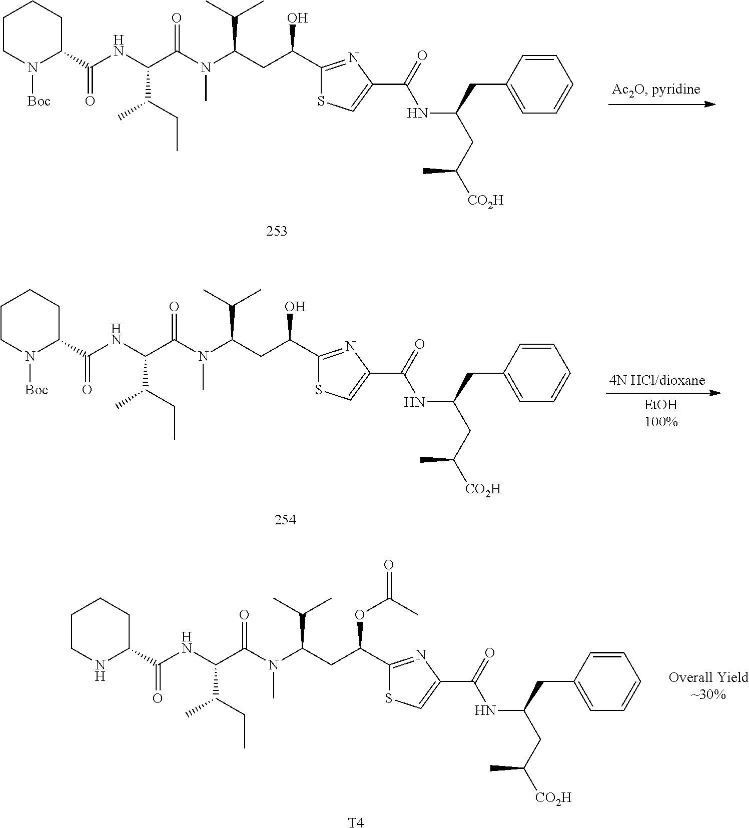 [SMILES: CC[C@H](C)[C@H](NC(=O)[C@H]1CCCCN1C(=O)OC(C)(C)C)C(=O)N(C)[C@H](C[C@@H](O)c1nc(C(=O)N[C@@H](Cc2ccccc2)C[C@H](C)C(=O)O)cs1)C(C)C ; CC[C@H](C)[C@H](NC(=O)[C@H]1CCCCN1C(=O)OC(C)(C)C)C(=O)N(C)[C@H](C[C@@H](O)c1nc(C(=O)N[C@@H](Cc2ccccc2)C[C@H](C)C(=O)O)cs1)C(C)C ; CC[C@H](C)[C@H](NC(=O)[C@H]1CCCCN1)C(=O)N(C)[C@H](C[C@@H](OC(C)=O)c1nc(C(=O)N[C@@H](Cc2ccccc2)C[C@H](C)C(=O)O)cs1)C(C)C ; "T4"]

### Example 6

(R)-1-(Tert-butoxycarbonyl)piperidine-2-carboxylic acid (48 mg, 209 μmol), 2,3,4,5,6-pentafluorophenol (38 mg, 206 μmol), and dicyclohexylmethanediimine (60 mg, 291 μmol) in ethyl acetate (1 ml) was stirred for 48 h. The heterogeneous mixture was filtered and the solid was washed with ethyl acetate. This material was used crude in the subsequent reaction.

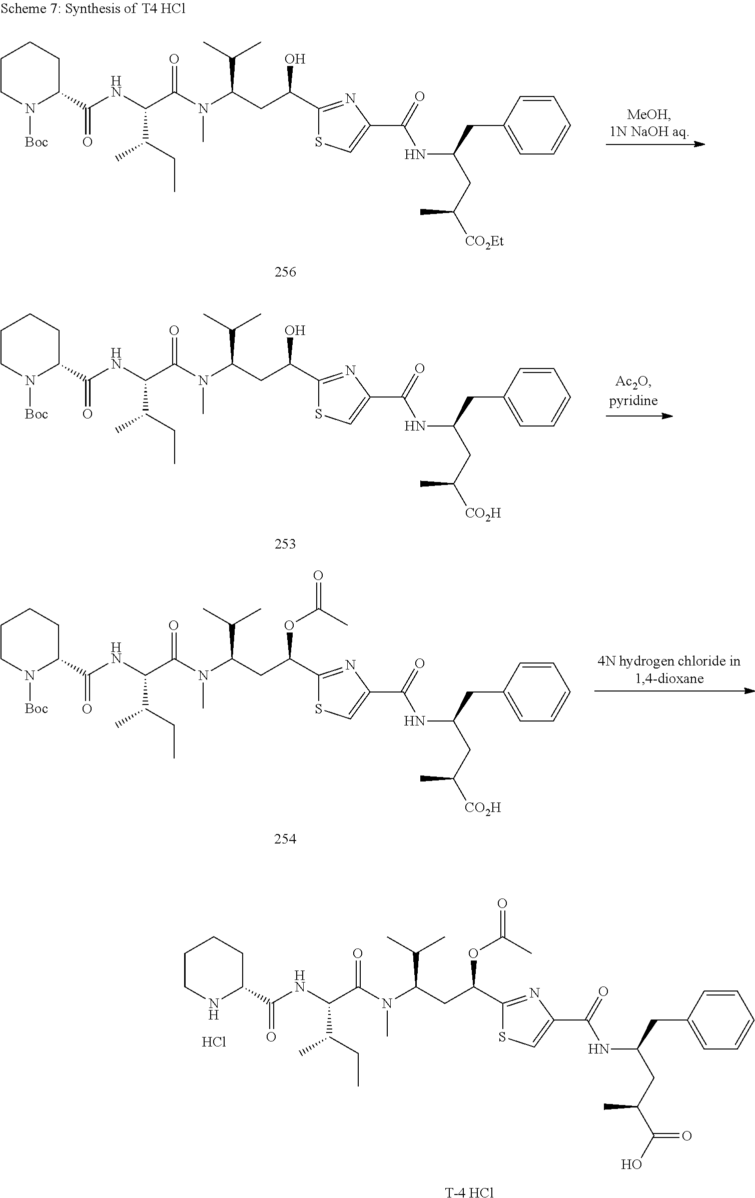

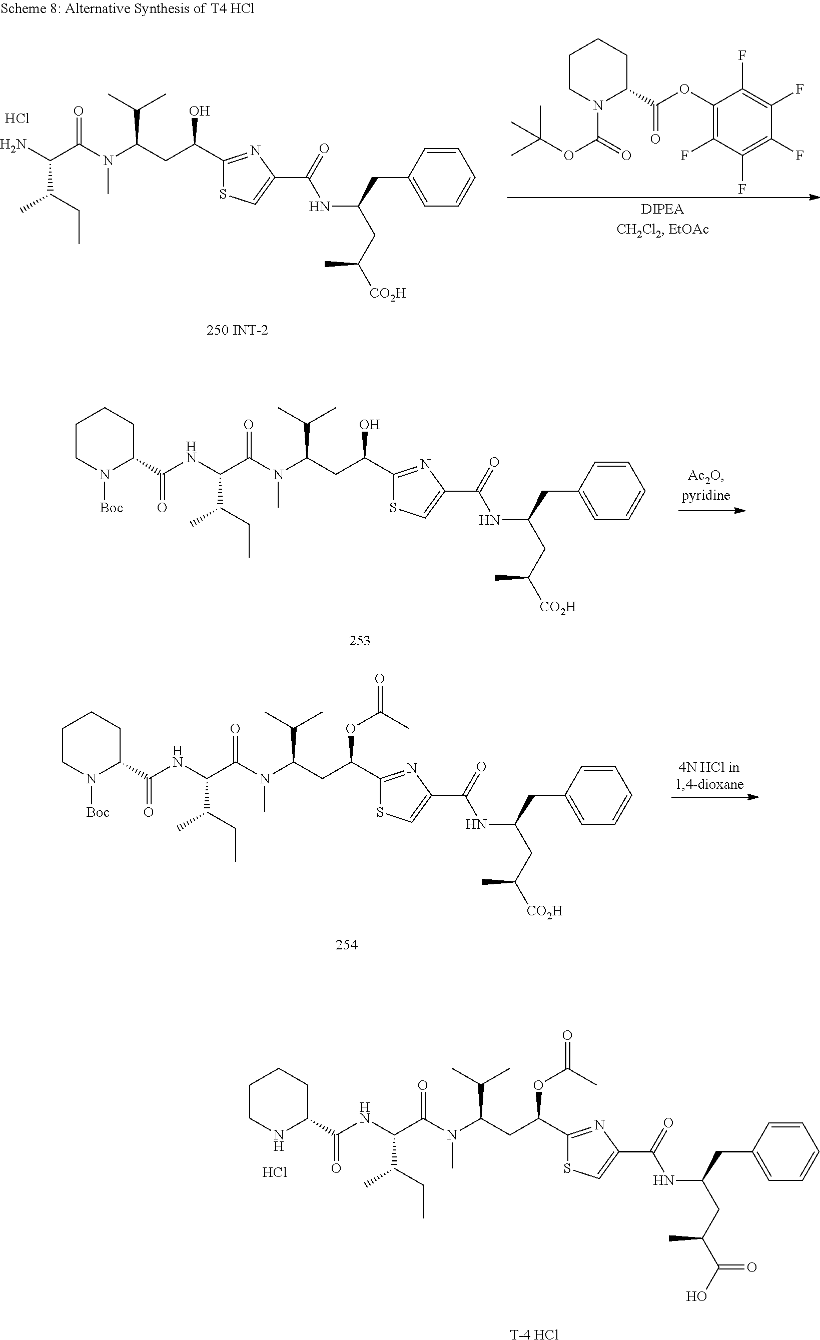

### Example 7

(R)-1-(Tert-butoxycarbonyl)piperidine-2-carboxylic acid (48 mg, 209 μmol), 2,3,4,5,6-pentafluorophenol (38 mg, 206 μmol), and dicyclohexylmethanediimine (60 mg, 291 μmol) in ethyl acetate (1 ml) was stirred for 48 h. The heterogeneous mixture was filtered and the solid was washed with ethyl acetate. This material was used crude in the subsequent reaction.

1-(Tert-butyl) 2-(perfluorophenyl) (R)-piperidine-1,2-dicarboxylate in ethyl acetate (2 ml) from GDP-131-077 was added to a solution of ethyl (2S,4R)-4-(2-((1R,3R)-3-((2S,3S)-2-amino-N,3-dimethylpentanamido)-1-hydroxy-4-methylpentyl)thiazole-4-carboxamido)-2-methyl-5-phenylpentanoate hydrochloride (255, 43.5 μmol from GDP-131-078) and diisopropylethylamine (0.05 ml, 287 μmol) in methylene chloride (2 ml). After stirring for 18 h, the solution was concentrated under reduced pressure and the residue was purified by flash chromatography (12 g silica) with methylene chloride:ethyl acetate as the eluent 100:0 to 50:50 over 25 min. The combined fractions were concentrated under reduced pressure and the residue was dissolved into 1:1 acetonitrile:water and lyophilized to afford 30 mg (86% yield over two steps) of tert-butyl (R)-2-(((2S,3S)-1-(((1R,3R)-1-(4-(((2R,4S)-5-ethoxy-4-methyl-5-oxo-1-phenylpentan-2-yl)carbamoyl)thiazol-2-yl)-1-hydroxy-4-methylpentan-3-yl)(methyl)amino)-3-methyl-1-oxopentan-2-yl)carbamoyl)piperidine-1-carboxylate, 256, as an off-white solid.

Acetic anhydride (0.10 ml, 106 μmol) tert-butyl (R)-2-(((2S,3S)-1-(((1R,3R)-1-(4-(((2R,4S)-5-ethoxy-4-methyl-5-oxo-1-phenylpentan-2-yl)carbamoyl)thiazol-2-yl)-1-hydroxy-4-methylpentan-3-yl)(methyl)amino)-3-methyl-1-oxopentan-2-yl)carbamoyl)piperidine-1-carboxylate (256, 30 mg, 37.5 μmol)in pyridine (0.50 ml). After stirring for 6 h, the solution was concentrated under a stream of air and the residue was purified by flash chromatography (12 g silica) with methylene chloride:ethyl acetate as the eluent 100:0 to 50:50 over 25 min. The combined fractions were concentrated under reduced pressure and the residue was dissolved into 1:1 acetonitrile:water and lyophilized to afford 30 mg (95% yield) of tert-butyl (R)-2-(((2S,3S)-1-(((1R,3R)-1-acetoxy-1-(4-(((2R,4S)-5-ethoxy-4-methyl-5-oxo-1-phenylpentan-2-yl)carbamoyl)thiazol-2-yl)-4-methyl pentan-3-yl)(methyl)amino)-3-methyl-1-oxopentan-2-yl)carbamoyl)piperidine-1-carboxylate, 257.

4 N hydrogen chloride in 1,4-dioxane (2 ml) was added to tert-butyl (R)-2-(((2S,3S)-1-(((1R,3R)-1-acetoxy-1-(4-(((2R,4S)-5-ethoxy-4-methyl-5-oxo-1-phenylpentan-2-yl)carbamoyl)thiazol-2-yl)-4-methylpentan-3-yl)(methyl)amino)-3-methyl-1-oxopentan-2-yl)carbamoyl)piperidine-1-carboxylate (257, 30 mg, 35.6 μmol). After stirring for 30 min, the solution was concentrated under a stream of air, diluted with 1:1 acetonitrile:water, and lyophilized to afford 28 mg (100% yield) of ethyl (2S,4R)-4-(2-((1R,3R)-3-((2S,3S)-2-amino-N,3-dimethylpentanamido)-1-acetoxy-4-methylpentyl)thiazole-4-carboxamido)-2-methyl-5-phenylpentanoate hydrochloride, 258, as a white solid.

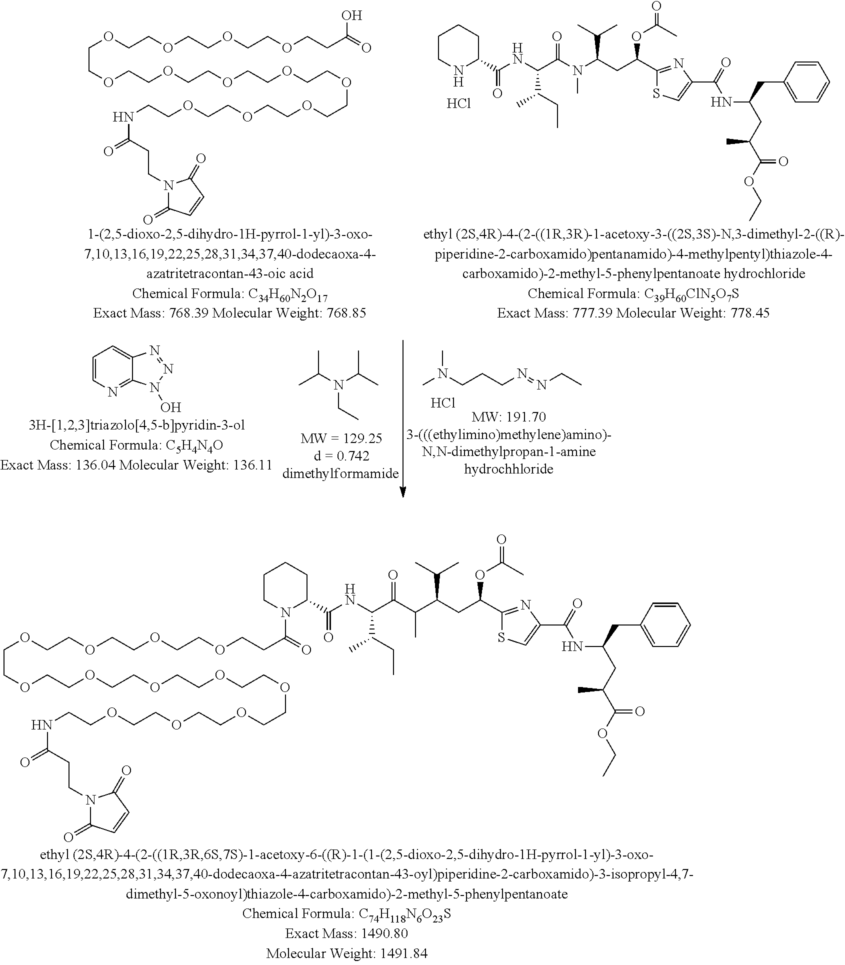

### Example 8

1-(2,5-Dioxo-2,5-dihydro-1H-pyrrol-1-yl)-3-oxo-7,10,13,16,19,22,25,28,31,34,37,40-dodecaoxa-4-azatritetracontan-43-oic (23 mg, 29.9 μmol), ethyl (2S,4R)-4-(2-((1R,3R)-1-acetoxy-3-((2S,3S)—N,3-dimethyl-2-((R)-piperidine-2-carboxamido)pentanamido)-4-methylpentyl)thiazole-4-carboxamido)-2-methyl-5-phenylpentanoate hydrochloride (12 mg, 15.4 μmol), 3H-[1,2,3]triazolo[4,5-b]pyridin-3-ol (6 mg, 44.1 μmol), 3-(((ethylimino)methylene)amino)-N,N-dimethylpropan-1-amine hydrochloride (33 mg, 172 μmol) and diisopropylethylamine (0.05 ml, 287 μmol) in dimethylformamide (0.30 ml) was stirred for 18 h. The solution was purified by reverse phase HPLC to afford 9 mg (39% yield) of ethyl (2S,4R)-4-(2-((1R,3R,6 S,7 S)-1-acetoxy-64(R)-1-(1-(2,5-dioxo-2,5-dihydro-1H-pyrrol-1-yl)-3-oxo-7,10,13,16,19,22,25,28,31,34,37,40-dodecaoxa-4-azatritetracontan-43-oyl)piperidine-2-carboxamido)-3-isopropyl-4,7-dimethyl-5-oxononyl)thiazole-4-carboxamido)-2-methyl-5-phenylpentanoate as a clear oil.

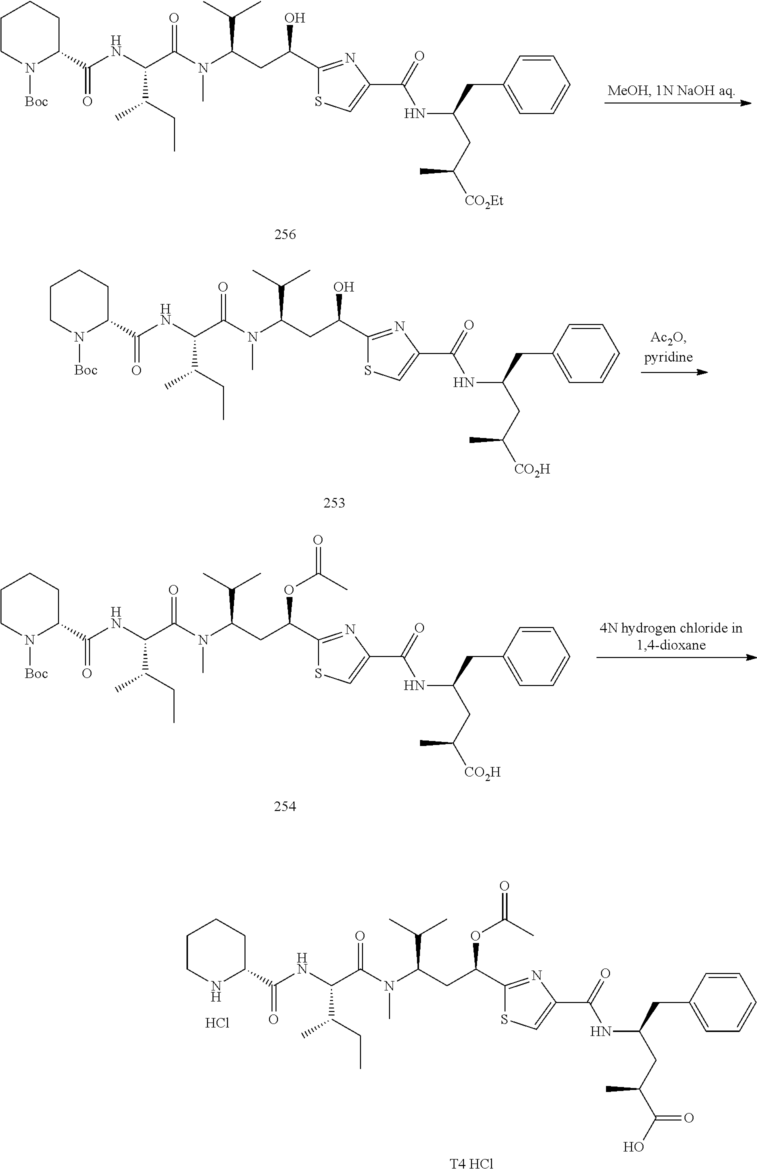

### Example 9

1 N aqueous sodium hydroxide (0.20 ml, 200 μmol) was added to a solution of tert-butyl (R)-2-(((2S,3S)-1-(((1R,3R)-1-(4-(((2R,4S)-5-ethoxy-4-methyl-5-oxo-1-phenylpentan-2-yl)carbamoyl)thiazol-2-yl)-1-hydroxy-4-methylpentan-3-yl)(methyl)amino)-3-methyl-1-oxopentan-2-yl)carbamoyl)piperidine-1-carboxylate (256, 53 mg, 66.2 μmol) in methanol (1 ml). After stirring for 18 h, the solution was concentrated under a stream of air to afford (2S,4R)-4-(2-((1R,3R)-3-((2S,3S)-2-((R)-1-(tert-butoxycarbonyl)piperidine-2-carboxamido)-N,3-dimethylpentanamido)-1-hydroxy-4-methylpentyl)thiazole-4-carboxamido)-2-methyl-5-phenylpentanoic acid, 253. This material was used crude in the subsequent reaction.

Acetic anhydride (0.50 ml, 5.29 mmol) was added to a solution of (2S,4R)-4-(2-((1R,3R)-3-((2S,3S)-2-((R)-1-(tert-butoxycarbonyl)piperidine-2-carboxamido)-N,3-dimethylpentanamido)-1-hydroxy-4-methylpentyl)thiazole-4-carboxamido)-2-methyl-5-phenylpentanoic acid (253, crude material from GDP-131-05, ca. 66.2 μmol) in pyridine (2.0 ml, 24.8 mmol). After stirring for 2 h, the solution was concentrated under a stream of air. The residue was flash chromatographed on silica gel (12 g) with methylene chloride:methanol 100:0 to 80:20 as the eluent over a 20 minute interval to afford (2S,4R)-4-(2-((1R,3R)-1-acetoxy-3-((2S,3S)-2-((R)-1-(tert-butoxycarbonyl)piperidine-2-carboxamido)-N,3-dimethylpentanamido)-4-methylpentyl)thiazole-4-carboxamido)-2-methyl-5-phenylpentanoic acid, 254, as a film which was used crude in the subsequent reaction.

4 N hydrogen chloride in dioxane (2 ml) was added to (2S,4R)-4-(2-((1R,3R)-1-acetoxy-3-((2S,3S)-2-((R)-1-(tert-butoxycarbonyl)piperidine-2-carboxamido)-N,3-dimethylpentanamido)-4-methylpentyl)thiazole-4-carboxamido)-2-methyl-5-phenylpentanoic acid, 254. After stirring for 30 min, the solution was concentrated under a stream of air and purified by reverse phase HPLC. After lyophilizing the fractions that contained the product, the white solid was diluted with 1:1 acetonitrile:water and 1 drop 1 N aqueous hydrogen chloride was added. The solution frozen and was lyophilized to yield 22 mg (44% yield over 3 steps) of (2S,4R)-4-(2-((1R,3R)-1-acetoxy-3-((2S,3S)—N,3-dimethyl-2-((R)-piperidine-2-carboxamido)pentanamido)-4-methylpentyethiazole-4-carboxamido)-2-methyl-5-phenylpentanoic acid hydrochloride, T4HCl, as a tan solid.

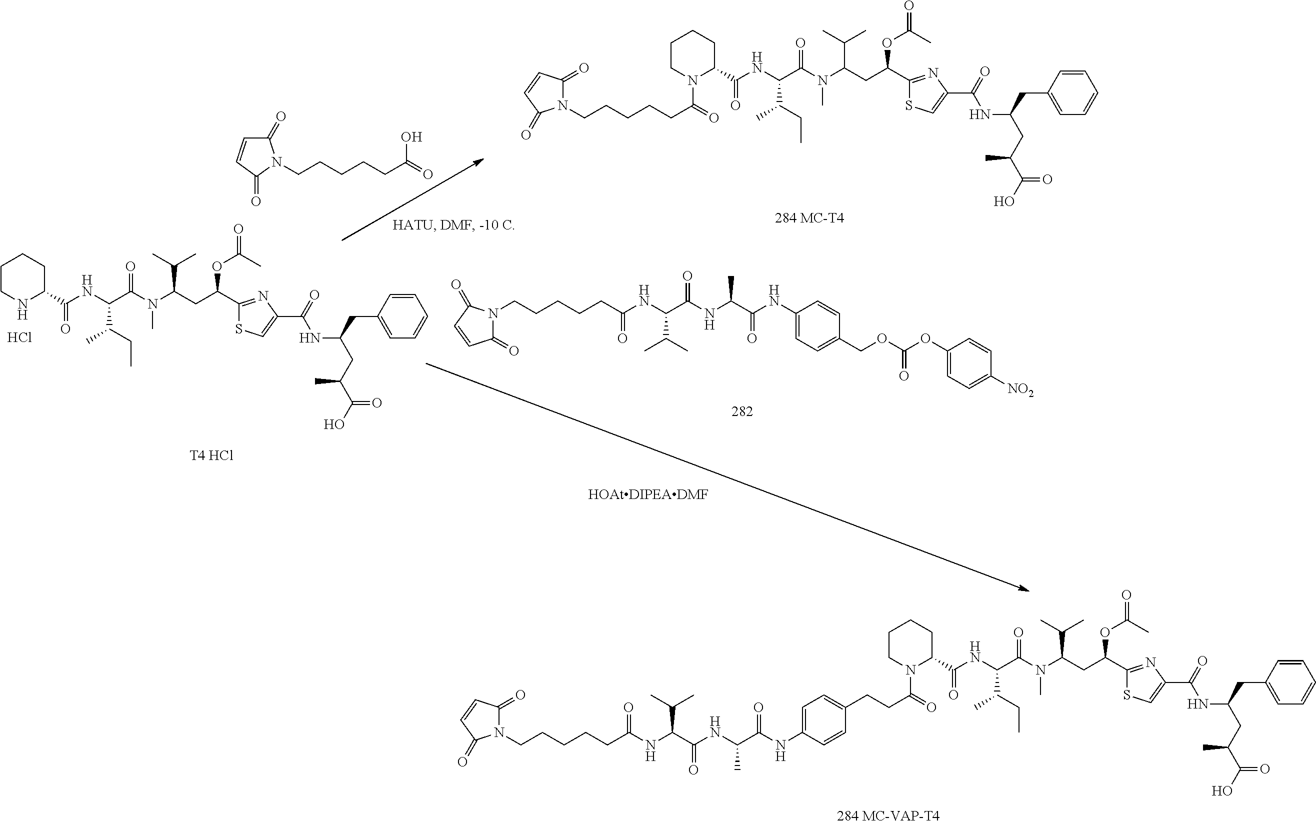

### Example 10

6-(2,5-Dioxo-2,5-dihydro-1H-pyrrol-1-yl)hexanoic acid (14.6 mg, 69.1 μmol) and HATU (14.9 mg, 39.2 μmol) in dimethylformamide (0.1 ml) was stirred at −10° C. for 30 min. The solution was added to diisopropylethylamine (0.02 ml, 115 μmol) and (2S,4R)-4-(2-((1R,3R)-1-acetoxy-3-((2S,3S)—N,3-dimethyl-2-((R)-piperidine-2-carboxamido)pentanamido)-4-methylpentyl)thiazole-4-carboxamido)-2-methyl-5-phenylpentanoic acid (T4, 3.5 mg, 4.66 μmol). After stirring for 20 min at −10° C., the brine/ice bath was removed and stirring for continued for an addition 20 min. The solution was purified by HPLC.

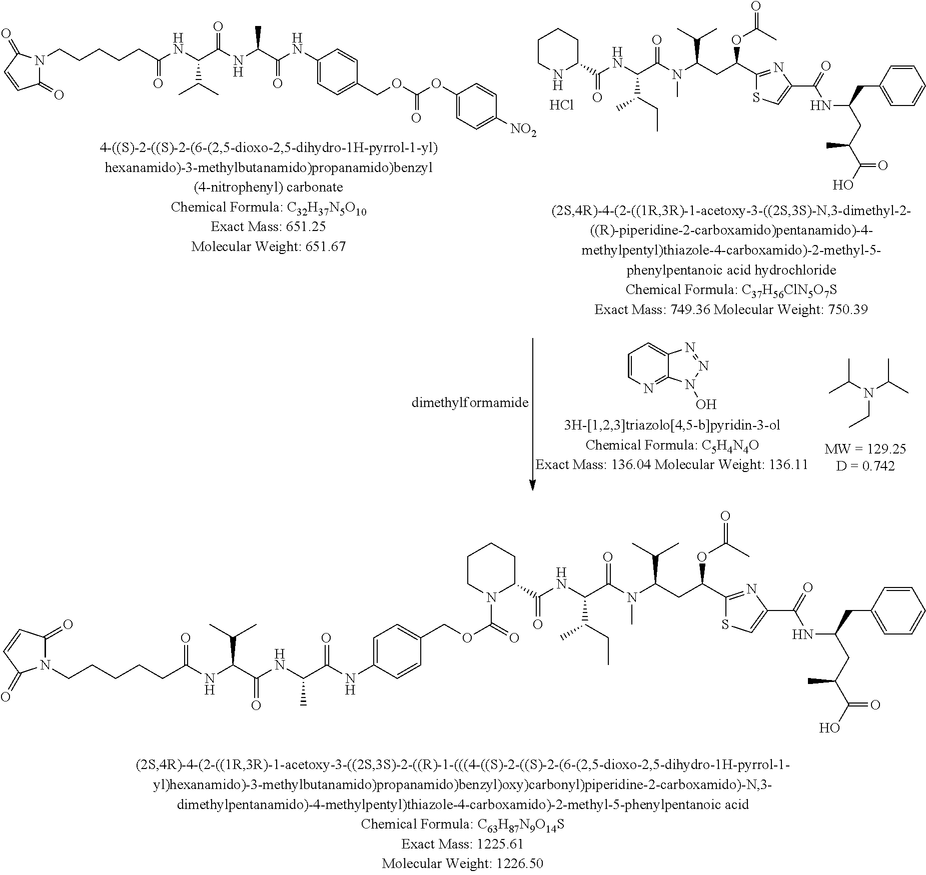

### Example 11

(2S,4R)-4-(2-((1R,3R)-1-acetoxy-3-((2S,3S)—N,3-dimethyl-2-((R)-piperidine-2-carboxamido)pentanamido)-4-methylpentyl)thiazole-4-carboxamido)-2-methyl-5-phenylpentanoic acid hydrochloride (T4HCl, 12 mg, 16.0 μmol), 4-((S)-2-((S)-2-(6-(2,5-dioxo-2,5-dihydro-1H-pyrrol-1-yl)hexanamido)-3-methylbutanamido)propanamido)benzyl(4-nitrophenyl) carbonate (24 mg, 36.8 μmol), diisopropylethylamine (0.10 ml, 574 μmol), and 3H-[1,2,3]triazolo[4,5-b]pyridin-3-ol (2 mg, 14.6 μmol) in dimethylformamide (0.50 ml) was stirred for 18 h. The solution was purified by reverse phase prep HPLC to yield 12 mg (61% yield) of (2S,4R)-4-(2-((1R,3R)-1-acetoxy-3-((2S,3S)-2-((R)-1-(((4-((S)-2-((S)-2-(6-(2,5-dioxo-2,5-dihydro-1H-pyrrol-1-yl)hexanamido)-3-methylbutanamido)propanamido)benzyl)oxy)carbonyl)piperidine-2-carboxamido)-N,3-dimethylpentanamido)-4-methylpentyl)thiazole-4-carboxamido)-2-methyl-5-phenylpentanoic acid, 263, as a white solid.

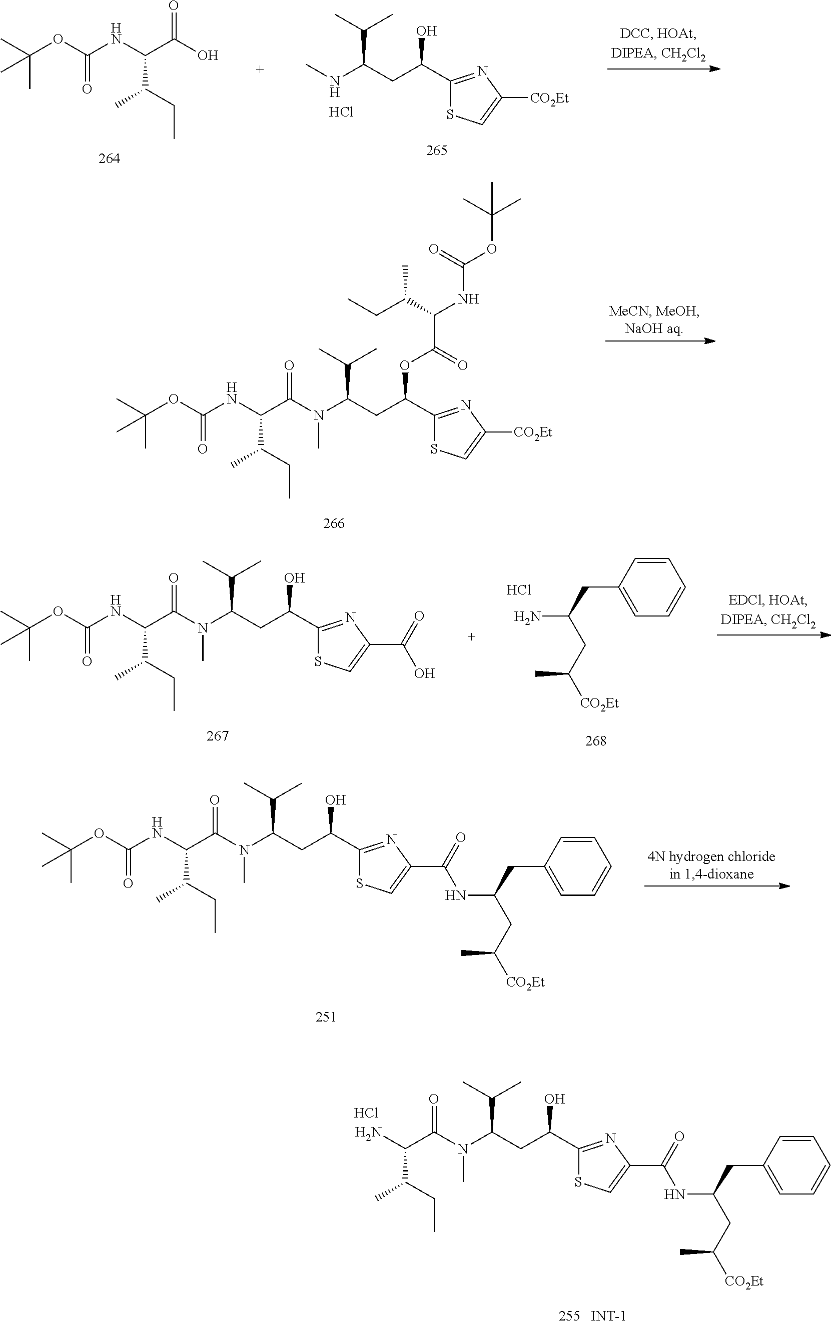

### Example 12

Ethyl 2-((1R,3R)-1-hydroxy-4-methyl-3-(methylamino)pentyl)thiazole-4-carboxylate hydrochloride (265, 638 mg, 1.98 mmol), (tert-butoxycarbonyl)-L-isoleucine (264, 4.8 g, 20.8 mmol), 3H-[1,2,3]triazolo[4,5-b]pyridin-3-ol (0.8 g, 5.88 mmol), dicyclohexylmethanediimine (5.1 g, 24.7 mmol), diisopropylethylamine (0.5 ml, 2.87 mmol) in methylene chloride (100 ml) was stirred for 18 h. The heterogeneous mixture was filtered and the filtrate was concentrated under reduced pressure. Methylene chloride was added to the residue and the solid was removed by filtration. The filtrate was flash chromatographed on silica gel (80 g) with methylene chloride:ethyl acetate as the eluent 100:0 to 50:50 over 25 min to afford 1686 mg (120% yield likely impure with dicyclohexylurea) of ethyl 2-((6S,9R,11R,14S)-6,14-di((S)-sec-butyl)-9-isopropyl-2,2,8,18,18-pentamethyl-4,7,13,16-tetraoxo-3,12,17-trioxa-5,8,15-triazanonadecan-11-yl)thiazole-4-carboxylate, 266, as a viscous yellow oil.

### Example 13

Ethyl 2-((6S,9R,11R,14S)-6,14-di((S)-sec-butyl)-9-isopropyl-2,2,8,18,18-pentamethyl-4,7,13,16-tetraoxo-3,12,17-trioxa-5,8,15-triazanonadecan-11-yl)thiazole-4-carboxylate (266, 96 mg, 135 μmol) and 1 N aqueous sodium hydroxide (0.50 ml, 500 μmol) in 1:1:1 methanol:acetonitrile:water (3 ml) was stirred for 18 h. The solution was brought to an acidic pH with 1 N aqueous hydrogen chloride, frozen, and lyophilized. The residue was diluted with methylene chloride and filtered. The solid was collected to afford 2-((1R,3R)-3-((2S,3S)-2-((tert-butoxycarbonyl)amino)-N,3-dimethylpentanamido)-1-hydroxy-4-methylpentyl)thiazole-4-carboxylic acid, 267, as a white solid that was used crude in the subsequent step.

### Example 14

2-((1R,3R)-3-((2S,3S)-2-((tert-butoxycarbonyeamino)-N,3-dimethylpentanamido)-1-hydroxy-4-methylpentyl)thiazole-4-carboxylic acid (267, 134 mg, 284 μmol), ethyl (2S,4R)-4-amino-2-methyl-5-phenylpentanoate hydrochloride (268, 84 mg, 309 μmol), 3-(((ethylimino)methylene)amino)-N,N-dimethylpropan-1-amine hydrochloride (104 mg, 543 μmol), 3H-[1,2,3]triazolo[4,5-b]pyridin-3-ol (22 mg, 162 μmol), and diisopropylethylamine (0.10 ml, 574 μmol) in methylene chloride (2 ml) was stirred for 18 h. The solution was directly flash chromatographed on silica gel (40 g) with methylene chloride:ethyl acetate as the eluent 100:0 to 50:50 over 25 minutes to afford 173 mg (88% yield) of (2R,4S)-5-ethoxy-4-methyl-5-oxo-1-phenylpentan-2-yl 2-((1R,3R)-3-((2S,3S)-2-((tert-butoxycarbonyl)amino)-N,3-dimethylpentanamido)-1-hydroxy-4-methylpentyl)thiazole-4-carboxylate, 251, as a white solid after lyophilization.

### Example 15

4 N Hydrogen chloride in 1,4-dioxane (2 ml) was added to (2R,4S)-5-ethoxy-4-methyl-5-oxo-1-phenylpentan-2-yl 2-((1R,3R)-3-((2S,3S)-2-((tert-butoxycarbonyeamino)-N,3-dimethylpentanamido)-1-hydroxy-4-methylpentyl)thiazole-4-carboxylate (251, 42 mg, 60.9 μmol). After stirring for 2 h, the solution was evaporated under a stream of air, diluted with 1:1 acetonitrile:water, and lyophilized to afford 38 mg (100% yield) of ethyl (2S,4R)-4-(2-((1R,3R)-3-((2S,3S)-2-amino-N,3-dimethylpentanamido)-1-hydroxy-4-methylpentyl)thiazole-4-carboxamido)-2-methyl-5-phenylpentanoate hydrochloride, 255 as a white solid.

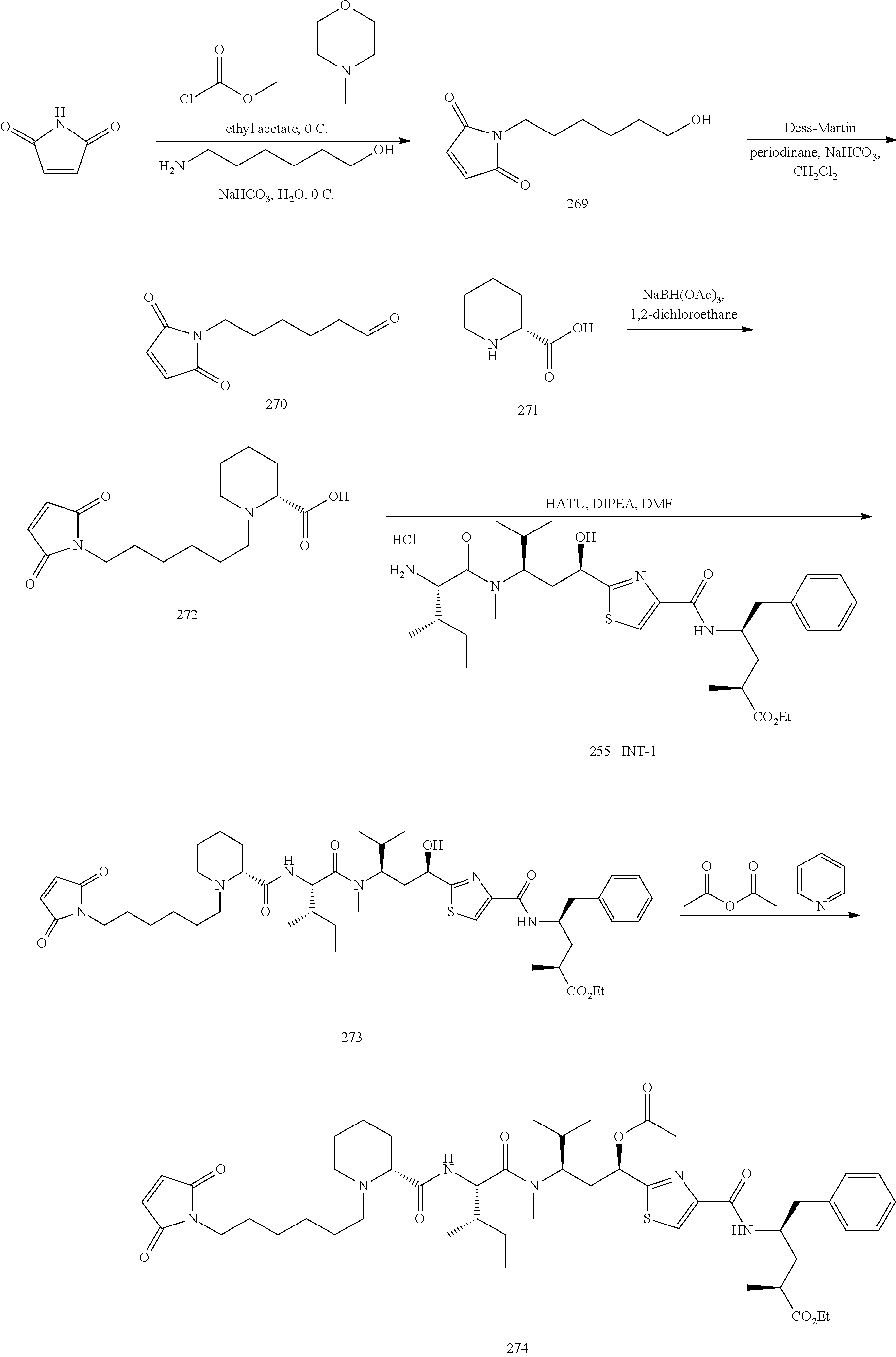

### Example 16

Methyl chloroformate (1.0 ml, 13.0 mmol) was slowly added dropwise to a solution of H-pyrrole-2,5-dione (1.0 g, 10.3 mmol) and N-methylmorpholine (1.5 ml, 13.6 mmol) in ethyl acetate (10 ml) at 0° C. After stirring for 30 min, 6-aminohexan-1-ol (1.4 g, 11.9 mmol) was added followed by the addition of saturated aqueous sodium bicarbonate (2 ml). After stirring for an additional 30 minutes, the solution was extracted with ethyl acetate. The combined organic extracts were dried over anhydrous sodium sulfate, filtered, and concentrated under reduced pressure. The residue was flash chromatographed on silica gel (40 g) with methylene chloride:ethylacetate as the eluent 100:0 to 0:100 over 20 min to afford 0.5 g (25% yield) of 1-(6-hydroxyhexyl)-1H-pyrrole-2,5-dione, 269, as a clear oil.

### Example 17

1-(6-Hydroxyhexyl)-1H-pyrrole-2,5-dione (269, 0.5 g, 2.54 mmol), DessMartin periodinane (2.2 g, 5.19 mmol), and sodium bicarbonate (3.8 g, 45.2 mmol) in methylene chloride (20 ml) was stirred for 2 h. The heterogeneous mixture was filtered and the filtrate was directly flash chromatographed on silica gel (12 g) with methylene chloride:ethyl acetate as the eluent 100:0 to 80:20 over 10 min to afford 0.3 g (61% yield) of 6-(2,5-dioxo-2,5-dihydro-1H-pyrrol-1-yl)hexanal, 270, as a clear oil.

### Example 18

6-(2,5-Dioxo-2,5-dihydro-1H-pyrrol-1-yl)hexanal (270, 0.3 g, 1.54 mmol) and (R)-piperidine-2-carboxylic acid (271, 0.5 g, 3.87 mmol) in 1,2-dichloroethane (10 ml) was stirred for 20 min. Sodium triacetoxyborohydride (1.6 g, 7.55 mmol) was added. After stirring for 1 h, the heterogeneous mixture was filtered and the filtrate was directly flash chromatographed on silica gel (12 g) with methylene chloride:methanol as the eluent 100:0 to 80:20 over 10 min to afford 0.1 g (21% yield) of (R)-1-(6-((tert-butoxycarbonyeamino)hexyl)piperidine-2-carboxylic acid, 272, as a white solid after lyophilization.

Diisopropylethylamine (0.05 ml, 287 μmol) was added to a heterogeneous mixture of (R)-1-(6-(2,5-dioxo-2,5-dihydro-1H-pyrrol-1-yl)hexyl)piperidine-2-carboxylic acid (272, 12 mg, 38.9 μmol) and HATU (24 mg, 63.1 μmol) in dimethylformamide (0.20 ml). The solution immediately became homogeneous. After standing for 15 min, the solution was added to ethyl (2S,4R)-4-(2-((1R,3R)-3-((2S,3S)-2-amino-N,3-dimethylpentanamido)-1-hydroxy-4-methylpentyl)thiazole-4-carboxamido)-2-methyl-5-phenylpentanoate hydrochloride (255, 17 mg, 27.2 μmol). After standing for 30 min, the solution was blown dry with a stream of air. This product, ethyl (2S,4R)-4-(2-((1R,3R)-3-((2S,3S)-2-((R)-1-(6-(2,5-dioxo-2,5-dihydro-1H-pyrrol-1-yl)hexyl)piperidine-2-carboxamido)-N,3-dimethylpentanamido)-1-hydroxy-4-methylpentyl)thiazole-4-carboxamido)-2-methyl-5-phenylpentanoate, 273, was used crude in the subsequent step.

Acetic anhydride (0.20 ml, 2.12 mmol) was added to a solution of ethyl (2S,4R)-4-(2-((1R,3R)-3-((2S,3S)-2-((R)-1-(6-(2,5-dioxo-2,5-dihydro-1H-pyrrol-1-yl)hexyl)piperidine-2-carboxamido)-N,3-dimethylpentanamido)-1-hydroxy-4-methylpentyl)thiazole-4-carboxamido)-2-methyl-5-phenylpentanoate (273, material from GDP-150-039, ca. 27.2 μmol) in pyridine (1 ml). After stirring for 2 h, ice was added to the solution. Pyridine added to the maleimide so this should be precooled prior to quenching. The solution was directly purified by reverse phase HPLC to afford 3.1 mg (12% yield) of ethyl (2S,4R)-4-(2-((1R,3R)-1-acetoxy-3-((2S,3S)-2-((R)-1-(6-(2,5-dioxo-2,5-dihydro-1H-pyrrol-1-yl)hexyl)piperidine-2-carboxamido)-N,3-dimethylpentanamido)-4-methylpentyl)thiazole-4-carboxamido)-2-methyl-5-phenylpentanoate, 274, as a white solid.

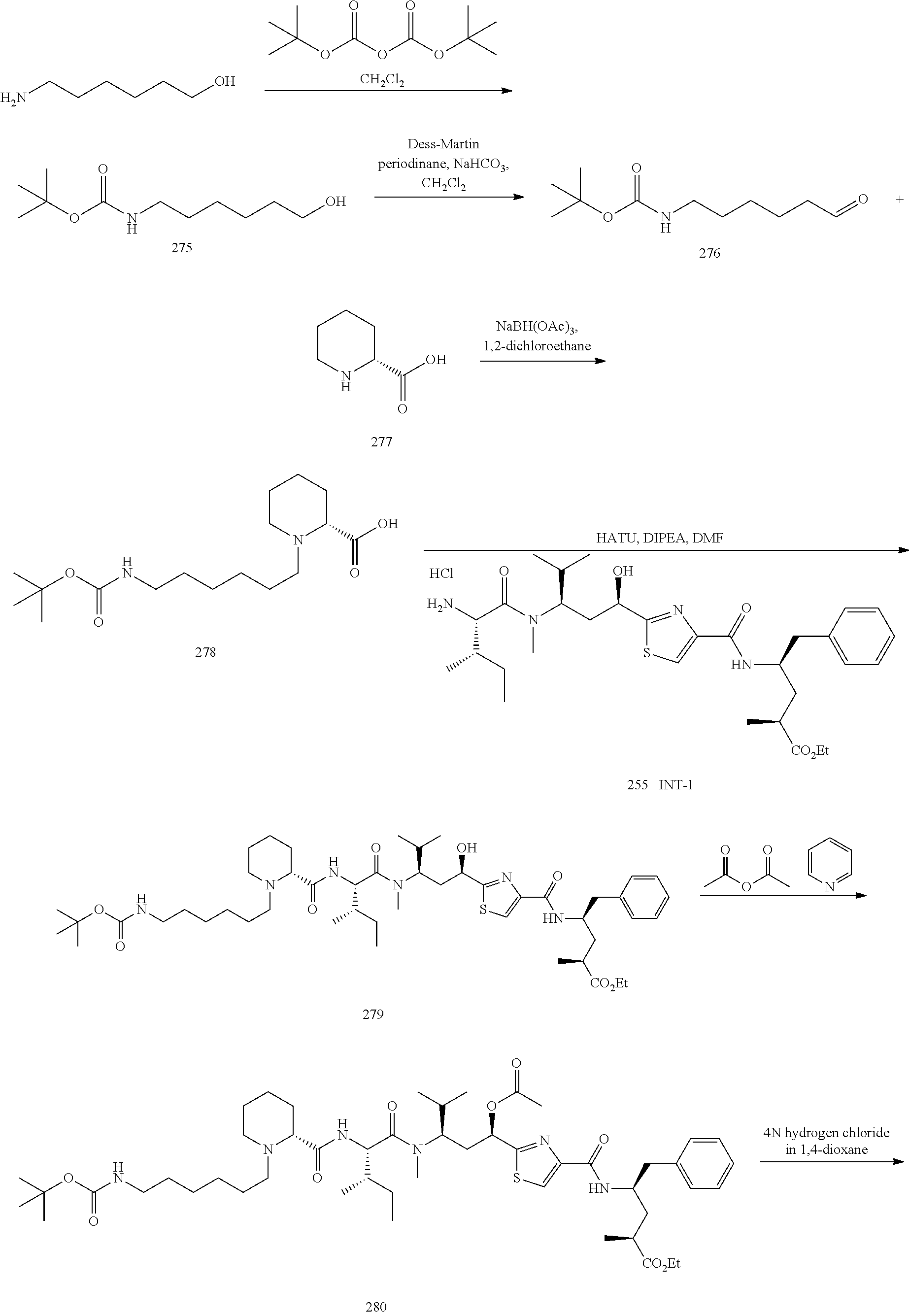

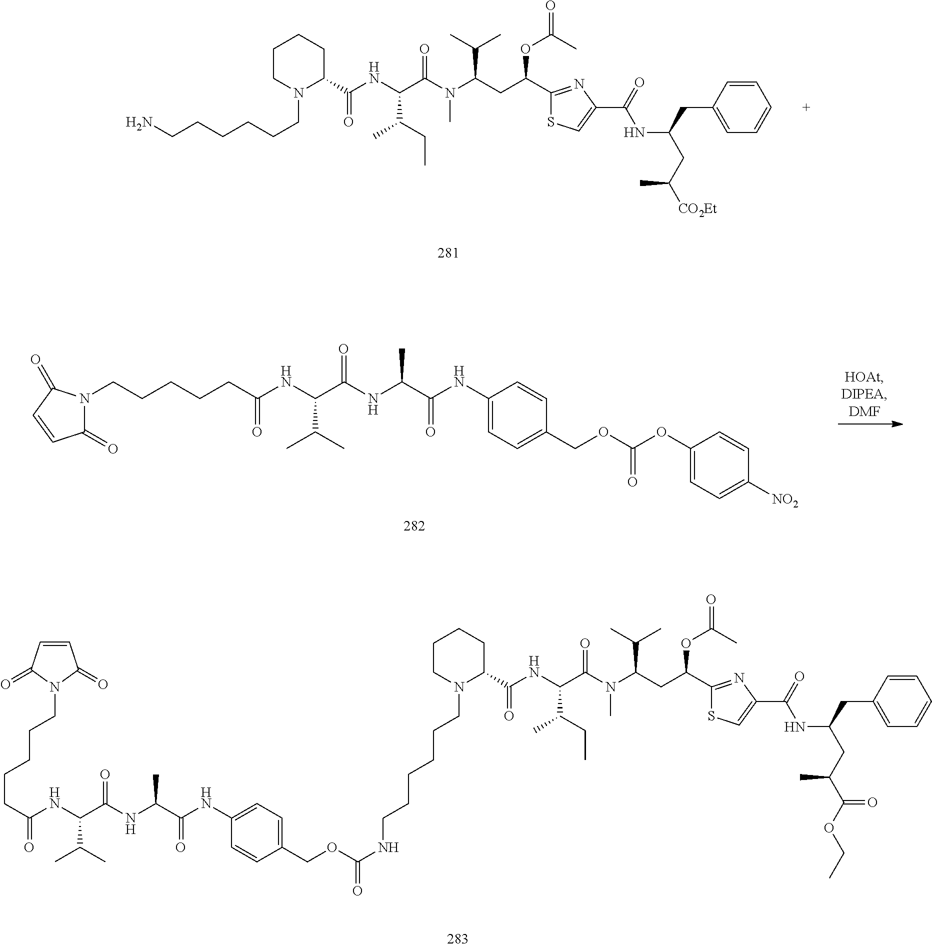 [SMILES: CCOC(=O)[C@@H](C)C[C@H](Cc1ccccc1)NC(=O)c1csc([C@@H](C[C@H](C(C)C)N(C)C(=O)[C@@H](NC(=O)[C@H]2CCCCN2CCCCCCN)[C@@H](C)CC)OC(C)=O)n1 ; CC(C)[C@H](NC(=O)CCCCCN1C(=O)C=CC1=O)C(=O)N[C@@H](C)C(=O)Nc1ccc(COC(=O)Oc2ccc([N+](=O)[O-])cc2)cc1 ; CCOC(=O)[C@@H](C)C[C@H](Cc1ccccc1)NC(=O)c1csc([C@@H](C[C@H](C(C)C)N(C)C(=O)[C@@H](NC(=O)[C@H]2CCCCN2CCCCCCNC(=O)OCc2ccc(NC(=O)[C@H](C)NC(=O)[C@@H](NC(=O)CCCCCN3C(=O)C=CC3=O)C(C)C)cc2)[C@@H](C)CC)OC(C)=O)n1]

### Example 19

Tert-butyl (6-hydroxyhexyl)carbamate (275, 300 mg, 1.38 mmol), Dess-Martin periodinane (918 mg, 2.16 mmol), and sodium bicarbonate (1.6 g, 19.0 mmol) in methylene chloride (10 ml) was stirred for 2 h. The heterogeneous mixture was filtered and the filtrate was directly flash chromatographed on silica gel (12 g) with methylene chloride:ethyl acetate as the eluent 100:0 to 80:20 over 10 min to afford 210 mg (71% yield) of tert-butyl (6-oxohexyl)carbamate, 276, as a clear oil.

Tert-butyl (6-oxohexyl)carbamate (276, 210 mg, 975 μmol) and (R)-piperidine-2-carboxylic acid (216 mg, 1.67 mmol) in 1,2-dichloroethane (4 ml) was stirred for 10 min. Sodium triacetoxyborohydride (317, 1.50 mmol) was added. After stirring for 1 h, the heterogeneous mixture was filtered and the filtrate was directly flash chromatographed on silica gel (12 g) with methylene chloride:methanol as the eluent 100:0 to 80:20 over 10 min to afford 168 mg (52% yield) of (R)-1-(6-((tert-butoxycarbonyl)amino)hexyl)piperidine-2-carboxylic acid, 278, as a white solid after lyophilization.

(R)-1-(64(Tert-butoxycarbonyl)amino)hexyppiperidine-2-carboxylic acid (278, 12 mg, 36.5 μmol) and HATU (24 mg, 63.1 μmol) in dimethylformamide (0.20 ml) stood for 15 min. The solution was added to ethyl (2S,4R)-4-(2-((1R,3R)-3-((2S,3S)-2-amino-N,3-dimethylpentanamido)-1-hydroxy-4-methylpentyl)thiazole-4-carboxamido)-2-methyl-5-phenylpentanoate hydrochloride (17 mg, 27.2 μmol) and diisopropylethylamine (0.05 ml, 287 μmol). After standing for 30 min, the solution was blown dry with a stream of air. This product, ethyl (2S,4R)-4-(2-((1R,3R)-3-((2S,3S)-2-((R)-1-(6-((tert-butoxycarbonyl)amino)hexyl)piperidine-2-carboxamido)-N,3-dimethylpentanamido)-1-hydroxy-4-methylpentyl)thiazole-4-carboxamido)-2-methyl-5-phenylpentanoate, 279, was used crude in the subsequent step.

Acetic anhydride (0.20 ml, 2.12 mmol) was added to a solution of ethyl (2S,4R)-4-(2-((1R,3R)-3-((2S,3S)-2-((R)-1-(6-((tert-butoxycarbonyeamino)hexyl)piperidine-2-carboxamido)-N,3-dimethylpentanamido)-1-hydroxy-4-methylpentyl)thiazole-4-carboxamido)-2-methyl-5-phenylpentanoate (279, material from GDP-150-041, ca. 27.2 μmol) in pyridine (0.50 ml). After stirring for 4 h, the solution was diluted with water and directly purified by reverse phase HPLC to afford 14 mg (55% yield over 2 steps) of ethyl (2S,4R)-4-(2-((1R,3R)-1-acetoxy-3-((2S,3S)-2-((R)-1-(6-((tert-butoxycarbonyl)amino)hexyl)piperidine-2-carboxamido)-N,3-dimethylpentanamido)-4-methylpentyl)thiazole-4-carboxamido)-2-methyl-5-phenylpentanoate, 280, as a white solid.

4 N Hydrogen chloride in 1,4-dioxane (2 ml) was added to ethyl (2S,4R)-4-(2-((1R,3R)-1-acetoxy-3-((2S,3S)-2-((R)-1-(6-((tert-butoxycarbonyl)amino)hexyl)piperidine-2-carboxamido)-N,3-dimethylpentanamido)-4-methylpentyl)thiazole-4-carboxamido)-2-methyl-5-phenylpentanoate (280, 14 mg, 14.9 μmol) was stirred for 1 h. The solution was blown dry with a stream of air and the residue was diluted with 1:1 acetonitrile:water, frozen, and lyophilized to yield 13 mg (100% yield) of ethyl (2S,4R)-4-(2-((1R,3R)-1-acetoxy-3-((2S,3S)-2-((R)-1-(6-aminohexyl)piperidine-2-carboxamido)-N,3-dimethylpentanamido)-4-methylpentyl)thiazole-4-carboxamido)-2-methyl-5-phenylpentanoate, 281, as a white solid.

Ethyl (2S,4R)-4-(2-((1R,3R)-1-acetoxy-3-((2S,3S)-2-((R)-1-(6-aminohexyl)piperidine-2-carboxamido)-N,3-dimethylpentanamido)-4-methylpentyl)thiazole-4-carboxamido)-2-methyl-5-phenylpentanoate (281, 13 mg, 15.5 μmol), 4-((S)-2-((S)-2-(6-(2,5-dioxo-2,5-dihydro-1H-pyrrol-1-yl)hexanamido)-3-methylbutanamido)propanamido)benzyl (4-nitrophenyl)carbonate (282, 15 mg, 23.0 μmol), 3H-[1,2,3]triazolo[4,5-b]pyridin-3-ol (5 mg, 36.7 μmol), and diisopropylethylamine (0.05 ml, 287 μmol) in dimethylformamide (0.20 ml) was stirred for 18 h. The solution was diluted with water and directly purified by reverse phase HPLC to afford 2.6 mg (12% yield) of ethyl (2S,4R)-4-(2-((1R,3R)-1-acetoxy-3-((2S,3S)-2-((R)-1-(6-((((44(S)-24(S)-2-(6-(2,5-dioxo-2,5-dihydro-1H-pyrrol-1-yl)hexanamido)-3-methylbutanamido)propanamido)benzyl)oxy)carbonyl)amino)hexyl)piperidine-2-carboxamido)-N,3-dimethylpentanamido)-4-methylpentyl)thiazole-4-carboxamido)-2-methyl-5-phenylpentanoate, 283, as a white solid. The remaining material was impure or had lost the acetate during the reaction.

### Example 20

### Alternative Synthesis of T4

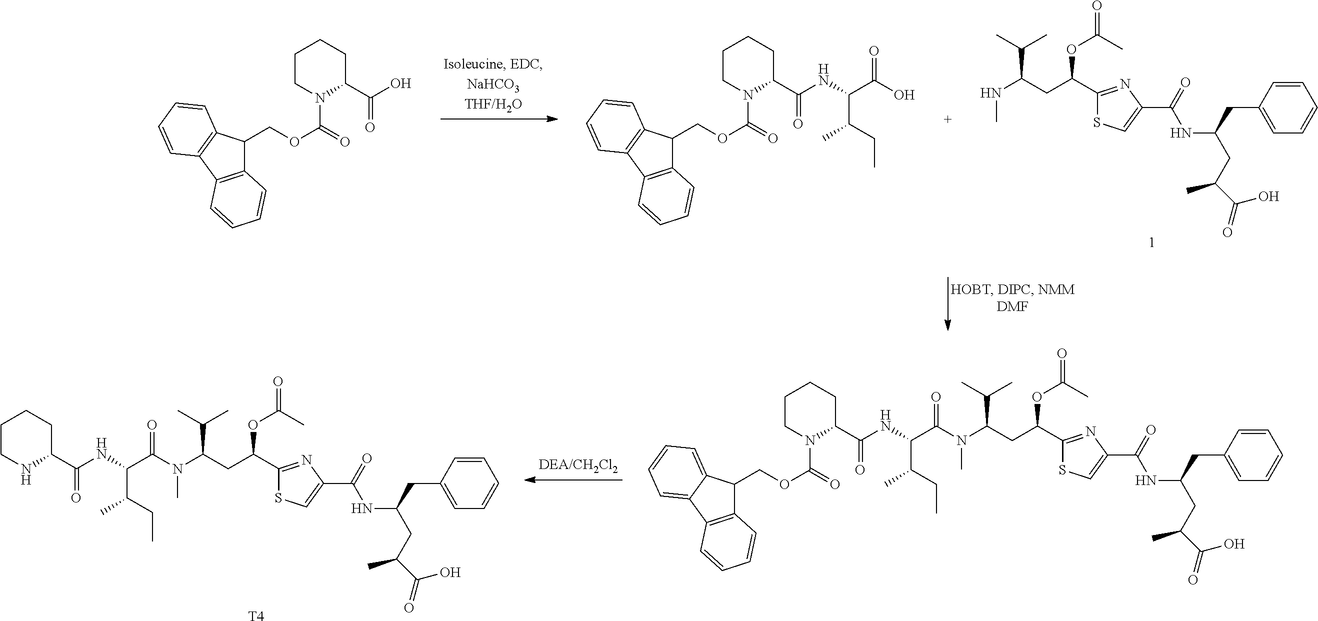 [SMILES: O=C(O)[C@H]1CCCCN1C(=O)OCC1c2ccccc2-c2ccccc21 ; CC[C@H](C)[C@H](NC(=O)[C@H]1CCCCN1C(=O)OCC1c2ccccc2-c2ccccc21)C(=O)O ; CN[C@H](C[C@@H](OC(C)=O)c1nc(C(=O)N[C@@H](Cc2ccccc2)C[C@H](C)C(=O)O)cs1)C(C)C ; CC[C@H](C)[C@H](NC(=O)[C@H]1CCCCN1)C(=O)N(C)[C@H](C[C@@H](OC(C)=O)c1nc(C(=O)N[C@@H](Cc2ccccc2)C[C@H](C)C(=O)O)cs1)C(C)C ; CC[C@H](C)[C@H](NC(=O)[C@H]1CCCCN1C(=O)OCC1c2ccccc2-c2ccccc21)C(=O)N(C)[C@H](C[C@@H](OC(C)=O)c1nc(C(=O)N[C@@H](Cc2ccccc2)C[C@H](C)C(=O)O)cs1)C(C)C ; "T4"]

Fmoc-T4 was prepared by coupling Fmoc-D-2-piperidinecarboxylic acid to isoleucine in the presence of EDC and sodium bicarbonate, then coupling the resulting Fmoc-D-Pip-Ile-OH to the N-methylvaline intermediate 1 (purchased from Concortis) by mixing with 1 equivalent of HOBT and DIPC in DMF followed by addition of 2.5 equivalents of NMM. The reaction mixture was stirred overnight and purified by flash chromatography on silica gel using a gradient of hexane and ethyl acetate. Evaporation of solvent gave Fmoc-T4 as a yellow oil. The Fmoc-T4 was then deprotected by treatment with 20% DEA in methylene chloride for 30 minutes to give T4, which was purified by preparative HPLC on a C18 reverse phase column eluted with acetonitrile/water.

### Example 21

### Synthesis of 6-(2,5-dioxopyrrolyl)hexanoyl-T4 [MC-T4] and 39-(3,4-dibromo-2,5-dioxopyrrolidinyl)-3,6,9,12,15,18,21,24,27,30,33,36-dodecaoxanonatriacontanoyl-T4 [dBrPEG-T4]

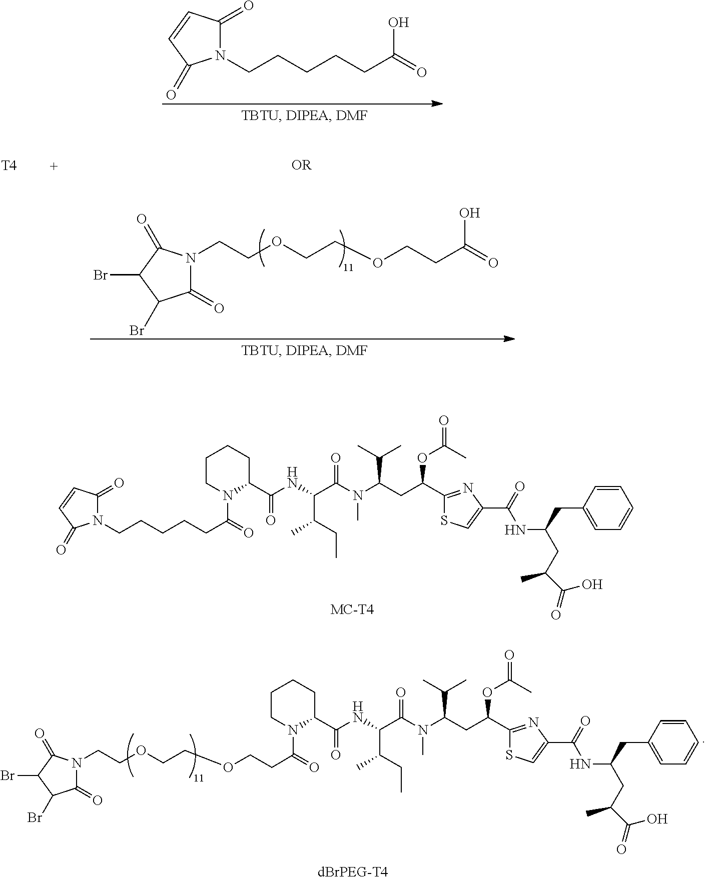

Coupling of T4 to the MC or dBrPEG linkers described in Example 2 and 3 respectively was performed by activating the linkers with 1 equivalent of TBTU in the presence of 2 equivalents of DIPEA in DMF, then coupling with the T4 for 72 hours at room temperature. Purification by preparative C18 HPLC (acetonitrile-water gradient) gave MC-T4 or dBrPEG-T4 suitable for conjugation to antibodies.

Similar syntheses using other linkers give the corresponding linker-T4 conjugates. Similar syntheses using T3, MMAF, or other cytotoxins with a basic amine give the corresponding linker-cytotoxin conjugates. Similar syntheses using amine-terminated linkers and cytotoxins with a carboxyl group, activating the cytotoxin in the same manner as the linker was activated in the above Example, give other linker-cytotoxin conjugates.

### Example 22

### Synthesis of 39-(2,5-dioxo-3,4-bis(2-pyridylsulfanyl)pyrrolyl)-3,6,9,12,15,18,21,24,27,30,33,36-dodecaoxanonatriacontanoyl-MMAF [dPSPEG-MMAF]

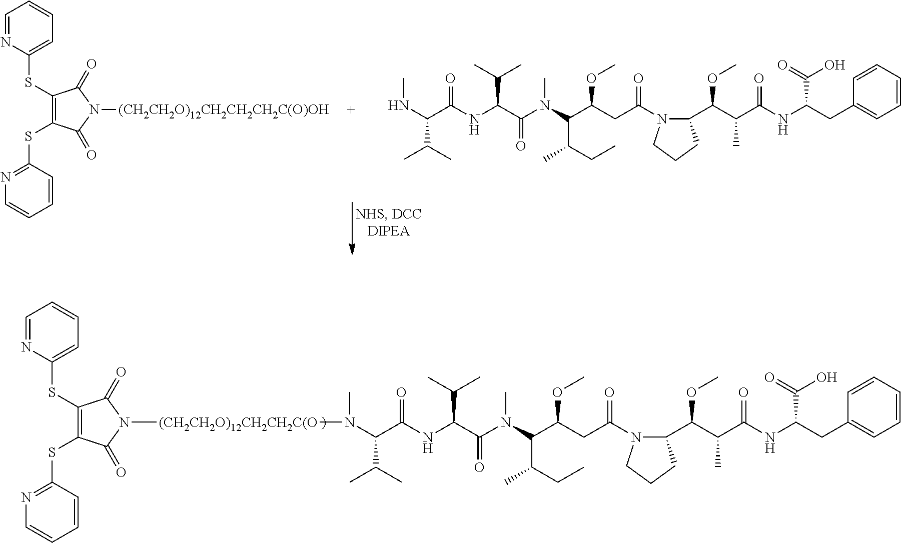

39-(2,5-Dioxo-3,4-bis(pyridin-2-ylthio)-2,5-dihydro-1H-pyrrol-1-yl)-3,6,9,12,15,18,21,24,27,30,33,36-dodecaoxanonatriacontanoic acid was added to a clean, flame-dried 50 mL round bottom flask, and the carboxylic acid was activated with NHS in 3 mL of DMF in the presence of DCC. MMAF was predissolved in about 1 mL DMF and transferred to the NHS-activated acid via 22 gauge needle. DIPEA was added to the reaction mixture and stirred overnight. The crude reaction mixture was purified by reverse-phase HPLC on a 21.2 mm×50 mm Agilent PREP-C18 column at a flow rate of 35 mL/min over 20 column volumes (about 30 minutes of gradient time). Enriched fractions were identified, pooled and lyophilized to give the dPSPEG-MMAF conjugate as a white semi-solid.

Similar syntheses using other linkers give the corresponding linker-MMAF conjugates. Similar syntheses using T3, T4 or other cytotoxins such as CTX-I′, CTX-II′, CTX-III′, CTX-IV′, CTX-V′, CTX-VI′, CTX-VII′ and CTX-VIII′ with a basic amine give the corresponding linker-cytotoxin conjugates, such as dPSPEG-T4. Similar syntheses using amine-terminated linkers and cytotoxins with a carboxyl group, activating the cytotoxin in the same manner as the linker was activated in the above Example, give other linker-cytotoxin conjugates.

### Synthesis of Antibody-Drug Conjugates

### Example 23

### Synthesis of trastuzumab-dTSPEG-MMAF ADC

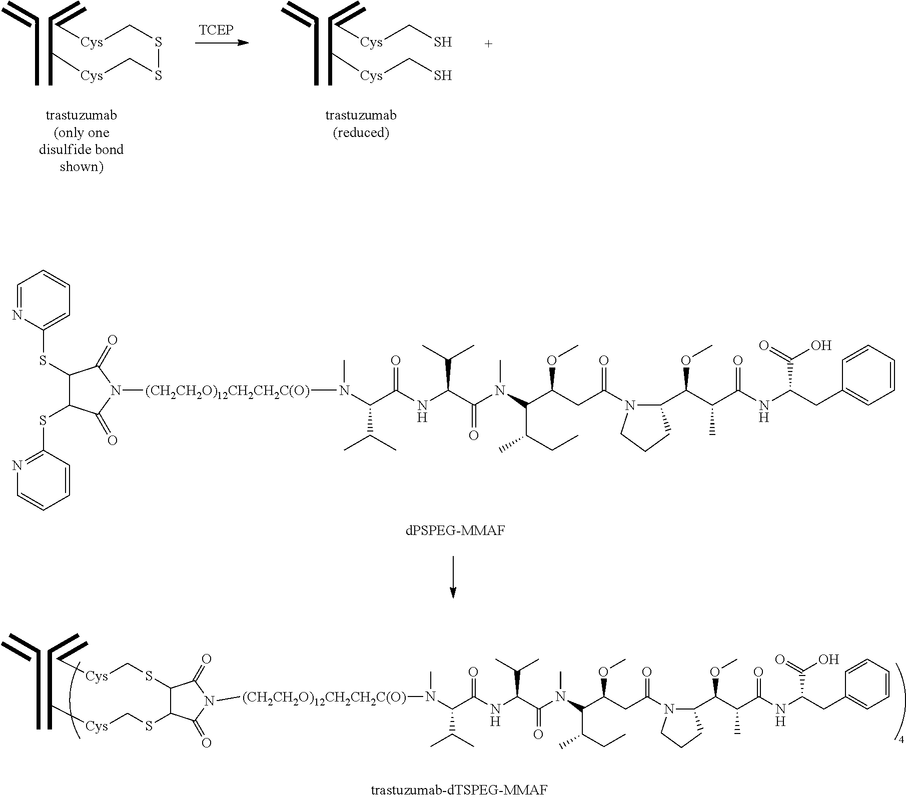

Trastuzumab, 1 mL of a 20 mg/mL solution in pH 7.4 PBS (Gibco Mg and Ca free) with 1 mM DTPA, is loaded into a sterile 1.7 mL Eppendorf tube, then 2.75 equivalents of TCEP hydrochloride (Sigma ampule 0.5M concentration), is added and the mixture incubated at 37° C. for 1 hour to give an average of 4 free thiol pairs per trastuzumab (this can be verified by Ellman's colorimetric assay see Ellman, “Tissue sulfhydryl groups”, Arch. Biochem. Biophys, 1959, 82, 70-77 or later papers referring to this assay). The reduced antibody solution is cooled in an ice-bath at about 0° C. for 15 minutes; then a solution of about 4 equivalents of dPSPEG-MMAF in dimethylsulfoxide is added and the mixture incubated at 37° C. for 2 hours (or at 4° C. for 20 hours). The resulting trastuzumab-dTSPEG-MMAF ADC is purified by size-exclusion chromatography (GE AKTA pure chromatographic system) or PD10 desalting column.

Similar syntheses using other linker-cytotoxin conjugates, such as dPSPEG-T4, and/or other antibodies, such as 18-2A (a murine IgG2a antibody), give the corresponding ADCs.

As shown in the representative Figures, the ADCs prepared from the method of the present application provides the products with significant homogeneity as shown by HIC traces, when compared with the ADCs prepared by conventional methods that provide inhomogeneous ADCs with multiple products and positional isomers.

### Assays

ADCs of this invention are tested for potency and selectivity in vitro by determining their cytotoxicity in cancer cell lines of interest, such as those cancer cell lines expressing the antigen corresponding to the antibody portion of the ADC and similar cancer cell lines lacking the antigen. They are tested for potency and safety in vivo in such animal models as the mouse subcutaneous cancer xenograft and mouse orthotopic cancer xenograft models well known to those of skill in the art of cancer research.

### Example 24

### Cytotoxicity of Trastuzumab ADCs Compared to Trastuzumab

The cytotoxicity of two ADCs where trastuzumab was conjugated to the currently used cytotoxin MMAF through an MC linker [trastuzumab-MC-MMAF] was compared to the cytotoxicity of trastuzumab alone in HER2-positive and HER2-negative tumor cells. In the HER2-negative tumor cells, the IC₅₀ for both ADCs and for trastuzumab itself was >500 nM; however, in the HER2-positive tumor cells, while the IC₅₀ for trastuzumab itself was still >500 nM, the two trastuzumab-MC-MMAF ADCs had IC₅₀s of 0.009 nM and 0.018 nM. These results suggest that ADCs are considerably more potent than their parental antibodies.

### Example 25

### Cytotoxicity of T1 and T2 Compared to MMAF

The cytotoxicity of tubulysins T1 and T2 was compared to the cytotoxicity of MMAF using the BT474 (HER2+) cell line in a standard cellular cytotoxicity assay. In these cells, MMAF had an IC₅₀ of 93 nM, T1 had an IC₅₀ of 11 nM, and T2 had an IC₅₀ of <0.1 nM, showing that these tubulysins are considerably more potent than MMAF. These results suggest that that the N-conjugable tubulysins T3 and T4 are of similar potency to non-N-conjugable tubulysins T1 and T2, and considerably more potent than MMAF. These results and the results of Example 24 suggest that tubulysin ADCs are considerably more potent than MMAF ADCs, and will be effective anticancer agents.

### Example 26

### Binding Affinity of ADCs for Antigen-Expressing Cells

Binding of the antibodies and ADCs to antigen-expressing cells are measured using a cell ELISA. Sarcoma cells transduced to express the target (F279 cells for HER2, F244 cells for CD98) are plated the day at 5000 cells per well in a 384-well plate. The following day, antibodies are serially diluted in a separate plate, and then transferred to the cell plate, which has previously had media removed by aspiration. After a 2 hour incubation at room temperature, the plate is washed with wash buffer (DPBS at pH7.4 with 0.1% bovine serum albumin) and then 25 μL horseradish peroxidase-labeled secondary antibody diluted in media is added and incubated for 30 minutes at room temperature. The plate is then washed and 15 μL of a chemiluminescent substrate (Pierce catalog #37069) is added; and the plate is read in a plate-based luminescence reader. Trastuzumab and trastuzumab ADCs (trastuzumab-MC-MMAF, trastuzumab-MC-T4, trastuzumab-dTSPEG-MMAF, and trastuzumab-dTSPEG-T4) demonstrated comparable affinity for F277 cells; and 18-2A and 18-2A ADCs (18-2A-MC-MMAF, 18-2A-MC-T4, 18-2A-dTSPEG-MMAF, and 18-2A-dTSPEG-T4) demonstrated comparable affinity for F244 cells, indicating that conjugation of the drug payloads do not affect antigen binding.

The ADCs disclosed in Table 1 are found to provide comparable affinity for F244 cells, also suggesting that conjugation of the drug payloads with the antibody do not affect antigen binding.

### Example 27

### Potency of ADCs Against Antigen-Expressing Cells

The potency of ADCs for inhibition of tumor cell growth was tested in cell proliferation assays. The Ramos (B-cell lymphoma) and BT474 (HER2+ human breast carcinoma) cell lines were seeded into 96 well half-area plates the day before drug treatment at 3000 and 5000 cells per well respectively. ADCs and controls were serially diluted in a master plate, and then transferred to the cell plates, which were incubated at 37 degrees Celsius and 5% CO₂ for 3 days. The cells were quantitated by measuring the level of ATP in the wells using the ATPLite 1Step kit (Perkin Elmer catalog #50-904-9883) as described by the manufacturer. The 18-2A ADCs (18-2A-MC-MMAF, 18-2A-MC-T4, 18-2A-dTSPEG-MMAF, and 18-2A-dTSPEG-T4) were approximately equipotent and considerably more potent than the parent 18-2A antibody in Ramos cells, while the trastuzumab ADCs (trastuzumab-MC-MMAF, trastuzumab-MC-T4, trastuzumab-dTSPEG-MMAF, and trastuzumab-dTSPEG-T4) were approximately equipotent and considerably more potent than the parent trastuzumab antibody in BT474 cells.

The ADCs disclosed in Table 1 are found to be similarly equipotent and are considerably more potent that the parent antibodies in BT474 cells.

### Example 28

### Efficacy of ADCs in Murine Xenograft Models

The Ramos cell xenograft model:

The Ramos cell line was obtained from ATCC and cultured according to the supplier's protocols. 4-6 Week-old immunodeficient female mice (Taconic C.B-17 scid) were subcutaneously injected on the right flank with 1×10⁷ viable cells in a mixture of PBS (without magnesium or calcium) and BD Matrigel (BD Biosciences) at a 1:1 ratio. The injected total volume per mouse was 200 μL with 50% being Matrigel. Once the tumor reached a size of 65-200 mm³, mice were randomized. ADCs were formulated in PBS and administered once intravenously at a dose of 1 mg/Kg into the lateral tail vein, and body weights and tumors were measured twice weekly. Tumor volume was calculated as described in van der Horst et al., “Discovery of Fully Human Anti-MET Monoclonal Antibodies with Antitumor Activity against Colon Cancer Tumor Models In Vivo”, Neoplasia, 2009, 11, 355-364. The experiments were performed on groups of 8 animals per experimental point. The negative control group received HB 121 (an IgG2a-negative antibody) and free MMAF or T4, as appropriate, at a concentration equimolar to the concentration that would be released by the ADCs, while the positive control group received 18-2A. The 18-2A ADCs with the linkers of this invention (18-2A-dTSPEG-MMAF and 18-2A-dTSPEG-T4) demonstrated slightly more but comparable TGI than the comparator ADCs (18-2A-MC-MMAF and 18-2A-MC-T4, respectively), and more TGI than the parent 18-2A antibody, while all demonstrated significant TGI compared to the control. No toxicity was observed based on animal weight measurements.

The BT474 cell xenograft model:

### Example 29

The BT474 cell line was obtained from ATCC and cultured according to the supplier's protocols. 4-6 Week-old immunodeficient female mice (Taconic C.B-17 scid) were implanted with a β-estradiol pellet 3 days before being subcutaneously injected on the right flank with 1×10⁷ viable cells in a mixture of PBS (without magnesium or calcium) and BD Matrigel (BD Biosciences) at a 1:1 ratio. The injected total volume per mouse was 200 μL with 50% being Matrigel. Once the tumor reached a size of 100-150 mm³, mice were randomized. ADCs were formulated in PBS and administered once intravenously at a dose of 1 mg/Kg into the lateral tail vein, and body weights and tumors were measured twice weekly. Tumor volume was calculated as described in van der Horst et al., cited above. The experiments were performed on groups of 8 animals per experimental point. The negative control group received HB 121 and free MMAF or T4, as appropriate, at a concentration equimolar to the concentration that would be released by the ADCs, while the positive control group received trastuzumab at 1 mg/Kg. The trastuzumab ADCs with the linkers of this invention (trastuzumab-dTSPEG-MMAF and trastuzumab-dTSPEG-T4) demonstrated comparable TGI to than the comparator ADCs (trastuzumab-MC-MMAF and trastuzumab-MC-T4, respectively), and slightly more TGI than the parent trastuzumab, while all demonstrated significant TGI compared to the control. No toxicity was observed based on animal weight measurements.

Similarly, the ADCs disclosed in Table 1 are found to have no toxicity based on animal weight measurements using the same protocols.

Similar tests are conducted with other cancers (those expressing different antigens) and ADCs where the antibody corresponds to the antigen expressed by the cancer.

### Example 30

### Screening Protocol for Bifunctional Linkers

General: Create new entries in the discovery portal database for selected conjugates. Purge all buffers and stock solutions with argon prior to use to remove residual oxygen. Freeze/thaw antibody solution to remove oxygen. Keep buffers and samples tightly sealed throughout the duration of the experiment.

Preparation of Linker Stock Solutions:

1. Purge DMSO used for preparing linker stock solutions with argon prior to use.
2. Use 4 dram clear glass vials (w/green screw caps) for linker stock solutions.
3. Prepare at least 1 mL of fresh linker stock solutions @ 2 mM in DMSO.
4. Clearly label each stock solution with sample name, ID & MW from the excel spreadsheet.
5. Set up the stock solutions in the rack labeled “linker screening samples.”
6. Prepare separate samples for LC/MS analysis of stock solutions in auto sampler tubes by diluting 20 μL of 10 mM stock into 180 μL of MeOH.
7. LC/MS analysis will be done prior to completion of the experiment.

Preparation of IGN523 (Purge all buffers with argon prior to use):

1. Obtain 60 mg of IGN523 from PD and buffer exchange into 50 mM Borate pH 8.
2. Dilute to final concentration of 5 mg/mL or 33 uM (12 mL total vol.) in Borate buffer pH 8.
3. Add 6 molar eq. of freshly prepared TCEP in water (48 μL from a 50 mM stock soln).
4. Incubate at 37° C. for 2.5 h in a sealed 15 mL falcon tube.
5. Remove 200 μL aliquot and cap with IAC for SDS-PAGE and LC/MS analysis.
6. Aliquot 400 μL each into 28 small (0.5 mL) eppendorf tubes and cool to 4° C.
7. Add 44 μL of each linker from 2 mM DMSO stock solutions to a final [linker]=200 μM.
8. Include DMSO and buffer controls (44 μL of each).

### 9. Incubate O.N. at 4° C.

ADC analysis:

1. Remove 20 μL aliquots and dilute with 80 μL PBS (degassed with argon) to 1 mg/mL final.
2. Run non-reducing SDS-PAGE (NO Heat).
3. Buffer exchange remaining conjugates into PBS pH 7.4 to stop the reactions. This step may be skipped and the samples may be freezed.
4. Dilute the conjugates to a final concentration of 2 mg/mL in PBS; pH 7.4; store at 4° C.
5. For reducing SDS-PAGE, treat samples with 5 molar eq. TCEP at 37° C. for 2 h to reduce interchain disulfides that may have reformed. Do not heat non-reducing gel samples.
6. Prioritize conjugates for LC/MS analysis based on SDS-PAGE results.
7. Select best bifunctional linkers for coupling to MMAF based on LC/MS results. This protocol can be scaled down as necessary.

### Example 30

### Protocol for Reduction and Purification of Herceptin for Conjugation to DBM(C6)-MMAF

The procedure determines the effect of purifying reduced antibody on conjugation efficiency.

Purge all buffers and DMSO stock solutions with Argon for 1 h prior to use.

1. Aliquot 1 mL of Herceptin or IGN 523 from 20 mg/mL stock into a 2 mL eppendorf tube.
2. Dilute with 1 mL 100 mM Borate (pH 8.4) to afford a 10 mg/mL stock solution (67 μM).
3. Prepare a 50 mM stock solution of TCEP in water.
4. Add 20 μL of TCEP to 2 mL of Herceptin and incubate at 37° C. for 3 h.
5. Aliquot into 4×0.5 mL eppendorf tubes and place 3 tubes in storage at −20° C.
6. Purify one 0.5 mL aliquot (˜5 mg) via SEC on Biorad using degassed PBS.
7. Collect monomeric antibody peak in a sealed tube (˜4 mL total volume) at 4° C.
8. Aliquot into 4 equal 1 mL eppendorf tubes (1 mg/mL).
9. Add 6 eq of the linkers listed below from 2 mM stock solutions in DMSO to each tube.

DBM(C6)-MMAF

BRM(C6)-MMAF

NEM

DMSO control.

### 10. Incubate at 4 deg. for 48 h.

### 11. Analyze by HIC, SDS-PAGE and LC/MS.

FIG. 9 shows the Potency of T2 and T4 in Tubulin Polymerization Assay.

The ability of T2 and T4 and T4 to inhibit microtubule formation was determined using a commercially available assay kit from Cytoskeleton (cat #BK007R) based on the procedure described in Tong, T., Ji, J., Jin, S., Li, X., Fan, W., Song, Y., Wang, M., Liu, Z., Wu, M. and Zhan, Q. (2005). Gadd45a expression induces Bim dissociation from the cytoskeleton and translocation to mitochondria. Mol. Cell. Biol. 25, 4488-4500.

### Standard Protocol:

### Step 1: Antibody Disulfide Reduction:

A) Dilute antibody to 15 mg/ml (0.1. mM IgG) in PBS pH 7.4.
B) Prepare a fresh 20 mM (5.7 mg/ml) stock solution of TCEP in H₂O.
C) Add 25 μL of TCEP stock soln. from B) to 1 mL of antibody from A) (finbal TCEP 0.5 mM).
D) Incubate at 37° C. for 2 hr. Check for free thiol using DTNB test.
E) Aliquot the reduced antibody into 4 tubes (250 μL each).

### Step 2: Payload Conjugation to Antibody:

A) Prepare 10 mM stock solution of linker-payload in DMSO. Use of DMA, DMF or CH₃CN is acceptable.
B) Add 12.5 μL (5 eq.) of stock solution from A0 t each tube of reduced mAb (0.5 mM final).
C) Incubate O.N. at 4° C. or 4 hr. at RT. Check for free thiol using DTNB.
D) Run analytical HIC to determine DAR and homogeneity.

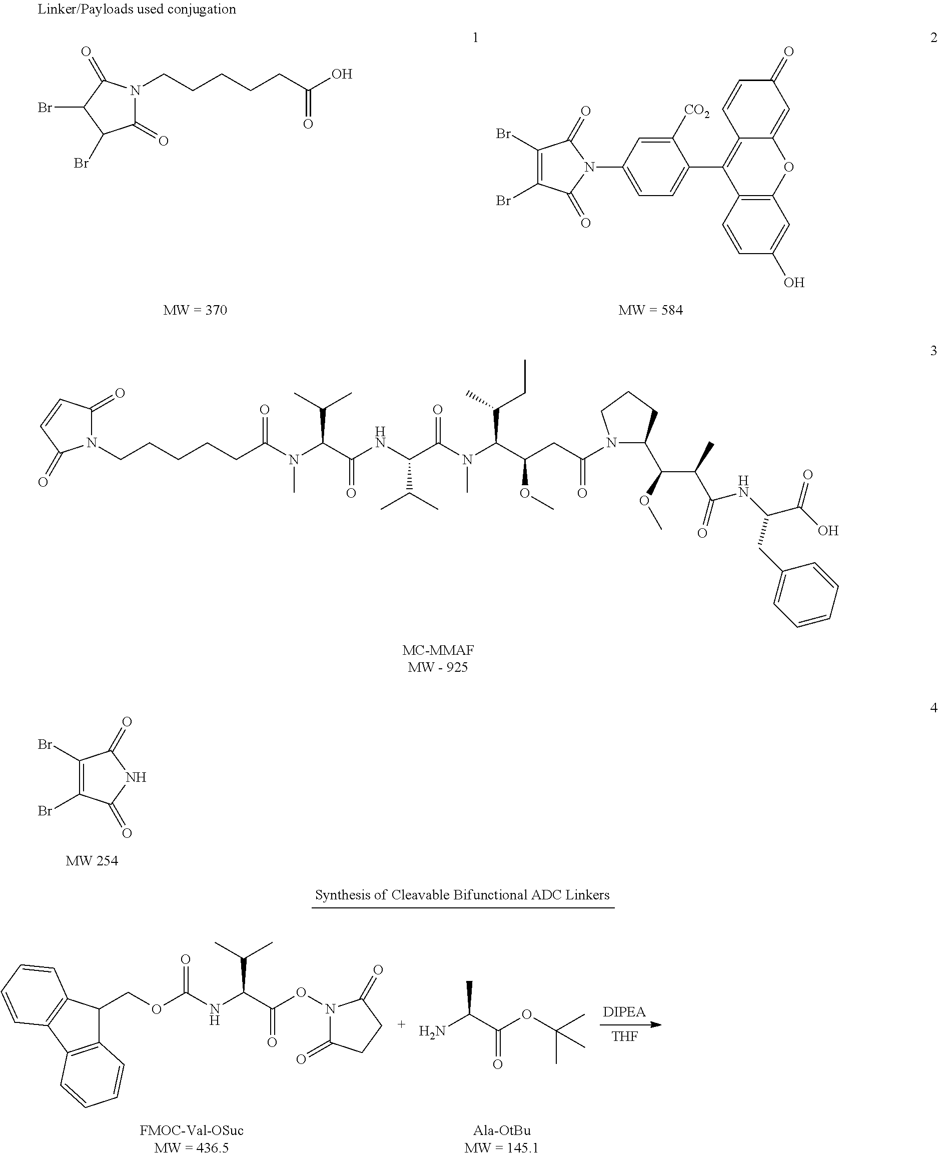

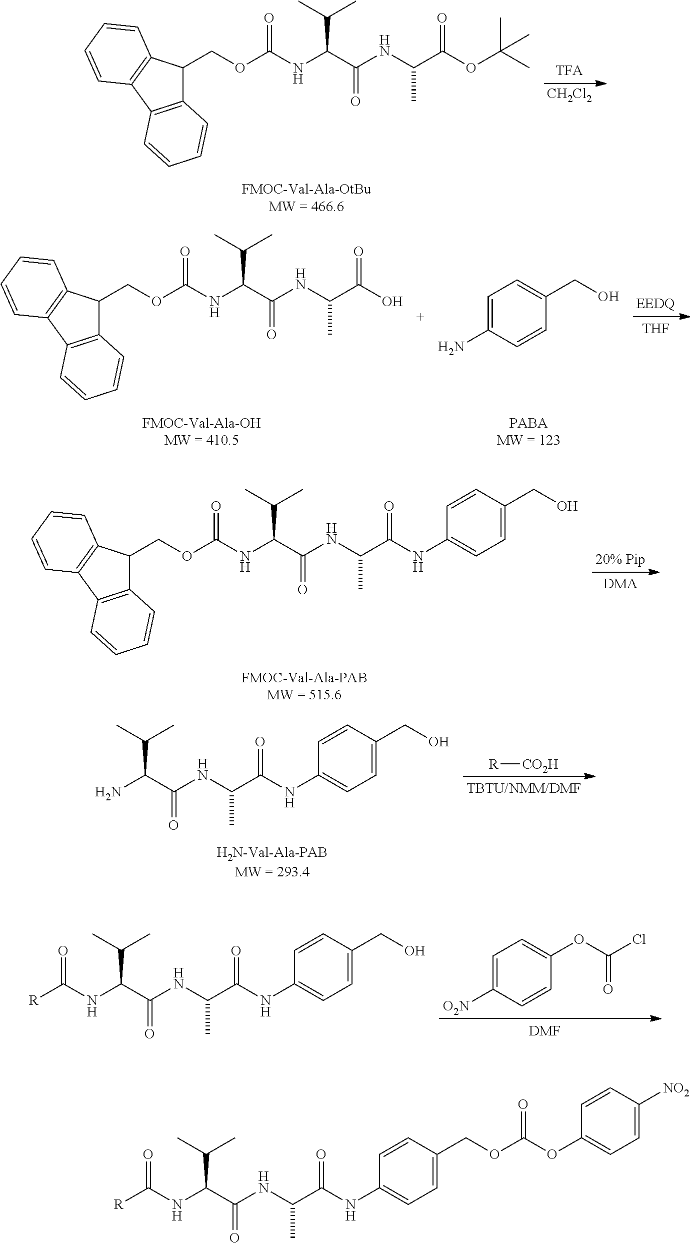

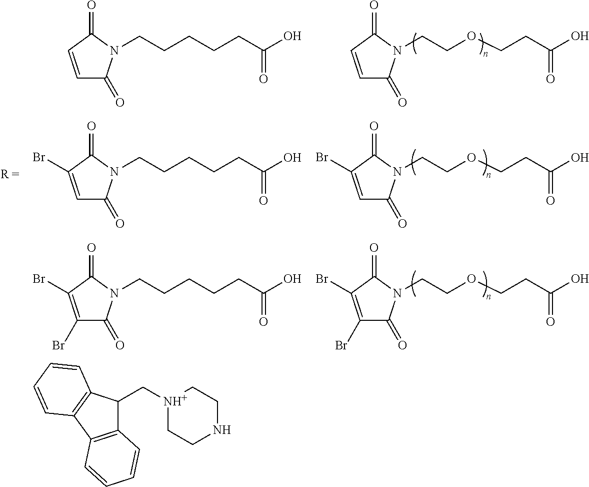

### T2 ADCs Prepared:

| Reagent Code | Reagent Name |
|---|---|
| T003M0001-AK-05 | C1.18.4:MPEG12-VAP-EDA:T2 |
| G006-AN-05 | Herceptin:MC3-PEG12-EDA:T2 |
| G006-AM-05 | Herceptin:MC-VAP-EDA:T2 |
| G006-AK-05 | Herceptin:MPEG12-VAP-EDA:T2 |
| G006-AJ-05 | Herceptin:MPEG12-EDA:T2 |
| G005-AN-05 | IGN523:MC3-PEG12-EDA:T2 |
| G005-AM-05 | IGN523:MC-VAP-EDA:T2 |
| G005-AK-05 | IGN523:MPEG12-VAP-EDA:T2 |
| G005-AJ-05 | IGN523:mPEG12-EDA:T2 |
| T029M0004-AK-05 | R29-67-7A:MPEG12-VAP-EDA:T2 |
| T029M0005-AK-05 | R29-7-1C:MPEG12-VAP-EDA:T2 |

### T2 ADC Structure Key:

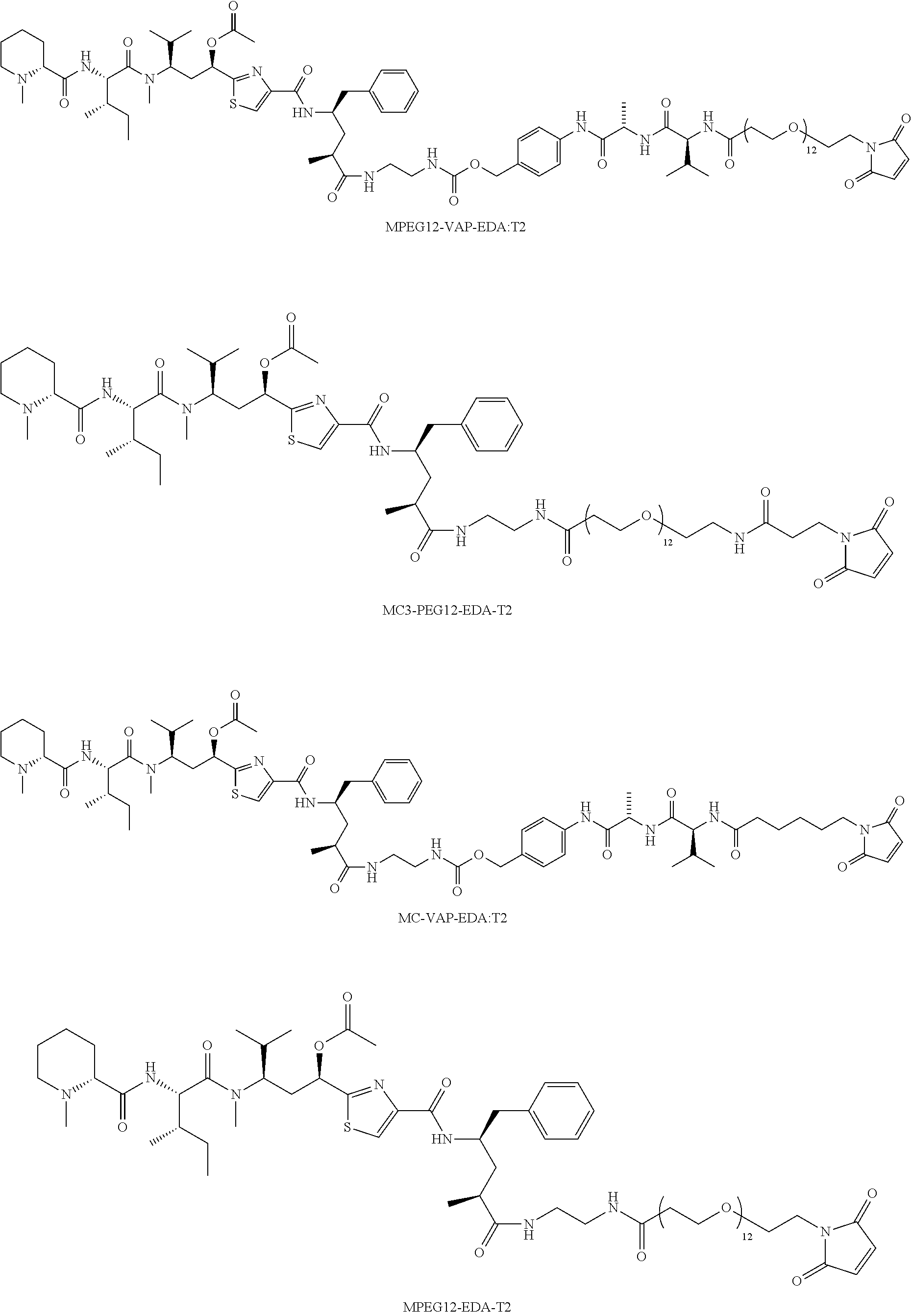

T4 ADCs Synthesized: Antibody:linker: T4

### C1.18.4:MC-VAP:T4

C1.18.4_muV/K_hG1/K:MC-VAP-HA:T4
C1.18.4_muV/K_hG1/K:MMC:T4
Chimeric C1.18.4_hG1:MPEG12:T4
Chimeric C1.18.4_hG1:MC-VAP:T4
Chimeric C1.18.4_hG1:MC-VCP:T4
Herceptin:mPEG12:T4

### Herceptin:MC-VAP:T4

### Herceptin:MC-VAP-HA:T4

### Herceptin:MMC:T4

### Herceptin: MC-VCP:T4

### IGN523:MC:T4

IGN523:mPEG12:T4

### IGN523:MC-VAP:T4

### IGN523:MC-VAP-HA:T4

### IGN523:MMC:T4

### R29-7-1C:MC-VAP:T4

### R53-4-228B:MC-VAP:T4

### T4 ADC Structure Key:

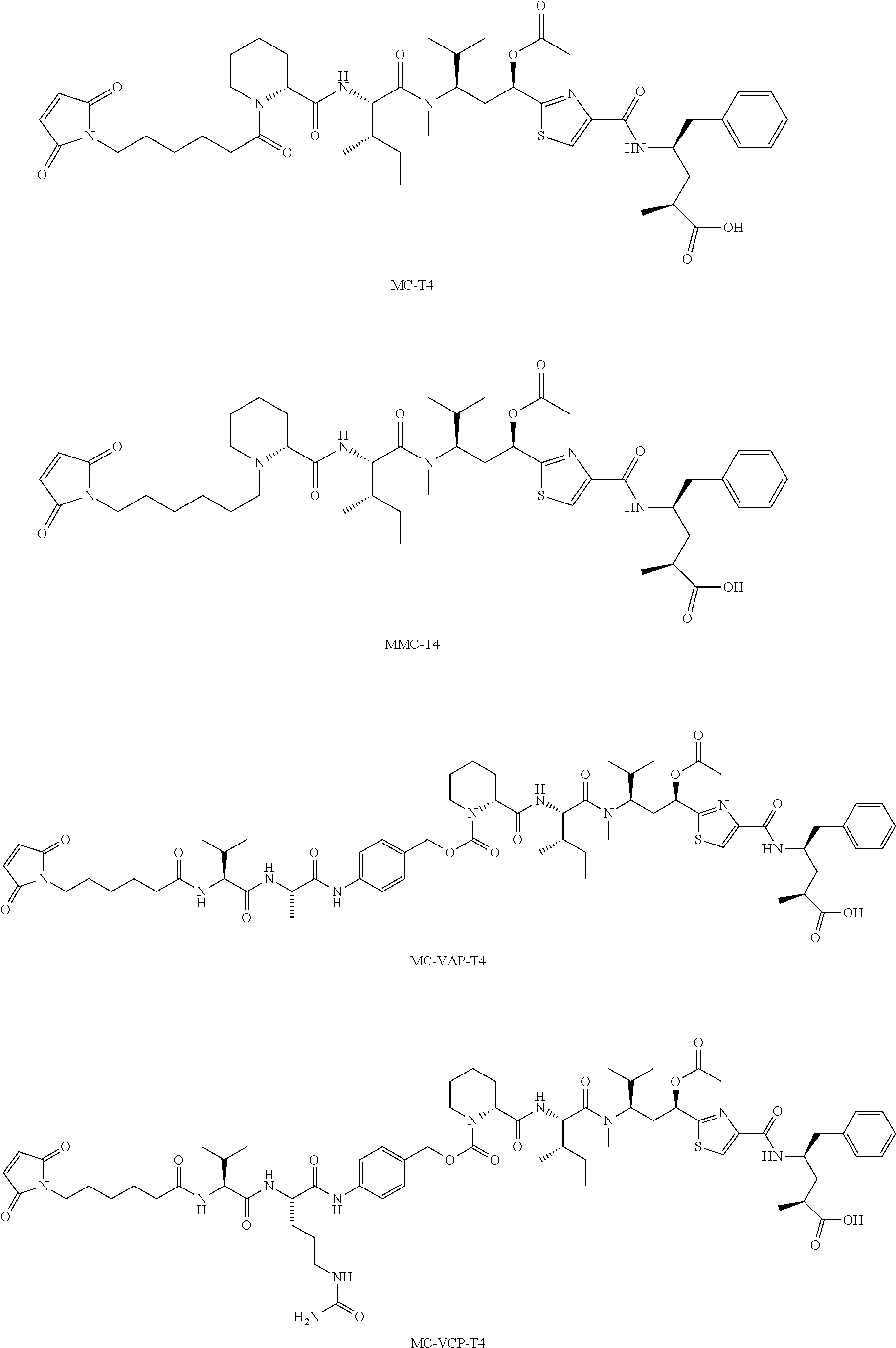

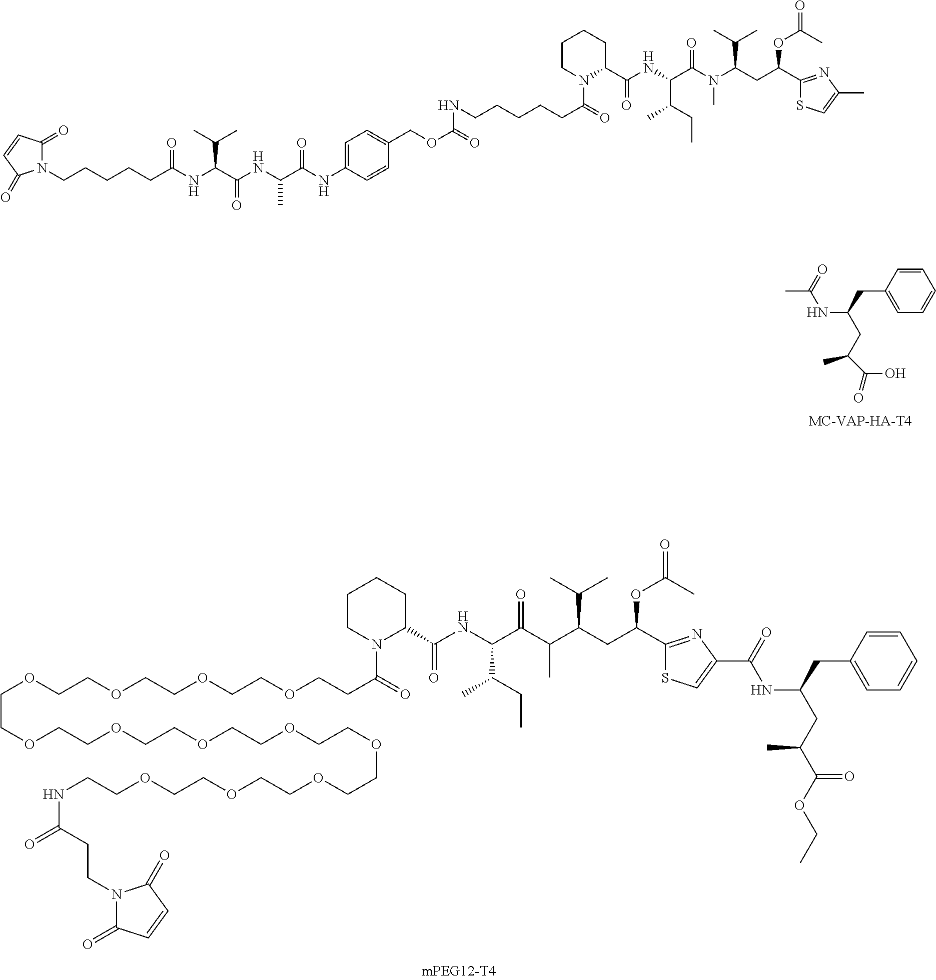

### T4 Analogs and Linkers:

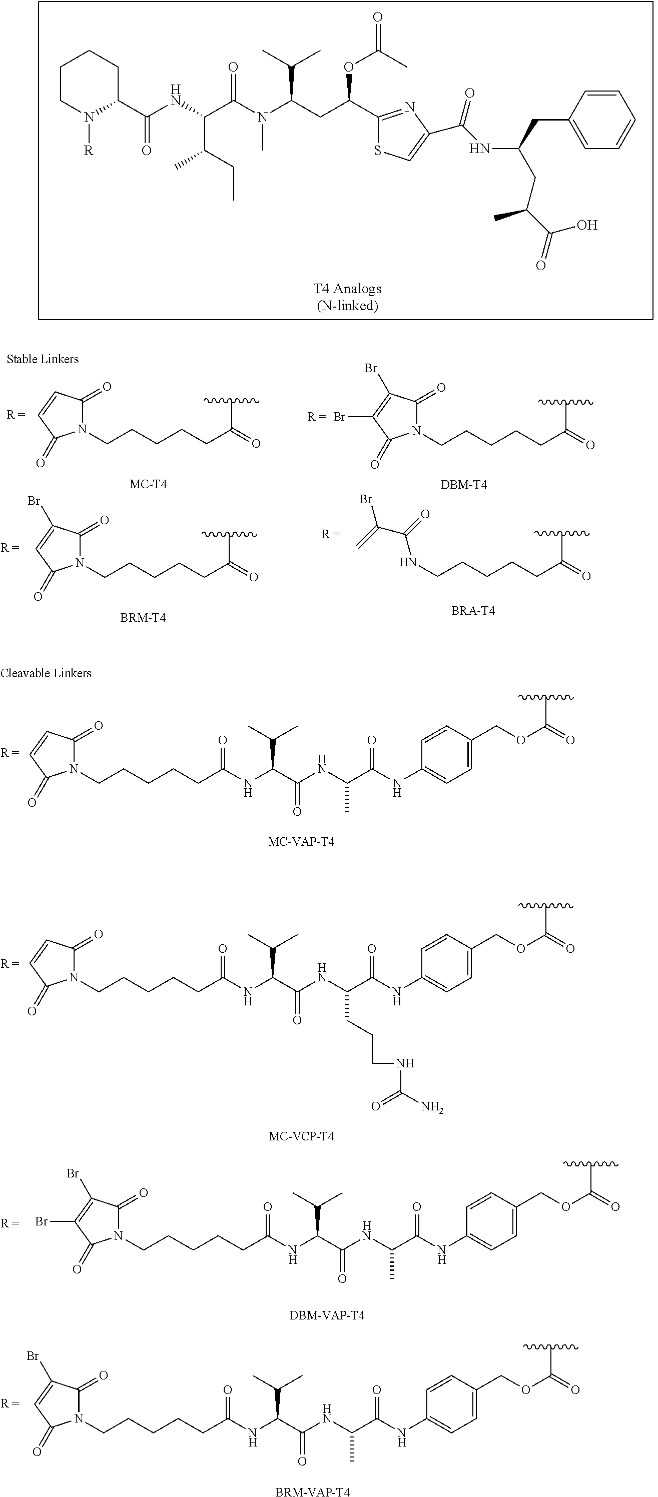

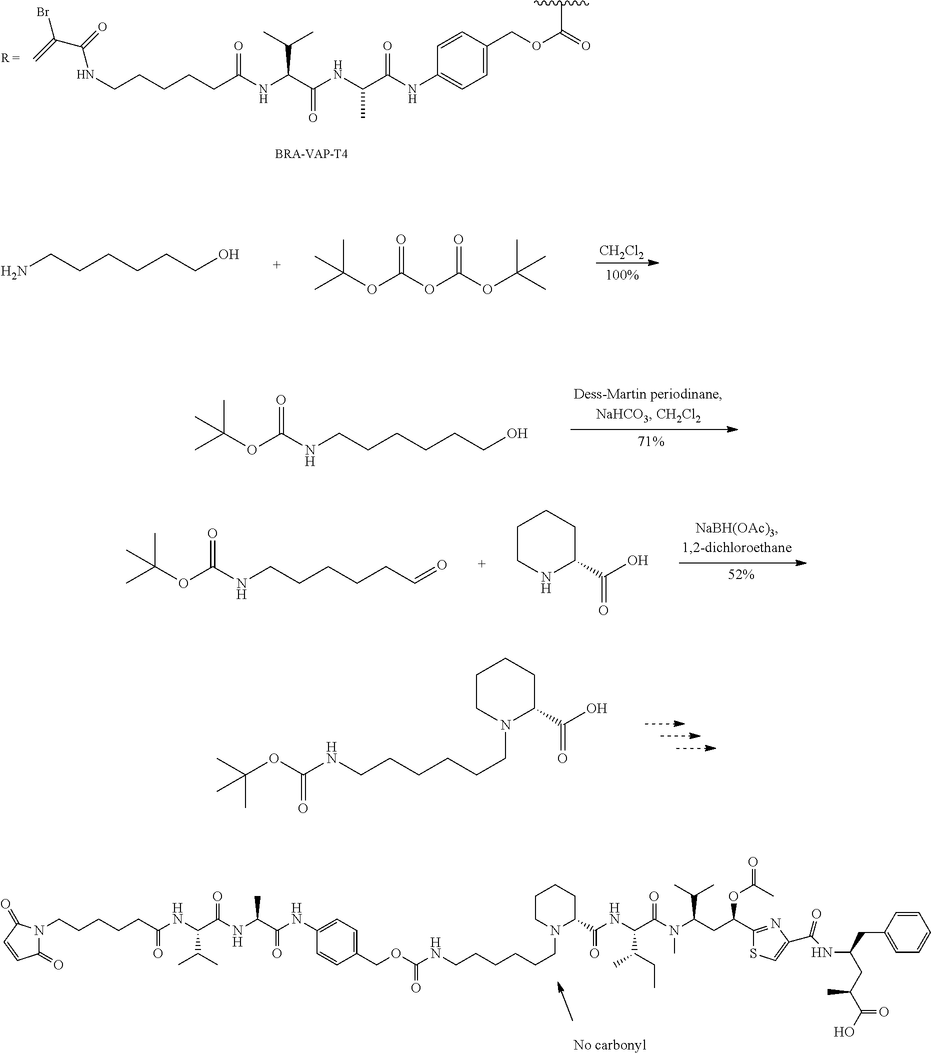

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

What is claimed is:

1. An antibody-drug conjugate (ADC) of the formula:
A [symbol] PD-(L¹)ₐ-(L²)_{b}-(L³)_{c}CTX)ₘ]ₙ
wherein:
A is an antibody;
PD is a pyrrole-2,5-dione or derivative thereof, a pyrrolidine-2,5-dione or derivative thereof;
CTX is a cytotoxin;
each L¹, L² and L³ is independently a linker selected from the group consisting of —O—, —C(O)—, —S—, —S(O)—, —S(O)₂—, —NH—, —NCH₃—, —(CH₂)_{q}—, —NH(CH₂)₂NH—, —OC(O)—, —CO₂—, —NHCH₂CH₂C(O)—, —C(O)NHCH₂CH₂NH—, —NHCH₂C(O)—, —NHC(O)—, —C(O)NH—, —NCH₃C(O)—, —C(O)NCH₃—, —(CH₂CH₂O)ₚ—, —(CH₂CH₂O)ₚCH₂CH₂—, —CH₂CH₂—(CH₂CH₂O)ₚ—, —OCH(CH₂O—)₂—, cyclopentanyl, cyclohexanyl, unsubstituted phenylenyl, phenylenyl substituted by 1 or 2 substituents selected from the group consisting of halo, CF₃—, CF₃O—, CH₃O—, —C(O)OH, —C(O)OC₁₋₃alkyl, —C(O)CH₃, —CN, —NH—, —NH₂, —O—, —OH, —NHCH₃, —N(CH₃)₂, —C₁₋₃alkyl and -(AA)ᵣ-;
a, b and c are each independently 0, 1, 2 or 3, provided that at least one of a, b or c is 1;
each p is independently an integer of 1 to 14;
each q is independently an integer from 1 to 12;
each AA is independently an amino acid;
each r is 1 to 12; and
m is an integer of 1 to 4; and n is an integer of 1 to 4;
with the proviso that when -(L¹)ₐ-(L²)_{b}-(L³)_{c}- together is —(CH₂)₁₋₁₂— or —(CH₂CH₂O)₁₋₁₂CH₂CH₂— then L¹, L² and L³ are not bonded to CTX by an amide bond.

2. The ADC of claim 1, wherein:
each L¹, L² and L³ is independently selected from the group consisting of (CH₂)_{q}—, —NH(CH₂)₂NH—, —OC(O)—, —CO₂—, NHCH₂CH₂C(O)—, —C(O)NHCH₂CH₂NH—, —C(O)NHCH₂CH₂—, —NHCH₂C(O)—, —NHC(O)—, —C(O)NH—, —NCH₃C(O)—, —C(O)NCH₃—, —C(O)CH₂CH₂—, —(CH₂CH₂O)ₚ—, —(OCH₂CH₂)ₚ—, —(CH₂CH₂O)ₚCH₂CH₂—, —CH₂CH₂—(CH₂CH₂O)ₚ—, —OCH₂(p-C₆H₄)—NH—, —OCH₂(o-C₆H₄)—NH—, —NH-(p-C₆H₄)—CH₂O—, —NH-(o-C₆H₄)—CH₂O—;
and -(AA)ᵣ-;
a, b and c are each independently 0, 1 or 2;
each p, q and r is independently 1, 2, 3 or 4;
m is 1; and
n is an integer of 1 to 4.

3. The ADC of claim 1 or 2, wherein:
each L¹, L² and L³ is independently selected from the group consisting of (CH₂)_{q}—, —NH(CH₂)₂NH—, —OC(O)—, —CO₂—, —NHCH₂CH₂C(O)—, —C(O)NHCH₂CH₂NH—, —NHCH₂C(O)—, —NHC(O)—, —C(O)NH—, —NCH₃C(O)—, —OCH(CH₂O—)₂— and —C(O)NCH₃—;
a, b and c are each independently 0, 1 or 2;
each p and q is independently 1 or 2;
m is 1; and n is an integer of 1 to 4.

4. The ADC of claim 3, wherein:
each L¹, L² and L³ is independently selected from the group consisting of —NH(CH₂)₂NH—, —NHCH₂CH₂C(O)—, —C(O)NHCH₂CH₂NH—, —NHCH₂C(O)—, —NHC(O)—, —C(O)NH—, —NCH₃C(O)—, —OCH(CH₂O—)₂— and —C(O)NCH₃—;
a, b and c are each independently 0 or 1;
m is 1; and n is an integer of 1 to 4.

5. The ADC of any one of claims 1 to 4, wherein:
each L¹, L² and L³ is independently selected from the group consisting of —NHC(O)—, —C(O)NH—, —(CH₂CH₂O)ₚ, —(CH₂CH₂O)ₚCH₂CH₂—, —CH₂CH₂—(CH₂CH₂O)ₚ—, —OCH(CH₂O—)₂— and -(AA)ᵣ-;
a, b and c are each independently 0 or 1;
each p and r is independently 1, 2 or 3;
m is 1; and n is an integer of 1 to 4.

6. The ADC of any one of claims 1 to 5, wherein:
each AA is an amino acid selected from the group consisting of Ala, Arg, Asn, Asp, Cys, Glu, Gln, Gly, His, Ile, Lys, Met, Phe, Pro, Ser, Thr, Trp, Tyr and Val.

7. The ADC of any one of claims 1 to 5, wherein:
(AA)ᵣ is a single amino acid selected from the group consisting of Gly, Arg, Val, Ala, Cys, Gln, Leu, Ile, Lys and Ser or their N-methylated analogues.

8. The ADC of any one of claims 1 to 5, wherein:
(AA)ᵣ is selected from the group consisting of Ala-Val, Val-Ala, Gly-Gly, Gly-Arg, Gly-Val, Gly-Ala, Gly-Cys, Gly-Gln, Gly-Ile, Lys-Leu, Gly-Lys, Val-Arg, Ala-Cit, Val-Cit and Gly-Ser or their N-methylated analogues.

9. The ADC of any one of claims 1 to 5, wherein:
(AA)ᵣ is selected from the group consisting of Gly-Gly-Gly, Gly-Arg-Gly, Gly-Val-Gly, Gly-Ala-Gly, Gly-Cys-Gly, Gly-Gln-Gly, Gly-Ile-Gly, Lys-Leu-Gly, Gly-Lys-Gly and Gly-Ser-Gly or their N-methylated analogues.

10. The ADC of any one of claims 1 to 5, wherein:
(AA)ᵣ is selected from the group consisting of Ala-Ala, Ala-Gly, Ala-Arg, Ala-Val, Ala-Ala, Ala-Cys, Ala-Gln, Ala-Ile, Ala-Leu, Ala-Lys, Ala-Cit and Ala-Ser or their N-methylated analogues.

11. The ADC of any one of claims 1 to 5, wherein:
(AA)ᵣ is selected from the group consisting of Ala-Ala-Ala, Ala-Gly-ALa, Ala-Arg-Ala, Ala-Val-Ala, Ala-Ala-Ala, Ala-Cys-Ala, Ala-Gln-Ala, Ala-Ile-Ala, Ala-Leu-Ala, Ala-Lys-Ala and Ala-Ser-Ala or their N-methylated analogues.

12. The ADC of any one of claims 1 to 11, wherein the antibody (A) is a monoclonal antibody or a humanized antibody.

13. The ADC of claim 12, wherein the antibody is specific to a cancer antigen.

14. The ADC of any one of claims 1 to 11, wherein the antibody is selected from the group consisting of alemtuzumab, bevacizumab, brentuximab, cetuximab, gemtuzumab, ipilimumab, ofatumumab, panitumumab, rituximab, tositumomab, inotuzumab, glembatumumab, lovortumumab, milatuzumab and trastuzumab.

15. The ADC of any one of claims 1 to 14, where CTX is an auristatin residue, a derivative of an auristatin residue, a tubulysin residue or a derivative of a tubulysin residue.

16. The ADC of any one of claims 1 to 14, wherein the CTX residue comprises the formula: 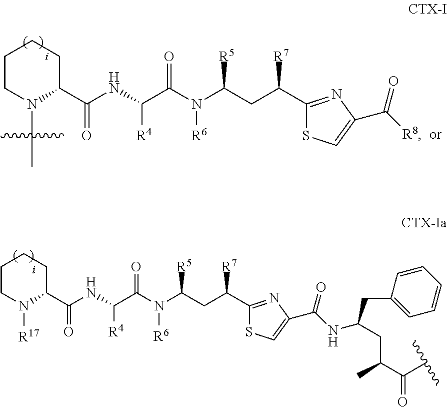
wherein:
i is 0 or 1;
R⁴ is a C₁₋₆alkyl; R⁵ is a C₁₋₆alkyl; R⁶ is C₁₋₆alkyl;
R⁷ is selected from the group consisting of C₁₋₆alkyl, —OC₁₋₆alkyl, —OC(O)C₁₋₆alkyl, —OC(O)NHC₁₋₆alkyl and —OC(O)NHC₆₋₁₀aryl; and
R⁸ is selected from the group consisting of OH, —OC₁₋₆alkyl, —CO₂C₁₋₆alkyl, —CO₂C₆₋₁₀aryl, —CH(C₁₋₆alkyl)CO₂R^{c}, —CH(C₆₋₁₀aryl)CO₂R^{c}, —NH—CH(C₅H₆)₂, —NHC₁₋₆alkyl, —NH(CH₂)₃—CO₂R^{c}, —NH(CH₂CH₂)₂C₆₋₁₀aryl, —NHCH(CH₂C₆₋₁₀aryl)CH₂CH(CH₃)CO₂R^{c} and —NHCH(CH₂CO₂R^{c})CH₂-p-C₆H₄—NHC₁₋₆alkyl; where each R^{c} is independently H or C₁₋₆alkyl; and
R¹⁷ is selected from the group consisting of H, —CH₃ and —C(O)CH₃.

17. The ADC of any one of claims 1 to 14, wherein the CTX residue comprises the formula: 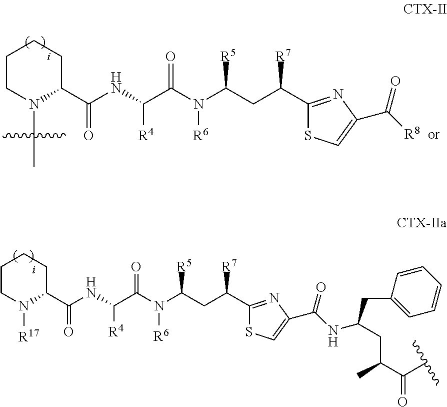
wherein:
i is 0 or 1;
R⁴ is a C₁₋₆alkyl; R⁵ is a C₁₋₆alkyl;
R⁶ is selected from the group consisting of C₁₋₆alkyl and C₆₋₁₀aryl;
R⁷ is selected from the group consisting of C₁₋₆alkyl, —OC₁₋₆alkyl, —NHC(O)C₁₋₆alkyl, —OC(O)C₁₋₆alkyl, —OC(O)C₆₋₁₀aryl, —OC(O)NHC₁₋₆alkyl and —OC(O)NHC₆₋₁₀aryl; and
R⁸ is selected from the group consisting of OH, —OC₁₋₆alkyl, —CO₂C₁₋₆alkyl, —CO₂C₆₋₁₀aryl, —CH(C₁₋₆alkyl)CO₂R^{c}, —CH(C₆₋₁₀aryl)CO₂R^{c}, —NH—CH(C₅H₆)₂, —NHC₁₋₆alkyl, —NH(CH₂)₃—CO₂R^{c}, —NH(CH₂CH₂)₂C₆₋₁₀aryl, —NHCH(CH₂C₆₋₁₀aryl)CH₂CH(CH₃)CO₂R^{c}, —NHCH(CO₂R^{c})CH₂-p-C₆H₄—NH₂, —NHCH(CO₂R^{c})CH₂-p-C₆H₄—NHC₁₋₆alkyl and —NHCH(CH₂CO₂R^{c})CH₂-p-C₆H₄—NHC₁₋₆alkyl; where each R^{c} is independently selected from the group consisting of H, C₁₋₆alkyl and C₆₋₁₀aryl; and
R¹⁷ is selected from the group consisting of H, —CH₃ and —C(O)CH₃.

18. The ADC of any one of claims 1 to 14, wherein the CTX residue comprises the formula: 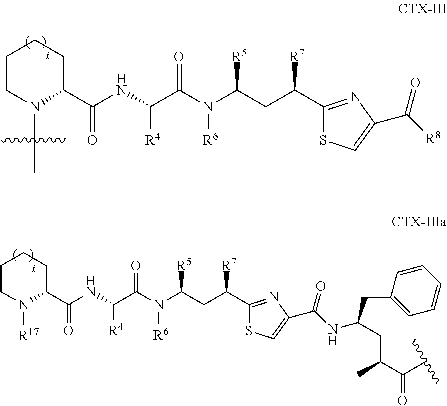
wherein:
i is 0 or 1;
R⁴ is a C₁₋₆alkyl or C₆₋₁₀aryl;
R⁵ is a C₁₋₆alkyl or C₆₋₁₀aryl;
R⁶ is selected from the group consisting of C₁₋₆alkyl-Y, —C₆₋₁₀aryl-Y, —CH₂OCOC₁₋₆alkyl-Y, —C₆₋₁₂aryl-Y, —CH₂CO₂C₁₋₆alkyl-Y, —CH₂CONHC₁₋₆alkyl-Y, —CO₂C₁₋₆alkyl-Y, CH(—CO₂H)(C₁₋₆alkyl)-Y, CH(—CO₂C₁₋₃alkyl)(C₁₋₆alkyl)-Y and CH(C₁₋₆alkyl)CO₂C₁₋₆alkyl-Y, wherein Y is H or is selected from the group consisting of NH₂, —OH, SH and —COOH wherein, with the exception where Y is H, Y is optionally attached to the linker L¹, L² and/or L³;
R⁷ is selected from the group consisting of C₁₋₆alkyl, —OC₁₋₆alkyl, —NHC(O)C₁₋₆alkyl, —OC(O)C₁₋₆alkyl, —OC(O)C₆₋₁₀aryl, —OC(O)NHC₁₋₆alkyl and —OC(O)NHC₆₋₁₀aryl; or R⁷ is a bond to the linker L¹, L² and/or L³; and
R⁸ is selected from the group consisting of —OH, —OC₁₋₆alkyl, —CO₂C₁₋₆alkyl, —CO₂C₆₋₁₀aryl, —CH(C₁₋₆alkyl)CO₂R^{c}, —CH(C₆₋₁₀aryl)CO₂R^{c}, —NH—CH(C₅H₆)₂, —NHC₁₋₆alkyl, —NH(CH₂)₃—CO₂R^{c}, —NH(CH₂CH₂)₂C₆₋₁₀aryl, —NHCH(CH₂C₆₋₁₀aryl)CH₂CH(CH₃)CO₂R^{c}, —NHCH(CH₂CH(CH₃)COORc)CH₂-p-C₆H₄—NHC(O)CH(NHC(O)(CH₂)₅NHRc)(CH₂)₄NHRc, —NHCH(CO₂R^{c})CH₂-p-C₆H₄—NH₂, —NHCH(CO₂R^{c})CH₂-p-C₆H₄—NHC₁₋₆alkyl, —NHCH(CH₂CO₂R^{c})CH₂-p-C₆H₄—NHC₁₋₆alkyl, —NHCH(CH₂CH₂CO₂R^{c})CH₂-p-C₆H₄—NHC₁₋₆alkyl and —NHCH(CH₂CH(CH₃)CO₂R^{c})CH₂-p-C₆H₄—NHC₁₋₆alkyl; where each R^{c} is independently selected from the group consisting of H, C₁₋₆alkyl and C₆₋₁₀aryl; and
R¹⁷ is selected from the group consisting of H, —CH₃ and —C(O)CH₃.

19. The ADC of claim 18, wherein the CTX residue of the formula CTX-III or CTX-IIIa:
where i is 1;
R⁴ is a C₁₋₆alkyl; R⁵ is a C₁₋₃alkyl;
R⁶ is selected from the group consisting of C₁₋₃alkyl, —CH₂OCOC₁₋₃alkyl, —CH₂CO₂C₁₋₃alkyl, —CH₂CONHC₁₋₃alkyl, —CH(C₁₋₃alkyl)CO₂H and —CH(C₁₋₃alkyl)CO₂C₁₋₃alkyl;
R⁷ is selected from the group consisting of —OC₁₋₃alkyl, —NHC(O)C₁₋₃alkyl, —OC(O)C₁₋₃alkyl, —OC(O)-phenyl, —OC(O)NHC₁₋₆alkyl and —OC(O)NHC₆₋₁₀aryl; and
for CTX-III, R⁸ is selected from the group consisting of —NH(CH₂CH₂)₂-phenyl, —NHCH(CH₂-phenyl)CH₂CH(CH₃)CO₂R^{c}, —NHCH(CO₂R^{c})CH₂-p-C₆H₄—NHC₁₋₃alkyl, —NHCH(CH₂CO₂R^{c})CH₂-p-C₆H₄—NHC₁₋₃alkyl, —NHCH(CH₂CH₂CO₂R^{c})CH₂-p-C₆H₄—NHC₁₋₃alkyl and —NHCH(CH₂CH(CH₃)CO₂R^{c})CH₂-p-C₆H₄—NHC₁₋₃alkyl; and wherein R^{c} is H or C₁₋₃alkyl.

20. The ADC of claim 18, wherein the CTX residue of the formula CTX-III or CTX-IIIa:
where i is 1;
R⁴ is a C₁₋₆alkyl; R⁵ is a C₁₋₃alkyl;
R⁶ is selected from the group consisting of C₁₋₃alkyl, —CH₂CO₂C₁₋₃alkyl and —CH(C₁₋₃alkyl)CO₂C₁₋₃alkyl;
R⁷ is selected from the group consisting of —OC₁₋₃alkyl, —NHC(O)C₁₋₃alkyl, —OC(O)C₁₋₃alkyl, —OC(O)-phenyl, —OC(O)NHC₁₋₆alkyl and —OC(O)NHC₆₋₁₀aryl; and
for CTX-III, R⁸ is selected from the group consisting of —NH(CH₂CH₂)₂-phenyl, —NHCH(CO₂R^{c})CH₂-p-C₆H₄—NHC₁₋₃alkyl and —NHCH(CH₂CH₂CO₂R^{c})CH₂-p-C₆H₄—NHC₁₋₃alkyl;
and wherein R^{c} is H or C₁₋₃alkyl.

21. The ADC of any one of claims 1 to 14, wherein the CTX residue comprises the formula: 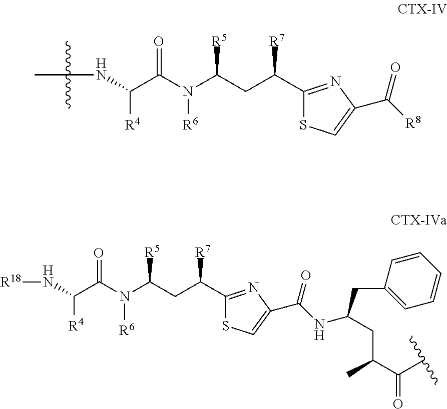
where:
R⁴ is a C₁₋₆alkyl or C₆₋₁₀aryl; R⁵ is a C₁₋₆alkyl or C₆₋₁₀aryl;
R⁶ is selected from the group consisting of C₁₋₆alkyl, C₆₋₁₀aryl, —CH₂OCOC₁₋₆alkyl, —CH₂CO₂C₁₋₆alkyl, —CH₂CONHC₁₋₆alkyl, —CO₂C₁₋₆alkyl, —CH(C₁₋₆alkyl)CO₂H and —CH(C₁₋₆alkyl)CO₂C₁₋₆alkyl;
R⁷ is selected from the group consisting of halo, C₁₋₆alkyl, —OC₁₋₆alkyl, —NHC(O)C₁₋₆alkyl, —OC(O)C₁₋₆alkyl, —OC(O)C₆₋₁₀aryl, —OC(O)NHC₁₋₆alkyl and —OC(O)NHC₆₋₁₀aryl; or R⁷ is a bond to the linker L¹, L² and/or L³; and
R⁸ is selected from the group consisting of —OH, —OC₁₋₆alkyl, —CH(C₁₋₆alkyl)CO₂R^{c}, —CH(C₆₋₁₀aryl)CO₂R^{c}, —NH—CH(C₅H₆)₂, —NHC₁₋₆alkyl, —NH(CH₂)₃—CO₂R^{c}, —NH(CH₂CH₂)₂C₆₋₁₀aryl, —NHCH(CH₂C₆₋₁₀aryl)CH₂CH(CH₃)CO₂R^{c}, —NHCH(CH₂CH(CH₃)CO₂R^{c})CH₂-p-C₆H₄—NHC(O)CH(NHC(O)(CH₂)₅NHR^{c})(CH₂)₄NHR^{c}, —NHCH(CO₂R^{c})CH₂-p-C₆H₄, —NHCH(CO₂R^{c})CH₂-p-C₆H₄—NH₂, —NHCH(CH₂CO₂R^{c})CH₂-phenyl, —NHCH(CH₂CO₂R^{c})CH₂-p-C₆H₄—NH₂, —NHCH(CH₂CH₂CO₂R^{c})CH₂-phenyl, —NHCH(CH₂CH₂CO₂R^{c})CH₂-p-C₆H₄—NH₂, —NHCH(CH₂CH(CH₃)CO₂R^{c})CH₂-phenyl, —NHCH(CH₂CH(CH₃)CO₂R^{c})CH₂-p-C₆H₄—NH₂, —NHCH(CO₂R^{c})CH₂-p-C₆H₄—NH₂, —NHCH(CO₂R^{c})CH₂-p-C₆H₄—NHC₁₋₆alkyl, —NHCH(CH₂CO₂R^{c})CH₂-p-C₆H₄—NHC₁₋₆alkyl, —NHCH(CH₂CH₂CO₂R^{c})CH₂-p-C₆H₄—NHC₁₋₆alkyl and —NHCH(CH₂CH(CH₃)CO₂R^{c})CH₂-p-C₆H₄—NHC₁₋₆alkyl; wherein each R^{c} is independently selected from the group consisting of H, C₁₋₆alkyl and C₆₋₁₀aryl; and
R¹⁸ is selected from the group consisting of H, —CH₃ and —C(O)CH₃.

22. The ADC of claim 18, wherein the CTX is a residue of the formula CTX-IV or CTX-IVa:
wherein: R⁴ is a C₁₋₆alkyl; R⁵ is a C₁₋₆alkyl;
R⁶ is selected from the group consisting of C₁₋₃alkyl, —CH₂OCOC₁₋₃alkyl, —CH₂CO₂C₁₋₃alkyl, —CH₂CONHC₁₋₃alkyl and CH(C₁₋₆alkyl)CO₂C₁₋₃alkyl;
R⁷ is selected from the group consisting of C₁₋₆alkyl, —OC₁₋₆alkyl, —NHC(O)C₁₋₃alkyl, —OC(O)C₁₋₃alkyl, —OC(O)phenyl, —OC(O)NHC₁₋₆alkyl and —OC(O)NHC₆₋₁₀aryl; and
for CTX-IV, R⁸ is selected from the group consisting of —NH—CH(C₅H₆)₂, —NHC₁₋₆alkyl, —NH(CH₂)₃—CO₂R^{c}, —NH(CH₂CH₂)₂-phenyl, —NHCH(CH₂-phenyl)CH₂CH(CH₃)CO₂R^{c}, —NHCH(CO₂R^{c})CH₂-phenyl, —NHCH(CH₂CO₂R^{c})CH₂-phenyl and —NHCH(CO₂R^{c})CH₂-p-C₆H₄—NHC₁₋₃alkyl; wherein each R^{c} is independently selected from the group consisting of H and C₁₋₃alkyl.

23. The ADC of claim 18, wherein the CTX is a residue of the formula CTX-IV or CTX-IVa:
wherein: R⁴ is a C₁₋₆alkyl; R⁵ is a C₁₋₆alkyl; R⁶ is C₁₋₃alkyl;
R⁷ is selected from the group consisting of C₁₋₆alkyl, —OC₁₋₆alkyl, —OC(O)C₁₋₃alkyl, OC(O)NHC₁₋₆alkyl and —OC(O)NHC₆₋₁₀aryl; and
for CTX-IV, R⁸ is selected from the group consisting of —NH—CH(C₅H₆)₂, —NH(CH₂CH₂)₂-phenyl, —NHCH(CO₂R^{c})CH₂-phenyl and —NHCH(CO₂R^{c})CH₂-p-C₆H₄—NHC₁₋₃alkyl; wherein each Rc is independently selected from the group consisting of H and C₁₋₃alkyl.

24. The ADC of any one of claims 1 to 14, wherein the CTX residue comprises the structure: 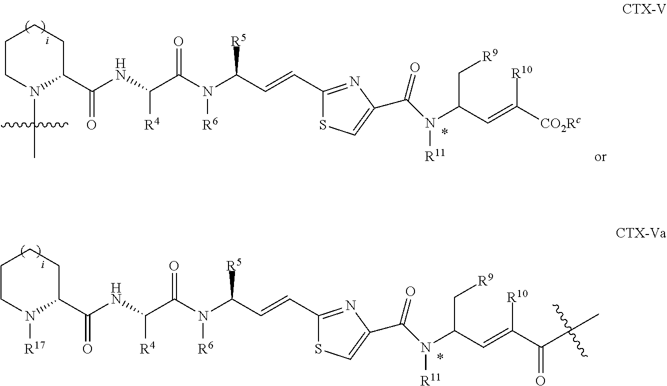
wherein:
R⁴ is a C₁₋₆alkyl or C₆₋₁₀aryl; R⁵ is a C₁₋₆alkyl or C₆₋₁₀aryl;
R⁶ is H or is selected from the group consisting of C₁₋₆alkyl, C₆₋₁₀aryl, —CH₂OCOC₁₋₆alkyl, —CH₂CO₂C₁₋₆alkyl, —CH₂CONHC₁₋₆alkyl, —CO₂C₁₋₆alkyl, CH(C₁₋₆alkyl)CO₂H and CH(C₁₋₆alkyl)CO₂C₁₋₆alkyl;
R⁹ is selected from the group consisting C₁₋₆alkyl, -phenyl, 1-naphthyl and 2-napthyl, wherein each -phenyl, 1-naphthyl and 2-naphthyl group is unsubstituted or substituted by 1 or 2 substituents selected from the group consisting of halo, cyano, nitro, CF₃—, CF₃O—, CH₃O—, —C(O)CH₃, —NH₂, —OH, —SH, —NHCH₃, —N(CH₃)₂, —SMe and C₁₋₃alkyl; and
R¹⁰ is selected from the group consisting of C₁₋₃alkyl, C₂₋₆alkenyl, —O—C₁₋₃alkyl and —OC₆₋₁₀aryl;
R¹¹ is H or C₁₋₃alkyl;
R¹⁷ is selected from the group consisting of H, —CH₃ and —C(O)CH₃;
wherein R^{c} is selected from the group consisting of H, C₁₋₆alkyl and C₆₋₁₀aryl; and
wherein * designates an R chiral center, an S chiral center or a mixture of R and S isomers.

25. The ADC of claim 24, wherein the CTX residue is of the formula CTX-V or CTX-Va:
wherein: R⁴ is a C₁₋₃alkyl; R⁵ is a C₁₋₃alkyl;
R⁶ is selected from the group consisting of C₁₋₃alkyl, —CH₂OCOC₁₋₃alkyl, —CH₂CO₂C₁₋₃alkyl, —CO₂C₁₋₃alkyl and CH(C₁₋₃alkyl)CO₂C₁₋₃alkyl;
R⁹ is selected from the group consisting C₁₋₆alkyl, -phenyl, 1-naphthyl and 2-napthyl, wherein each -phenyl, 1-naphthyl and 2-naphthyl is unsubstituted or substituted by 1 or 2 substituents selected from the group consisting of CF₃—, CH₃O—, —C(O)CH₃, —NHCH₃, —N(CH₃)₂ and —C₁₋₃alkyl; and
R¹⁰ is selected from the group consisting of C₁₋₃alkyl, C₂₋₆alkenyl, —O—C₁₋₃alkyl and O-phenyl.

26. The ADC of claim 25, wherein the CTX residue is of the formula CTX-V or CTX-Va:
wherein: R⁴ is a C₁₋₃alkyl; R⁵ is a C₁₋₃alkyl; R⁶ is C₁₋₃alkyl;
R⁹ is selected from the group consisting C₁₋₆alkyl, -phenyl, 1-naphthyl and 2-napthyl; and
R¹⁰ is selected from the group consisting of C₁₋₃alkyl and C₂₋₆alkenyl.

27. The ADC of any one of claims 1 to 14, wherein the CTX residue comprises the structure: 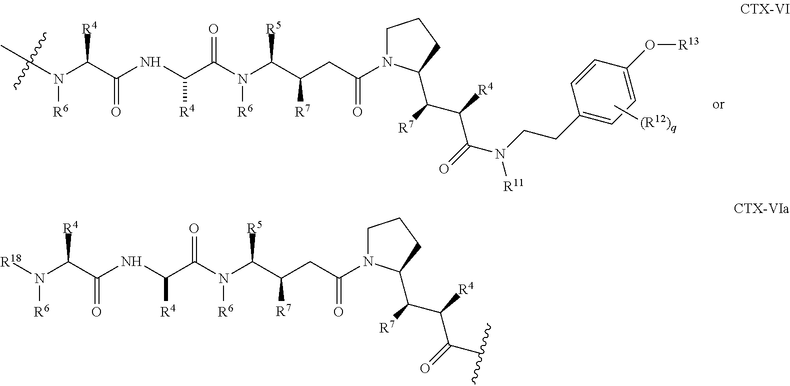
wherein:
each R⁴ is independently a C₁₋₆alkyl or C₆₋₁₀aryl;
R⁵ is a C₁₋₆alkyl or C₆₋₁₀aryl;
each R⁶ is independently selected from the group consisting of H, C₁₋₆alkyl, C₆₋₁₀aryl, —CH₂OCOC₁₋₆alkyl, —CH₂CO₂C₁₋₆alkyl, —CH₂CONHC₁₋₆alkyl, —CO₂C₁₋₆alkyl, CH(C₁₋₆alkyl)CO₂H and CH(C₁₋₆alkyl)CO₂C₁₋₆alkyl;
each R⁷ is independently selected from the group consisting of —CN, —OC₁₋₆alkyl, C₁₋₆alkyl, —NHC(O)C₁₋₆alkyl, —OC(O)C₁₋₆alkyl, —OC(O)C₆₋₁₀aryl, —OC(O)NHC₁₋₆alkyl and —OC(O)NHC₆₋₁₀aryl;
R¹¹ is H or C₁₋₃alkyl;
each R¹² is independently selected from the group consisting of halo, cyano, nitro, CF₃—, CF₃O—, CH₃O—, —CO₂H, —NH₂, —OH, —SH, —NHCH₃, —N(CH₃)₂, —SMe, —C₁₋₃alkyl and —C₆₋₁₀aryl;
R¹³ is H or is selected from the group consisting of C₁₋₃alkyl, —CF₃, —C₁₋₃alkyl-phenyl and —C₆₋₁₀aryl;
R¹⁸ is selected from the group consisting of H, —CH₃ and —C(O)CH₃; and
q is 0, 1 or 2.

28. The ADC of claim 27, wherein the CTX residue is of the formula CTX-VI or CTX-VIa:
wherein: each R⁴ is independently a C₁₋₃alkyl; R⁵ is a —C₁₋₃alkyl;
each R⁶ is independently selected from the group consisting of H, C₁₋₆alkyl, —CH₂OCOC₁₋₆alkyl, —CH₂CO₂C₁₋₃ alkyl, —CH(C₁₋₃alkyl)CO₂H and CH(C₁₋₃ alkyl)CO₂C₁₋₃ alkyl;
each R⁷ is independently selected from the group consisting of —OC₁₋₃alkyl, C₁₋₃alkyl, —NHC(O)C₁₋₃alkyl, —OC(O)C₁₋₃alkyl, —OC(O)C₆₋₁₀aryl, —OC(O)NHC₁₋₆alkyl and —OC(O)NHC₆₋₁₀aryl;
R¹¹ is H or C₁₋₃alkyl;
each R¹² is independently selected from the group consisting of halo, CF₃—, CF₃O—, CH₃O—, —NHCH₃, —N(CH₃)₂ and C₁₋₃alkyl;
R¹³ is H or is selected from the group consisting of C₁₋₃alkyl, —CF₃, —C₁₋₃alkyl-phenyl.

29. The ADC of claim 28, wherein the CTX residue is of the formula CTX-VI or CTX-VIa:
wherein: each R⁴ is independently a C₁₋₃alkyl; R⁵ is a C₁₋₃alkyl;
each R⁶ is independently H or C₁₋₆alkyl;
each R⁷ is independently selected from the group consisting of —OC₁₋₃alkyl, —OC(O)C₁₋₃alkyl, —OC(O)C₆₋₁₀aryl, —OC(O)NHC₁₋₆alkyl and —OC(O)NHC₆₋₁₀aryl;
R¹¹ is H or C₁₋₃alkyl;
each R¹² is independently selected from the group consisting of CF₃O—, CH₃O— and C₁₋₃alkyl;
R¹³ is H or is selected from the group consisting of C₁₋₃alkyl, —CF₃ and —C₁₋₃alkyl-phenyl.

30. The ADC of any one of claims 1 to 14, wherein the CTX residue comprises the structure: 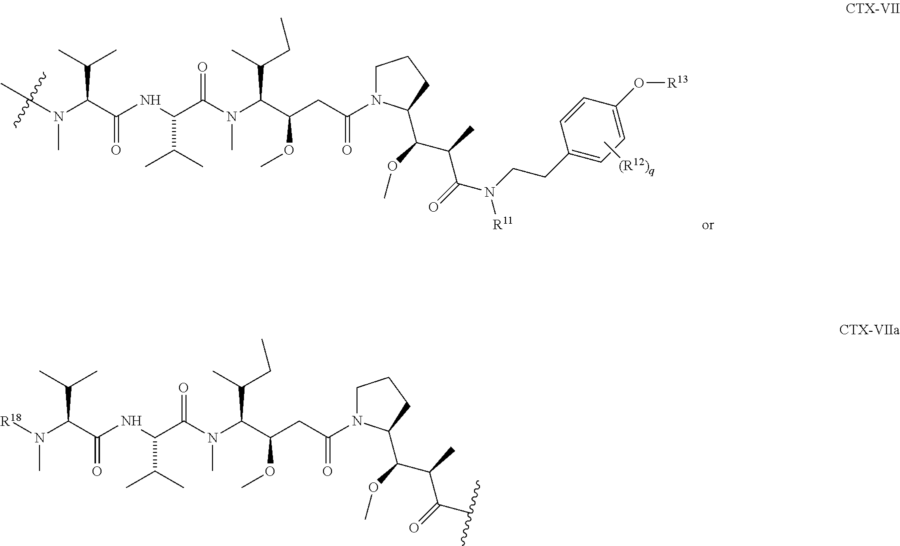
wherein:
R¹¹ is H or C₁₋₃alkyl;
each R¹² is independently selected from the group consisting of halo, cyano, nitro, CF₃—, CF₃O—, CH₃O—, —CO₂H, —NH₂, —OH, —SH, —NHCH₃, —N(CH₃)₂, —SMe, C₁₋₃alkyl and C₆₋₁₀aryl;
R¹³ is H or is selected from the group consisting of C₁₋₃alkyl, —CF₃, —C₁₋₂alkyl-phenyl and C₆₋₁₀aryl;
R¹⁸ is selected from the group consisting of H, —CH₃ and —C(O)CH₃; and
q is 0, 1 or 2.

31. The ADC of claim 30, wherein the CTX residue is of the formula CTX-VII:
wherein: R¹¹ is H;
R¹² is selected from the group consisting of CF₃—, CF₃O—, CH₃O—, —CO₂H, —NHCH₃, —N(CH₃)₂, —C₁₋₃alkyl and phenyl;
R¹³ is H or is selected from the group consisting of C₁₋₃alkyl, —C₁₋₂alkyl-phenyl and phenyl; and q is 1.

32. The ADC of claim 30, wherein the CTX residue is of the formula CTX-VII:
wherein: R¹¹ is H and
R¹³ is H, C₁₋₃alkyl or —C₁₋₂alkyl-phenyl; and q is 0.

33. The ADC of any one of claims 1 to 14, wherein the CTX residue comprises the structure: 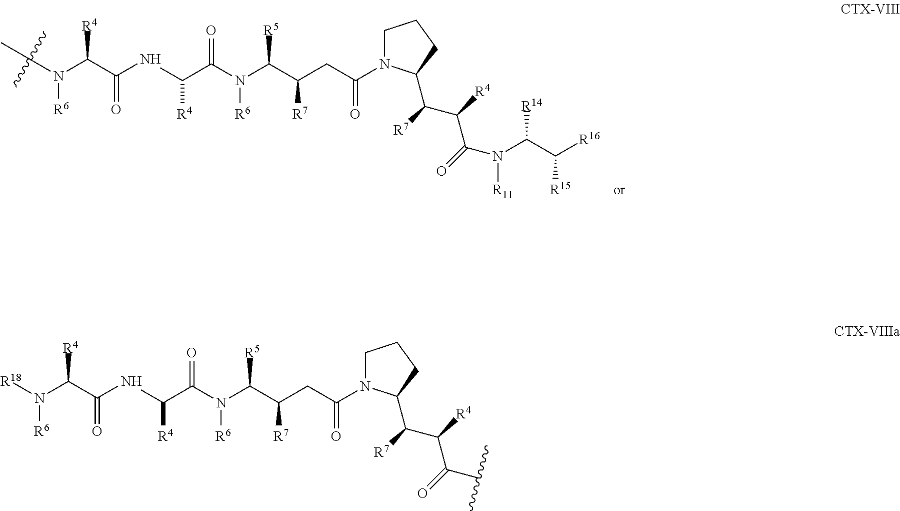
wherein:
each R⁴ is independently a C₁₋₆alkyl or C₆₋₁₀aryl;
R⁵ is a C₁₋₆alkyl or C₆₋₁₀aryl;
each R⁶ is independently selected from the group consisting of H, C₁₋₆alkyl, C₆₋₁₀aryl, —CH₂OCOC₁₋₆alkyl, —CH₂CO₂C₁₋₆alkyl, —CH₂CONHC₁₋₆alkyl, —CO₂C₁₋₆alkyl, CH(C₁₋₆alkyl)CO₂H and CH(C₁₋₆alkyl)CO₂C₁₋₆alkyl;
each R⁷ is independently selected from the group consisting of —CN, —OC₁₋₆alkyl, C₁₋₆alkyl, —NHC(O)C₁₋₆alkyl, —OC(O)C₁₋₆alkyl, —OC(O)C₆₋₁₀aryl, —OC(O)NHC₁₋₆alkyl and —OC(O)NHC₆₋₁₀aryl;
R¹¹ is H or C₁₋₃alkyl;
R¹⁴ is selected from the group consisting of C₁₋₃alkyl and C₆₋₁₀aryl;
R¹⁵ is H or is selected from the group consisting of OH, NH₂, —NHCH₃, C₁₋₃alkyl, —OC₁₋₃alkyl and —OC₆₋₁₀aryl;
R¹⁶ is selected from the group consisting of C₁₋₆alkyl, C₆₋₁₀aryl and heteroaryl; and
R¹⁸ is selected from the group consisting of H, —CH₃ and —C(O)CH₃.

34. The ADC of claim 33, wherein the CTX residue is of the formula CTX-VIII or CTX-VIIIa:
wherein: each R⁴ is independently a C₁₋₃alkyl; R⁵ is a C₁₋₃alkyl;
each R⁶ is independently selected from the group consisting of H, C₁₋₆alkyl, —CH₂OCOC₁₋₆alkyl, —CH₂CO₂C₁₋₃alkyl, CH(C₁₋₃alkyl)CO₂H and CH(C₁₋₃alkyl)CO₂C₁₋₃alkyl;
each R⁷ is independently selected from the group consisting of —OC₁₋₃alkyl, C₁₋₃alkyl, —NHC(O)C₁₋₃alkyl, —OC(O)C₁₋₃alkyl, —OC(O)C₆₋₁₀aryl, —OC(O)NHC₁₋₆alkyl and —OC(O)NHC₆₋₁₀aryl;
R¹¹ is H or C₁₋₃alkyl;
R¹⁴ is C₁₋₃alkyl;
R¹⁵ is H or is selected from the group consisting of OH, NH₂, —NHCH₃ and —OC₁₋₃alkyl; and
R¹⁶ is C₆₋₁₀aryl.

35. The ADC of claim 33, wherein the CTX residue is of the formula CTX-VIII or CTX-VIIIa:
wherein: each R⁴ is independently a C₁₋₃alkyl; R⁵ is a C₁₋₃alkyl;
each R⁶ is independently H or C₁₋₆alkyl;
each R⁷ is independently selected from the group consisting of —OC₁₋₃alkyl, —OC(O)C₁₋₃alkyl, —OC(O)C₆₋₁₀aryl, —OC(O)NHC₁₋₆alkyl and —OC(O)NHC₆₋₁₀aryl;
R¹¹ is H or C₁₋₃alkyl;
R¹⁴ is C₁₋₃alkyl;
R¹⁵ is selected from the group consisting of —OH, NH₂ and —NHCH₃; and
R¹⁶ is C₆₋₁₀aryl.

36. The ADC of any one of claims 1 to 35, wherein the CTX is tubulysin T3 or tubulysin T4.

37. The ADC of any one of claims 1 to 36 where PD is selected from the group consisting of: 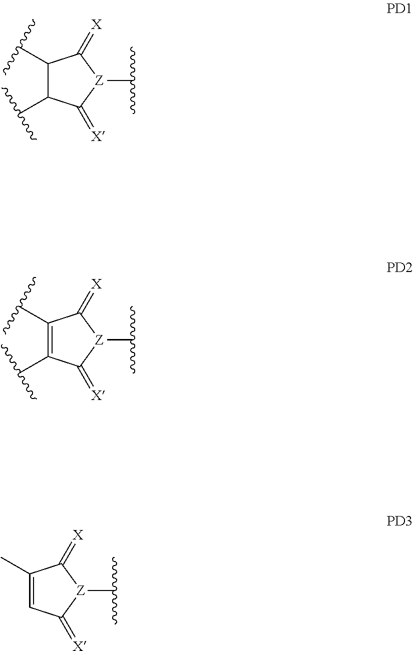
where:
X is O, S or NR¹ where R¹ is H or C₁₋₃alkyl;
X′ is O, S or NR² where R² is H or C₁₋₃alkyl; and
Z is selected from the group consisting of N—, CH—, CR³— and CR³—CR⁴R⁵— where R³, R⁴ and
R⁵ are each independently H or C₁₋₃alkyl.

38. The ADC of any one of claims 1 to 37, wherein:
A is selected from the group consisting of alemtuzumab, bevacizumab, cetuximab, ipilimumab, ofatumumab, anitumumab, rituximab, tositumomab, inotuzumab, glembatumumab, lovortumumab, milatuzumab and trastuzumab;
PD is a pyrrole-2,5-dione, a pyrrolidine-2,5-dione;
each L¹, L² and L³ is independently selected from the group consisting of —NHC(O)—, —C(O)NH—, —(CH₂CH₂O)ₚ—, —(CH₂CH₂O)ₚCH₂CH₂—, —CH₂CH₂—(CH₂CH₂O)ₚ— and -(AA)ᵣ- where the AA is selected from the group consisting of Gly, Arg, Val, Ala, Cys, Gln, Leu, Ile, Lys and Ser or their N-methylated analogues;
a, b and c are each independently 0 or 1;
each p and r is independently 1 or 2; m is 1; and n is 1, 2, 3 or 4; and
CTX is a tubulysin residue or derivative thereof, or an auristatin residue or a derivative thereof.

39. The ADC of any one of claims 1 to 37, wherein:
A is selected from the group consisting of alemtuzumab, bevacizumab, cetuximab, ipilimumab, ofatumumab, anitumumab, rituximab, tositumomab, inotuzumab, glembatumumab, lovortumumab, milatuzumab and trastuzumab;
PD is a pyrrole-2,5-dione, a pyrrolidine-2,5-dione;
each L¹, L² and L³ is independently a linker selected from the group consisting of (CH₂)_{q}—, —OCH(CH₂O—)₂—, —NH(CH₂)₂NH—, —NHCH₂CH₂C(O)—, —C(O)NHCH₂CH₂NH—, —NHCH₂C(O)—, —NHC(O)—, —C(O)NH—, —NCH₃C(O)—, —C(O)NCH₃—, cyclopentanyl, cyclohexanyl, unsubstituted phenylenyl, phenylenyl substituted by 1 or 2 substituents selected from the group consisting of halo, CH₃O—, —C(O)OC₁₋₃alkyl, —C(O)CH₃, —NHCH₃, —N(CH₃)₂, —C₁₋₃alkyl; and -(AA)ᵣ-; where the AA is selected from the group consisting of Gly, Arg, Val, Ala, Cys, Gln, Leu, Ile, Lys and Ser or their N-methylated analogues;
a, b and c are each independently 0 or 1;
each p and r is independently 1 or 2; m is 1; and
n is 1, 2, 3 or 4; and CTX is a tubulysin residue or derivative thereof, or an auristatin residue or a derivative thereof.

40. The ADC of any one of claims 1 to 37, wherein:
A is selected from the group consisting of alemtuzumab, bevacizumab, cetuximab, ipilimumab, ofatumumab, anitumumab, rituximab, tositumomab, inotuzumab, glembatumumab, lovortumumab, milatuzumab and trastuzumab;
PD is a pyrrole-2,5-dione, a pyrrolidine-2,5-dione;
each L¹, L² and L³ is independently selected from the group consisting of —NHC(O)—, —OCH(CH₂O—)₂, —C(O)NH—, —(CH₂CH₂O)ₚ, —(CH₂CH₂O)ₚCH₂CH₂—, —CH₂CH₂—(CH₂CH₂O)ₚ— and -(AA)ᵣ- where the AA is selected from the group consisting of Gly, Arg, Val, Ala, Cys, Gln, Leu, Ile, Lys and Ser or their N-methylated analogues;
a, b and c are each independently 0 or 1;
each p and r is independently 1 or 2; m is 1;
n is 1, 2, 3 or 4; and
CTX is a tubulysin residue selected from the compound of the formulae CTX-III, CTX-IIIa, CTX-IV, CTX-IVa, CTX-V, CTX-Va, CTX-VI, CTX-VIa, CTX-VII, CTXVIIa, CTX-VIII and CTX-VIIIa.

41. The ADC of any one of claims 1 to 37, wherein:
A is selected from the group consisting of alemtuzumab, bevacizumab, cetuximab, ipilimumab, ofatumumab, anitumumab, rituximab, tositumomab, inotuzumab, glembatumumab, lovortumumab, milatuzumab and trastuzumab;
PD is a pyrrole-2,5-dione, a pyrrolidine-2,5-dione;
each L¹, L² and L³ is independently selected from the group consisting of —NHC(O)—, —C(O)NH—, —(CH₂CH₂O)ₚ, —(CH₂CH₂O)ₚCH₂CH₂—, —OCH(CH₂O—)₂ and —CH₂CH₂—(CH₂CH₂O)ₚ—;
a, b and c are each 1; each p and r is independently 1 or 2; m is 1;
n is 1, 2 or 3; and
CTX is a tubulysin residue selected from the compound of the formulae CTX-III, CTX-IIIa, CTX-IV, CTX-IVa, CTX-V, CTX-Va, CTX-VI, CTX-VIa, CTXVII and CTX-VIIa.

42. The antibody-drug conjugate recited in Table 1 and a pharmaceutically acceptable excipient thereof.

43. A pharmaceutical composition containing an antibody-drug conjugate of any one of claims 1 to 42.

44. A method of treating a cancer by administering to a human suffering therefrom an effective amount of an antibody-drug conjugate of any one of claims 1 to 42 or a pharmaceutical composition of claim 43.

45. A linker-cytotoxin conjugate of formula A, B or C: 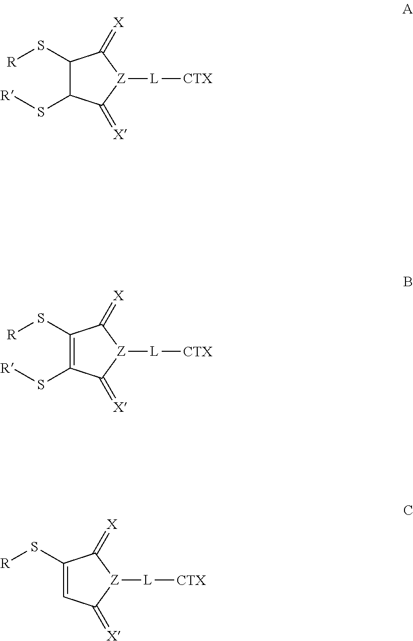
where each R and R′ is independently selected from the group consisting of C₁₋₆alkyl optionally substituted with halo or hydroxyl; phenyl optionally substituted with halo, hydroxyl, carboxyl, C₁₋₃alkoxycarbonyl, or C₁₋₃alkyl; naphthyl optionally substituted with halo, hydroxyl, carboxyl, C₁₋₃alkoxycarbonyl, or C₁₋₃alkyl; 2-pyridyl optionally substituted with halo, hydroxyl, carboxyl, C₁₋₃alkoxycarbonyl or C₁₋₃alkyl; C₁₋₆alkylsulfonyloxy, C₂₋₄₀cycloalkylsulfonyloxy, C₆₋₁₀arylsulfonyloxy; C₁₋₃alkyl-S—, C₆₋₁₀aryl-S— and C₆₋₁₀heteroaryl-S—;
X is O, S or NR¹ where R¹ is H or C₁₋₃alkyl;
X′ is O, S or NR² where R² is H or C₁₋₃alkyl;
Z is selected from the group consisting of N—, CH—, CR³— and CR³—CR⁴R⁵— where R³, R⁴ and
R⁵ are each independently H or C₁₋₃alkyl.
L is a linker defined by -(L¹)ₐ-(L²)_{b}-(L³)_{c}-, wherein each L¹, L² and L³ is independently a linker selected from the group consisting of —O—, —C(O)—, —S—, —S(O)—, —S(O)₂—, —NH—, —NCH₃—, —(CH₂)_{q}—, —NH(CH₂)₂NH—, —OC(O)—, —CO₂—, —NHCH₂CH₂C(O)—, —C(O)NHCH₂CH₂NH—, —NHCH₂C(O)—, —NHC(O)—, —C(O)NH—, —NCH₃C(O)—, —C(O)NCH₃—, —(CH₂CH₂O)ₚ—, —(CH₂CH₂O)ₚCH₂CH₂—, —CH₂CH₂—(CH₂CH₂O)ₚ—, —OCH(CH₂O—)₂—, cyclopentanyl, cyclohexanyl, unsubstituted phenylenyl, phenylenyl substituted by 1 or 2 substituents selected from the group consisting of halo, CF₃—, CF₃O—, CH₃O—, —C(O)OH, —C(O)OC₁₋₃alkyl, —C(O)CH₃, —CN, —NH₂, —OH, —NHCH₃, —N(CH₃)₂, —C₁₋₃alkyl and -(AA)ᵣ-;
a, b and c are each independently 0, 1, 2 or 3, provided that at least one of a, b or c is 1;
each p is independently an integer of 1 to 14;
each q is independently an integer from 1 to 12;
each AA is independently an amino acid;
each r is 1 to 12; and
CTX is a cytotoxin bonded to L by an amide bond.

46. The linker-cytotoxin conjugate of claim 45 where CTX is an auristatin, a calicheamicin, a maytansinoid or a tubulysin, or a derivative thereof.

47. The linker-cytotoxin conjugate of claim 46 where CTX is monomethylauristatin E, monomethylauristatin F, calicheamicin γ, mertansine, tubulysin T3 or tubulysin T4, or a derivative thereof.

48. A linker of formula AA, BB or CC: 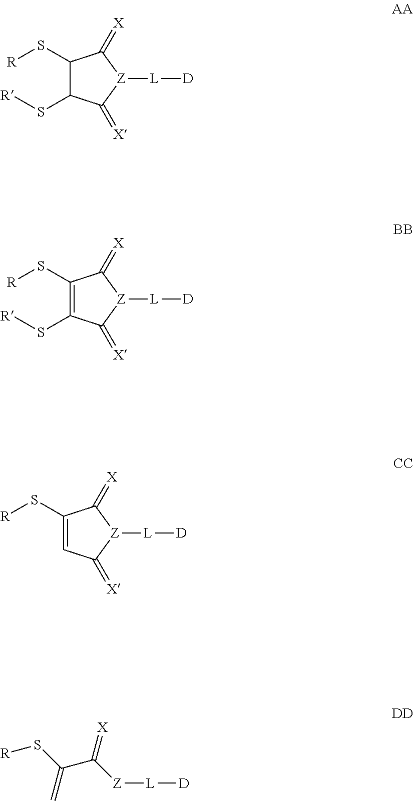
where each R and R′ is independently selected from the group consisting of C₁₋₆alkyl optionally substituted with halo or hydroxyl; phenyl optionally substituted with halo, hydroxyl, carboxyl, C₁₋₃alkoxycarbonyl, or C₁₋₃alkyl; naphthyl optionally substituted with halo, hydroxyl, carboxyl, C₁₋₃alkoxycarbonyl, or C₁₋₃alkyl; or 2-pyridyl optionally substituted with halo, hydroxyl, carboxyl, C₁₋₃alkoxycarbonyl or C₁₋₃alkyl; C₁₋₆alkylsulfonyloxy, C₂₋₁₀cycloalkylsulfonyloxy, C₆₋₁₀arylsulfonyloxy;
L is a linker defined by -(L¹)ₐ-(L²)_{b}-(L³)_{c}-, wherein each L¹, L² and L³ is independently a linker selected from the group consisting of —O—, —C(O)—, —S—, —S(O)—, —S(O)₂—, —NH—, —NCH₃—, (CH₂)_{q}—, —NH(CH₂)₂NH—, —OC(O)—, —CO₂—, —NHCH₂CH₂C(O)—, —C(O)NHCH₂CH₂NH—, —NHCH₂C(O)—, —NHC(O)—, —C(O)NH—, —NCH₃C(O)—, —C(O)NCH₃—, —(CH₂CH₂O)ₚ—, —(CH₂CH₂O)ₚCH₂CH₂—, —CH₂CH₂—(CH₂CH₂O)ₚ—, —OCH(CH₂O—)₂—, cyclopentanyl, cyclohexanyl, unsubstituted phenylenyl, phenylenyl substituted by 1 or 2 substituents selected from the group consisting of halo, CF₃—, CF₃O—, CH₃O—, —C(O)OH, —C(O)OC₁₋₃alkyl, —C(O)CH₃, —CN, —NH₂, —OH, —NHCH₃, —N(CH₃)₂, C₁₋₃alkyl, and -(AA)ᵣ-;
a, b and c are each independently 0, 1, 2 or 3, provided that at least one of a, b or c is 1;
each p is independently an integer of 1 to 14;
each q is independently an integer from 1 to 12;
each AA is independently an amino acid;
each r is 1 to 12;
D is carboxyl, C₁₋₆alkoxycarbonyl or amino, and
m is an integer of 1 to 12.

49. A linker of formula AAA, BBB, CCC or DDD: 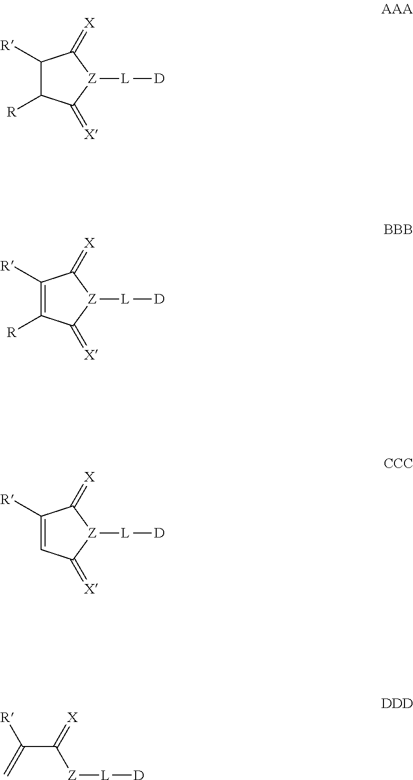
where each R and R′ is independently selected from the group consisting of chloro, bromo, iodo, C₁₋₆alkylsulfonyloxy, C₂₋₁₀cycloalkylsulfonyloxy, C₆₋₁₀arylsulfonyloxy;
L is a linker defined by -(L¹)ₐ-(L²)_{b}-(L³)_{c}-, wherein each L¹, L² and L³ is independently a linker selected from the group consisting of —O—, —C(O)—, —S—, —S(O)—, —S(O)₂—, —NH—, —NCH₃—, (CH₂)_{q}—, —NH(CH₂)₂NH—, —OC(O)—, —CO₂—, —NHCH₂CH₂C(O)—, —C(O)NHCH₂CH₂NH—, —NHCH₂C(O)—, —NHC(O)—, —C(O)NH—, —NCH₃C(O)—, —C(O)NCH₃—, —(CH₂CH₂O)ₚ—, —(CH₂CH₂O)ₚCH₂CH₂—, —CH₂CH₂—CH₂CH₂O)ₚ—, —OCH(CH₂O—)₂—, cyclopentanyl, cyclohexanyl, unsubstituted phenylenyl, phenylenyl substituted by 1 or 2 substituents selected from the group consisting of halo, CF₃—, CF₃O—, CH₃O—, —C(O)OH, —C(O)OC₁₋₃alkyl, —C(O)CH₃, —CN, —NH₂, —OH, —NHCH₃, —N(CH₃)₂, C₁₋₃alkyl and -(AA)ᵣ-;
a, b and c are each independently 0, 1, 2 or 3, provided that at least one of a, b or c is 1;
each p is independently an integer of 1 to 14;
each q is independently an integer from 1 to 12;
each AA is independently an amino acid;
each r is 1 to 12; and D is carboxyl, C₁₋₆alkoxycarbonyl or amino.

50. The linker of claim 49, wherein:
each R and R′ is independently selected from the group consisting of H, Cl, Br and I and iodo; and
L is selected from the group consisting of —(CH₂)₁₋₅C(O)-Val-Ala-NH-(p-C₆H₄)—CH₂OC(O)-(p-C₆H₄)—NO₂, —(CH₂CH₂O)₁₋₁₂—(CH₂CH₂)C(O)-Val-Ala-NH-(p-C₆H₄)—CH₂OC(O)-(p-C₆H₄)—NO₂, —(CH₂)₁₋₅C(O)-Val-Cit-NH-(p-C₆H₄)—CH₂OC(O)-(p-C₆H₄)—NO₂, —(CH₂CH₂O)₁₋₁₂—(CH₂CH₂)C(O)-Val-Cit-NH-(p-C₆H₄)—CH₂OC(O)-(p-C₆H₄)—NO₂.

51. A pharmaceutical composition comprising the antibody-drug conjugate compound of any one of claims 1 to 42 or a pharmaceutically acceptable salt thereof, and a pharmaceutically acceptable diluents, carrier or excipient.

52. A cytotoxin selected from the group consisting of: 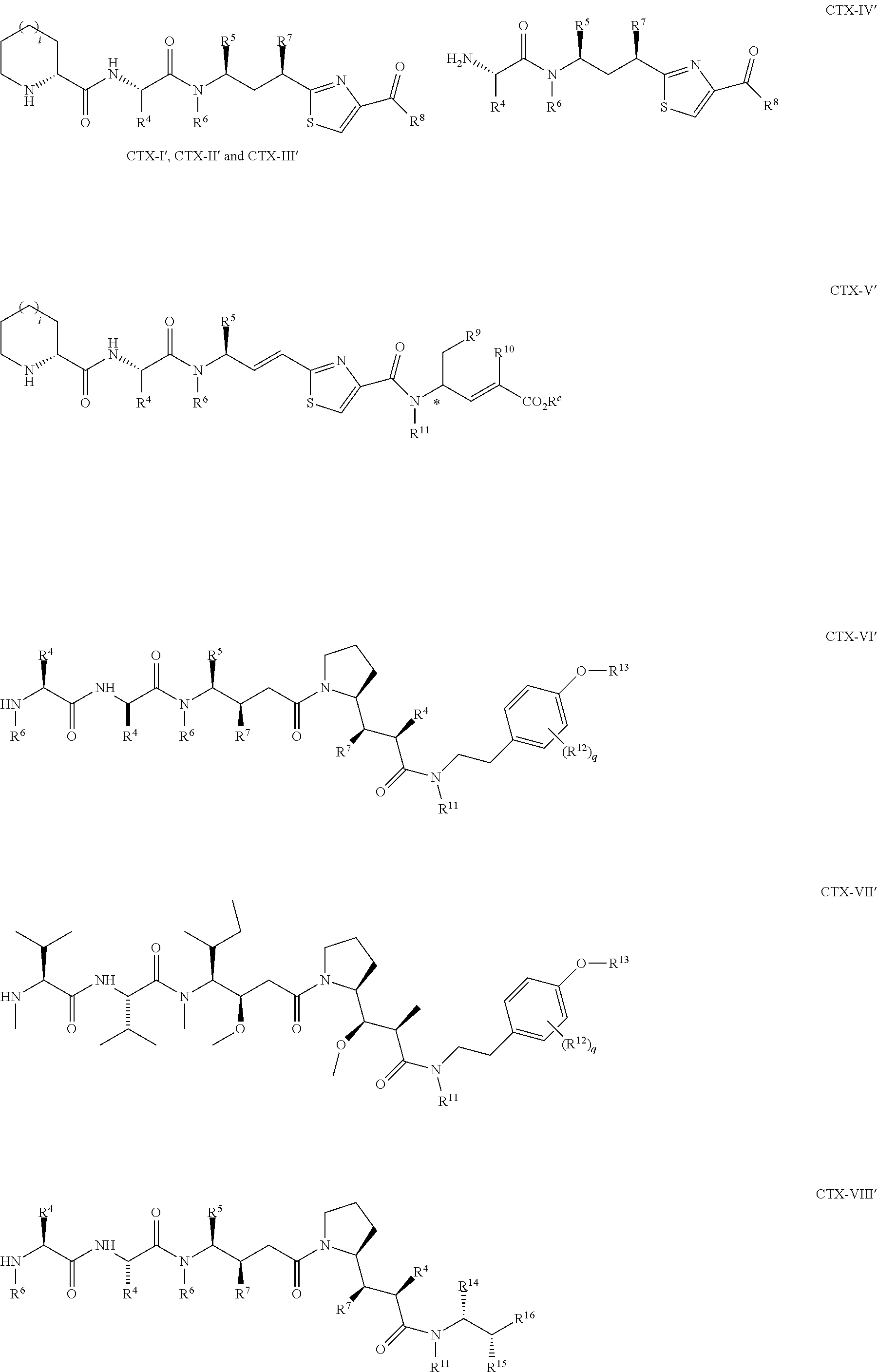
wherein the variables i, q, R⁴, R⁵, R⁶, R⁷, R⁸, R⁹, R¹⁰, R¹¹, R¹², R¹³, R¹⁴, R¹⁵ an, R¹⁶ are as defined herein in the corresponding cytotoxin conjugated residues CTX-I, CTX-II, CTX-III, CTX-IV, CTX-V, CTX-VI, CTX-VII and CTX-VIII, respectively.
